SEQUENCE LISTING (478 sequences)

SEQ ID NO: 1 - protein, 300 aa, unidentified
  REGION 1..300: note = protein encoded by maize nf-ya3
MPVILREMEDHSVHPMSKSNHGSLSGNGYEMKHSGHKVCDRDSSSESDRSHQEASAASESSPNEHTSTQSDNDEDHGKDNQDTMKPVLSLGKEGSAFLAPKLHYSPSFACIPYTSDAYYSAVGVLTGYPPHAIVHPQQNDTTNTPGMLPVEPAEEPIYVNAKQYHAILRRRQTRAKLEAQNKMVKNRKPYLHESRHRHAMKRARGSGGRFLNTKQLQEQNQQYQASSGSLCSKIIANSIISQSGPTCTPSSGTAGASTAGQDRSCLPSVGFRPTTNFSDQGRGGLKLAVIGMQQRVSTIR

SEQ ID NO: 2 - protein, 126 aa, unidentified
  REGION 1..126: note = protein encoded by maize hb75
MMIPARHMPPTMIVRNGGAAYGSSSALSLGQPNLMDNQQLQFQQALQQQHLLLDQIPATTAESCDNTGRGGGGRGSDPLADEFESKSGSENVDGVSVDDQDDPNQRPSKKKRYHRHTLHQIQEMEA

SEQ ID NO: 3 - RNA, 23 nt, synthetic construct
  source 1..23: mol_type = other RNA
aagcattaaagaaagcggcgagg

SEQ ID NO: 4 - RNA, 23 nt, synthetic construct
  source 1..23: mol_type = other RNA
gaggaggtatttatgcagcgggg

SEQ ID NO: 5 - RNA, 23 nt, synthetic construct
  source 1..23: mol_type = other RNA
gagaagaatcaaattcccggcgg

SEQ ID NO: 6 - RNA, 23 nt, synthetic construct
  source 1..23: mol_type = other RNA
aaggtagcgtgagtatttagggg

SEQ ID NO: 7 - RNA, 23 nt, synthetic construct
  source 1..23: mol_type = other RNA
tccatgtagaattaatgacgagg

SEQ ID NO: 8 - RNA, 23 nt, synthetic construct
  source 1..23: mol_type = other RNA
ctgctgggtaacactctcgccgg

SEQ ID NO: 9 - RNA, 23 nt, synthetic construct
  source 1..23: mol_type = other RNA
atctgcagtgctctacaggtagg

SEQ ID NO: 10 - RNA, 23 nt, synthetic construct
  source 1..23: mol_type = other RNA
accagttcccagccgacgttagg

SEQ ID NO: 11 - RNA, 23 nt, synthetic construct
  source 1..23: mol_type = other RNA
gatttcagctctcaacggcacgg

SEQ ID NO: 12 - RNA, 23 nt, synthetic construct
  source 1..23: mol_type = other RNA
gttgttggttacccagccagtgg

SEQ ID NO: 13 - RNA, 23 nt, synthetic construct
  source 1..23: mol_type = other RNA
gagcgcttgttcttaccctgagg

SEQ ID NO: 14 - RNA, 23 nt, synthetic construct
  source 1..23: mol_type = other RNA
gagtaagtacactcgaagcttgg

SEQ ID NO: 15 - RNA, 23 nt, synthetic construct
  source 1..23: mol_type = other RNA
tcgtaacgagacacccgcgacgg

SEQ ID NO: 16 - RNA, 23 nt, synthetic construct
  source 1..23: mol_type = other RNA
aggttccgtatgtacgcctgggg

SEQ ID NO: 17 - RNA, 23 nt, synthetic construct
  source 1..23: mol_type = other RNA
aaataggcaacggactgcagggg

SEQ ID NO: 18 - RNA, 23 nt, synthetic construct
  source 1..23: mol_type = other RNA
agtttagccaggagaaacggagg

SEQ ID NO: 19 - RNA, 23 nt, synthetic construct
  source 1..23: mol_type = other RNA
acctcttgtgacgactgacgagg

SEQ ID NO: 20 - RNA, 23 nt, synthetic construct
  source 1..23: mol_type = other RNA
gcgatgagtaatccaagtggtgg

SEQ ID NO: 21 - RNA, 23 nt, synthetic construct
  source 1..23: mol_type = other RNA
ttttcatcacaggatcgatatgg

SEQ ID NO: 22 - RNA, 23 nt, synthetic construct
  source 1..23: mol_type = other RNA
aatccagtctgtttgggaggagg

SEQ ID NO: 23 - RNA, 23 nt, synthetic construct
  source 1..23: mol_type = other RNA
acctggaggtagtttaatcgtgg

SEQ ID NO: 24 - RNA, 23 nt, synthetic construct
  source 1..23: mol_type = other RNA
tacccttgtaggtatactggagg

SEQ ID NO: 25 - RNA, 23 nt, synthetic construct
  source 1..23: mol_type = other RNA
cctcgcgatgtcttgcacggtgg

SEQ ID NO: 26 - RNA, 23 nt, synthetic construct
  source 1..23: mol_type = other RNA
ggttgctttggagtggtctgcgg

SEQ ID NO: 27 - RNA, 23 nt, synthetic construct
  source 1..23: mol_type = other RNA
tgaggggatctggattccggagg

SEQ ID NO: 28 - RNA, 23 nt, synthetic construct
  source 1..23: mol_type = other RNA
cgaggcgtggagcaaggtacggg

SEQ ID NO: 29 - RNA, 23 nt, synthetic construct
  source 1..23: mol_type = other RNA
ttatgcataccagtgctgggagg

SEQ ID NO: 30 - RNA, 23 nt, synthetic construct
  source 1..23: mol_type = other RNA
gacccaagagctcatcacaaagg

SEQ ID NO: 31 - RNA, 23 nt, synthetic construct
  source 1..23: mol_type = other RNA
cttccgtagatagaatacggcgg

SEQ ID NO: 32 - RNA, 23 nt, synthetic construct
  source 1..23: mol_type = other RNA
tttcaccggaacatgtagcgtgg

SEQ ID NO: 33 - RNA, 23 nt, synthetic construct
  source 1..23: mol_type = other RNA
gaactacggtctaacgactgcgg

SEQ ID NO: 34 - RNA, 23 nt, synthetic construct
  source 1..23: mol_type = other RNA
tcgttagaccgtagttccggcgg

SEQ ID NO: 35 - RNA, 23 nt, synthetic construct
  source 1..23: mol_type = other RNA
accgtacgtaccctctctggcgg

SEQ ID NO: 36 - RNA, 23 nt, synthetic construct
  source 1..23: mol_type = other RNA
tcccaagctgtaaagtccagtgg

SEQ ID NO: 37 - RNA, 23 nt, synthetic construct
  source 1..23: mol_type = other RNA
gtggacaggtgtcacccacgagg

SEQ ID NO: 38 - RNA, 23 nt, synthetic construct
  source 1..23: mol_type = other RNA
tcgaagcgtgtgaatccggtggg

SEQ ID NO: 39 - RNA, 23 nt, synthetic construct
  source 1..23: mol_type = other RNA
cgttaatcagaagttcaatgagg

SEQ ID NO: 40 - RNA, 23 nt, synthetic construct
  source 1..23: mol_type = other RNA
agctagtggtttgcttaacaggg

SEQ ID NO: 41 - RNA, 23 nt, synthetic construct
  source 1..23: mol_type = other RNA
cgaccataaatctggaaaggagg

SEQ ID NO: 42 - RNA, 23 nt, synthetic construct
  source 1..23: mol_type = other RNA
ctcctttccagatttatggtcgg

SEQ ID NO: 43 - RNA, 23 nt, synthetic construct
  source 1..23: mol_type = other RNA
tcagaccatgacgtccggggcgg

SEQ ID NO: 44 - RNA, 23 nt, synthetic construct
  source 1..23: mol_type = other RNA
gcatcagaccatgacgtccgggg

SEQ ID NO: 45 - RNA, 23 nt, synthetic construct
  source 1..23: mol_type = other RNA
gagcatcagagcatgacgtcggg

SEQ ID NO: 46 - RNA, 23 nt, synthetic construct
  source 1..23: mol_type = other RNA
cgcccttttctttgatggagggg

SEQ ID NO: 47 - RNA, 23 nt, synthetic construct
  source 1..23: mol_type = other RNA
aaggcctatatggtagctgaagg

SEQ ID NO: 48 - RNA, 23 nt, synthetic construct
  source 1..23: mol_type = other RNA
gttccccgagcaaaatcatgtgg

SEQ ID NO: 49 - RNA, 23 nt, synthetic construct
  source 1..23: mol_type = other RNA
ggaaaacagtgagctctccgtgg

SEQ ID NO: 50 - RNA, 23 nt, synthetic construct
  source 1..23: mol_type = other RNA
ggctccttgcacgccttgagggg

SEQ ID NO: 51 - RNA, 23 nt, synthetic construct
  source 1..23: mol_type = other RNA
acagcgtccgtcgaaccggaggg

SEQ ID NO: 52 - RNA, 23 nt, synthetic construct
  source 1..23: mol_type = other RNA
gtttccatttgtatgcagcgagg

SEQ ID NO: 53 - RNA, 23 nt, synthetic construct
  source 1..23: mol_type = other RNA
gagctcacttcacttgtagccgg

SEQ ID NO: 54 - RNA, 23 nt, synthetic construct
  source 1..23: mol_type = other RNA
ggatgatatctgctctggcctgg

SEQ ID NO: 55 - RNA, 23 nt, synthetic construct
  source 1..23: mol_type = other RNA
gacggcaacacctctgatggcgg

SEQ ID NO: 56 - RNA, 23 nt, synthetic construct
  source 1..23: mol_type = other RNA
ggaggactccccgccatcagagg

SEQ ID NO: 57 - RNA, 23 nt, synthetic construct
  source 1..23: mol_type = other RNA
cgctctctcgagacgacaggagg

SEQ ID NO: 58 - RNA, 23 nt, synthetic construct
  source 1..23: mol_type = other RNA
gatgatgctcgatctctggccgg

SEQ ID NO: 59 - RNA, 23 nt, synthetic construct
  source 1..23: mol_type = other RNA
tatagacactaggcttcgtgtgg

SEQ ID NO: 60 - RNA, 23 nt, synthetic construct
  source 1..23: mol_type = other RNA
caggatcgaatgaagtgcagtgg

SEQ ID NO: 61 - RNA, 23 nt, synthetic construct
  source 1..23: mol_type = other RNA
agctccatttatagcagacgagg

SEQ ID NO: 62 - RNA, 23 nt, synthetic construct
  source 1..23: mol_type = other RNA
cggcggatgggaactgccagagg

SEQ ID NO: 63 - RNA, 23 nt, synthetic construct
  source 1..23: mol_type = other RNA
tagaagaatcggcgaccggaagg

SEQ ID NO: 64 - RNA, 23 nt, synthetic construct
  source 1..23: mol_type = other RNA
aatctttcgcgtagaagaatcgg

SEQ ID NO: 65 - RNA, 23 nt, synthetic construct
  source 1..23: mol_type = other RNA
tatatgggtagatcgcagggtgg

SEQ ID NO: 66 - RNA, 23 nt, synthetic construct
  source 1..23: mol_type = other RNA
gctagtgagactcttacgggcgg

SEQ ID NO: 67 - RNA, 23 nt, synthetic construct
  source 1..23: mol_type = other RNA
cggccagtataattgatccgtgg

SEQ ID NO: 68 - RNA, 23 nt, synthetic construct
  source 1..23: mol_type = other RNA
agtataattgatccgtggtgcgg

SEQ ID NO: 69 - RNA, 23 nt, synthetic construct
  source 1..23: mol_type = other RNA
tcgctgtaaagatagtcccgcgg

SEQ ID NO: 70 - RNA, 23 nt, synthetic construct
  source 1..23: mol_type = other RNA
gctgtatggatggttaaccatgg

SEQ ID NO: 71 - RNA, 23 nt, synthetic construct
  source 1..23: mol_type = other RNA
atgactgcaagatcgtaagcagg

SEQ ID NO: 72 - RNA, 23 nt, synthetic construct
  source 1..23: mol_type = other RNA
catcatcacgctattagttgagg

SEQ ID NO: 73 - RNA, 23 nt, synthetic construct
  source 1..23: mol_type = other RNA
cgttcagagtcggacacctgggg

SEQ ID NO: 74 - RNA, 23 nt, synthetic construct
  source 1..23: mol_type = other RNA
gggtcgaattcactaccaagagg

SEQ ID NO: 75 - RNA, 23 nt, synthetic construct
  source 1..23: mol_type = other RNA
cgggtaacggaaagaccggacgg

SEQ ID NO: 76 - RNA, 23 nt, synthetic construct
  source 1..23: mol_type = other RNA
gaactagggtttaaagccgccgg

SEQ ID NO: 77 - RNA, 23 nt, synthetic construct
  source 1..23: mol_type = other RNA
ccactcgagtagggttctgccgg

SEQ ID NO: 78 - RNA, 23 nt, synthetic construct
  source 1..23: mol_type = other RNA
gagtggaagttggaggattgggg

SEQ ID NO: 79 - RNA, 23 nt, synthetic construct
  source 1..23: mol_type = other RNA
tagaacaagggaactccctgggg

SEQ ID NO: 80 - RNA, 23 nt, synthetic construct
  source 1..23: mol_type = other RNA
gcggcagcattcctaatcagggg

SEQ ID NO: 81 - RNA, 23 nt, synthetic construct
  source 1..23: mol_type = other RNA
tagctagtatagcctgcaggtgg

SEQ ID NO: 82 - RNA, 23 nt, synthetic construct
  source 1..23: mol_type = other RNA
cctttaatttttcggcagcgcgg

SEQ ID NO: 83 - RNA, 23 nt, synthetic construct
  source 1..23: mol_type = other RNA
attcccggcgcatgccgaagtgg

SEQ ID NO: 84 - RNA, 23 nt, synthetic construct
  source 1..23: mol_type = other RNA
cgagggagagtcagagtagtagg

SEQ ID NO: 85 - RNA, 23 nt, synthetic construct
  source 1..23: mol_type = other RNA
tgtgattccttcctacagggggg

SEQ ID NO: 86 - RNA, 23 nt, synthetic construct
  source 1..23: mol_type = other RNA
gagatagcgatccagaccgaggg

SEQ ID NO: 87 - RNA, 23 nt, synthetic construct
  source 1..23: mol_type = other RNA
acttttcaaacccatcactgtgg

SEQ ID NO: 88 - RNA, 23 nt, synthetic construct
  source 1..23: mol_type = other RNA
tgtccaccgctcccgctgcttgg

SEQ ID NO: 89 - RNA, 23 nt, synthetic construct
  source 1..23: mol_type = other RNA
gagttgttcttggcgatcggggg

SEQ ID NO: 90 - RNA, 23 nt, synthetic construct
  source 1..23: mol_type = other RNA
catttttttctcacctcccgtgg

SEQ ID NO: 91 - RNA, 23 nt, synthetic construct
  source 1..23: mol_type = other RNA
ctggaacacgatctgcctggtgg

SEQ ID NO: 92 - RNA, 23 nt, synthetic construct
  source 1..23: mol_type = other RNA
gcactggaacacgatctgcctgg

SEQ ID NO: 93 - RNA, 23 nt, synthetic construct
  source 1..23: mol_type = other RNA
ctttagcagcgaaggaagcgcgg

SEQ ID NO: 94 - RNA, 23 nt, synthetic construct
  source 1..23: mol_type = other RNA
cctgtcgcgtgtgtgctgaaagg

SEQ ID NO: 95 - RNA, 23 nt, synthetic construct
  source 1..23: mol_type = other RNA
acaccggaattacaatacgaagg

SEQ ID NO: 96 - RNA, 23 nt, synthetic construct
  source 1..23: mol_type = other RNA
cagcaaccaaggttatctcgagg

SEQ ID NO: 97 - RNA, 23 nt, synthetic construct
  source 1..23: mol_type = other RNA
agaactgaccaatcgaggagggg

SEQ ID NO: 98 - RNA, 23 nt, synthetic construct
  source 1..23: mol_type = other RNA
gaaatacgtcgaagcaactgagg

SEQ ID NO: 99 - RNA, 23 nt, synthetic construct
  source 1..23: mol_type = other RNA
acactagtttgtttaccccgggg

SEQ ID NO: 100 - RNA, 23 nt, synthetic construct
  source 1..23: mol_type = other RNA
atgccacctcgataatgtcacgg

SEQ ID NO: 101 - RNA, 23 nt, synthetic construct
  source 1..23: mol_type = other RNA
tcacgtcacgatgtcttgcacgg

SEQ ID NO: 102 - RNA, 23 nt, synthetic construct
  source 1..23: mol_type = other RNA
acatcgtgacgtgaggccaaggg

SEQ ID NO: 103 - RNA, 23 nt, synthetic construct
  source 1..23: mol_type = other RNA
gtagtatctgattctccgcgtgg

SEQ ID NO: 104 - RNA, 23 nt, synthetic construct
  source 1..23: mol_type = other RNA
tggctgccgctaatcaagcgggg

SEQ ID NO: 105 - RNA, 23 nt, synthetic construct
  source 1..23: mol_type = other RNA
actgtgagccgcaccgcacccgg

SEQ ID NO: 106 - RNA, 23 nt, synthetic construct
  source 1..23: mol_type = other RNA
agtggcggggcgtctgctctcgg

SEQ ID NO: 107 - RNA, 23 nt, synthetic construct
  source 1..23: mol_type = other RNA
ggacgggaattggactaaggggg

SEQ ID NO: 108 - RNA, 23 nt, synthetic construct
  source 1..23: mol_type = other RNA
cggaccaaccttcgtgaggacgg

SEQ ID NO: 109 - RNA, 23 nt, synthetic construct
  source 1..23: mol_type = other RNA
gctgagtttcgttctctggccgg

SEQ ID NO: 110 - RNA, 23 nt, synthetic construct
  source 1..23: mol_type = other RNA
attcggtcccttctccggccggg

SEQ ID NO: 111 - RNA, 23 nt, synthetic construct
  source 1..23: mol_type = other RNA
tcgtactcagacgtggactgcgg

SEQ ID NO: 112 - RNA, 23 nt, synthetic construct
  source 1..23: mol_type = other RNA
aatgacgcagtcacgcagcaggg

SEQ ID NO: 113 - RNA, 23 nt, synthetic construct
  source 1..23: mol_type = other RNA
aacgtgaagtgtttctcaggggg

SEQ ID NO: 114 - RNA, 23 nt, synthetic construct
  source 1..23: mol_type = other RNA
caccacaaagggttacacaaagg

SEQ ID NO: 115 - RNA, 23 nt, synthetic construct
  source 1..23: mol_type = other RNA
aggtaatattactcacgcagtgg

SEQ ID NO: 116 - RNA, 23 nt, synthetic construct
  source 1..23: mol_type = other RNA
ttaacccctctatataaccctgg

SEQ ID NO: 117 - RNA, 23 nt, synthetic construct
  source 1..23: mol_type = other RNA
ctaccaaaggctacacacgaggg

SEQ ID NO: 118 - RNA, 23 nt, synthetic construct
  source 1..23: mol_type = other RNA
cctaccaaaggctacacacgagg

SEQ ID NO: 119 - RNA, 23 nt, synthetic construct
  source 1..23: mol_type = other RNA
aaacctcccaagacacacgaagg

SEQ ID NO: 120 - RNA, 23 nt, synthetic construct
  source 1..23: mol_type = other RNA
attcgctcgtccgatcaggaagg

SEQ ID NO: 121 - RNA, 23 nt, synthetic construct
  source 1..23: mol_type = other RNA
aggccacaagctccaccacgggg

SEQ ID NO: 122 - RNA, 23 nt, synthetic construct
  source 1..23: mol_type = other RNA
caaacaaccagtcatccgagagg

SEQ ID NO: 123 - RNA, 23 nt, synthetic construct
  source 1..23: mol_type = other RNA
cacgacctggagaacgacgtcgg

SEQ ID NO: 124 - RNA, 23 nt, synthetic construct
  source 1..23: mol_type = other RNA
acagagtccaacaacttccacgg

SEQ ID NO: 125 - RNA, 23 nt, synthetic construct
  source 1..23: mol_type = other RNA
tcgccgctggacatcgacagcgg

SEQ ID NO: 126 - RNA, 23 nt, synthetic construct
  source 1..23: mol_type = other RNA
atgaacgtggtcctcaacggcgg

SEQ ID NO: 127 - RNA, 23 nt, synthetic construct
  source 1..23: mol_type = other RNA
tgactcccctgacctaacgtcgg

SEQ ID NO: 128 - RNA, 23 nt, synthetic construct
  source 1..23: mol_type = other RNA
tgactcccctgacctaacgtcgg

SEQ ID NO: 129 - RNA, 23 nt, synthetic construct
  source 1..23: mol_type = other RNA
gttgaacacctggaacgcggcgg

SEQ ID NO: 130 - RNA, 23 nt, synthetic construct
  source 1..23: mol_type = other RNA
ttcagctctcaacggcacggcgg

SEQ ID NO: 131 - RNA, 23 nt, synthetic construct
  source 1..23: mol_type = other RNA
gctaatggtaggaagaggggagg

SEQ ID NO: 132 - RNA, 23 nt, synthetic construct
  source 1..23: mol_type = other RNA
tccttgcaagtcaacaatggcgg

SEQ ID NO: 133 - RNA, 23 nt, synthetic construct
  source 1..23: mol_type = other RNA
cgggacccgatgtcacgtagcgg

SEQ ID NO: 134 - RNA, 23 nt, synthetic construct
  source 1..23: mol_type = other RNA
cgagagcgaacagaacacgccgg

SEQ ID NO: 135 - RNA, 23 nt, synthetic construct
  source 1..23: mol_type = other RNA
gaacttgtagatgtcgacgtcgg

SEQ ID NO: 136 - RNA, 23 nt, synthetic construct
  source 1..23: mol_type = other RNA
cacgaccaggacgagcactgcgg

SEQ ID NO: 137 - RNA, 23 nt, synthetic construct
  source 1..23: mol_type = other RNA
acgtagtccgacatgaacggcgg

SEQ ID NO: 138 - RNA, 23 nt, synthetic construct
  source 1..23: mol_type = other RNA
tacgtcatcaccacctacgacgg

SEQ ID NO: 139 - RNA, 23 nt, synthetic construct
  source 1..23: mol_type = other RNA
tcatttataggtaagagaggggg

SEQ ID NO: 140 - RNA, 23 nt, synthetic construct
  source 1..23: mol_type = other RNA
gagggacaggagcttcatggtgg

SEQ ID NO: 141 - RNA, 23 nt, synthetic construct
  source 1..23: mol_type = other RNA
aatccaactgaagtggacagagg

SEQ ID NO: 142 - RNA, 23 nt, synthetic construct
  source 1..23: mol_type = other RNA
aatcgtggtagagaactcgacgg

SEQ ID NO: 143 - RNA, 23 nt, synthetic construct
  source 1..23: mol_type = other RNA
caatggaaattaccacttggggg

SEQ ID NO: 144 - RNA, 23 nt, synthetic construct
  source 1..23: mol_type = other RNA
ggatttgtggtgaattaccgtgg

SEQ ID NO: 145 - RNA, 23 nt, synthetic construct
  source 1..23: mol_type = other RNA
gtcctgcatcaggagcctcgagg

SEQ ID NO: 146 - RNA, 23 nt, synthetic construct
  source 1..23: mol_type = other RNA
agagtgtagagatagcgtcgcgg

SEQ ID NO: 147 - RNA, 23 nt, synthetic construct
  source 1..23: mol_type = other RNA
aggggcggttcaggtaattgggg

SEQ ID NO: 148 - RNA, 23 nt, synthetic construct
  source 1..23: mol_type = other RNA
tgggtgctgctgaaacatgatgg

SEQ ID NO: 149 - RNA, 23 nt, synthetic construct
  source 1..23: mol_type = other RNA
gagggtgtccgggaaccacgcgg

SEQ ID NO: 150 - RNA, 23 nt, synthetic construct
  source 1..23: mol_type = other RNA
cttccgtagatagaatacggcgg

SEQ ID NO: 151 - RNA, 23 nt, synthetic construct
  source 1..23: mol_type = other RNA
gaaggagatgatctaccgcgcgg

SEQ ID NO: 152 - RNA, 23 nt, synthetic construct
  source 1..23: mol_type = other RNA
agcctgaattgtgaacaaggcgg

SEQ ID NO: 153 - RNA, 23 nt, synthetic construct
  source 1..23: mol_type = other RNA
gagccgttcaaggtcgacagcgg

SEQ ID NO: 154 - RNA, 23 nt, synthetic construct
  source 1..23: mol_type = other RNA
aagcgacctcacagtcactgggg

SEQ ID NO: 155 - RNA, 23 nt, synthetic construct
  source 1..23: mol_type = other RNA
ggagccaacacattgccatgggg

SEQ ID NO: 156 - RNA, 23 nt, synthetic construct
  source 1..23: mol_type = other RNA
ctctacaagtttgggcacagcgg

SEQ ID NO: 157 - RNA, 23 nt, synthetic construct
  source 1..23: mol_type = other RNA
tcagacgatgatgacatggaagg

SEQ ID NO: 158 - RNA, 23 nt, synthetic construct
  source 1..23: mol_type = other RNA
ggacgacgatttcgtcaacgggg

SEQ ID NO: 159 - RNA, 23 nt, synthetic construct
  source 1..23: mol_type = other RNA
cagagaaggactggacaaggcgg

SEQ ID NO: 160 - RNA, 23 nt, synthetic construct
  source 1..23: mol_type = other RNA
acacatcaacagtgacacaaagg

SEQ ID NO: 161 - RNA, 23 nt, synthetic construct
  source 1..23: mol_type = other RNA
caagcactgcgacaacaacgagg

SEQ ID NO: 162 - RNA, 23 nt, synthetic construct
  source 1..23: mol_type = other RNA
ataatgcaatgcaatacaggcgg

SEQ ID NO: 163 - RNA, 23 nt, synthetic construct
  source 1..23: mol_type = other RNA
caagcactgcgacaacaacgagg

SEQ ID NO: 164 - RNA, 23 nt, synthetic construct
  source 1..23: mol_type = other RNA
gatcagcttccagatcggggcgg

SEQ ID NO: 165 - RNA, 23 nt, synthetic construct
  source 1..23: mol_type = other RNA
ctgccgaaggatgtaaatggggg

SEQ ID NO: 166 - RNA, 23 nt, synthetic construct
  source 1..23: mol_type = other RNA
ctgccgaaggatgtaaatggggg

SEQ ID NO: 167 - RNA, 23 nt, synthetic construct
  source 1..23: mol_type = other RNA
cgtgaccgtgatgactaccgtgg

SEQ ID NO: 168 - RNA, 23 nt, synthetic construct
  source 1..23: mol_type = other RNA
gaagggccaagattgagcagtgg

SEQ ID NO: 169 - RNA, 23 nt, synthetic construct
  source 1..23: mol_type = other RNA
gctcggcacccggttccccgcgg

SEQ ID NO: 170 - RNA, 23 nt, synthetic construct
  source 1..23: mol_type = other RNA
acagttccagagatcaacagcgg

SEQ ID NO: 171 - RNA, 23 nt, synthetic construct
  source 1..23: mol_type = other RNA
tccgagtgagaagaagatggtgg

SEQ ID NO: 172 - RNA, 23 nt, synthetic construct
  source 1..23: mol_type = other RNA
gcccccgaagctgagcaccgtgg

SEQ ID NO: 173 - RNA, 23 nt, synthetic construct
  source 1..23: mol_type = other RNA
agtctctagtagctccagagcgg

SEQ ID NO: 174 - RNA, 23 nt, synthetic construct
  source 1..23: mol_type = other RNA
cttaccggagcaccaccagacgg

SEQ ID NO: 175 - RNA, 23 nt, synthetic construct
  source 1..23: mol_type = other RNA
cgactcccatgctgcccgtgtgg

SEQ ID NO: 176 - RNA, 23 nt, synthetic construct
  source 1..23: mol_type = other RNA
cgtaccaaaaatcccaccggcgg

SEQ ID NO: 177 - RNA, 23 nt, synthetic construct
  source 1..23: mol_type = other RNA
tcgccaaggacaccacaacacgg

SEQ ID NO: 178 - RNA, 23 nt, synthetic construct
  source 1..23: mol_type = other RNA
aagcttgtggatgatccccgggg

SEQ ID NO: 179 - RNA, 23 nt, synthetic construct
  source 1..23: mol_type = other RNA
gctccatttatagcagacgaggg

SEQ ID NO: 180 - RNA, 23 nt, synthetic construct
  source 1..23: mol_type = other RNA
aggaacgggaatcatcaggaggg

SEQ ID NO: 181 - RNA, 23 nt, synthetic construct
  source 1..23: mol_type = other RNA
gtcctgcatcaggagcctcgagg

SEQ ID NO: 182 - RNA, 23 nt, synthetic construct
  source 1..23: mol_type = other RNA
gctgctcaggagcaaagcagcgg

SEQ ID NO: 183 - RNA, 23 nt, synthetic construct
  source 1..23: mol_type = other RNA
ttggcctcatcaaacgaggaggg

SEQ ID NO: 184 - RNA, 23 nt, synthetic construct
  source 1..23: mol_type = other RNA
gatgcagccagatggaacagcgg

SEQ ID NO: 185 - RNA, 23 nt, synthetic construct
  source 1..23: mol_type = other RNA
agtcgttcttgagctcattgtgg

SEQ ID NO: 186 - RNA, 23 nt, synthetic construct
  source 1..23: mol_type = other RNA
tatatgggtagatcgcagggtgg

SEQ ID NO: 187 - RNA, 23 nt, synthetic construct
  source 1..23: mol_type = other RNA
gaggagatgatggcgttcggcgg

SEQ ID NO: 188 - RNA, 23 nt, synthetic construct
  source 1..23: mol_type = other RNA
ggaacactggaactgaaccgagg

SEQ ID NO: 189 - RNA, 23 nt, synthetic construct
  source 1..23: mol_type = other RNA
agatcgtcctagctagagagagg

SEQ ID NO: 190 - RNA, 23 nt, synthetic construct
  source 1..23: mol_type = other RNA
ggtggaggagatgtacctggagg

SEQ ID NO: 191 - RNA, 23 nt, synthetic construct
  source 1..23: mol_type = other RNA
cataacgtgaaatgtcaaagcgg

SEQ ID NO: 192 - RNA, 23 nt, synthetic construct
  source 1..23: mol_type = other RNA
acagcaccacctgcaaacagcgg

SEQ ID NO: 193 - RNA, 23 nt, synthetic construct
  source 1..23: mol_type = other RNA
tcaccccagaactccatcgacgg

SEQ ID NO: 194 - RNA, 23 nt, synthetic construct
  source 1..23: mol_type = other RNA
tccatgaaggagaagcatggtgg

SEQ ID NO: 195 - RNA, 23 nt, synthetic construct
  source 1..23: mol_type = other RNA
gtcgtcgcaatggagttctgggg

SEQ ID NO: 196 - RNA, 23 nt, synthetic construct
  source 1..23: mol_type = other RNA
tgtctagtttgggacatacgcgg

SEQ ID NO: 197 - RNA, 23 nt, synthetic construct
  source 1..23: mol_type = other RNA
gagccttcaagcataagacatgg

SEQ ID NO: 198 - RNA, 23 nt, synthetic construct
  source 1..23: mol_type = other RNA
acaactggttcatcaaccagcgg

SEQ ID NO: 199 - RNA, 23 nt, synthetic construct
  source 1..23: mol_type = other RNA
atgagcaagaagctgcagggcgg

SEQ ID NO: 200 - RNA, 23 nt, synthetic construct
  source 1..23: mol_type = other RNA
cttctggaacgacatcgcggcgg

SEQ ID NO: 201 - RNA, 23 nt, synthetic construct
  source 1..23: mol_type = other RNA
cttggtagcgaagaagacgaggg

SEQ ID NO: 202 - RNA, 23 nt, synthetic construct
  source 1..23: mol_type = other RNA
cgagaacaagacgccatgggcgg

SEQ ID NO: 203 - RNA, 23 nt, synthetic construct
  source 1..23: mol_type = other RNA
gagaggttgagcaagaaccgagg

SEQ ID NO: 204 - RNA, 23 nt, synthetic construct
  source 1..23: mol_type = other RNA
cagcatccacgacatcaccacgg

SEQ ID NO: 205 - RNA, 23 nt, synthetic construct
  source 1..23: mol_type = other RNA
ggaggtgaattcctctggtgagg

SEQ ID NO: 206 - RNA, 23 nt, synthetic construct
  source 1..23: mol_type = other RNA
acttttcaaacccatcactgtgg

SEQ ID NO: 207 - RNA, 23 nt, synthetic construct
  source 1..23: mol_type = other RNA
gggaggtgagaaaaaaatggggg

SEQ ID NO: 208 - RNA, 23 nt, synthetic construct
  source 1..23: mol_type = other RNA
tccatctcaaggctccacagtgg

SEQ ID NO: 209 - RNA, 23 nt, synthetic construct
  source 1..23: mol_type = other RNA
cttccggttccatcccaccgagg

SEQ ID NO: 210 - RNA, 23 nt, synthetic construct
  source 1..23: mol_type = other RNA
tcacatctaagctagcatcgcgg

SEQ ID NO: 211 - RNA, 23 nt, synthetic construct
  source 1..23: mol_type = other RNA
gcgtccacggtggaagacaaggg

SEQ ID NO: 212 - RNA, 23 nt, synthetic construct
  source 1..23: mol_type = other RNA
gtatgatatgtatcctggtgcgg

SEQ ID NO: 213 - RNA, 23 nt, synthetic construct
  source 1..23: mol_type = other RNA
gagggcggtgttcggcccagcgg

SEQ ID NO: 214 - RNA, 23 nt, synthetic construct
  source 1..23: mol_type = other RNA
agggtacttcctgtcacgagagg

SEQ ID NO: 215 - RNA, 23 nt, synthetic construct
  source 1..23: mol_type = other RNA
tgaagccagtattgtccttgggg

SEQ ID NO: 216 - RNA, 23 nt, synthetic construct
  source 1..23: mol_type = other RNA
gttgctgtacctgagaactgtgg

SEQ ID NO: 217 - RNA, 23 nt, synthetic construct
  source 1..23: mol_type = other RNA
tagggcgtttcaagaaagggcgg

SEQ ID NO: 218 - RNA, 23 nt, synthetic construct
  source 1..23: mol_type = other RNA
caatgaatccatgagcagagcgg

SEQ ID NO: 219 - RNA, 23 nt, synthetic construct
  source 1..23: mol_type = other RNA
ggaggcagatccgacgaacgagg

SEQ ID NO: 220 - RNA, 23 nt, synthetic construct
  source 1..23: mol_type = other RNA
aatgcccctagacactgccaggg

SEQ ID NO: 221 - RNA, 23 nt, synthetic construct
  source 1..23: mol_type = other RNA
gacctctctaacctacgtagggg

SEQ ID NO: 222 - RNA, 23 nt, synthetic construct
  source 1..23: mol_type = other RNA
ggagccaagatatagcacgtcgg

SEQ ID NO: 223 - RNA, 23 nt, synthetic construct
  source 1..23: mol_type = other RNA
gatggcggcgaatcgcacagggg

SEQ ID NO: 224 - RNA, 23 nt, synthetic construct
  source 1..23: mol_type = other RNA
catcagtagagcaagagcgacgg

SEQ ID NO: 225 - RNA, 23 nt, synthetic construct
  source 1..23: mol_type = other RNA
gagaagcgaaggaagcaacgggg

SEQ ID NO: 226 - RNA, 23 nt, synthetic construct
  source 1..23: mol_type = other RNA
cttttgcaaacaaagaaccgtgg

SEQ ID NO: 227 - RNA, 23 nt, synthetic construct
  source 1..23: mol_type = other RNA
tgatgcaacgcaacccaaggagg

SEQ ID NO: 228 - RNA, 23 nt, synthetic construct
  source 1..23: mol_type = other RNA
agcattgttgtgaatggtagggg

SEQ ID NO: 229 - RNA, 23 nt, synthetic construct
  source 1..23: mol_type = other RNA
cctaggatcgaatgacagcgcgg

SEQ ID NO: 230 - RNA, 23 nt, synthetic construct
  source 1..23: mol_type = other RNA
agggcggacattcatcaaggggg

SEQ ID NO: 231 - RNA, 23 nt, synthetic construct
  source 1..23: mol_type = other RNA
agcaatcttggtgatcagggtgg

SEQ ID NO: 232 - RNA, 23 nt, synthetic construct
  source 1..23: mol_type = other RNA
ctgtaaaagtggctacacctagg

SEQ ID NO: 233 - RNA, 23 nt, synthetic construct
  source 1..23: mol_type = other RNA
caggtcgacatcctcgacgacgg

SEQ ID NO: 234 - RNA, 23 nt, synthetic construct
  source 1..23: mol_type = other RNA
gtggtggtgaccacatacgaggg

SEQ ID NO: 235 - RNA, 23 nt, synthetic construct
  source 1..23: mol_type = other RNA
tttagagagagagagagacgggg

SEQ ID NO: 236 - RNA, 23 nt, synthetic construct
  source 1..23: mol_type = other RNA
ctttgaagcaaagaatgctgagg

SEQ ID NO: 237 - RNA, 23 nt, synthetic construct
  source 1..23: mol_type = other RNA
agatcagatgcaattaaccgcgg

SEQ ID NO: 238 - RNA, 23 nt, synthetic construct
  source 1..23: mol_type = other RNA
tggatgtcaagtttgtgttgcgg

SEQ ID NO: 239 - RNA, 23 nt, synthetic construct
  source 1..23: mol_type = other RNA
aggccacaagctccaccacgggg

SEQ ID NO: 240 - RNA, 23 nt, synthetic construct
  source 1..23: mol_type = other RNA
ggaggccacaagctccaccacgg

SEQ ID NO: 241 - RNA, 23 nt, synthetic construct
  source 1..23: mol_type = other RNA
cacgacctggagaacgacgtcgg

SEQ ID NO: 242 - RNA, 23 nt, synthetic construct
  source 1..23: mol_type = other RNA
gagaagaatcaaattcccggcgg

SEQ ID NO: 243 - RNA, 23 nt, synthetic construct
  source 1..23: mol_type = other RNA
tcgccgctggacatcgacagcgg

SEQ ID NO: 244 - RNA, 23 nt, synthetic construct
  source 1..23: mol_type = other RNA
ggcatgggtcatgatgacgacgg

SEQ ID NO: 245 - RNA, 23 nt, synthetic construct
  source 1..23: mol_type = other RNA
tgactcccctgacctaacgtcgg

SEQ ID NO: 246 - RNA, 23 nt, synthetic construct
  source 1..23: mol_type = other RNA
ggccaccacagtgacgacgacgg

SEQ ID NO: 247 - RNA, 23 nt, synthetic construct
  source 1..23: mol_type = other RNA
gttgaacacctggaacgcggcgg

SEQ ID NO: 248 - RNA, 23 nt, synthetic construct
  source 1..23: mol_type = other RNA
gcacgtccacctccgccgcgtgg

SEQ ID NO: 249 - RNA, 23 nt, synthetic construct
  source 1..23: mol_type = other RNA
gctaatggtaggaagaggggagg

SEQ ID NO: 250 - RNA, 23 nt, synthetic construct
  source 1..23: mol_type = other RNA
gggtggaaccggaaccccggtgg

SEQ ID NO: 251 - RNA, 23 nt, synthetic construct
  source 1..23: mol_type = other RNA
cgggacccgatgtcacgtagcgg

SEQ ID NO: 252 - RNA, 23 nt, synthetic construct
  source 1..23: mol_type = other RNA
gggccatggggatctacgagcgg

SEQ ID NO: 253 - RNA, 23 nt, synthetic construct
  source 1..23: mol_type = other RNA
gaacttgtagatgtcgacgtcgg

SEQ ID NO: 254 - RNA, 23 nt, synthetic construct
  source 1..23: mol_type = other RNA
gtttagccaggagaaacggaggg

SEQ ID NO: 255 - RNA, 23 nt, synthetic construct
  source 1..23: mol_type = other RNA
acgtagtccgacatgaacggcgg

SEQ ID NO: 256 - RNA, 23 nt, synthetic construct
  source 1..23: mol_type = other RNA
gttcatgtcggactacgtggcgg

SEQ ID NO: 257 - RNA, 23 nt, synthetic construct
  source 1..23: mol_type = other RNA
tcatttataggtaagagaggggg

SEQ ID NO: 258 - RNA, 23 nt, synthetic construct
  source 1..23: mol_type = other RNA
agtaggcaactaagacaaagtgg

SEQ ID NO: 259 - RNA, 23 nt, synthetic construct
  source 1..23: mol_type = other RNA
aatccaactgaagtggacagagg

SEQ ID NO: 260 - RNA, 23 nt, synthetic construct
  source 1..23: mol_type = other RNA
agacgagggatcattcagcaggg

SEQ ID NO: 261 - RNA, 23 nt, synthetic construct
  source 1..23: mol_type = other RNA
caatggaaattaccacttggggg

SEQ ID NO: 262 - RNA, 23 nt, synthetic construct
  source 1..23: mol_type = other RNA
gcggtggctttggtaccgagcgg

SEQ ID NO: 263 - RNA, 23 nt, synthetic construct
  source 1..23: mol_type = other RNA
gtcctgcatcaggagcctcgagg

SEQ ID NO: 264 - RNA, 23 nt, synthetic construct
  source 1..23: mol_type = other RNA
acaaacgaaagagagagaggcgg

SEQ ID NO: 265 - RNA, 23 nt, synthetic construct
  source 1..23: mol_type = other RNA
aggggcggttcaggtaattgggg

SEQ ID NO: 266 - RNA, 23 nt, synthetic construct
  source 1..23: mol_type = other RNA
gcaggagacgtcaaggcccgtgg

SEQ ID NO: 267 - RNA, 23 nt, synthetic construct
  source 1..23: mol_type = other RNA
gagggtgtccgggaaccacgcgg

SEQ ID NO: 268 - RNA, 23 nt, synthetic construct
  source 1..23: mol_type = other RNA
tgtgccacagcagctccacgagg

SEQ ID NO: 269 - RNA, 23 nt, synthetic construct
  source 1..23: mol_type = other RNA
agcctgaattgtgaacaaggcgg

SEQ ID NO: 270 - RNA, 23 nt, synthetic construct
  source 1..23: mol_type = other RNA
gaaggagatgatctaccgcgcgg

SEQ ID NO: 271 - RNA, 23 nt, synthetic construct
  source 1..23: mol_type = other RNA
gagccgttcaaggtcgacagcgg

SEQ ID NO: 272 - RNA, 23 nt, synthetic construct
  source 1..23: mol_type = other RNA
caccaagctcgagacaagcaggg

SEQ ID NO: 273 - RNA, 23 nt, synthetic construct
  source 1..23: mol_type = other RNA
ggagccaacacattgccatgggg

SEQ ID NO: 274 - RNA, 23 nt, synthetic construct
  source 1..23: mol_type = other RNA
gtggacaggtgtcacccacgagg

SEQ ID NO: 275 - RNA, 23 nt, synthetic construct
  source 1..23: mol_type = other RNA
tcagacgatgatgacatggaagg

SEQ ID NO: 276 - RNA, 23 nt, synthetic construct
  source 1..23: mol_type = other RNA
atcgtctgactgctcacgtgagg

SEQ ID NO: 277 - RNA, 23 nt, synthetic construct
  source 1..23: mol_type = other RNA
cagagaaggactggacaaggcgg

SEQ ID NO: 278 - RNA, 23 nt, synthetic construct
  source 1..23: mol_type = other RNA
agagaaggactggacaaggcggg

SEQ ID NO: 279 - RNA, 23 nt, synthetic construct
  source 1..23: mol_type = other RNA
caagcactgcgacaacaacgagg

SEQ ID NO: 280 - RNA, 23 nt, synthetic construct
  source 1..23: mol_type = other RNA
gaggcccgtgaagatcttggcgg

SEQ ID NO: 281 - RNA, 23 nt, synthetic construct
  source 1..23: mol_type = other RNA
caagcactgcgacaacaacgagg

SEQ ID NO: 282 - RNA, 23 nt, synthetic construct
  source 1..23: mol_type = other RNA
gaggcccgtgaagatcttggcgg

SEQ ID NO: 283 - RNA, 23 nt, synthetic construct
  source 1..23: mol_type = other RNA
ctgccgaaggatgtaaatggggg

SEQ ID NO: 284 - RNA, 23 nt, synthetic construct
  source 1..23: mol_type = other RNA
agaatgcctatgggacctggtgg

SEQ ID NO: 285 - RNA, 23 nt, synthetic construct
  source 1..23: mol_type = other RNA
cgtgaccgtgatgactaccgtgg

SEQ ID NO: 286 - RNA, 23 nt, synthetic construct
  source 1..23: mol_type = other RNA
atgggcgttctgcttccaggagg

SEQ ID NO: 287 - RNA, 23 nt, synthetic construct
  source 1..23: mol_type = other RNA
gctcggcacccggttccccgcgg

SEQ ID NO: 288 - RNA, 23 nt, synthetic construct
  source 1..23: mol_type = other RNA
acagcgtccgtcgaaccggaggg

SEQ ID NO: 289 - RNA, 23 nt, synthetic construct
  source 1..23: mol_type = other RNA
tccgagtgagaagaagatggtgg

SEQ ID NO: 290 - RNA, 23 nt, synthetic construct
  source 1..23: mol_type = other RNA
agccggaaccggaagagacgagg

SEQ ID NO: 291 - RNA, 23 nt, synthetic construct
  source 1..23: mol_type = other RNA
agtctctagtagctccagagcgg

SEQ ID NO: 292 - RNA, 23 nt, synthetic construct
  source 1..23: mol_type = other RNA
tgtgttcaatgacatgagctggg

SEQ ID NO: 293 - RNA, 23 nt, synthetic construct
  source 1..23: mol_type = other RNA
cgactcccatgctgcccgtgtgg

SEQ ID NO: 294 - RNA, 23 nt, synthetic construct
  source 1..23: mol_type = other RNA
gccaggtgacgcggccacacggg

SEQ ID NO: 295 - RNA, 23 nt, synthetic construct
  source 1..23: mol_type = other RNA
tcgccaaggacaccacaacacgg

SEQ ID NO: 296 - RNA, 23 nt, synthetic construct
  source 1..23: mol_type = other RNA
tggcggcgtcacggaccaggagg

SEQ ID NO: 297 - RNA, 23 nt, synthetic construct
  source 1..23: mol_type = other RNA
gctccatttatagcagacgaggg

SEQ ID NO: 298 - RNA, 23 nt, synthetic construct
  source 1..23: mol_type = other RNA
gcaagatcgagatgttcaagcgg

SEQ ID NO: 299 - RNA, 23 nt, synthetic construct
  source 1..23: mol_type = other RNA
gtcctgcatcaggagcctcgagg

SEQ ID NO: 300 - RNA, 23 nt, synthetic construct
  source 1..23: mol_type = other RNA
tgggtgactgagagccagagggg

SEQ ID NO: 301 - RNA, 23 nt, synthetic construct
  source 1..23: mol_type = other RNA
ttggcctcatcaaacgaggaggg

SEQ ID NO: 302 - RNA, 23 nt, synthetic construct
  source 1..23: mol_type = other RNA
gccattggcctcatcaaacgagg

SEQ ID NO: 303 - RNA, 23 nt, synthetic construct
  source 1..23: mol_type = other RNA
agtcgttcttgagctcattgtgg

SEQ ID NO: 304 - RNA, 23 nt, synthetic construct
  source 1..23: mol_type = other RNA
ttggagcaataacttgacgatgg

SEQ ID NO: 305 - RNA, 23 nt, synthetic construct
  source 1..23: mol_type = other RNA
gcggcggttctggaaccagacgg

SEQ ID NO: 306 - RNA, 23 nt, synthetic construct
  source 1..23: mol_type = other RNA
ggaacactggaactgaaccgagg

SEQ ID NO: 307 - RNA, 23 nt, synthetic construct
  source 1..23: mol_type = other RNA
gaggagatgatggcgttcggcgg

SEQ ID NO: 308 - RNA, 23 nt, synthetic construct
  source 1..23: mol_type = other RNA
ggacgtggacgaggagatgatgg

SEQ ID NO: 309 - RNA, 23 nt, synthetic construct
  source 1..23: mol_type = other RNA
agatcgtcctagctagagagagg

SEQ ID NO: 310 - RNA, 23 nt, synthetic construct
  source 1..23: mol_type = other RNA
cctagctagagagaggcaggagg

SEQ ID NO: 311 - RNA, 23 nt, synthetic construct
  source 1..23: mol_type = other RNA
cataacgtgaaatgtcaaagcgg

SEQ ID NO: 312 - RNA, 23 nt, synthetic construct
  source 1..23: mol_type = other RNA
gcaaaggtctaatgacagtgtgg

SEQ ID NO: 313 - RNA, 23 nt, synthetic construct
  source 1..23: mol_type = other RNA
tcaccccagaactccatcgacgg

SEQ ID NO: 314 - RNA, 23 nt, synthetic construct
  source 1..23: mol_type = other RNA
ccggtctttccgttacccggcgg

SEQ ID NO: 315 - RNA, 23 nt, synthetic construct
  source 1..23: mol_type = other RNA
gtcgtcgcaatggagttctgggg

SEQ ID NO: 316 - RNA, 23 nt, synthetic construct
  source 1..23: mol_type = other RNA
agaactccattgcgacgacgagg

SEQ ID NO: 317 - RNA, 23 nt, synthetic construct
  source 1..23: mol_type = other RNA
gagccttcaagcataagacatgg

SEQ ID NO: 318 - RNA, 23 nt, synthetic construct
  source 1..23: mol_type = other RNA
gaaagggaagctccccaaggagg

SEQ ID NO: 319 - RNA, 23 nt, synthetic construct
  source 1..23: mol_type = other RNA
atgagcaagaagctgcagggcgg

SEQ ID NO: 320 - RNA, 23 nt, synthetic construct
  source 1..23: mol_type = other RNA
cctttgctaagaaatcccaccgg

SEQ ID NO: 321 - RNA, 23 nt, synthetic construct
  source 1..23: mol_type = other RNA
cttggtagcgaagaagacgaggg

SEQ ID NO: 322 - RNA, 23 nt, synthetic construct
  source 1..23: mol_type = other RNA
cacgccctcgagaaggacgtggg

SEQ ID NO: 323 - RNA, 23 nt, synthetic construct
  source 1..23: mol_type = other RNA
gagaggttgagcaagaaccgagg

SEQ ID NO: 324 - RNA, 23 nt, synthetic construct
  source 1..23: mol_type = other RNA
gccgagagaagaaagcagagagg

SEQ ID NO: 325 - RNA, 23 nt, synthetic construct
  source 1..23: mol_type = other RNA
ggaggtgaattcctctggtgagg

SEQ ID NO: 326 - RNA, 23 nt, synthetic construct
  source 1..23: mol_type = other RNA
tatcaccgtttcctcaccagagg

SEQ ID NO: 327 - RNA, 23 nt, synthetic construct
  source 1..23: mol_type = other RNA
gggaggtgagaaaaaaatggggg

SEQ ID NO: 328 - RNA, 23 nt, synthetic construct
  source 1..23: mol_type = other RNA
gaagcggtagaggtcgacctcgg

SEQ ID NO: 329 - RNA, 23 nt, synthetic construct
  source 1..23: mol_type = other RNA
gcgggttctggaaggccacgggg

SEQ ID NO: 330 - RNA, 23 nt, synthetic construct
  source 1..23: mol_type = other RNA
gatggcgaagattggcgagaggg

SEQ ID NO: 331 - RNA, 23 nt, synthetic construct
  source 1..23: mol_type = other RNA
gcgtccacggtggaagacaaggg

SEQ ID NO: 332 - RNA, 23 nt, synthetic construct
  source 1..23: mol_type = other RNA
aagacccttgtcttccaccgtgg

SEQ ID NO: 333 - RNA, 23 nt, synthetic construct
  source 1..23: mol_type = other RNA
gagggcggtgttcggcccagcgg

SEQ ID NO: 334 - RNA, 23 nt, synthetic construct
  source 1..23: mol_type = other RNA
ggctcctcttccccgcacggcgg

SEQ ID NO: 335 - RNA, 23 nt, synthetic construct
  source 1..23: mol_type = other RNA
tgaagccagtattgtccttgggg

SEQ ID NO: 336 - RNA, 23 nt, synthetic construct
  source 1..23: mol_type = other RNA
aatgaagccagtattgtccttgg

SEQ ID NO: 337 - RNA, 23 nt, synthetic construct
  source 1..23: mol_type = other RNA
tagggcgtttcaagaaagggcgg

SEQ ID NO: 338 - RNA, 23 nt, synthetic construct
  source 1..23: mol_type = other RNA
atggagggctgctcaaatcgcgg

SEQ ID NO: 339 - RNA, 23 nt, synthetic construct
  source 1..23: mol_type = other RNA
ggaggcagatccgacgaacgagg

SEQ ID NO: 340 - RNA, 23 nt, synthetic construct
  source 1..23: mol_type = other RNA
gtgcaagacatcgtgacgtgagg

SEQ ID NO: 341 - RNA, 23 nt, synthetic construct
  source 1..23: mol_type = other RNA
gcagaatggagattatccagagg

SEQ ID NO: 342 - RNA, 23 nt, synthetic construct
  source 1..23: mol_type = other RNA
gacctctctaacctacgtagggg

SEQ ID NO: 343 - RNA, 23 nt, synthetic construct
  source 1..23: mol_type = other RNA
gatggcggcgaatcgcacagggg

SEQ ID NO: 344 - RNA, 23 nt, synthetic construct
  source 1..23: mol_type = other RNA
gggagaacagagctaagcaacgg

SEQ ID NO: 345 - RNA, 23 nt, synthetic construct
  source 1..23: mol_type = other RNA
gagaagcgaaggaagcaacgggg

SEQ ID NO: 346 - RNA, 23 nt, synthetic construct
  source 1..23: mol_type = other RNA
gggtgagcagaagaagaaggagg

SEQ ID NO: 347 - RNA, 23 nt, synthetic construct
  source 1..23: mol_type = other RNA
  modified_base 3..4: note = thymine; mod_base = OTHER
  modified_base 15: mod_base = OTHER; note = thymine
  modified_base 20: note = thymine; mod_base = OTHER
gattgacaaaaggctgcggtcgg

SEQ ID NO: 348 - RNA, 23 nt, synthetic construct
  source 1..23: mol_type = other RNA
  modified_base 16..17: mod_base = OTHER; note = thymine
  modified_base 12: mod_base = OTHER; note = thymine
  modified_base 4: note = thymine; mod_base = OTHER
  modified_base 8: note = thymine; mod_base = OTHER
caataaatgggtaccttgacagg

SEQ ID NO: 349 - RNA, 23 nt, synthetic construct
  source 1..23: mol_type = other RNA
cctaggatcgaatgacagcgcgg

SEQ ID NO: 350 - RNA, 23 nt, synthetic construct
  source 1..23: mol_type = other RNA
aacgggctgagagaacgaggagg

SEQ ID NO: 351 - RNA, 23 nt, synthetic construct
  source 1..23: mol_type = other RNA
agcaatcttggtgatcagggtgg

SEQ ID NO: 352 - RNA, 23 nt, synthetic construct
  source 1..23: mol_type = other RNA
cctcttcctcgagagctctgcgg

SEQ ID NO: 353 - RNA, 23 nt, synthetic construct
  source 1..23: mol_type = other RNA
caggtcgacatcctcgacgacgg

SEQ ID NO: 354 - RNA, 23 nt, synthetic construct
  source 1..23: mol_type = other RNA
cgcgcaactagccagcagagcgg

SEQ ID NO: 355 - RNA, 23 nt, synthetic construct
  source 1..23: mol_type = other RNA
tttagagagagagagagacgggg

SEQ ID NO: 356 - RNA, 23 nt, synthetic construct
  source 1..23: mol_type = other RNA
caagaacactaagagaacaaagg

SEQ ID NO: 357 - RNA, 23 nt, synthetic construct
  source 1..23: mol_type = other RNA
tcatctccgactgcacacagggg

SEQ ID NO: 358 - RNA, 23 nt, synthetic construct
  source 1..23: mol_type = other RNA
agatcagatgcaattaaccgcgg

SEQ ID NO: 359 - RNA, 28 nt, synthetic construct
  source 1..28: mol_type = other RNA
tttatgcagcggggggccgggagcagtg

SEQ ID NO: 360 - RNA, 28 nt, synthetic construct
  source 1..28: mol_type = other RNA
ttttgtttttggttgggaggccacaagc

SEQ ID NO: 361 - RNA, 28 nt, synthetic construct
  source 1..28: mol_type = other RNA
tttgttctccgccgccgtccacgcgtct

SEQ ID NO: 362 - RNA, 28 nt, synthetic construct
  source 1..28: mol_type = other RNA
tttgattcttctcccctaaatactcacg

SEQ ID NO: 363 - RNA, 28 nt, synthetic construct
  source 1..28: mol_type = other RNA
ttttcttgaaaattgggttcaacggatg

SEQ ID NO: 364 - RNA, 28 nt, synthetic construct
  source 1..28: mol_type = other RNA
tttcttgaaaattgggttcaacggatgg

SEQ ID NO: 365 - RNA, 28 nt, synthetic construct
  source 1..28: mol_type = other RNA
ttttgttgttagcgttaccaagatcata

SEQ ID NO: 366 - RNA, 28 nt, synthetic construct
  source 1..28: mol_type = other RNA
tttgttgttagcgttaccaagatcatag

SEQ ID NO: 367 - RNA, 28 nt, synthetic construct
  source 1..28: mol_type = other RNA
tttacgacgtgaccggctccgactccaa

SEQ ID NO: 368 - RNA, 28 nt, synthetic construct
  source 1..28: mol_type = other RNA
tttcctcgcaagaccacgtcctcatcca

SEQ ID NO: 369 - RNA, 28 nt, synthetic construct
  source 1..28: mol_type = other RNA
tttcaagctctcctcccctcttcctacc

SEQ ID NO: 370 - RNA, 28 nt, synthetic construct
  source 1..28: mol_type = other RNA
ttttgattattctgctggtctcgtctga

SEQ ID NO: 371 - RNA, 28 nt, synthetic construct
  source 1..28: mol_type = other RNA
ttttgagtggtgtatctgcccctggcct

SEQ ID NO: 372 - RNA, 28 nt, synthetic construct
  source 1..28: mol_type = other RNA
tttgagtggtgtatctgcccctggcctt

SEQ ID NO: 373 - RNA, 28 nt, synthetic construct
  source 1..28: mol_type = other RNA
tttggggacgggaggggaaggtctgctt

SEQ ID NO: 374 - RNA, 28 nt, synthetic construct
  source 1..28: mol_type = other RNA
ttttggggacgggaggggaaggtctgct

SEQ ID NO: 375 - RNA, 28 nt, synthetic construct
  source 1..28: mol_type = other RNA
tttgctgtgcataggtagtacatacatc

SEQ ID NO: 376 - RNA, 28 nt, synthetic construct
  source 1..28: mol_type = other RNA
tttcatttcatccatcagcaggtggtgt

SEQ ID NO: 377 - RNA, 28 nt, synthetic construct
  source 1..28: mol_type = other RNA
ttttcatcacaggatcgatatggtgaca

SEQ ID NO: 378 - RNA, 28 nt, synthetic construct
  source 1..28: mol_type = other RNA
tttcatcacaggatcgatatggtgacag

SEQ ID NO: 379 - RNA, 28 nt, synthetic construct
  source 1..28: mol_type = other RNA
tttgccttgtggtcatcttcggggtcat

SEQ ID NO: 380 - RNA, 28 nt, synthetic construct
  source 1..28: mol_type = other RNA
tttggaacaagcccatctgagtctttta

SEQ ID NO: 381 - RNA, 28 nt, synthetic construct
  source 1..28: mol_type = other RNA
tttttccgcagaccactccaaagcaacc

SEQ ID NO: 382 - RNA, 28 nt, synthetic construct
  source 1..28: mol_type = other RNA
ttttccgcagaccactccaaagcaaccg

SEQ ID NO: 383 - RNA, 28 nt, synthetic construct
  source 1..28: mol_type = other RNA
tttgcgcagagaagaatcggcgaccgga

SEQ ID NO: 384 - RNA, 28 nt, synthetic construct
  source 1..28: mol_type = other RNA
ttttgcgcagagaagaatcggcgaccgg

SEQ ID NO: 385 - RNA, 28 nt, synthetic construct
  source 1..28: mol_type = other RNA
tttgcggttcttggcttggcctcctggt

SEQ ID NO: 386 - RNA, 28 nt, synthetic construct
  source 1..28: mol_type = other RNA
tttctttaatggagcgagggtgagccta

SEQ ID NO: 387 - RNA, 28 nt, synthetic construct
  source 1..28: mol_type = other RNA
ttttgcaggagagaagacgttgttctcc

SEQ ID NO: 388 - RNA, 28 nt, synthetic construct
  source 1..28: mol_type = other RNA
tttgcaggagagaagacgttgttctccg

SEQ ID NO: 389 - RNA, 28 nt, synthetic construct
  source 1..28: mol_type = other RNA
tttctgcctaggtctgattcttccaaga

SEQ ID NO: 390 - RNA, 28 nt, synthetic construct
  source 1..28: mol_type = other RNA
tttcgccagctgctgccgggaacgtgaa

SEQ ID NO: 391 - RNA, 28 nt, synthetic construct
  source 1..28: mol_type = other RNA
tttgccccgtggccagctccgcctcgcc

SEQ ID NO: 392 - RNA, 28 nt, synthetic construct
  source 1..28: mol_type = other RNA
tttcctgatccgtactcctctctggctc

SEQ ID NO: 393 - RNA, 28 nt, synthetic construct
  source 1..28: mol_type = other RNA
tttcgaagcgtgtgaatccggtgggcga

SEQ ID NO: 394 - RNA, 28 nt, synthetic construct
  source 1..28: mol_type = other RNA
tttacctgtttcctttggcgcgacctcg

SEQ ID NO: 395 - RNA, 28 nt, synthetic construct
  source 1..28: mol_type = other RNA
tttgttcttaaggccggcaataatttta

SEQ ID NO: 396 - RNA, 28 nt, synthetic construct
  source 1..28: mol_type = other RNA
tttttttaaatgcaaataggtgaggcta

SEQ ID NO: 397 - RNA, 28 nt, synthetic construct
  source 1..28: mol_type = other RNA
ttttatgtcttctcagtaagccgccagt

SEQ ID NO: 398 - RNA, 28 nt, synthetic construct
  source 1..28: mol_type = other RNA
tttatgtcttctcagtaagccgccagta

SEQ ID NO: 399 - RNA, 28 nt, synthetic construct
  source 1..28: mol_type = other RNA
tttggcatctactcactgtttgttctgt

SEQ ID NO: 400 - RNA, 28 nt, synthetic construct
  source 1..28: mol_type = other RNA
tttcgatcggaggcacattcggtgtagg

SEQ ID NO: 401 - RNA, 28 nt, synthetic construct
  source 1..28: mol_type = other RNA
tttcggtaggtggtgccgtcgtcctcga

SEQ ID NO: 402 - RNA, 28 nt, synthetic construct
  source 1..28: mol_type = other RNA
tttcggtgtaggtgatgattctgttgtt

SEQ ID NO: 403 - RNA, 28 nt, synthetic construct
  source 1..28: mol_type = other RNA
tttacttcagcttttattctttgcatat

SEQ ID NO: 404 - RNA, 28 nt, synthetic construct
  source 1..28: mol_type = other RNA
ttttattctttgcatatatatttttagc

SEQ ID NO: 405 - RNA, 28 nt, synthetic construct
  source 1..28: mol_type = other RNA
ttttccctcccgttcacggttccactgc

SEQ ID NO: 406 - RNA, 28 nt, synthetic construct
  source 1..28: mol_type = other RNA
tttccctcccgttcacggttccactgct

SEQ ID NO: 407 - RNA, 28 nt, synthetic construct
  source 1..28: mol_type = other RNA
tttctgctttatttttttggtgacgacg

SEQ ID NO: 408 - RNA, 28 nt, synthetic construct
  source 1..28: mol_type = other RNA
ttttctgctttatttttttggtgacgac

SEQ ID NO: 409 - RNA, 28 nt, synthetic construct
  source 1..28: mol_type = other RNA
tttcttctcttcggcaagctgctgcaag

SEQ ID NO: 410 - RNA, 28 nt, synthetic construct
  source 1..28: mol_type = other RNA
tttctgtttgtttggatccgagtgagaa

SEQ ID NO: 411 - RNA, 28 nt, synthetic construct
  source 1..28: mol_type = other RNA
ttttccgtttctttgcttggcctgtggc

SEQ ID NO: 412 - RNA, 28 nt, synthetic construct
  source 1..28: mol_type = other RNA
tttccgtttctttgcttggcctgtggcg

SEQ ID NO: 413 - RNA, 28 nt, synthetic construct
  source 1..28: mol_type = other RNA
tttggtacgccttgcttgtgcagctccg

SEQ ID NO: 414 - RNA, 28 nt, synthetic construct
  source 1..28: mol_type = other RNA
ttttggtacgccttgcttgtgcagctcc

SEQ ID NO: 415 - RNA, 28 nt, synthetic construct
  source 1..28: mol_type = other RNA
tttcccttcttccacgtctgcgcacgct

SEQ ID NO: 416 - RNA, 28 nt, synthetic construct
  source 1..28: mol_type = other RNA
ttttcgccggcaaaggctcctccacagc

SEQ ID NO: 417 - RNA, 28 nt, synthetic construct
  source 1..28: mol_type = other RNA
tttatagcagacgagggaaaaaactgga

SEQ ID NO: 418 - RNA, 28 nt, synthetic construct
  source 1..28: mol_type = other RNA
ttttttccctcgtctgctataaatggag

SEQ ID NO: 419 - RNA, 28 nt, synthetic construct
  source 1..28: mol_type = other RNA
tttctcagcttctctccggttccttcct

SEQ ID NO: 420 - RNA, 28 nt, synthetic construct
  source 1..28: mol_type = other RNA
tttcgcgtagaagaatcggcgaccggaa

SEQ ID NO: 421 - RNA, 28 nt, synthetic construct
  source 1..28: mol_type = other RNA
tttgatgaggccaatggccagcaatggc

SEQ ID NO: 422 - RNA, 28 nt, synthetic construct
  source 1..28: mol_type = other RNA
tttggagggaagaagggcgagggtgcgg

SEQ ID NO: 423 - RNA, 28 nt, synthetic construct
  source 1..28: mol_type = other RNA
tttctggaagtcgttcttgagctcattg

SEQ ID NO: 424 - RNA, 28 nt, synthetic construct
  source 1..28: mol_type = other RNA
tttgcgcagcgcgcggtgtcagcgacgc

SEQ ID NO: 425 - RNA, 28 nt, synthetic construct
  source 1..28: mol_type = other RNA
tttggccaccatgaggcatcagaggccc

SEQ ID NO: 426 - RNA, 28 nt, synthetic construct
  source 1..28: mol_type = other RNA
tttgcactactacctaaccaccaccacc

SEQ ID NO: 427 - RNA, 28 nt, synthetic construct
  source 1..28: mol_type = other RNA
tttatcgctccgaattcctctattcggt

SEQ ID NO: 428 - RNA, 28 nt, synthetic construct
  source 1..28: mol_type = other RNA
tttgcagaaaagccccttctttatcgct

SEQ ID NO: 429 - RNA, 28 nt, synthetic construct
  source 1..28: mol_type = other RNA
tttcccttgaacaaaagaaaattagctg

SEQ ID NO: 430 - RNA, 28 nt, synthetic construct
  source 1..28: mol_type = other RNA
ttttcttttgttcaagggaaaggacagc

SEQ ID NO: 431 - RNA, 28 nt, synthetic construct
  source 1..28: mol_type = other RNA
ttttggagcaaaaagggataatgcaggg

SEQ ID NO: 432 - RNA, 28 nt, synthetic construct
  source 1..28: mol_type = other RNA
tttggagcaaaaagggataatgcagggg

SEQ ID NO: 433 - RNA, 28 nt, synthetic construct
  source 1..28: mol_type = other RNA
tttaaagccgccgggtaacggaaagacc

SEQ ID NO: 434 - RNA, 28 nt, synthetic construct
  source 1..28: mol_type = other RNA
tttccgttacccggcggctttaaaccct

SEQ ID NO: 435 - RNA, 28 nt, synthetic construct
  source 1..28: mol_type = other RNA
tttagcggcagcttcgaactggggggtg

SEQ ID NO: 436 - RNA, 28 nt, synthetic construct
  source 1..28: mol_type = other RNA
tttagggtttagcggcagcttcgaactg

SEQ ID NO: 437 - RNA, 28 nt, synthetic construct
  source 1..28: mol_type = other RNA
ttttttttttgaaatcttaagccctgtg

SEQ ID NO: 438 - RNA, 28 nt, synthetic construct
  source 1..28: mol_type = other RNA
tttttttttgaaatcttaagccctgtgt

SEQ ID NO: 439 - RNA, 28 nt, synthetic construct
  source 1..28: mol_type = other RNA
tttggtgaacgccggtgggatttcttag

SEQ ID NO: 440 - RNA, 28 nt, synthetic construct
  source 1..28: mol_type = other RNA
tttcgttgcatctctttgtataatactg

SEQ ID NO: 441 - RNA, 28 nt, synthetic construct
  source 1..28: mol_type = other RNA
ttttaggccagaataaactccagccact

SEQ ID NO: 442 - RNA, 28 nt, synthetic construct
  source 1..28: mol_type = other RNA
tttaggccagaataaactccagccactt

SEQ ID NO: 443 - RNA, 28 nt, synthetic construct
  source 1..28: mol_type = other RNA
tttcacctgaactgaacagcgcctcctc

SEQ ID NO: 444 - RNA, 28 nt, synthetic construct
  source 1..28: mol_type = other RNA
ttttgccttgttctgggtggggtctctc

SEQ ID NO: 445 - RNA, 28 nt, synthetic construct
  source 1..28: mol_type = other RNA
tttttctacttacatatgtgtgccacag

SEQ ID NO: 446 - RNA, 28 nt, synthetic construct
  source 1..28: mol_type = other RNA
ttttctacttacatatgtgtgccacagt

SEQ ID NO: 447 - RNA, 28 nt, synthetic construct
  source 1..28: mol_type = other RNA
tttcgtcgacatcagcatccaccatagc

SEQ ID NO: 448 - RNA, 28 nt, synthetic construct
  source 1..28: mol_type = other RNA
tttgaccccacccgctcccgagaagctt

SEQ ID NO: 449 - RNA, 28 nt, synthetic construct
  source 1..28: mol_type = other RNA
tttcgcgggctcgctcgcttatatagac

SEQ ID NO: 450 - RNA, 28 nt, synthetic construct
  source 1..28: mol_type = other RNA
tttatttctgcgtgatgttggtacgggg

SEQ ID NO: 451 - RNA, 28 nt, synthetic construct
  source 1..28: mol_type = other RNA
tttccgtcgccacctaccacgtgaacct

SEQ ID NO: 452 - RNA, 28 nt, synthetic construct
  source 1..28: mol_type = other RNA
tttcagcacacacgcgacaggaaggttc

SEQ ID NO: 453 - RNA, 28 nt, synthetic construct
  source 1..28: mol_type = other RNA
tttgtgttttgtggttaatgcagacatt

SEQ ID NO: 454 - RNA, 28 nt, synthetic construct
  source 1..28: mol_type = other RNA
tttacaatgtctgcattaaccacaaaac

SEQ ID NO: 455 - RNA, 28 nt, synthetic construct
  source 1..28: mol_type = other RNA
ttttcacttgaagtttgacgatgcacaa

SEQ ID NO: 456 - RNA, 28 nt, synthetic construct
  source 1..28: mol_type = other RNA
tttgtgcaaattatagaaaagaagagca

SEQ ID NO: 457 - RNA, 28 nt, synthetic construct
  source 1..28: mol_type = other RNA
ttttgagtgagtcggcgatgaatctcac

SEQ ID NO: 458 - RNA, 28 nt, synthetic construct
  source 1..28: mol_type = other RNA
tttgagtgagtcggcgatgaatctcacg

SEQ ID NO: 459 - RNA, 28 nt, synthetic construct
  source 1..28: mol_type = other RNA
ttttcaaatggccattttccgcaatcag

SEQ ID NO: 460 - RNA, 28 nt, synthetic construct
  source 1..28: mol_type = other RNA
tttcaaatggccattttccgcaatcaga

SEQ ID NO: 461 - RNA, 28 nt, synthetic construct
  source 1..28: mol_type = other RNA
tttgactagtgaaagttgctttagtcat

SEQ ID NO: 462 - RNA, 28 nt, synthetic construct
  source 1..28: mol_type = other RNA
tttagtcattttactaagtaagaacgac

SEQ ID NO: 463 - RNA, 28 nt, synthetic construct
  source 1..28: mol_type = other RNA
tttggtgggggcactgtgagccgcaccg

SEQ ID NO: 464 - RNA, 28 nt, synthetic construct
  source 1..28: mol_type = other RNA
tttctctgctgtggtctttcttcctcgc

SEQ ID NO: 465 - RNA, 28 nt, synthetic construct
  source 1..28: mol_type = other RNA
tttggacgttcttgctcttattcttgtt

SEQ ID NO: 466 - RNA, 28 nt, synthetic construct
  source 1..28: mol_type = other RNA
ttttggacgttcttgctcttattcttgt

SEQ ID NO: 467 - RNA, 28 nt, synthetic construct
  source 1..28: mol_type = other RNA
tttctgggggcgaagggaggggagggga

SEQ ID NO: 468 - RNA, 28 nt, synthetic construct
  source 1..28: mol_type = other RNA
tttccatttccttgcccttgccagccag

SEQ ID NO: 469 - RNA, 28 nt, synthetic construct
  source 1..28: mol_type = other RNA
tttctggaggaggcaaccggagggacta

SEQ ID NO: 470 - RNA, 28 nt, synthetic construct
  source 1..28: mol_type = other RNA
tttcaaggggcggaggttggtatcattt

SEQ ID NO: 471 - RNA, 28 nt, synthetic construct
  source 1..28: mol_type = other RNA
tttgcttcgttgcaggcaggcccgtcca

SEQ ID NO: 472 - RNA, 28 nt, synthetic construct
  source 1..28: mol_type = other RNA
tttgagggctcgtcgtctaaggatcctg

SEQ ID NO: 473 - RNA, 28 nt, synthetic construct
  source 1..28: mol_type = other RNA
tttcagcctcccctccctcacaagctct

SEQ ID NO: 474 - RNA, 28 nt, synthetic construct
  source 1..28: mol_type = other RNA
tttcctagcccaatataatgaggggtgg

SEQ ID NO: 475 - RNA, 28 nt, synthetic construct
  source 1..28: mol_type = other RNA
tttgtgttccagagatcttcgtcgttct

SEQ ID NO: 476 - RNA, 28 nt, synthetic construct
  source 1..28: mol_type = other RNA
tttggtaggttggtggaagggcgaggca

SEQ ID NO: 477 - RNA, 28 nt, synthetic construct
  source 1..28: mol_type = other RNA
tttttttttaccgcacccctgtgtgcag

SEQ ID NO: 478 - RNA, 28 nt, synthetic construct
  source 1..28: mol_type = other RNA
ttttttttaccgcacccctgtgtgcagt

(19) United States
(12) Patent Application Publication
(10) Pub. No.: US 2026/0357281 A1
(43) Pub. Date: Nov. 12, 2026

(54) NUE REGULONS CONSERVED MODEL-TO-CROP ENHANCE MACHINE LEARNING PREDICTIONS OF NITROGEN USE EFFICIENCY IN LAB AND FIELD

(71) Applicant: New York University, New York, NY (US)

(72) Inventors: Ji Huang, New York, NY (US); Tim Jeffers, New York, NY (US); Amari Hill, New York, NY (US); Gloria Coruzzi, New York, NY (US); Chia-Yi Cheng, Taipei (TW)

(21) Appl. No.: 19/658,242

(22) Filed: Apr. 24, 2026

Related U.S. Application Data
(63) Continuation-in-part of application No. 17/818,968, filed on Aug. 10, 2022.
(60) Provisional application No. 63/232,060, filed on Aug. 11, 2021.
(60) Provisional application No. 63/793,851, filed on Apr. 24, 2025.

(51) Int. Cl. C12N 15/82; C07K 14/415

(52) U.S. Cl. CPC C12N 15/8262; C07K 14/415

(57) ABSTRACT

Described herein are methods and compositions for identifying and validating model-to-crop GRN regulons that control nitrogen use efficiency (NUE). Fine-scale time-course nitrogen response transcriptome analyses in maize and Arabidopsis revealed a conserved temporal nitrogen-response cascade, which was used to infer time-based transcription factor (TF)—target interactions in nitrogen-regulated GRNs. Direct targets of selected maize TFs were experimentally validated, enabling pruning of high-confidence regulatory edges. Machine-learning models trained on conserved nitrogen-responsive genes were used to rank TF regulons associated with NUE. Conserved regulons improved prediction of NUE traits across species. The disclosure further provides methods for modifying expression of identified TFs to enhance NUE in plants.

### CROSS-REFERENCE TO RELATED APPLICATIONS

This application claims priority to U.S. provisional application No. 63/793,851, filed Apr. 24, 2025, and this application is also a continuation-in-part of U.S. patent application Ser. No. 17/818,968, filed Aug. 10, 2022, which claims priority to U.S. provisional application No. 63/232,060, filed Aug. 11, 2021. the entire disclosures of each of which are incorporated herein by reference.

### STATEMENT REGARDING FEDERALLY SPONSORED RESEARCH

This invention was made with government support under 1339362 awarded by the National Science Foundation. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 17, 2026, is named “058636.00870.xml” and is 428,915 bytes in size. The sequence listing contained in this .XML file is part of the specification and is hereby incorporated by reference herein in its entirety.

### RELATED INFORMATION

Improving agronomic traits in staple crops like maize is essential to meet the demands of a growing global population. Identifying key genes in crops that influence these traits such as nitrogen use efficiency (NUE) is important to both agriculture and the environment. In the age of genomics, gene regulatory networks (GRNs) and machine learning are two promising new approaches for this task. However, these approaches have been difficult to navigate and integrate in large and complex genomes of crop plants and even more challenging to functionally validate.

By contrast, GRNs inference and machine learning approaches can generate hypothesis to test in the well-studied model species Arabidopsis. This includes identifying genes of importance to a trait using machine learning and inferring their regulatory connections via network inference (e.g. TF→gene→trait).

Machine learning methods can successfully identify key genes important for traits using the increasing amount of omics data (Ma et al. 2014; Cheng et al. 2021). At the same time, GRNs provide a systematic approach to improving agronomic traits by elucidating transcription factor (TF)-to-gene regulation (Krouk et al. 2013; Springer et al. 2019). However, the challenge remains in transferring the GRN knowledge from model-to-crop and applying the findings in field settings (Ferrier et al. 2011; Liu et al. 2022). There is accordingly an ongoing and unmet need for improved methods of identifying genes and regulator proteins such as transcription factors that contribute to NUE, modifying such genes and proteins to improve NUE and other growth properties of plants. The present disclosure is pertinent to these needs.

### BRIEF SUMMARY

This disclosure demonstrates, among other aspects, integration of GRNs with machine learning and model-to-crop orthology mapping to enhance nitrogen use efficiency (NUE), a key factor for maize yield (Lassaletta et al. 2014) and environmental sustainability (Zhang et al. 2015; Gao and Cabrera Serrenho 2023).

This disclosure applies and integrates machine learning and network inference to generate and test hypotheses for TF→gene(s)→trait to show how nitrogen signaling affects the trait NUE. Previous GRN studies were conducted in the model species Arabidopsis, for which N-signaling and its links to genes involved in N-uptake & assimilation have been well-studied. An aspect of this disclosure includes identification and validation of high-confidence N-GRNs controlling NUE using fine-scale time-series data in maize, as we did for Arabidopsis (Varala 2018, Brooks 2019). The cross-species comparison enables identification of N-regulatory network modules conserved across a C3-C4 divide. In non-limiting aspects the disclosure includes but is not limited to i) identification and validation of high-quality nitrogen gene regulatory networks (N-GRNs) maize, ii) identification of N-responsive gene orthologs in maize and Arabidopsis that control NUE traits using machine learning, iii) functional validation of conserved N-regulons (TF-to-target modules) (Aibar et al. 2017; Guillotin et al. 2023) whose expression can predict for the NUE trait in each species. In an example, the disclosure provides a model-to-crop study that provides inferred time-based N-GRNs in maize (shoot and root) using RNA from a fine time-scale N-response experimental design that adapts in part a study in Arabidopsis (Varala et al. 2018; Brooks et al. 2019). Using a Just-In-Time (JIT) analysis of this data enabled us to identify a temporal cascade of N-responses conserved between model-to-crop. The disclosure reveals that this time-series data allowed identification of a time-based N-response GRN in maize, which we validated and refined using precision/recall analysis. We did this by generating data for 23 maize TF whose genome-wide targets were validated using the cell-based Transient Assay Reporting Genome-wide Effects of Transcription factors (TARGET) assay (Bargmann et al. 2013; Brooks et al. 2019, from which the entire description is incorporated herein by reference), modified for maize. To identify the network modules of importance to NUE, we integrated high-confidence N-GRNs with machine learning feature importance values into a NUE Regulon score. This enabled us to identify and validate an NUE Regulon conserved model-to-crop (AtDIV1 and ZmMYB34/R3) that can accurately predict NUE trait in left out samples. Thus, the described approach for identifying cross-species NUE Regulons reveals genes that can be modified for NUE improvement via transgenics, genome-editing or molecular breeding. This disclosure thus describes an integrated system-biology approach that combines time-resolved gene regulatory networks, machine learning, and model-to-crop gene conservation to identify transcription factors that control NUE in maize. Using fine-scale nitrogen response data and cross-species comparisons with Arabidopsis, the disclosure defines conserved regulatory “NUE regulons” whose gene expression accurately predicts field-level NUE. The approach prioritizes key transcription factors, validates their regulatory targets experimentally, and provides methods for modifying these genes—through breeding, transgenics, or genome editing—to improve crop performance under low-nitrogen conditions.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. A temporal cascade of N-responsive “Just-in-Time” genes uncovers N-related GO process conserved between maize and Arabidopsis. N-treatment (+N/−N) of maize was used to monitor ¹⁵N-uptake (A & B) and RNA-seq data collected across a fine-scale time-course (5 min-2 hrs) (C-E) in shoots and roots (see Methods). Maize (A) shoot and (B) root tissues exhibit a linear increase in total N-uptake, as detected by ¹⁵N analysis. Maize genes regulated by N as a function of time (NxTime genes) were identified by Spline modeling of RNA-seq N-response curves, compared to controls (see Methods). Each NxTime gene was assigned to a Just-in-Time (JIT) bin, based on the first time-point that N-response was >2-fold (see Methods). (C) An example maize NxTime gene, ZmGLN2 (Zm00001d026501) encoding a cytosolic glutamine synthetase, was assigned to the 20 min JIT bin. (D) All maize NxTime genes were chronologically arranged into JIT bins spanning early (5 min) to late (120 min) time-points. For a model-to-crop comparison, this same NxTime and JIT analysis pipeline was performed on RNA-seq data from Arabidopsis exposed to an identical time-series N-treatment (Varala et al. 2018). JIT bins for each species show a cascade of GO term enrichment (p-values <0.05) highlighted in blue. (E) Key GO terms shared by JIT bins in maize and Arabidopsis are categorized by tissue specificity: shared in shoots, roots, or both tissues. These shared GO terms include nitrogen assimilation and processes in N-signaling, including hormone responses.

FIG. 2. Direct regulated targets of 23 maize TFs validated in the cell-based TARGET assay are enriched in NxTime genes identified in shoots or roots of maize plants. (A) 23 NxTime TFs, spanning early to late JIT bins in shoots or roots were validated by adapting the medium through-put cell-based TF-TARGET assay developed in Arabidopsis (Brooks et al. 2019) (B-C) Validated TF-target genes of 23 TFs in maize cells show significant overlap with NxTime genes in (B) shoots or (C) roots of whole maize plants, with darker colors indicating a more significant overlap with whole tissue N-responses, as determined by Fisher Exact Tests. Highlighted examples include: NLP3 whose direct targets validated in maize cells are highly enriched in NxTime genes identified in both shoots and roots of maize seedlings (black links); MYB21 whose direct targets validated in maize cells are primarily enriched in shoot NxTime genes of maize seedlings (green links); HB54 whose direct targets validated in maize cells are primarily associated with root NxTime genes in maize seedlings (brown links). (D) Collectively, the 23 maize NxTime TFs validated via the TARGET assay in maize cells directly regulate ~85-90% of NxTime genes and N-pathway genes identified in whole maize seedlings.

FIG. 3. Generation of high-confidence time-based N-GRNs using Dynamic Factor Graph (DFG) and network pruning using TF-target validation data. (A) The DFG method uses fine-scale time-series data to predict future target gene expression based on prior expression data of target genes and transcription factors (TFs) (Krouk et al. 2010, 2013). Using left-out data to test and refine the models, DFG scores each TF's influence on target gene expression, where higher values indicate more confident TF-target edge predictions. (B) Precision-Recall (PR) analysis: maize shoot DFG network, PR performed using validated TF-target edges for 21 maize TFs in the TARGET cell-based TF assay (FIG. 2). PR analysis of the maize root DFG network performed using validated TF-target edges from 10 maize TFs using the TARGET assay (FIG. 2). (C) Area Under the Precision Recall (AUPR) Analysis: DFG-inferred networks significantly outperform random networks (p-value <0.001, permutation test; see Methods section). Precision Cutoffs: Precision cut-offs of 0.33 for shoot and 0.45 for root network were selected based on the flattening points of the PR curves, based on the validated TFs in shoots (21 TFs) and roots (10 TFs). These precision cut-offs determined from the validated TFs, were then used to prune the predicted TF-targets for ALL the TFs in shoot (203 TFs) and root (196 TFs).

FIG. 4. A temporal N-responsive “ensemble” network of the N-uptake/assimilation pathway in maize shoots. In this temporally resolved N-response network, edges connect NxTime TFs (organized by their Just-in-Time bins) with genes in the nitrogen-uptake/assimilation pathway. The “ensemble” network consists of 92 TFs. This “ensemble” network exclusively includes high-confidence TF-target edges from the DFG network (green), validated TF-target edges of 14/23 maize TF validated by the TARGET TF assay (pink), and TF-target edges confirmed by ChIP-Seq for 10 TFs (purple) (Tu et al. 2020). Maize TFs with orthologs in the Arabidopsis Yeast-One-Hybrid TF-DNA binding dataset (Gaudinier et al. 2018) or the Arabidopsis shoot NxTime gene list (Varala et al. 2018) are labeled with a black border line.

FIG. 5. “Network Walking” uncovers a novel role for KNOTTED1 (KN1) in maize nitrogen signaling. (A) A diagram depicting the Network Walking method that links KN1's direct targets to indirect in planta targets. For detailed explanation of Network Walking method see: (Brooks et al. 2019, 2021; Huang et al. 2023). (B) Hierarchical structure of the KN1 regulatory network for N-responsive genes. KN1-direct targets include validated using the TARGET system are encompassed within a pink rectangle. The dashed green rectangle within the pink rectangle denotes KN1-direct targets validated in TARGET and in plant kn1 mutant (Bolduc et al. 2012). KN1 directly regulates ten TF₂ₛ, which provide direct links to KN1's indirect targets (encompassed in green rectangle). These 10 TF₂ₛ which link KN1 to its indirect targets are validated by: (i) the maize TARGET assays (pink edges) (FIG. 2), (ii) high-confidence edges in the “pruned” DFG network (green edges) (FIG. 3), or (iii) by ChIP-Seq (purple edges) (Tu et al. 2020). The indirect targets of KN1, encompassed within in the green rectangle, consist of the overlap between in planta mis-regulated genes from the kn1 mutant (Bolduc et al. 2012) and NxTime genes in shoots. Genes are linked to their GO annotations by grey edges. GO terms found for genes mis-regulated in kn1 maize mutants in planta, appear as purple hexagons while novel nitrogen pathway related GO terms discovered for KN1 in this disclosure are shown in green hexagons.

FIG. 6. Model-to-Crop conserved NxTime genes are significantly more effective in predicting NUE outcomes maize field samples (Tables 1 & 2). Out of 353 conserved NxTime genes, 331 genes are present in the training and testing dataset from Cheng et al 2021. Using a selection of 331 conserved NxTime shoot genes, XGBoost was used to predict the maize NUE traits using RNA-seq and NUE data from 16 maize lines grown under-N/+N field conditions (Cheng et al. 2021). To evaluate the power of the XGBoost model to accurately predict the NUE trait based on the mRNA levels of the XGBoost gene models learned. To this end, Pearson's correlation (r), was calculated between XGBoost predicted NUE value vs. actual NUE value (in left out samples). (A) The r value was calculated for XGBoost models learned; i) using the 331 out of 353 NxTime genes conserved model-to-crop, ii) using an equivalent number of random maize NxTime genes, and iii) the equivalent number of randomly expressed maize genes. P-values derived from a Wilcoxon signed-rank test, show that the NxTime genes conserved model-to-crop are significantly more effective at predicting NUE in field grown maize. (B) Using genes from the 331 conserved NxTime genes in XGBoost to learn gene models predictive of NUE, significantly improves NUE predictions, compared to using an equivalent number of random genes in maize. ***: p-value <0.001 (Wilcoxon signed-rank test). The XG-Boost scores for these 331 maize genes-of-importance to NUE are listed in Tables 1 & 2. Table 1 lists the XGBoost scores of the 34 maize TFs-of-importance to NUE. Table 2 lists the XGBoost scores of non TFs of importance to NUE (319 genes).

FIG. 7. Maize NUE Regulon integrates network connectivity with machine learning scores to prioritize TFs of importance to NUE (Table 3). (A) NUE Regulon's architecture integrates TF-target edges from pruned high-confidence DFG time-based networks (Figure. 3), maize TF-targets validated in maize cells via TARGET (FIG. 2), and

ChIP-Seq data for two maize TFs (Tu et al. 2020). Using these network edges, the NUE Regulon score for each TF was calculated as the product of the TF's own NUE score times the sum of all its targets NUE scores. (B) TFs within NUE-centric subnetwork are ranked by their NUE Regulon scores (Table 3). The Arabidopsis orthologs of the top ranked maize TFs are listed in the last column of FIG. 7. The top-rank maize TFs, ZmMYBR3, ZmMYB34, and their ortholog AtDIV1 have been validated for TF-target genes using the maize cell-based TARGET system (FIG. 8).

FIG. 8. A model-to-crop conserved NUE Regulon significantly enhances predictions of NUE in both maize and Arabidopsis (Tables 4 & 5). (A) Discovery of the conserved NUE Regulon shows that the orthologous TFs in maize (ZmMYB34/R3) and Arabidopsis (AtDIV1) and share a significant number of orthologous targets, validated by the plant cell-based TARGET assay. This TF network regulon includes 24 gene targets in maize and 23 orthologous gene targets in Arabidopsis, identified by overlapping genes in the conserved orthogroups with Arabidopsis or maize NxTime genes. (B-C) Using genes from these cross-species NUE regulon in XGBoost to learn gene models predictive of NUE, significantly improves NUE predictions, compared to using an equivalent number of conserved NxTime genes in (B) maize and (C) Arabidopsis. ***: p-value <0.001 (Wilcoxon signed-rank test). Table 4 lists the XGBoost scores of the TF-NUE Regulon genes validated in the TARGET TF plant cell assay in each species, maize and Arabidopsis. Table 5 lists the 25 genes in the ZmMYB34/R3 Regulon validated in the TARGET assay in maize cells and the in planta validated functions if available.

FIG. 9. Summary figure of NUE Regulon.

FIG. 10. Gene expression principal component analysis (PCA) shows that PC1 is associated with nitrogen treatment, and PC2 is associated with time in the maize shoot and root NxTime dataset. (A) Timeline of the experiment. (B) Details of treatment and sample collection times. (C) Eight-day-old maize seedlings before and after endosperm removal. (D) PCA plot of gene expression data from maize shoot NxTime samples. (E) PCA plot of gene expression data from maize root NxTime samples. (F-G) Correlation of principal components (PCs) with experimental variables in maize (F) shoot and (G) root NxTime data. Variables include Ntreatment (Nitrogen-treated or KCl control), time (sampling from 5 min to 120 min), and replication (biological replicate 1, 2, or 3). Pearson correlation coefficients (PCC) were calculated between PCs and the three variables.

FIG. 11. Analysis pipeline for identifying NxTime genes in the maize data. (A) Flowchart illustrating the analysis of the maize NxTime dataset. (B) Venn diagram displaying the number of maize shoot and root NxTime genes and their overlaps.

FIG. 12. The maize NxTime genes in shoot and root not only have significant overlap with published datasets, but also identify new N responsive genes. (A) Overlap heatmap of NxTime genes with six N-response datasets in maize. The p-values and the odds ratios were calculated using Fisher exact test in R. The p-values were then adjusted by ‘p.adjust(method=“BH”)’ function in R. (B) Detailed experimental set up for the eight N-response datasets: “NxT_shoot” and “NxT root” (this disclosure); “Wang_nitrate” (Wang et al. 2020); “Ge_nitrate” (Ge et al. 2020); “Monsanto_leaf” (Yang et al. 2011); “Cheng_leaf” (Cheng et al. 2021); “Ammonium_root” (Buoso et al. 2021); “NGWAS” (Xing et al. 2024). (C) Percentage of NxTime root genes and their Just-In-Time categories that are not found in Wang et al, 2020. (D) Percentage of NxTime root genes and their Just-In-Time categories that are not found in Ge et al, 2020. (E) Percentage of NxTime root genes and their Just-In-Time categories that are not found in Buoso et al, 2021. (F) Percentage of NxTime root genes and their Just-In-Time categories that are not found in Cheng et al, 2021.

FIG. 13. The functional nitrogen pathway genes in maize show significant activation by nitrogen treatment. (A-B) Density plots of log 2 fold change (log 2FC) for nitrogen treatment versus control (+N/−N) in maize (A) shoot and (B) root, highlighting the significant activation of N-pathway genes. All NxTime genes in shoots or roots are colored gray, while N-pathway genes are highlighted in green. P-values for each density plot were calculated using the Kolmogorov-Smirnov test in R. (C-D) Heatmap of gene expression log 2FC (+N/−N), showing the timely activation of nitrogen metabolism pathway components. Time is displayed from T5 (5 min) to T120 (120 min) across columns, with genes' log 2FC plotted by rows. Nitrogen pathway genes are annotated and color-coded into five distinct components.

FIG. 14. Sixteen out of twenty-six maize root nitrogen pathway genes show a positive correlation with 15N uptake. The Pearson correlation coefficient was calculated using the ‘cor.test( )’ function in R, and p-values were adjusted using the ‘p.adjust (method=“BH”)’ function in R.

FIG. 15. The identified maize seedling NxTime genes in this disclosure also express in adult tissues. (A) Expression heatmap of shoot NxTime genes throughout leaf developmental stages. Columns are ordered from seedling to adult stages. The expression of 2,732 shoot NxTime genes was clustered into seven groups using K-means clustering. (B) Expression heatmap of root NxTime genes throughout root developmental stages, with columns ordered from seedling to adult stages. The expression of 2,294 root NxTime genes was clustered into eight groups using K-means clustering. N-pathway genes are labeled on the right of each heatmap and color-coded by function. Gene expression matrices (FPKM) were retrieved from qTeller (Woodhouse et al. 2021) and normalized using z-scores.

FIG. 16. Enriched GO terms for maize (A) shoot and (B) root JIT genes how a temporal cascade from early to late time points. GO terms from six time points are summarized and displayed using the GOsummaries package (Kolde and Vilo, 2015). Terms are visualized in red rectangles, with more enriched terms appearing in larger font sizes.

FIG. 17. The maize JIT genes are enriched in unique motif clusters similar to those in Arabidopsis JIT genes. Enriched motif clusters for the (A) maize and (B) Arabidopsis JIT genes. The 80 motif clusters were retrieved from the published dataset (Brooks et al. 2019). The motif enrichment analysis was performed using SEA from MEME suite (Bailey and Grant, 2021).

FIG. 18. The individual motif enrichment for the maize and Arabidopsis JIT genes exhibits a sequential pattern over time, including several known nitrogen regulators. Enrichment of individual TF motifs for the (A) maize and (B) Arabidopsis JIT genes. The JASPAR2022 Plant Core motifs (Castro-Mondragon et al. 2022) were tested for enrichment. (C) The motifs and the references for the experimentally verified nitrogen regulators. The motif enrichment analysis was performed by SEA from MEME suite (Bailey and Grant. 2021).

FIG. 19. In the maize NxTime dataset, the DFG method performs significantly better than random networks and has the highest AUPR values compared to the other three tested methods. Validation of maize (A) shoot and (B) root GRNs constructed by four methods, DFG (Brooks et al. 2019), GENIE3 (Huynh-Thu et al. 2010), dynGENIE3 (Huynh-Thu and Sanguinetti 2015) and Person's correlation (PCC). The maize TARGET datasets were used as gold-standard for AUPR calculation.

FIG. 20. The maize shoot DFG network has a better precision-recall curve than random networks, regardless of the validation TARGET TFs. Evaluation of the maize shoot DFG network's performance through split validation. TFs with TARGET data were divided into pruning (70%) and testing (30%) sets to assess performance. The red curve represents the precision-recall (PR) curve for the maize shoot DFG network, which significantly outperforms the best random network (highest AUPR marked as random_max). Statistical significance was confirmed with a p-value <0.001 using a permutation test. Random networks were repeated 1,000 times.

FIG. 21. The maize root DFG network has a better precision-recall curve than random networks, regardless of the validation TARGET TFs. Evaluation of the maize shoot DFG network's performance through split validation. TFs with TARGET data were divided into pruning (70%) and testing (30%) sets to assess performance. The red curve represents the precision-recall (PR) curve for the maize shoot DFG network, which significantly outperforms the best random network (highest AUPR marked as random_max). Statistical significance was confirmed with a p-value <0.001 using a permutation test. Random networks were repeated 1,000 times.

FIG. 22. Time-resolved gene regulatory network of the nitrogen pathway in maize root. Edges connect NxTime transcription factors (TFs), organized by their Just-in-Time bins, to nitrogen pathway genes. The network includes only high-confidence edges from the DFG network (green), edges validated by the TARGET system (pink), and edges confirmed by ChIP-Seq (purple) (Tu et al. 2020). TFs with orthologs in the Arabidopsis Yeast-One-Hybrid network (Gaudinier et al. 2018) or Arabidopsis root NxTime gene list (Varala et al. 2018) are labeled with a black border line.

FIG. 23. The KN1 in planta mis-regulated genes have a significant overlap with the maize NxTime shoot genes. The p-value was calculated by the Fisher's exact test in R. The in planta mis-regulated genes was retrieved from ConnecTF (Brooks et al. 2021) using data from (Bolduc et al. 2012).

FIG. 24. The protoplast KN1 BLRP-ChIP-Sep captures in planta binding and regulation. (A) Overlap between KN1 protoplast ChIP-Seq binding genes (this disclosure) and in planta ear tissue binding genes (Bolduc et al. 2012). (B) Overlap between KN1 protoplast ChIP-Seq binding genes and in planta RNA-Seq identified mis-regulated genes. (C) Protoplast ChIP-Seq binding signals are enriched at TSS region. (D) KN1 protoplast ChIP-Seq has similar binding motifs to ear ChIP-Seq.

FIG. 25. The maize TARGET dataset can be used to validate gene regulatory networks (GRNs) from other sources using AUPR analysis. (A) Forty-five GRNs (Zhou et al. 2020) were validated using genes directly regulated by the 23 TFs from TARGET results. The number of samples used for constructing each GRN is indicated in square brackets. Network AUPR values are shown as red dots, while the mean AUPR values for 1,000 random networks are displayed as gray dots. GRNs are ranked by their p-values, with the lowest p-value at the top. (B-C) Precision-recall curves for the “kremling2018_L3Tip” (B) and “kremling2018_GRoot” (C) networks as named by the Zhou et al. 2020, showing significant performance improvements over random networks.

FIG. 26. The eigengenes of conserved NxTime genes (331 out of 353) are significantly correlated with NUE traits, but not with other agronomic traits. (A) Eigengenes from the 331/353 conserved NxTime genes have significant correlation with NUE traits. (B) These eigengenes show no significant correlation with other traits. The eigengenes were calculated as the first principal component (PC) of the 331/353 genes' expression in the field leaf data from Cheng et al. 2021. The PC1 was calculated by the ‘prcomp( )’ function in R.

FIG. 27. The maize MYB34 and MYBR3 share a significant number of TARGET-validated genes, suggesting redundant functions between these two TFs. The p-value was calculated by the Fisher's exact test in R.

FIG. 28. The random forest model using 24 ZmMYB34/R3 regulon genes predicts significantly better than the same number of random conserved NxTime genes for Ear Diameter (ED) and Ear Length (EL). Other phenotypes include Ear Weight (EW), Kernel Row Number (KRN), Hundred Kernel Weight (HKW), Kernel Length (KL), Kernel Volume Weight (KVW), Kernel Thickness (KT) and Kernel With (KWD). The random gene selection was repeated 100 times (gray dots) (See Methods). The gene expression and phenotype data were from (Li et al. 2023)

FIG. 29. 35 Maize TFs have significant specificity and influence on learned genes-of-importance to NUE in field grown maize. A machine learning (ML)/double-TARGET TF-assay pipeline was used to identify & validate the targets of 35 maize TFs control genes-of-importance to NUE, and rank them based on their significance of their overlap with NUE genes-of-importance in maize for their NUE-specificity & NUE-influence, as described in the text. Panel A. Target TF validation and Bipartite Machine Learning of Field NUE; TFs (representing genes encoding the TFs) are shown in the box below the diagram. Panel B. NUT TF Prioritization diagram. Panel C. Functional Assessment of targets diagram.

### DETAILED DESCRIPTION

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example +/−10%.

This disclosure includes every amino acid sequence described herein and all nucleotide sequences encoding the amino acid sequence. Polynucleotide and amino acid sequences having from 80-99% similarity, inclusive, and including and all numbers and ranges of numbers there between, with the sequences provided here are included in the invention. All of the amino acid sequences described herein can include amino acid substitutions, such as conservative substitutions, that do not adversely affect the function of the protein that comprises the amino acid sequences. The disclosure includes all polynucleotide and amino acid sequences described herein, and every polynucleotide sequence referred to herein includes its complementary DNA sequence, and also includes the RNA equivalents thereof to the extent an RNA sequence is not given. Any sequence referred to by a database entry is incorporated herein by reference as the sequence exists in the database as of the effective filing date of this application or patent, including but not limited to database entries that are signified by an alphanumeric indicator that starts with “Zm.”

The disclosure includes all described methods of analyzing transcriptome data to predict a phenotype described herein, all machine learning approaches described herein that are used for analysis of gene expression changes using Nitrogen (N)-treatment that influences expression of N responsive genes, and extensions of those approaches to different genes, their protein products, and interspecies comparisons of transcriptome analysis and predictions of the influence of transcription factors on any phenotype.

In embodiments, based at least in part on the described analysis, the present disclosure provides compositions and methods for modifying plants and/or plant cells. The disclosure includes all types of plants and plant cells, and methods of modifying plants and plant cells, as described in US patent publication no. 20180127769, from which the description of plants, plant cells, and compositions and methods for modifying plants is incorporated herein by reference.

The compositions and methods relate to altering expression of one or a combination of the TFs.

Altering the expression can result in any change in the plant described herein. In embodiments, practicing a method of the disclosure results in an increase in N uptake, increased biomass, such as increased grain biomass, an increased harvest index, an increased Total nitrogen utilization (NUtE), an increased total Grain NUtE, or a combination thereof. Non-limiting demonstrations of these effects are as in the text and as shown in the figures. In embodiments, modification of only one, or only two, of the describes genes is sufficient to produce the described improved properties, relative to the same properties in plants that do not comprise the same modifications.

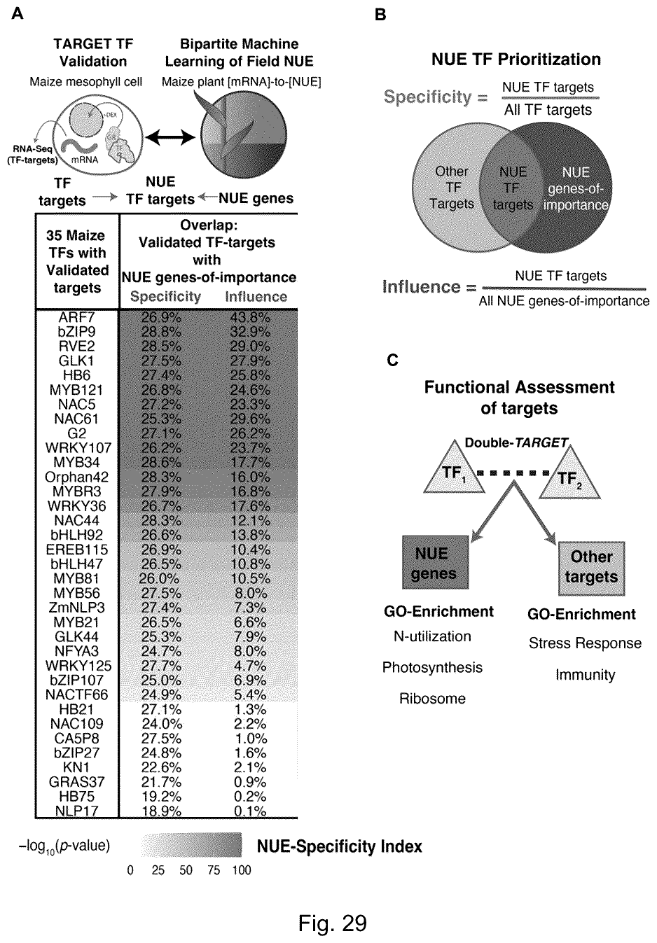

The TFs of the present disclosure include any TF that is referenced in the description (including tables) and in the figures including but not limited to those described by way of Table 1 and FIG. 29 panel A).

In examples, the disclosure includes overexpression, or disrupting or inhibiting expression of any described gene, or any combination of described genes.

In examples, the disclosure includes increasing expression of one or both of MYBR3 or MYB34.

In accordance with the invention/disclosure, means for disrupting or inhibiting expression of a TF that negatively regulates NUE in the maize plant include disruptive techniques such as gene knockouts, homologous recombination, insertions/deletions, frameshift mutations, use of RNAi agents and/or microRNAs, use of CRISPR editing enzymes or other designer nucleases, TALONS, zinc finger directed nucleases, prime editing, retron editing, and the like. In examples, a Type II CRISPR enzyme is used, such enzymes including Cas9, Cas12, and Cas13. In Examples, a Type I CRISPR enzyme is used, such as a Cas3 enzyme. A representative list of guide RNAs that can be used with CRISPR nucleases is provided in Tables 13, 14, 15, and 19. microRNAs or RNAi agents can be designed using the guide RNAs and their targets. The disrupting or inhibiting expression can involve blunt end or sticky end cleavage. In examples, one gene, or a combination of genes as described herein, may be disrupted, or their expression inhibited.

In examples, the gene that is disrupted or its expression is inhibited is at least one of the genes encoding a TF that is GLK44; G2; NFYA3; CA5P8; or KN1. (See Table 8)

In examples, a combination of genes, and optionally only a pair of genes, are disrupted or their expression is inhibited. Such combinations of genes comprise as non-limiting examples genes encoding a TF that is: G2 and GLK44; GLK44 and NFYA3; G2 and NFYA3; CA5P8 and GLK44; GLK44 and KN1; GLK44 and WRKY107; GLK44 and GRAS37; EREB115 and GLK44; CA5P8 and G2; or bHLH47 and GLK44. (See Table 10).

In accordance with the invention/disclosure, means for overexpressing a TF that positively regulates NUE in the maize plant include increasing copy number of the gene, and/or use of an introduced promoter (i.e., introducing a promoter that was not present at all or is introduced or relocated to a different location prior to modification). Representative promoters that can be used to overexpress a gene are provided in Table 20.

In examples, a gene that is overexpressed is at least one of MYB121; MYBR3; NAC61; HB6; GLK1; MYB56; WRKY36; MYB34; or NAC44; or ARF7. (See Table 7.)

In examples, a combination of genes, and optionally only a pair of genes, is overexpressed. Such combinations of genes comprise as non-limiting examples: genes encoding a TF that is MYB121 and MYBR3; MYB121 and NAC61; HB6 and MYB121; MYBR3 and NAC61; GLK1 and MYB121; HB6 and MYBR3; MYB121 and MYB56; MYB121 andWRKY36; MYB121 and MYB34; or GLK1 and MYBR3. (See Table 9.)

In examples, within a combination of genes, and optionally within only a pair of genes, at least one gene is disrupted or its expression is inhibited, and another gene is overexpressed. In representative examples, such pairs combinations include over expression of genes encoding a TF that is MYB121 and disrupting or inhibiting expression of GLK44; or over expression of MYB121 and disrupting or inhibiting expression of G2; or over expression of MYBR3 and disrupting or inhibiting expression of GLK44; or over expression of MYBR3 disrupting or inhibiting expression of G2; or over expression MYB121 and disrupting or inhibiting expression of NFYA3; or over expression of NAC61 and disrupting or inhibiting expression of GLK44; or over expression of MYB121 and disrupting or inhibiting expression of CA5P8; or over expression of MYBR3 and disrupting o inhibiting expression of NFYA3; or over expression of MYB121 and disrupting or inhibiting expression of KN1; or over expression of and MYB121 and disrupting or inhibiting expression of WRKY107. (See Table 11).

Aspects of the disclosure are illustrated by the following Statements:

Statement 1. In an example, the disclosure provides a modified maize plant, optionally grown under low nitrogen conditions, that exhibits increased nitrogen use efficiency (NUE), comprising:
(a) maize plant cells, and
(b) means for disrupting or inhibiting expression of at least one transcription factor (TF) that negatively regulates NUE in the maize plant cells and/or a means for overexpressing a TF that positively regulates NUE,
wherein, the growth under low nitrogen conditions induces increased NUE in the modified maize plant relative to its unmodified parent maize plant when grown under low nitrogen conditions.

Statement 2. The modified maize plant of Statement 1 in which the transcription factor TF is any TF of Table 1 and Fig. X, or any combination thereof.

Statement 3. The modified maize plant of Statement 1 or Statement 2, wherein the TF is MYBR3, MYB34 or a combination thereof.

Statement 4. The modified maize plant of any one of Statements 1-3, in which the modified maize plant or plant cells are a variety of maize, or wherein optionally the maize is an elite variety of maize.

Statement 5. The modified maize plant of any one of Statements 1-4, wherein the means for disrupting or inhibiting expression of TF in the modified maize plant is:
a) UniformMu transposon insertion in the 5′ untranslated region of the TF gene;
b) TF gene knockout;
c) TF frameshift mutation;
d) a microRNA that targets TF mRNA for degradation; or a clustered regularly interspaced short palindromic repeats (CRISPR) gene inactivation enzyme and a guide RNA that targets the TF mRNA for degradation.

Statement 6. The modified maize plant Statement 5 using a CRISPR Cas3, Cas9 or Cas4 based system.

Statement 7. The modified maize plant of any one of Statements 1-8 which exhibits at least one of: an increase in Nitrogen (N) uptake, increased biomass, an increased harvest index, an increased Total nitrogen utilization (NUtE), or an increased grain yield (total Grain NUtE) relative to its unmodified parent maize plant when grown under low nitrogen conditions.

Statement 8. The disclosure provides a method of growing a maize plant under conditions that induce increased NUE, comprising growing the modified maize plant of any one of Statements 1-7.

Statement 9. The disclosure provides a method for engineering a modified maize plant that exhibits increased nitrogen use efficiency (NUE) when grown under low nitrogen conditions, comprising:
a) modifying a maize plant cell using a means for disrupting or inhibiting expression of a transcription factor (TF) that negatively regulates NUE in the maize plant cells, and
b) generating a modified maize plant from the modified maize plant cells, and
c) growing the modified maize plant under low nitrogen conditions to confirm that the low nitrogen conditions induce increased NUE in the modified maize plant relative to its unmodified parent maize plant when grown under low nitrogen conditions.

Statement 9. The method of Statement 9 in which the transcription factor (TF) is MYBR3, MYB34 or a combination thereof.

Statement 10. The method of Statement 9 or Statement 10 in which the modified maize plant cells are from an elite variety of maize.

Statement 11. The method of Statement 9 or Statement10 in which the means for disrupting or inhibiting expression of the transcription factor (TF) in the modified maize plant is:
a) UniformMu transposon insertion in the 5′ untranslated region of the TF gene;
b) TF gene knockout;
c) TF frameshift mutation;
d) microRNA that targets TF mRNA for degradation; or
e) a clustered regularly interspaced short palindromic repeats (CRISPR) gene inactivation enzyme and a guide RNA that targets the TF mRNA for degradation.

Statement 12. The method of Statement 11 using a CRISPR Cas3, Cas9 or Cas4 based system.

“Low nitrogen conditions” and “reduced nitrogen conditions” as used herein means lower amounts of N-fertilizer than customarily, conventionally or ordinarily used for growing a particular crop. In examples, the disclosure includes reducing 50% or more in N-fertilizer use by, for instance, meeting the following performance targets: 1. >50% reduction in N₂O emissions relative to unmodified control; 2. >48% reduction (sorghum) or >55% reduction (corn) in N fertilizer use; 3. maintaining ≥100% yield relative with N fertilizer reduction; or 4. showing N₂O emissions reduction, N fertilizer reduction and yield maintenance over two field seasons.

In non-limiting examples, low nitrogen conditions include less nitrogen that have been previously used, for instance, in modern corn (maize) production, where nitrogen fertilizer is typically applied at rates ranging from about 120 to 280 kilograms of nitrogen per hectare (kg N/ha), depending on soil type, prior crop, yield goals, and local climate conditions. In high-yielding systems with little residual soil nitrogen, rates toward the upper end of this range are common, while fields with manure history, legumes as previous crops, or higher organic matter often require less. Thus, in examples, using a plant modified as described herein in low nitrogen conditions such as less than 120 N/ha enables any of the described features, including but not limited to an increase in Nitrogen (N) uptake, increased biomass, an increased harvest index, an increased Total nitrogen utilization (NUtE), or an increased grain yield (total Grain NUtE), relative to a maize plant that has not been modified to disrupt or inhibit expression of the nf-ya3 gene, or has any other genetic alteration described herein, or any combination thereof, relative to an unmodified of the same plant species of the modified plant when grown in the same low nitrogen conditions. In examples, previous approaches to growing high-yield maize systems involves application of approximately 1 lb of nitrogen for every bushel of expected yield, which converts to approximately 1 kg of nitrogen for every 56 kg of grain. In examples, using a plant modified as described herein in low nitrogen conditions such as less than approximately 1 kg of nitrogen for every 56 kg of grain results in one or more of the described features. In examples, a modified plant of this disclosure, if grown under low nitrogen conditions, exhibits increased NUE, wherein the growth under low nitrogen conditions is not more than 60-90 kg N/ha, inclusive, and including all numbers and ranges of numbers between 60-90, relative to NUE of a maize plant that has not been modified when grown under the same low nitrogen conditions.

In examples, the modified plant exhibits an improvement of any one or combination of the described features when field-grown, and such features are improved relative to a plant of the same species that does not have the modification, or has the same or a similar modification, but is not field-grown, i.e., the reference plant is grown in controlled conditions, such as a greenhouse or similar enclosure. In examples, the modified plant is an elite variety of maize. The meaning of elite variety of maize is known in the art, such as from White, et al. Diversity and heterotic patterns in North American proprietary dent maize germplasm. Crop Science 60, 100-114 (2020), and reference 10: Beckett, T. J., Morales, A. J., Koehler, K. L. & Rocheford, T. R. Genetic relatedness of previously Plant-Variety-Protected commercial maize inbreds. PLOS One 12, e0189277, doi: 10.1371/journal.pone.0189277 (2017), the disclosures of which are incorporated herein by reference.

The sequence of the protein encoded by maize nf-ya3 is:

(SEQ ID NO: 1)
MPVILREMEDHSVHPMSKSNHGSLSGNGYEMKHSGH
KVCDRDSSSESDRSHQEASAASESSPNEHTSTQSDN
DEDHGKDNQDTMKPVLSLGKEGSAFLAPKLHYSPSF
ACIPYTSDAYYSAVGVLTGYPPHAIVHPQQNDTTNT
PGMLPVEPAEEPIYVNAKQYHAILRRRQTRAKLEAQ
NKMVKNRKPYLHESRHRHAMKRARGSGGRFLNTKQL
QEQNQQYQASSGSLCSKIIANSIISQSGPTCTPSSG
TAGASTAGQDRSCLPSVGFRPTTNFSDQGRGGLKLA
VIGMQQRVSTIR

The sequence of the protein encoded by maize hb75 is:

(SEQ ID NO: 2)
MMIPARHMPPTMIVRNGGAAYGSSSALSLGQPNLMD
NQQLQFQQALQQQHLLLDQIPATTAESCDNTGRGGG
GRGSDPLADEFESKSGSENVDGVSVDDQDDPNQRPS
KKKRYHRHTLHQIQEMEA.

The disclosure includes a ML/double-TARGET pipeline for use in identifying and validating TFs that control genes-of-importance to NUE, which can be ranked based on NUE-specificity & NUE-influence (FIG. 29, panel A). The disclosure includes use of double-TARGET to show synergistic effects of TFs on genes-of-importance to NUE. Synergistic effects of TFs on trait outcomes have also been validated in Arabidopsis in planta (PMID: 17675404, 14576289). As represented by FIG. 29, the disclosure includes a high through-put experimental/computational approach to identify the specificity of a TF to NUE genes-of-importance and avoid off-target effects (FIG. 29, panel B). As proof-of-principle, we validated the regulated targets of 35 maize TFs-of-importance to NUE1, using our high throughput cell-based TARGET TF-assay (FIG. 29). We then used this data to rank each TF based on the significance of the overlap of its validated targets in maize cells with genes-of-importance to NUE in field (FIG. 29) discovered through Bipartite Machine learning (Shen, Coruzzi & Shasha 2024) on maize field NUE traits. The disclosure includes adapting this approach to double-TARGET assays to determine the specificity and influence of TF-pairs on NUE genes-of-importance (FIG. 29), incorporating synergistic effects of TFs into our models. This approach is expected to enable determining NUE TF pairs that affect other pathways. The disclosure includes calculating functional enrichment (e.g., GO, KEGG) in the “Other-target” list, i.e. TF targets that are not genes-of-importance to NUE (FIG. 29 panel C). This will allow identification of TF pairs whose non-NUE targets have a high enrichment of other pathways related positively or negatively to agricultural outcomes. The interaction between some pathways may be desirable; for example, the gene list from Bipartite Machine Learning (Shen, Coruzzi & Shasha, 2024) for NUE is enriched in photosynthesis genes and TFs regulating N utilization and photosynthesis have been shown to have higher yields. This approach will allow identification of TFs and TF pairs that specifically & significantly target genes of importance to NUE, which is expected to mitigate negative effects on other pathways.

In examples, the disclosure provides for modulation of the expression of one, two, three or more target nucleotide sequences (i.e., target genes) in a host cell, such as a plant protoplast. That is, the expression of a target nucleotide sequence of interest may be increased or decreased.

The target nucleotide sequences may be endogenous or exogenous in origin. By “modulate expression of a target gene” is intended that the expression of the target gene is increased or decreased relative to the expression level in a host cell that has not been altered by the methods described herein.

By “increased or overexpression” is intended that expression of the target nucleotide sequence is increased over expression observed in conventional transgenic lines for heterologous genes and over endogenous levels of expression for homologous genes. Heterologous or exogenous genes comprise genes that do not occur in the host cell of interest in its native state and include genes that have been recombinantly copied, and endogenous genes that have been operably linked to an introduced promoter. Homologous or endogenous genes are those that are natively present in the plant genome. Generally, expression of the target sequence is substantially increased. That is expression is increased at least about 25%-50%, preferably about 50%-100%, more preferably about 100%, 200% and greater.

By “decreased expression” or “underexpression” it is intended that expression of the target nucleotide sequence is decreased below expression observed in conventional transgenic lines for heterologous genes and below endogenous levels of expression for homologous genes. Generally, expression of the target nucleotide sequence of interest is substantially decreased. That is expression decreased by at least about 25%-50%, preferably about 50%-100%, more preferably about 100%, 200% and greater. “Disruption” of a gene generally means altering the sequence encoding a described protein encoded by the gene.” “Inhibiting expression” generally means targeting the mRNA encoding the protein such that no or less protein is translated, relative to translation of the protein where the mRNA encoding the protein is not targeted. In an example, mRNA is degraded by using a described guide RNA, such as those listed in Table 19.

Expression levels may be assessed by determining the level of a gene product by any method known in the art including, but not limited to determining the levels of the RNA and protein encoded by a particular target gene. For genes that encode proteins, expression levels may be determined, for example, by quantifying the amount of the protein present in plant cells, or in a plant or any portion thereof. Alternatively, it desired target gene encodes a protein that has a known measurable activity, then activity levels may be measured to assess expression levels.

Any method or delivery system may be used for the delivery and/or transfection of the nucleic acid vectors encoding any of the genes of interest of the present disclosure in the host cell, e.g., plant protoplast. The vectors may be delivered to the host cell either alone, or in combination with other agents. Transient expression systems may also be used. Homologous recombination may also be used.

Transfection may be accomplished by a wide variety of means, as is known to those of ordinary skill in the art. Such methods include, but are not limited to, Agrobacterium-mediated transformation (e.g., Komari et al., 1998, Curr. Opin. Plant Biol., 1:161), particle bombardment mediated transformation (e.g., Finer et al., 1999, Curr. Top. Microbiol. Immunol., 240:59), protoplast electroporation (e.g., Bates, 1999, Methods Mol. Biol., 111:359), viral infection (e.g., Porta and Lomonossoff, 1996, Mol. Biotechnol. 5:209), microinjection, and liposome injection. Other exemplary delivery systems that can be used to facilitate uptake by a cell of the nucleic acid include calcium phosphate and other chemical mediators of intracellular transport, microinjection compositions, and homologous recombination compositions (e.g., for integrating a gene into a preselected location within the chromosome of the cell). Alternative methods may involve, for example, the use of liposomes, electroporation, or chemicals that increase free (or “naked”) DNA uptake, transformation using viruses or pollen and the use of microprojection. Standard molecular biology techniques are common in the art (e.g., Sambrook et al., 1989, Molecular Cloning: A Laboratory Manual, 2nd ed., Cold Spring Harbor Laboratory Press, New York).

One of skill in the art will be able to select an appropriate vector for introducing the encoding nucleic acid sequence in a relatively intact state. Thus, any vector which will produce a host cell, e.g., plant protoplast, carrying the introduced encoding nucleic acid should be sufficient. The selection of the vector, or whether to use a vector, is typically guided by the method of transformation selected.

The transformation of plants cells in accordance with the disclosure may be carried out in essentially any of the various ways known to those skilled in the art of plant molecular biology. (See, for example, Methods of Enzymology, Vol. 153, 1987, Wu and Grossman, Eds., Academic Press, incorporated herein by reference).

Plant cells can comprise two or more nucleotide sequence constructs. Any means for producing a plant cell, e.g., protoplast, comprising the nucleotide sequence constructs described herein are encompassed by the present disclosure. For example, a nucleotide sequence encoding the modulator can be used to transform a plant cell at the same time as the nucleotide sequence encoding the precursor RNA. The nucleotide sequence encoding the precursor mRNA can be introduced into a plant cell that has already been transformed with the modulator nucleotide sequence. Likewise, viral vectors may be used to express gene products by various methods generally known in the art. Suitable plant viral vectors for expressing genes should be self-replicating, capable of systemic infection in a host, and stable. Additionally, the viruses should be capable of containing the nucleic acid sequences that are foreign to the native virus forming the vector.

### Homologous Recombination May be Used as a Method of Gene Inactivation.

The particular choice of a transformation technology will be determined by its efficiency to transform certain plant species as well as the experience and preference of the person practicing the disclosure with a particular methodology of choice. It will be apparent to the skilled person that the particular choice of a transformation system to introduce nucleic acid into plant cells is not essential to or a limitation of the disclosure, nor is the choice of technique for plant regeneration.

### Agrobacterium.

The nucleic acid sequences utilized in the present disclosure can be introduced into plant cells using Ti plasmids of Agrobacterium tumefaciens (A. tumefaciens), root-inducing (Ri) plasmids of Agrobacterium rhizogenes (A. rhizogenes), and plant virus vectors. For reviews of such techniques see, for example, Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9, and Horsch et al., 1985, Science, 227:1229.

In using an A. tumefaciens culture as a transformation vehicle, it is most advantageous to use a non-oncogenic strain of Agrobacterium as the vector carrier so that normal non-oncogenic differentiation of the transformed tissues is possible. It is also preferred that the Agrobacterium harbor a binary Ti plasmid system. Such a binary system comprises 1) a first Ti plasmid having a virulence region essential for the introduction of transfer DNA (T-DNA) into plants, and 2) a chimeric plasmid. The chimeric plasmid contains at least one border region of the T-DNA region of a wild-type Ti plasmid flanking the nucleic acid to be transferred. Binary Ti plasmid systems have been shown effective in the transformation of plant cells (De Framond, Biotechnology, 1983, 1:262; Hoekema et al., 1983, Nature, 303:179). Such a binary system is preferred because it does not require integration into the Ti plasmid of A. tumefaciens, which is an older methodology.

In some examples, a disarmed Ti-plasmid vector carried by Agrobacterium exploits its natural gene transferability (EP-A-270355, EP-A-01 16718, Townsend et al., 1984, NAR, 12:8711, U.S. Pat. No. 5,563,055).

Methods involving the use of Agrobacterium in transformation according to the present disclosure include, but are not limited to: 1) co-cultivation of Agrobacterium with cultured isolated protoplasts; 2) transformation of plant cells or tissues with Agrobacterium; or 3) transformation of seeds, apices or meristems with Agrobacterium.

In addition, gene transfer can be accomplished by in planta transformation by Agrobacterium, as described by Bechtold et al., (C. R. Acad. Sci. Paris, 1993, 316:1194). This approach is based on the vacuum infiltration of a suspension of Agrobacterium cells.

In certain examples, nucleic acid molecule is introduced into plant cells by infecting such plant cells, an explant, a meristem or a seed, with transformed A. tumefaciens as described above. Under appropriate conditions known in the art, the transformed plant cells are grown to form shoots, roots, and develop further into plants.

Other methods described herein, such as microprojectile bombardment, electroporation and direct DNA uptake can be used where Agrobacterium is inefficient or ineffective. Alternatively, a combination of different techniques may be employed to enhance the efficiency of the transformation process, e.g., bombardment with Agrobacterium-coated microparticles (EP-A-486234) or microprojectile bombardment to induce wounding followed by co-cultivation with Agrobacterium (EP-A-486233).

In some examples, cauliflower mosaic virus (CaMV) is used as a vector for introducing a desired nucleic acid into plant cells (U.S. Pat. No. 4,407,956). CaMV viral DNA genome can be inserted into a parent bacterial plasmid creating a recombinant DNA molecule which can be propagated in bacteria. After cloning, the recombinant plasmid again can be cloned and further modified by introduction of the desired nucleic acid sequence. The modified viral portion of the recombinant plasmid can then be excised from the parent bacterial plasmid, and used to inoculate the plant cells or plants.

### Mechanical and Chemical Means.

In some examples, a nucleic acid molecule of the disclosure is introduced into a plant cell using mechanical or chemical means. Exemplary mechanical and chemical means are provided below.

As used herein, the term “contacting” refers to any means of introducing a nucleic acid molecule into a plant cell, including chemical and physical means as described above. Preferably, contacting refers to introducing the nucleic acid or vector containing the nucleic acid into plant cells (including an explant, a meristem or a seed), via A. tumefaciens transformed with the nucleic acid molecule.

### Microinjection.

In one example, the nucleic acid molecule can be mechanically transferred into the plant cell by microinjection using a micropipette. See, e.g., WO 92/09696, WO 94/00583, EP 331083, EP 175966, Green et al., 1987, Plant Tissue and Cell Culture, Academic Press, Crossway et al., 1986, Biotechniques 4:320-334.

### Electroporation.

Electroporation can be used, in another set of examples, to deliver a nucleic acid to the cell (see, e.g., Fromm et al., 1985, PNAS, 82:5824). “Electroporation,” as used herein, is the application of electricity to a cell, such as a plant protoplast, in such a way as to cause delivery of a nucleic acid into the cell without killing the cell. Typically, electroporation includes the application of one or more electrical voltage “pulses” having relatively short durations (usually less than 1 second, and often on the scale of milliseconds or microseconds) to a media containing the cells. The electrical pulses typically facilitate the non-lethal transport of extracellular nucleic acids into the cells. The exact electroporation protocols (such as the number of pulses, duration of pulses, pulse waveforms, etc.), will depend on factors such as the cell type, the cell media, the number of cells, the substance(s) to be delivered, etc., and can be determined by those of ordinary skill in the art. Electroporation is discussed in greater detail in, e.g., EP 290395, WO 8706614, Riggs et al., 1986, Proc. Natl. Acad. Sci. USA 83:5602-5606; D'Halluin et al., 1992, Plant Cell 4:1495-1505). Other forms of direct DNA uptake can also be used in the methods provided herein, such as those discussed in, e.g., DE 4005152, WO 9012096, U.S. Pat. No. 4,684,611, Paszkowski et al., 1984, EMBO J. 3:2717-2722.

### Ballistic and Particle Bombardment.

Another method for introducing a nucleic acid molecule is high velocity ballistic penetration by small particles with the nucleic acid to be introduced contained either within the matrix of such particles, or on the surface thereof (Klein et al., 1987, Nature 327:70). Genetic material can be introduced into a cell using particle gun (“gene gun”) technology, also called microprojectile or microparticle bombardment. In this method, small, high-density particles (microprojectiles) are accelerated to high velocity in conjunction with a larger, powder-fired macroprojectile in a particle gun apparatus. The microprojectiles have sufficient momentum to penetrate cell walls and membranes, and can carry RNA or other nucleic acids into the interiors of bombarded cells. It has been demonstrated that such microprojectiles can enter cells without causing death of the cells, and that they can effectively deliver foreign genetic material into intact tissue. Bombardment transformation methods are also described in Sanford et al. (Techniques 3:3-16, 1991) and Klein et al. (Bio/Techniques 10:286, 1992). Although, typically only a single introduction of a new nucleic acid sequence(s) is required, this method particularly provides for multiple introductions.

Particle or microprojectile bombardment are discussed in greater detail in, e.g., the following references: U.S. Pat. No. 5,100,792, EP-A-444882, EP-A-434616; Sanford et al., U.S. Pat. No. 4,945,050; Tomes et al., 1995, “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al., 1988, Biotechnology 6:923-926.

### Other Methods.

In addition to the above, other physical methods for the transformation of plant cells are reviewed in the following and can be used in the methods provided herein. Oard, 1991, Biotech. Adv. 9:1-11. See generally, Weissinger et al., 1988, sAnn. Rev. Genet. 22:421-477; Sanford et al., 1987, Particulate Science and Technology 5:27-37; Christou et al., 1988, Plant Physiol. 87:671-674; McCabe et al., 1988, Bio/Technology 6:923-926; Finer and McMullen, 1991, In vitro Cell Dev. Biol. 27P: 175-182; Singh et al., 1998, Theor. Appl. Genet. 96:319-324; Datta et al., 1990, Biotechnology 8:736-740; Klein et al., 1988, Proc. Natl. Acad. Sci. USA 85:4305-4309; Klein et al., 1988, Biotechnology 6:559-563; Tomes, U.S. Pat. No. 5,240,855; Buising et al., U.S. Pat. Nos. 5,322,783 and 5,324,646; Klein et al., 1988, Plant Physiol. 91:440-444; Fromm et al., 1990, Biotechnology 8:833-839; Hooykaas-Van Slogteren et al., 1984, Nature (London) 311:763-764; Bytebier et al., 1987, Proc. Natl. Acad. Sci. USA 84:5345-5349; De Wet et al., 1985, The Experimental Manipulation of Ovule Tissues, ed. Chapman et al. (Longman, N.Y.), pp. 197-209; Kaeppler et al., 1990, Plant Cell Reports 9:415-418 and Kaeppler et al., 1992, Theor. Appl. Genet. 84:560-566; Li et al., 1993, Plant Cell Reports 12:250-255 and Christou and Ford, 1995, Annals of Botany 75:407-413; Osjoda et al., 1996, Nature Biotechnology 14:745-750; all of which are herein incorporated by reference.

### Nucleic Acid Constructs

The nucleic acid molecules of the disclosure may be provided in nucleotide sequence constructs or expression cassettes for expression in the plant cell of interest. The cassette will include 5′ and 3′ regulatory sequences operably linked to an encoding nucleotide sequence of the disclosure. As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

The expression cassette may additionally contain at least one additional gene to be co-transformed into the organism. Alternatively, the additional gene(s) can be provided on multiple expression cassettes.

In certain examples, an expression cassette can be used with a plurality of restriction sites for insertion of the sequences of the disclosure to be under the transcriptional regulation of the regulatory regions. The expression cassette can additionally contain selectable marker genes.

The expression cassette will generally include in the 5′-3′ direction of transcription, a transcriptional and translational initiation region, a DNA sequence of the disclosure, and a transcriptional and translational termination region functional in plants. The transcriptional initiation region, the promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a chimeric gene comprises a coding sequence operably linked to a transcription initiation region that is heterologous to the coding sequence.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al., 1991, Mol. Gen. Genet. 262:141-144; Proudfoot, 1991, Cell 64:671-674; Sanfacon et al., 1991, Genes Dev. 5:141-149; Mogen et al., 1990, Plant Cell 2:1261-1272; Munroe et al., 1990, Gene 91:151-158; Ballas et al., 1989, Nucleic Acids Res. 17:7891-7903; and Joshi et al., 1987, Nucleic Acid Res. 15:9627-9639.

In some examples, a nucleic acid can be delivered to the cell in a vector. As used herein, a “vector” is any vehicle capable of facilitating the transfer of the nucleic acid to the cell such that the nucleic acid can be processed and/or expressed in the cell. The vector may transport the nucleic acid to the cells with reduced degradation, relative to the extent of degradation that would result in the absence of the vector. The vector optionally includes gene expression sequences or other components (such as promoters and other regulatory elements) able to enhance expression of the nucleic acid within the cell. The disclosure also encompasses the cells transfected any described vectors.

Where appropriate, the gene(s) for enhanced expression may be optimized for expression in the transformed plant. That is, the genes can be synthesized using plant-preferred codons corresponding to the plant of interest. Methods are available in the art for synthesizing plant-preferred genes. See, for example, U.S. Pat. Nos. 5,380,831, and 5,436,391, and Murray et al., 1989, Nucleic Acids Res. 17:477-498.

Provided herein are host cells that contain a vector, e.g., a DNA plasmid and support the replication and/or expression of the vector. In some examples, host cells are monocotyledonous or dicotyledonous plant cells. In other examples monocotyledonous host cell is a maize host cell. In certain examples, the host cell utilized in the methods of the present disclosure are transiently transfected with the nucleic acid molecules of the disclosure.

In examples, the host cell utilized in the methods of the present disclosure is a plant protoplast. Plant protoplasts are plant cells that had their entire plant cell wall enzymatically removed prior to the introduction of the molecule of interest. The complete removal of the cell wall disrupts the connection between cells producing a homogenous suspension of individualized cells which allows more uniform and large scale transfection experiments. This comprises, but is not restricted to protoplast fusion, electroporation, liposome-mediated transfection, and polyethylene glycol-mediated transfection. Protoplast preparation is therefore a very reliable and inexpensive method to produce millions of cells.

Also provided herein are plant cells having the nucleotide sequence constructs of the disclosure. A further aspect of the present disclosure provides a method of making such a plant cell involving introduction of a vector including the construct into a plant cell. For integration of the construct into the plant genome, such introduction will be followed by recombination between the vector and the plant cell genome to introduce the sequence of nucleotides into the genome. RNA encoded by the introduced nucleic acid construct may then be transcribed in the cell and descendants thereof, including cells in plants regenerated from transformed material. A gene stably incorporated into the genome of a plant is passed from generation to generation to descendants of the plant, so such descendants should show the desired phenotype.

Optionally, germ line cells may be used in the methods described herein rather than, or in addition to, somatic cells. The term “germ line cells” refers to cells in the plant organism which can trace their eventual cell lineage to either the male or female reproductive cell of the plant. Other cells, referred to as “somatic cells” are cells which give rise to leaves, roots and vascular elements which, although important to the plant, do not directly give rise to gamete cells. Somatic cells, however, also may be used. With regard to callus and suspension cells which have somatic embryogenesis, many or most of the cells in the culture have the potential capacity to give rise to an adult plant. If the plant originates from single cells or a small number of cells from the embryogenic callus or suspension culture, the cells in the callus and suspension can therefore be referred to as germ cells. In the case of immature embryos which are prepared for treatment by the methods described herein, certain cells in the apical meristem region of the plant have been shown to produce a cell lineage which eventually gives rise to the female and male reproductive organs. With many or most species, the apical meristem is generally regarded as giving rise to the lineage that eventually will give rise to the gamete cells. An example of a non-gamete cell in an embryo would be the first leaf primordia in corn which is destined to give rise only to the first leaf and none of the reproductive structures.

### Promoters and Other Regulatory Sequences

In examples, a nucleic acid molecule of the disclosure is operably linked with a promoter. It may be desirable to introduce more than one copy of a polynucleotide into a plant cell for enhanced expression.

In general, promoters are found positioned 5′ (upstream) of the genes that they control. Thus, in the construction of promoter gene combinations, the promoter may be positioned upstream of the gene and at a distance from the transcription start site that approximates the distance between the promoter and the gene it controls in the natural setting. As is known in the art, some variation in this distance can be tolerated without loss of promoter function. Similarly, an example positioning of a regulatory element, such as an enhancer, with respect to a heterologous gene placed under its control reflects its natural position relative to the structural gene it naturally regulates.

Thus, the nucleic acid, in one example, is operably linked to a gene expression sequence, which directs the expression of the nucleic acid within the cell. A “gene expression sequence,” as used herein, is any regulatory nucleotide sequence, such as a promoter sequence or promoter-enhancer combination, which facilitates the efficient transcription and translation of the nucleotide sequence to which it is operably linked. The gene expression sequence may, for example, be a eukaryotic promoter or a viral promoter, such as a constitutive or inducible promoter.

A number of promoters can be used in the practice of the disclosure. The promoters can be selected based on the desired outcome. Generally, the nucleotide sequence and the modulator sequences can be combined with promoters of choice to alter gene expression if the target sequences in the tissue or organ of choice. Thus, the nucleotide sequence or modulator nucleotide sequence can be combined with constitutive, tissue-preferred, inducible, developmental, or other promoters for expression in plants depending upon the desired outcome.

The selection of a particular promoter and enhancer depends on what cell type is to be used and the mode of delivery. For example, a wide variety of promoters have been isolated from plants and animals, which are functional not only in the cellular source of the promoter, but also in numerous other plant species. There are also other promoters (e.g., viral and Ti-plasmid) which can be used. For example, these promoters include promoters from the Ti-plasmid, such as the octopine synthase promoter, the nopaline synthase promoter, the mannopine synthase promoter, and promoters from other open reading frames in the T-DNA, such as ORF7, etc. Promoters isolated from plant viruses include the 35S promoter from cauliflower mosaic virus. Promoters that have been isolated and reported for use in plants include ribulose-1,3-biphosphate carboxylase small subunit promoter, phaseolin promoter, etc. Thus, a variety of promoters and regulatory elements may be used in the expression vectors of the present disclosure.

Promoters useful in the compositions and methods provided herein include both natural constitutive and inducible promoters as well as engineered promoters. The CaMV promoters are examples of constitutive promoters. Other constitutive mammalian promoters include, but are not limited to, polymerase promoters as well as the promoters for the following genes: hypoxanthine phosphoribosyl transferase (“HPTR”), adenosine deaminase, pyruvate kinase, and alpha-actin.

Promoters useful as expression elements of the disclosure also include inducible promoters. Inducible promoters are expressed in the presence of an inducing agent. For example, a metallothionein promoter can be induced to promote transcription in the presence of certain metal ions. Other inducible promoters are known to those of ordinary skill in the art. The in vivo expression element can include, as necessary, 5′ non-transcribing and 5′ non-translating sequences involved with the initiation of transcription, and can optionally include enhancer sequences or upstream activator sequences.

For plant expression vectors, viral promoters that can be used in certain examples include the 35S RNA and 19S RNA promoters of CaMV (Brisson et al., Nature, 1984, 310:511; Odell et al., Nature, 1985, 313:810); the full-length transcript promoter from Figwort Mosaic Virus (FMV) (Gowda et al., 1989, J. Cell Biochem., 13D: 301) and the coat protein promoter to TMV (Takamatsu et al., 1987, EMBO J. 6:307). Alternatively, plant promoters such as the light-inducible promoter from the small subunit of ribulose bis-phosphate carboxylase (ssRUBISCO) (Coruzzi et al., 1984, EMBO J., 3:1671; Broglie et al., 1984, Science, 224:838); mannopine synthase promoter (Velten et al., 1984, EMBO J., 3:2723) nopaline synthase (NOS) and octopine synthase (OCS) promoters (carried on tumor-inducing plasmids of Agrobacterium tumefaciens) or heat shock promoters, e.g., soybean hsp17.5-E or hsp17.3-B (Gurley et al., 1986, Mol. Cell. Biol., 6:559; Severin et al., 1990, Plant Mol. Biol., 15:827) may be used. Exemplary viral promoters which function constitutively in eukaryotic cells include, for example, promoters from the simian virus, papilloma virus, adenovirus, human immunodeficiency virus, Rous sarcoma virus, cytomegalovirus, the long terminal repeats of Moloney leukemia virus and other retroviruses, and the thymidine kinase promoter of herpes simplex virus. Other constitutive promoters are known to those of ordinary skill in the art.

The particular promoter selected should be capable of causing sufficient expression to result in the production of an effective amount of structural gene product in the plant cell to cause upregulation of genes as compared to wild type. The promoters used in the vector constructs of the present disclosure may be modified, if desired, to affect their control characteristics. In certain examples, chimeric promoters can be used.

Promoters used in the nucleic acid constructs of the present disclosure can be modified, if desired, to affect their control characteristics. For example, the CaMV 35S promoter may be ligated to the portion of the ssRUBISCO gene that represses the expression of ssRUBISCO in the absence of light, to create a promoter which is active in leaves but not in roots. The resulting chimeric promoter may be used as described herein. For purposes of this description, the phrase “CaMV 35S” promoter thus includes variations of CaMV 35S promoter, e.g., promoters derived by means of ligation with operator regions, random or controlled mutagenesis, etc. Furthermore, the promoters may be altered to contain multiple “enhancer sequences” to assist in elevating gene expression.

An efficient plant promoter that may be used in specific examples is an “overproducing” or “overexpressing” plant promoter. Overexpressing plant promoters that can be used in the compositions and methods provided herein include the promoter of the small sub-unit (“ss”) of the ribulose-1,5-biphosphate carboxylase from soybean (e.g., Berry-Lowe et al., 1982, J. Molecular & App. Genet., 1:483), and the promoter of the chorophyll a-b binding protein. These two promoters are known to be light-induced in eukaryotic plant cells. For example, see Cashmore, Genetic Engineering of plants: An Agricultural Perspective, p. 29-38; Coruzzi et al., 1983, J. Biol. Chem., 258:1399; and Dunsmuir et al., 1983, J. Molecular & App. Genet., 2:285.

The promoters and control elements of, e.g., SUCS (root nodules; broadbean; Kuster et al., 1993, Mol Plant Microbe Interact 6:507-14) for roots can be used in compositions and methods provided herein to confer tissue specificity.

In certain examples, two promoter elements can be used in combination, such as, for example, (i) an inducible element responsive to a treatment that can be provided to the plant prior to N-fertilizer treatment, and (ii) a plant tissue-specific expression element to drive expression in the specific tissue alone.

Any promoter of other expression element described herein or known in the art may be used either alone or in combination with any other promoter or other expression element described herein or known in the art. For example, promoter elements that confer tissue specific expression of a gene can be used with other promoter elements conferring constitutive or inducible expression.

Following a described modification, a plant may be regenerated, e.g., from single cells, callus tissue or leaf discs, as is standard in the art. Almost any plant can be entirely regenerated from cells, tissues, and organs of the plant.

The modified plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

Normally, a plant cell is regenerated to obtain a whole plant from the transformation process. The term “growing” or “regeneration” as used herein means growing a whole plant from a plant cell, a group of plant cells, a plant part (including seeds), or a plant piece (e.g., from a protoplast, callus, or tissue part).

Parts obtained from the regenerated plant, such as flowers, seeds, leaves, branches, fruit, and the like are included in the disclosure, provided that these parts comprise cells comprising the isolated nucleic acid of the present disclosure. Progeny and variants, and mutants of the regenerated plants are also included within the scope of the disclosure, provided that these parts comprise the introduced nucleic acid sequences. Transgenic plants expressing the selectable marker can be screened for transmission of the nucleic acid of the present disclosure by, for example, standard immunoblot and DNA detection techniques. Transgenic lines are also typically evaluated on levels of expression of the heterologous nucleic acid. Expression at the RNA level can be determined initially to identify and quantitate expression-positive plants. Standard techniques for RNA analysis can be employed.

In examples, the disclosure provides a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present disclosure relative to a control plant (i.e., native, non-transgenic). Back-crossing to a parental plant and out-crossing with a non-transgenic plant are also contemplated.

Transformed plant cells which are derived by any of the above transformation techniques can be cultured to regenerate a whole plant which possesses the transformed genotype.

Plants cells transformed with a plant expression vector can be regenerated, e.g., from single cells, callus tissue or leaf discs according to standard plant tissue culture techniques. It is well known in the art that various cells, tissues, and organs from almost any plant can be successfully cultured to regenerate an entire plant.

The present disclosure also provides a plant comprising a plant cell as disclosed. Transformed seeds and plant parts are also encompassed.

In addition to a plant, the present disclosure provides any clone of such a plant, seed, selfed or hybrid progeny and descendants, and any part of any of these, such as cuttings, seed. The disclosure provides any plant propagule, that is any part which may be used in reproduction or propagation, sexual or asexual, including cuttings, seed and so on. Also encompassed by the disclosure is a plant which is a sexually or asexually propagated off-spring, clone or descendant of such a plant, or any part or propagule of said plant, off-spring, clone or descendant. Plant extracts and derivatives are also provided.

Engineered plants exhibiting the desired physiological and/or agronomic changes can be used directly in agricultural production.

Thus, provided herein are products derived from the transgenic plants or methods of producing transgenic plants provided herein. In certain examples, the products are commercial products. Some non-limiting examples include genetically engineered trees for e.g., the production of pulp, paper, paper products or lumber; tobacco, e.g., for the production of cigarettes, cigars, or chewing tobacco; crops, e.g., for the production of fruits, vegetables and other food, including grains, e.g., for the production of wheat, bread, flour, rice, corn; and canola, sunflower, e.g., for the production of oils or biofuels.

The TARGET system utilizes a nucleic acid encoding a chimeric protein comprising a transcription factor fused to a domain comprising an inducible cellular localization signal and an independently expressed selectable marker. Nucleic acids for use with the target system may be plasmids or other appropriate nucleic acid constructs as previously described. The TARGET system also comprises methods of measuring mRNA expression levels and may additionally comprise methods of detecting TF binding to gene targets.

The transcription factor component chimeric protein encoded by the nucleic acid construct may be, but is not limited to, one of those listed in FIG. 29. The transcription factor used is not limited to nuclear transcription factors, but may also include proteins that modulate mitochondrial or chloroplast gene expression.

The methods of the present disclosure comprise a step of detecting the level of mRNA expressed in the host cells of the disclosure.

In some examples, the level of mRNA expressed in host cells is determined by quantitative real-time PCR (qPCR), a method for DNA amplification in which fluorescent dyes are used to detect the amount of PCR product after each PCR cycle. (Higuchi et al., 1992; Simultaneous amplification and detection of specific DNA-sequences. Bio-Technology 10(4), 413-417].). The qPCR method has become the tool of choice for many scientists because of method's dynamic range, accuracy, high sensitivity, specificity and speed. Quantitative PCR is carried out in a thermal cycler with the capacity to illuminate each sample with a beam of light of a specified wavelength and detect the fluorescence emitted by the excited fluorochrome. The thermal cycler is also able to rapidly heat and chill samples thereby taking advantage of the physicochemical properties of the nucleic acids and DNA polymerase.

In some examples, the level of mRNA expressed in host cells is determined by high throughput sequencing (Next-generation sequencing; also ‘Next-gen sequencing’ or NGS).=NGS methods are highly parallelized processes that enable the sequencing of thousands to millions of molecules at once.

In some examples, the level of mRNA expressed in host cells is determined by gene microarrays. A microarray works by exploiting the ability of a given mRNA molecule to bind specifically to, or hybridize to, the DNA template from which it originated. By using an array containing many DNA samples, it can be determined in a single experiment, the expression levels of hundreds or thousands of genes within a cell by measuring the amount of mRNA bound to each site on the array. With the aid of a computer, the amount of mRNA bound to the spots on the microarray is precisely measured, generating a profile of gene expression in the cell.

In some examples, the method comprises detection of the level of TF binding to gene targets by ChIP-Seq analysis. ChIP-Seq analysis utilizes chromatin immunoprecipitation in parallel with DNA sequencing to map the binding sites of a TF or other protein of interest. First, protein interactions with chromatin are cross-linked and fragmented. Then, immunoprecipitation is used to isolate the TF with bound chromatin/DNA. The associated chromatin/DNA fragments are sequenced to determine the gene location of protein binding. Other assays known in the art may be used to detect the location of TF binding to genomic regions of DNA.

In some examples, gene networks conserved between Arabidopsis (or another model species) and a species of interest may be determined by a data mining approach. In this approach, Arabidopsis plants are grown under the same conditions as plants from another species of interest, including perturbation of environmental signals (e.g. nitrogen). RNA is then extracted from the roots and shoots of the plants, and cDNA synthesized from the extracted RNA. A microarray analysis and filtering approach may be used to determine the genes of each species regulated by the environmental signal when compared with control conditions. An ortholog analysis may then determine the genes orthologous between the two species. Data integration and network analysis then allows for the determination of a core translational network. In some examples, the response genes in a species of plant for which a protoplast system is not feasible may be discovered by using such a data mining approach, as described, in combination with the TARGET system for Arabidopsis or another species used as a model.

As discussed above, the TFs of the present disclosure include any TF that is referenced in the description, tables, or in the figures, including but not necessarily limited Table 1 and FIG. 29. In one representative example, the disclosure includes increasing or decreasing expression of one or both of MYBR3 or MYB34. Other genes for which the disclosure includes increasing or decreasing expression are described herein. In examples, expression of a described gene is disrupted, which may be achieved in one example by modifying a described gene within its coding sequence to inhibit or prevent translation of an encoded protein, or by modifying a regulatory sequence that operably linked to transcription of the described gene.

As discussed above, overexpression and underexpression of any one or combination of the described genes is included in the disclosure. As discussed above, overexpression of a particular gene can be accomplished by any method known in the art. For example, a plant cell may be transformed with a nucleic acid vector comprising the coding sequences of the desired gene operably linked to a promoter active in a plant cell such that the desired gene is expressed at levels higher than normal (i.e., levels found in a control/nontransgenic plant). The promoters can be constitutively active in all or some plant tissues or can be inducible. The under-expression of a desired gene can be accomplished by any method known in the art. For example, a gene may be knocked out, or mutated such that lower than normal levels of the gene product is produced in the transgenic cells or plant. For example, such mutations include frame-shift mutations or mutations resulting in a stop codon in the wild-type coding sequence, thus preventing expression of the gene product. Another exemplary mutation is the removal of the transcribed sequences from the plant genome, for example, by homologous recombination. Another method for under-expressing a gene is transgenically introducing an insertion or deletion into the transcribed sequence or an insertion or deletion upstream or downstream of the transcribed sequence such that expression of the gene product is decreased as compared to wild-type or other appropriate control. Additionally, microRNA (native or artificial) can be used to target a particular encoding mRNA for degradation, thus reducing the level of the expressed gene product in the transgenic plant cell. Another method for underexpression of a gene of interest is using clustered regularly interspaced short palindromic repeats (CRISPR) gene inactivation. A variety of suitable CRISPR systems for use in plants can be used, and include but are not necessarily limited to Cas3, Cas9, and Cas13 based systems, all of which are known in the art and can be adapted for the described purposes, such as by using a suitable CRISPR enzyme and guide RNA to target the described gene(s) and/or their regulatory elements, such as promoters.

The described compositions and methods can be used for any type of plant, such as monocots, dicots, gymnosperms, or plant cells. The term “plant cell” as used herein refers to protoplasts, gamete producing cells, and includes cells which regenerate into whole plants. Plant cells include but are not necessarily limited to cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. In non-limiting embodiments, the method is used for any species of woody, ornamental, decorative, crop, cereal, fruit, or vegetable plant. The method can be used on intact plants, isolated plant parts, and plant cells. In embodiments, the method is used with a seed, a suspension culture, an embryo, a meristematic plant region, callus tissue, a leaf, a root, a shoot, a gametophyte, a sporophyte, pollen, a microspore, or a protoplast. In embodiments, the plant or plant cells that are modified according to the disclosure are any member of the following genera/group: Artemisia, Acorns, Aegilops, Allium, Amborella, Antirrhinum, Apium, Arabidopsis, Arachis, Beta, Betula, Brassica, Cannabis, Capsicum, Ceratopteris, Citrus, Coffea, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Oryza, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia. In non-limiting embodiments, the modified plant or plant cells are from one or more so-called “elite” varieties of maize. Those skilled in the art will understand the meaning of the term “elite” in the context of maize varieties. In non-limiting examples, an elite variety is an inbred variety, which through breeding, has been developed to exhibit superior traits, and may be useful for hybrid development. In an example, an elite variety of maize exhibits one or more traits, examples of which are described herein, that are improved relative to their parental plants.

The disclosure includes seeds produced by any modified plant herein, and progeny of the plants and seeds. Articles of manufacture comprising the seeds and a container that contains the seeds are also provided. In embodiments, the articles of manufacture comprise kits.

The following Examples are intended to illustrate aspects of the disclosure but are not intended to be limiting.

### Examples

### Identification of a Temporal Cascade of Early-to-Late Nitrogen-Responsive Genes in Maize.

The quest to identify nitrogen (N)-gene regulatory networks (N-GRN) in maize began with microarray studies (Jiang et al. 2018), and later by RNA-seq analysis (Ge et al. 2020; Wang et al. 2020; Buoso et al. 2021). Recently, a combined analysis of N-response RNA-seq and phenotype data in the machine learning of N-response genes-of-importance to NUE in maize was described (Cheng et al. 2021). This disclosure expands on these GRN studies by exploiting time, an unexplored dimension of the N-GRN response in maize. As causality moves forward in time, fine-scale time-series RNA-seq data is a valuable source to derive predictive networks. In this disclosure, we implement and validate a time-inferred causal gene regulatory network (GRN) in maize. We then compare it to a parallel study in the model Arabidopsis (Varala et al. 2018) to reveal N-regulatory temporal networks conserved model-to-crop.

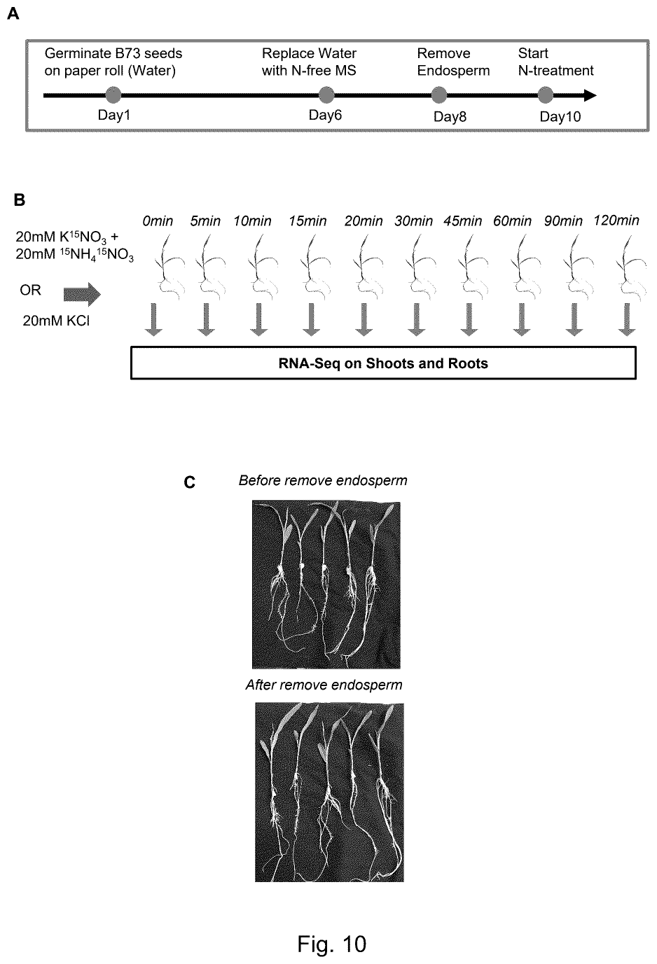

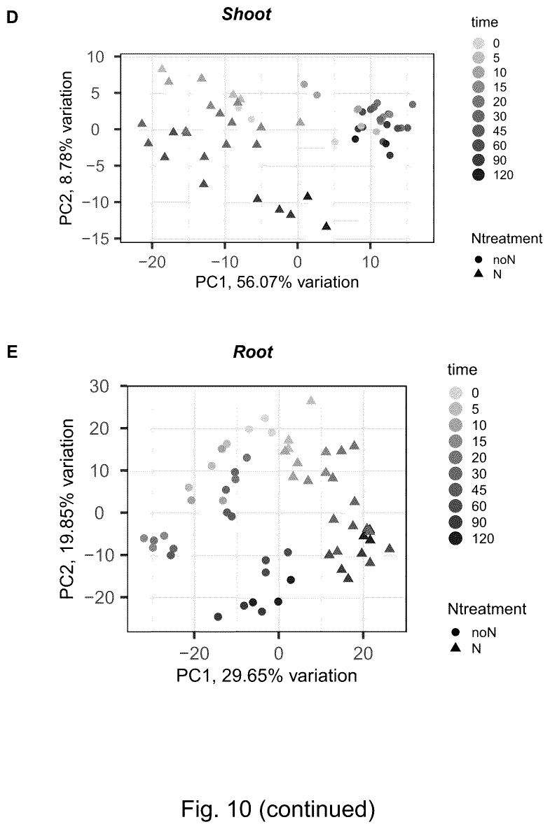

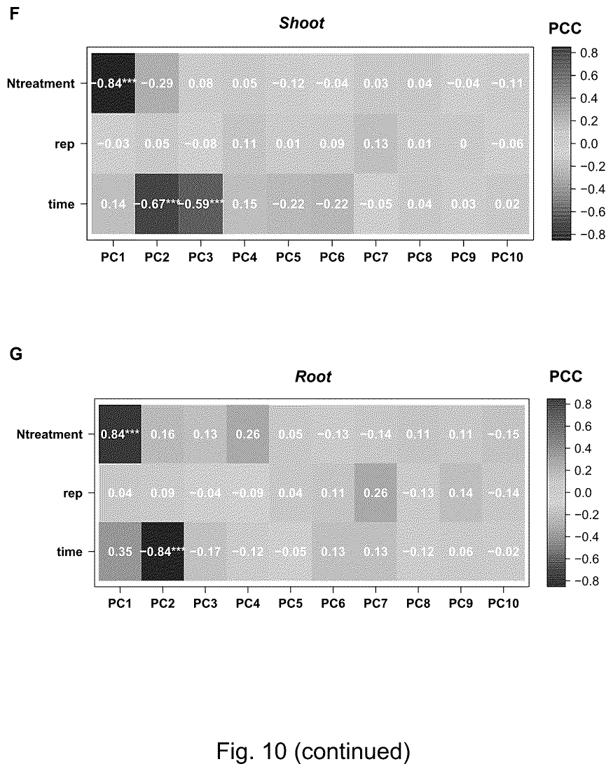

To uncover time-dependent effects of nitrogen (N)-treatment in maize, we conducted a fine-scale series of N-treatments over ten time-points (0-120 min), using a parallel design to the study we conducted in Arabidopsis (Varala et al 2018). This N-treatment (20 mM KNO₃+20 mM NH₄NO₃) in the widely used Murashige & Skoog media was compared to a control (20 mM KCl), as done previously in Arabidopsis (Varala et al. 2018) (FIG. 10). We measured N-related phenotypes including ¹⁵N-uptake and collected samples for RNA-seq analysis from shoots and roots of maize B73 seedlings (see Methods) (FIG. 10). This replicated experimental design allowed us to follow the time-dependent N-responses in maize, as well as those conserved model-to-crop.

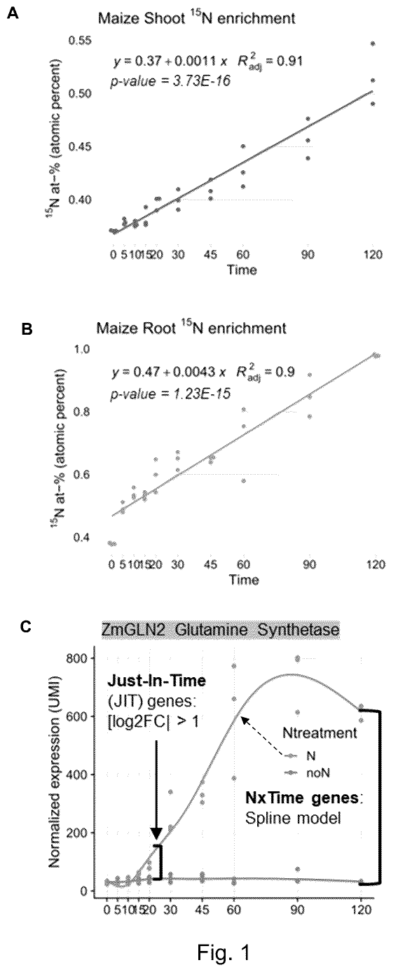

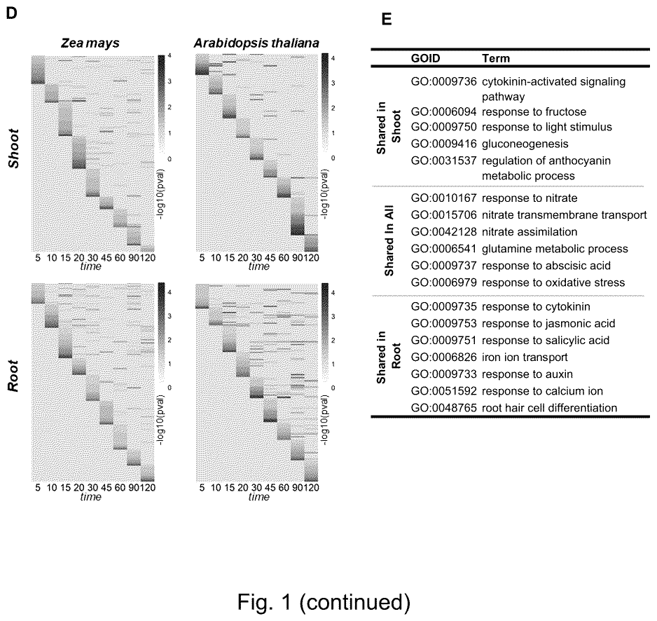

Our ¹⁵N enrichment analysis showed a linear increase in total nitrogen uptake (nitrate and ammonium) in shoots and roots of maize B73 seedlings (FIGS. 1A and 1B). Moreover, Principal Component Analysis (PCA) of gene expression data from shoots and roots (FIGS. 10D and 10E) showed that PC1 is strongly associated with N-treatment, and PC2 correlates significantly with time (FIGS. 10F and 10G). Thus, the effectiveness of our N-treatment experimental design is validated by both the PCA analyses of 15N enrichment and RNA-seq data

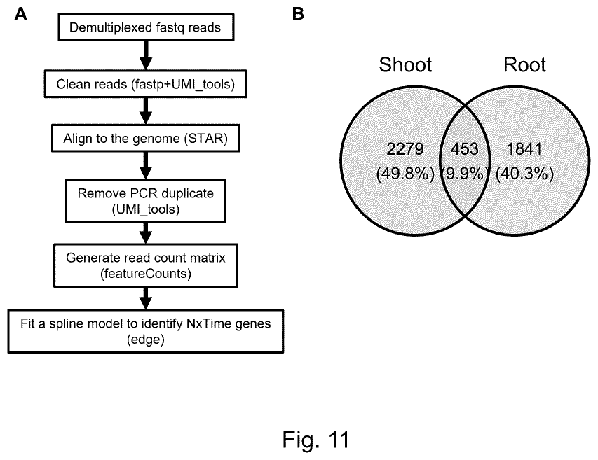

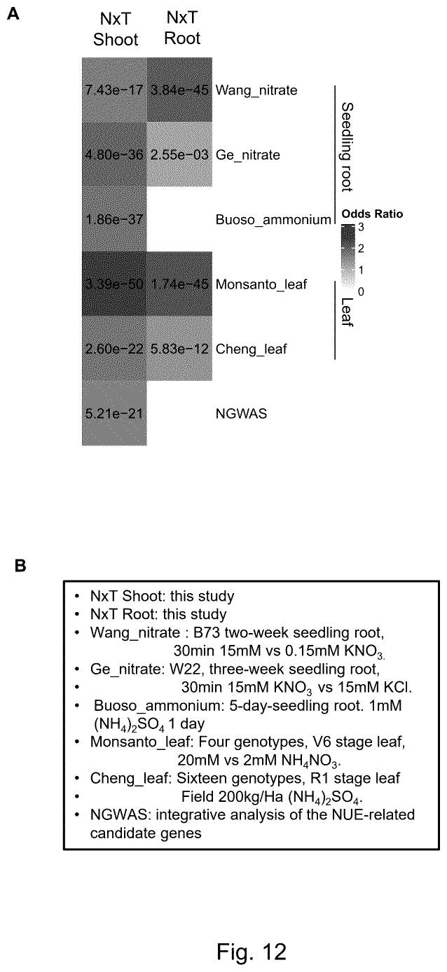

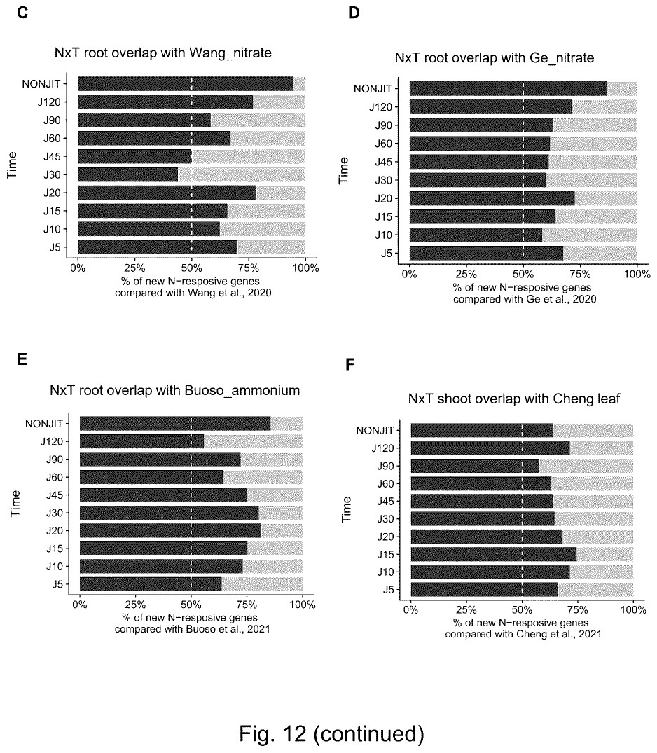

Leveraging our fine-scale time-course experimental design, we employed spline modeling (Storey et al. 2005) to identify genes regulated by N as a function of time (NxTime genes), as we previously did in Arabidopsis (Varala et al. 2018) (FIG. 11A). By smoothing gene expression using a natural cubic spline across time-samples, we could identify genes whose NxTime response curve is significantly different between N-treated vs. control KCl conditions (FIG. 1C). Spline analysis identified 2,732 NxTime response genes in maize shoots and 2,294 NxTime response genes in roots. Among these gene sets, 453 genes, including 55 transcription factors (TFs), are NxTime responsive in both shoot and root (FIG. 11B). A comparative analysis of the NxTime genes we uncovered in maize with published single time-point N-treatment datasets captures significant overlaps with other N-treatment studies in maize (FIG. 12A-B). 50% of the NxTime responsive maize genes identified in this disclosure are novel N-responders, not previously detected in single time-point studies of maize (FIG. 12C-F). Thus, our time-course RNA-Seq analysis identified novel and dynamic N-responsive genes in shoots and roots of maize.

Temporal N-Regulation of N-Uptake and Assimilation Pathway Genes is Consistent with ¹⁵N-Uptake in Planta.

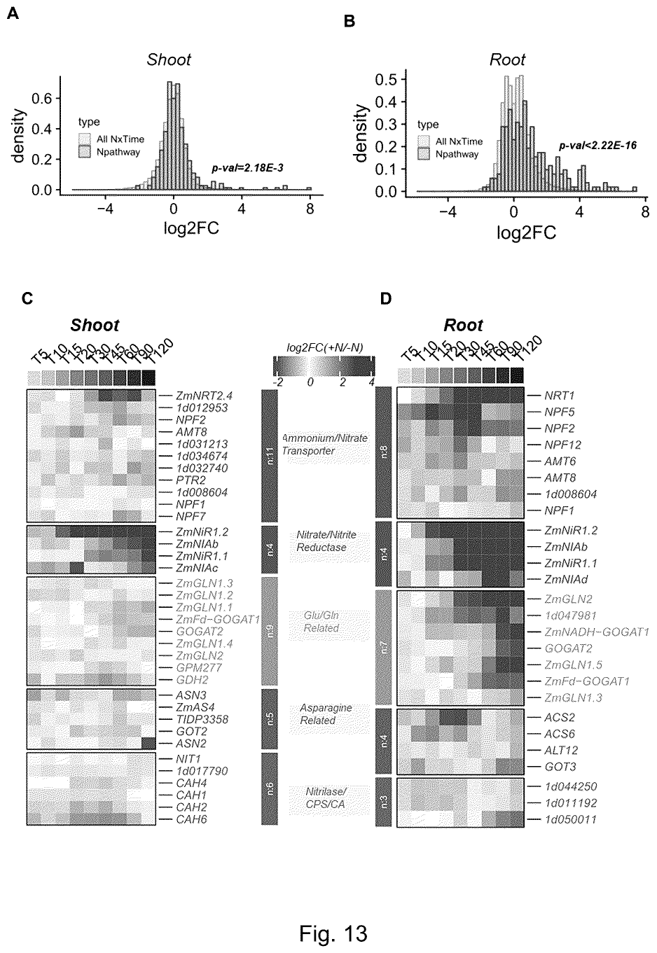

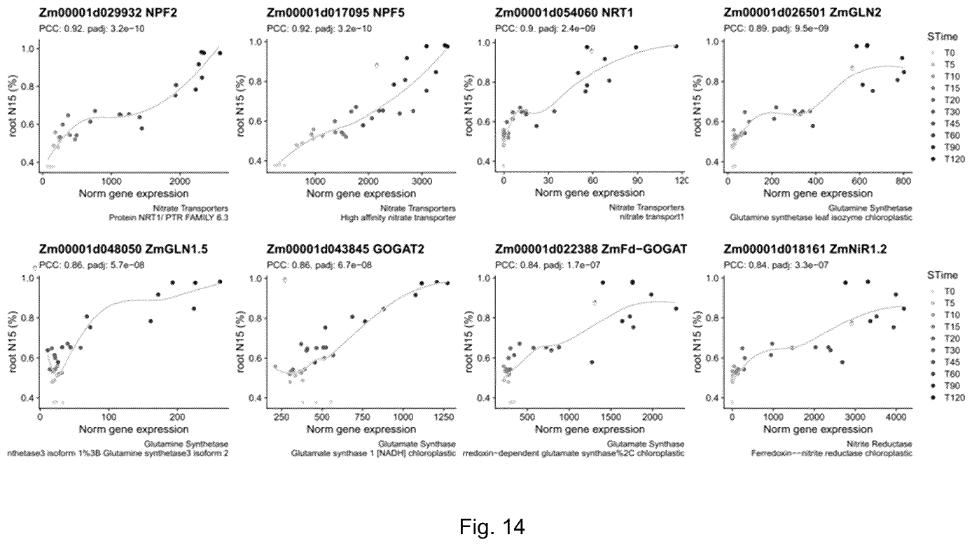

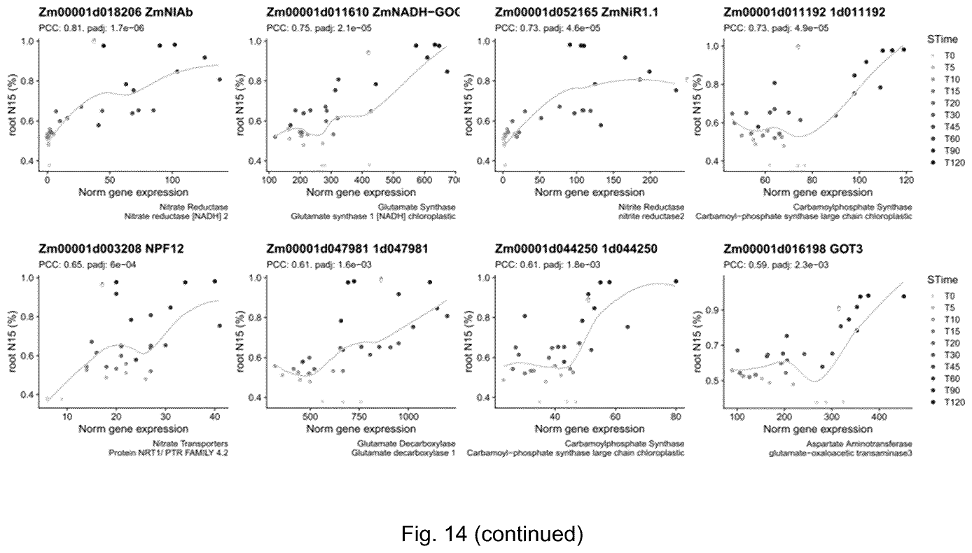

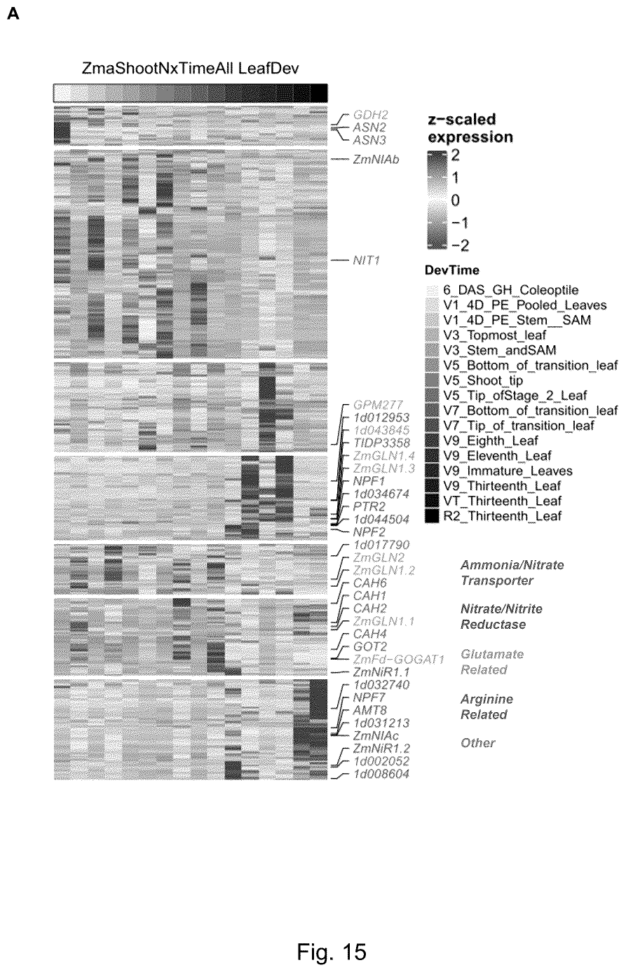

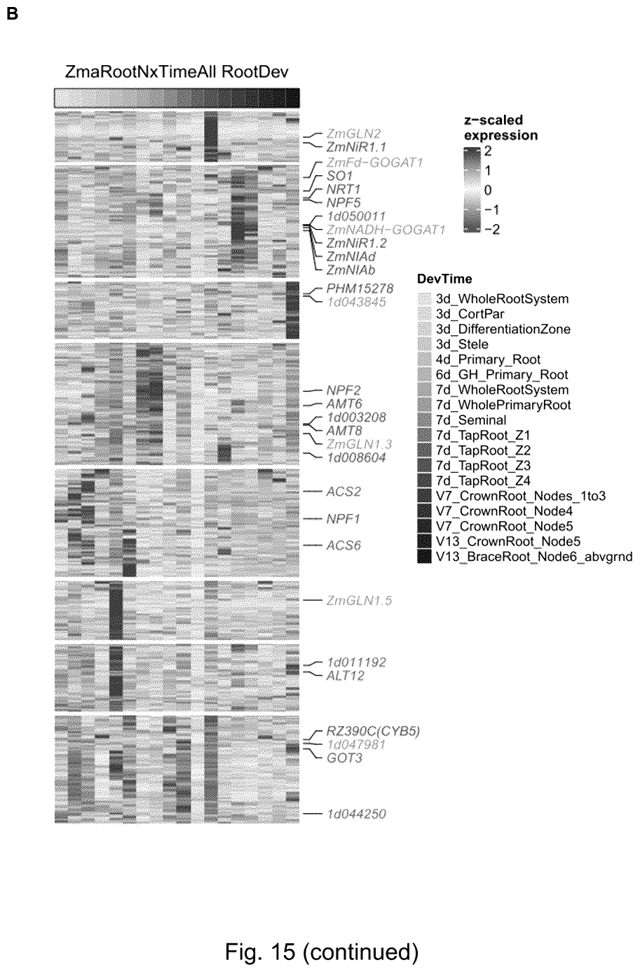

Our time-series N-treatment data of maize showed an increase of 15N uptake over time (FIGS. 1A & B). We thus asked whether genes involved in N-uptake/assimilation respond temporally to the increase in internal N-dose as it accumulates over time. Indeed, within our NxTime responsive gene lists, we identified 35 N-pathway genes in shoot and 29 in roots (See Method, FIG. 13). Moreover, we found that 16/29 N-pathway genes NxTime responsive expression pattern in roots are significantly correlated with our ¹⁵N uptake measurements (FIG. 14). The Pearson's correlation coefficients for all 16 NxTime responsive N-pathway genes are positive, suggesting that higher N-responsive expression of these genes leads to more nitrogen uptake. We also used the public maize gene atlas (Woodhouse et al. 2021) to explore the NxTime responsive N-pathway gene expression patterns in the maize adult stages (FIG. 15). In both shoot and root, the expression for N-pathway/NxTime responsive genes extends beyond the seedling stage into the adult stage, with some genes peaking in expression during the reproductive stage (FIG. 15). This underscores the potential of NxTime and N-pathway genes in plant improvement strategies related to N-uptake.

### Just-In-Time (JIT) Analysis Uncovers a Temporal N-Response Gene Cascade Conserved Between Maize and Arabidopsis

Our time-series N-response data gives us the opportunity to explore the temporal dynamics of early-to-late N-response genes in maize. To this end, we applied a Just-in-Time (JIT) analysis to our maize NxTime dataset, using the approach we developed in Arabidopsis (Varala et al. 2018). In this disclosure, this JIT approach categorizes genes into bins based on the earliest time-point at which they exhibit an absolute log 2 (Fold Change) bigger than one, when comparing N-treated versus control conditions over time (FIG. 1C). We identified genes responding to NxTime using spline analysis (Storey et al. 2005; Varala et al. 2018), and categorized genes into JIT bins as described in Varala et al 2018, with minor modifications described in Methods.

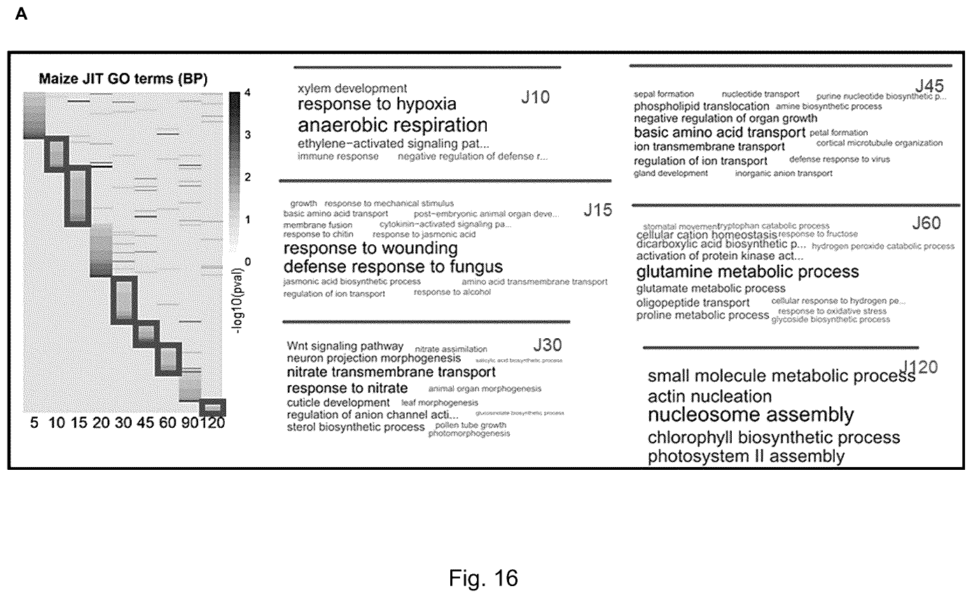

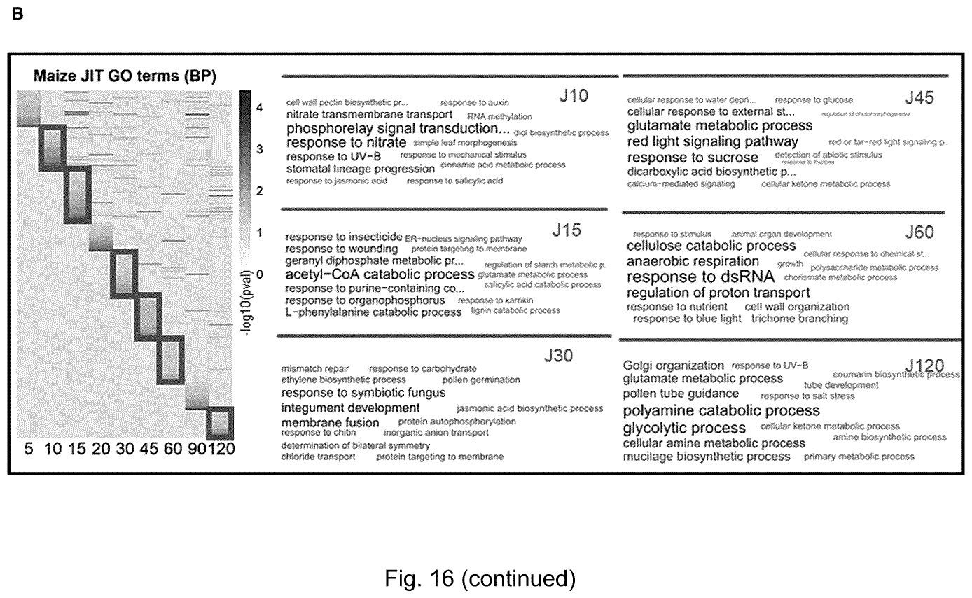

We next tested whether gene sets within each JIT bin in maize were enriched in shared Gene Ontology (GO) terms and/or cis-regulatory motifs. GO enrichment analysis of the JIT genes uncovered a temporal N-response gene cascade enriched in nitrogen-related functions. In shoots, nitrate transport showed enrichment at early JIT time points (5, 10, 15 min), while the glutamine metabolic process was enriched after 30 min, and photosynthesis terms emerged at later stages (120 min) (FIG. 16A). The trend of nitrate transport followed by glutamine/glutamate metabolic process is more obvious in maize roots, reflecting the sequential process of nitrogen uptake and assimilation in plants (FIG. 16B).

Next, we aimed to perform a comparative analysis of the temporal N-response cascade we uncovered in maize with the model Arabidopsis. To this end, we revisited the NxTime RNA-seq data sets we previously identified in Arabidopsis (Varala et al. 2018), using an identical JIT pipeline to our slightly modified scheme (see Methods). Our model-to-crop comparison revealed a remarkable similarity in temporal N-response patterns and enriched GO term in JIT N-response genes shared between maize and Arabidopsis, in both shoot and root tissues (FIG. 1D). Core processes in nitrogen metabolism and signaling were found to be common across the temporal N-response cascade in both species and tissues (FIG. 1E). These include responses to nitrate (GO: 0010167), nitrate assimilation (GO: 0042128), and the glutamine metabolic process (GO: 0006541). Furthermore, shared GO terms in the N-response cascade in shoots are related to energy and metabolism, such as the response to light stimulus (GO: 0009750). In roots, shared terms in the NxTime response cascade are related to phytohormones, such as response to cytokinin (GO: 0009735) and auxin (GO: 0009733). In summary, the comparative JIT analysis revealed a conserved temporal cascade of N-responsive functions shared between the model species, Arabidopsis and the crop, maize.

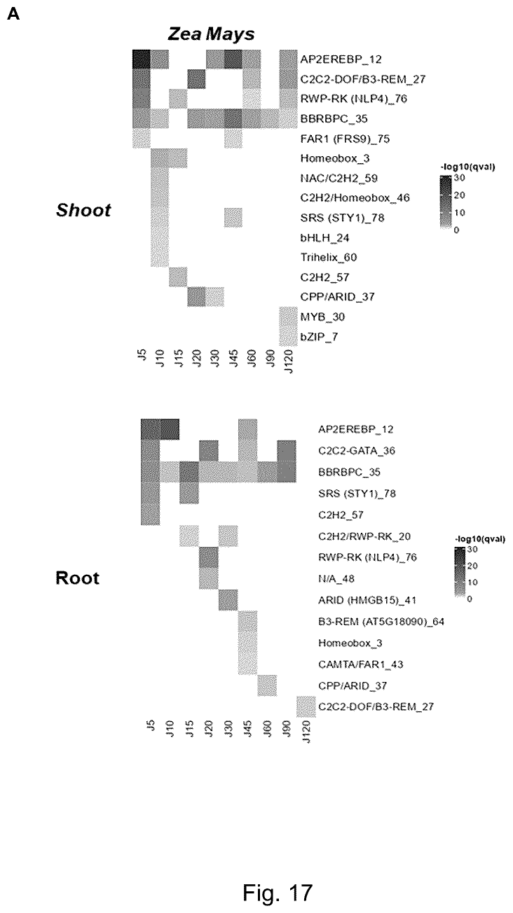

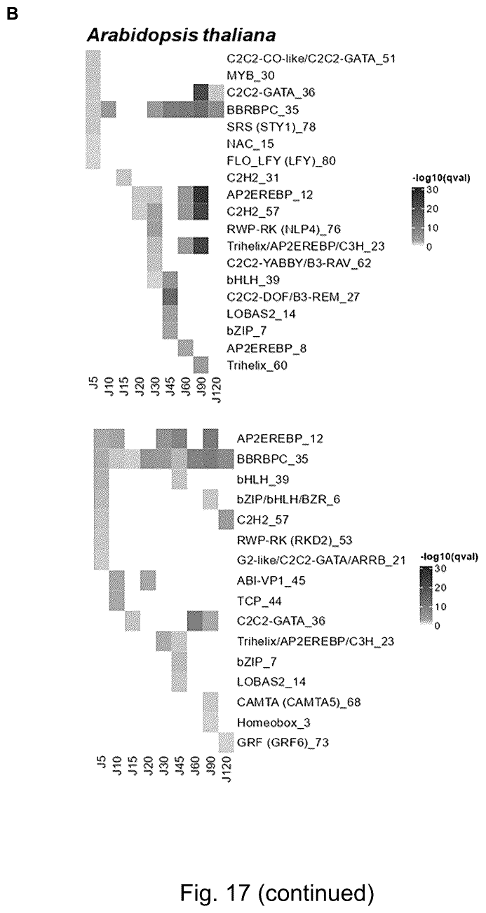

With regard to shared regulatory mechanisms, our analysis revealed that temporal N-response JIT genes in maize (this disclosure), are enriched in specific cis-motif clusters analogous to those in Arabidopsis (Brooks et al. 2019) (FIG. 17). The temporal pattern for enriched cis-motifs was more obvious in the maize root JIT genes, likely due to the roots' direct exposure to nitrogen treatments. Notably, cis-motifs for the BBRBPC TF family were prevalent across almost all JIT bins in shoots and roots in both species. The BBRBPC motif was found in several key nitrogen-pathway genes, such as ZmNiR1.2, ZmGLN1, ZmGLN1.5 and GOGAT2. The BBRBPC family is known for its role in cytokinin response regulation (Shanks et al. 2018). Cytokinin plays an important part in coordinating systematic nitrogen deficiency and demand signaling in Arabidopsis (Ruffel et al. 2011; Naulin et al. 2020). Moreover, BPCs are known to interact with the ABA signaling pathway (Mu et al. 2017) and the circadian clock (Lee et al. 2022), both of which are influenced by nitrogen availability (Gutierrez et al. 2008; Zhang et al. 2021; Contreras-López et al. 2022). Thus, the temporal N-response cascade across model-to is linked to cytokinin, ABA and circadian clock across model-to-crop.

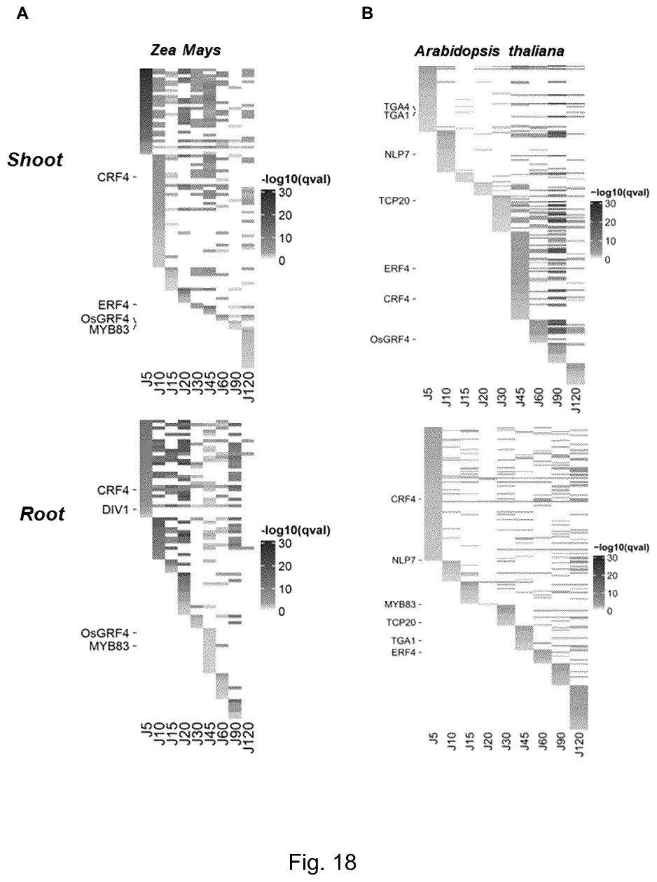

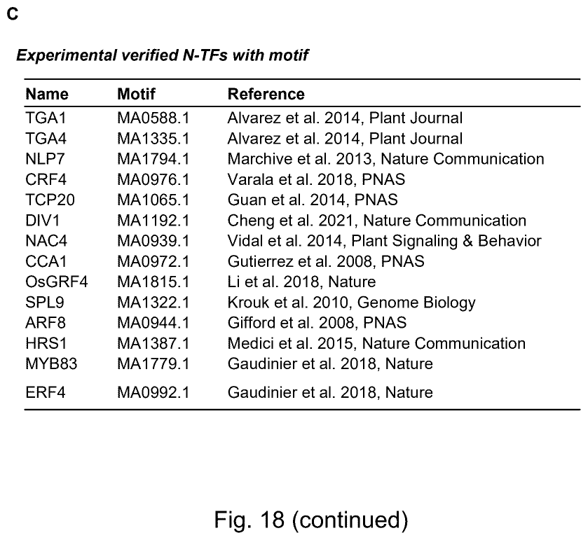

The cis-motif clusters enriched in the temporal N-response JIT gene bins aggregate multiple transcription factors (TFs) sharing similar position weight matrices (PWMs). To identify specific TF binding motifs, we conducted cis-element enrichment using individual TF cis-motifs from the core JASPAR PWM database (Castro-Mondragon et al. 2022) for JIT genes. Similar to our results from cis-motif cluster analysis (described above), we observed a temporal enrichment of cis-motifs at the individual TF level in both maize and Arabidopsis JIT bins (FIG. 18). This analysis identified cis-motifs for several TFs previously validated to be pivotal to N-signaling in Arabidopsis within JIT gene bins, including CRF4 (Varala et al. 2018), TCP20 (Guan et al. 2014), OsGRF4 (Li et al. 2018), and DIV1 (Cheng et al. 2021). In summary, our analyses emphasize a parallel temporal progression of cis-motifs enriched in the N-response cascade conserved between maize and Arabidopsis, suggesting a conserved mechanism across model-to-crop.

Validation of Maize TFs that Regulate Genes in the Temporal N-Response Cascade.

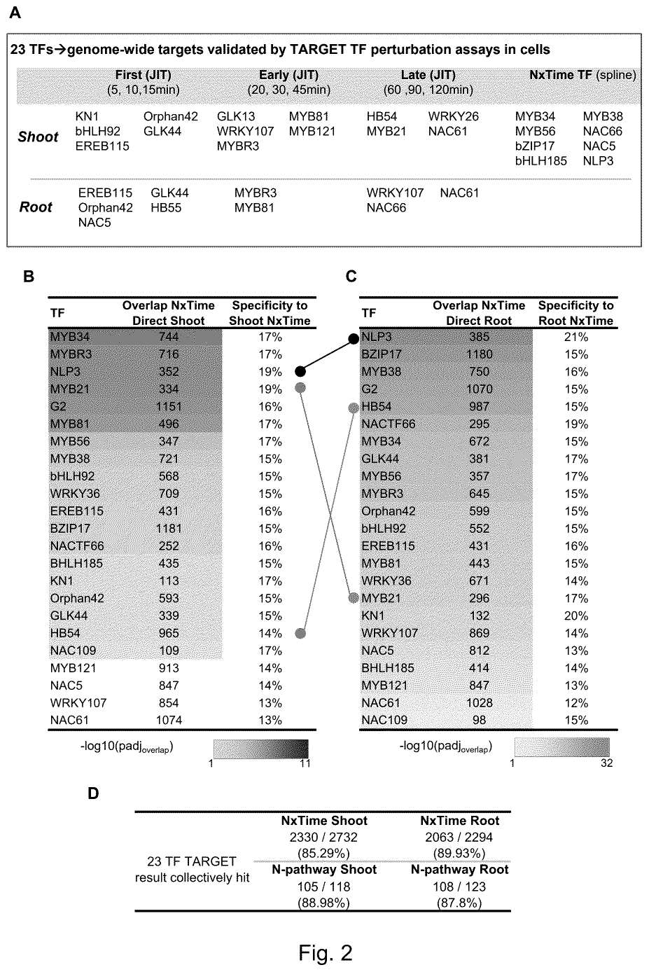

Our next goal was to identify and validate candidate TFs that regulate genes in the temporal N-response cascade in maize, shown in FIG. 1. Drawing from our list of NxTime and JIT genes in maize, we selected 23 TFs spanning early-to-late JIT bins in both shoot and root tissues (FIG. 2A). For validation, we selected TFs that span the N-temporal response cascade including first N-responder TFs (5-15 min), early-N responder TFs (20-45 min), or late N-responder TFs (60-120 min) (FIG. 2A). To uncover the direct genome-wide targets of these 23 maize TFs, we used the plant cell-based TARGET TF-perturbation system, a rapid medium through TF-target assay (Brooks et al. 2019), which overcomes the challenges of TF validation in maize transgenic plants. The TARGET assay (Transient Assay Reporting Genome-wide Effects of Transcription factors) can rapidly identify the direct regulated TF-targets in plant cells isolated from whole tissue, which has been validated in Arabidopsis (Bargmann et al. 2013; Varala et al. 2018; Brooks et al. 2019) and recently in rice (Shanks et al. 2022). In this disclosure of maize TFs, we adapted the TARGET TF-perturbation assay and TF expression vector to protoplasts derived from etiolated maize seedlings (See Methods). Briefly, protoplasts were isolated from etiolated leaves of maize seedlings. The maize protoplasts (p1107) were transfected with a plasmid that overexpresses a TF-Glucocorticoid (TF-GR) fusion protein expressed from a Ubiquitin (ZmaUBQ) (Bargmann et al. 2013; Brooks et al. 2019; Shanks et al. 2022). The plasmid also encodes a ZmaUBQ::RFP protein, which serves as a cell-sorting marker to isolate transfected cells using Fluorescence-activated Cell Sorting (FACS). The GR-TF fusion protein design allows for the controlled entry of the maize TF-GR fusion protein into the nucleus using dexamethasone (DEX). Additionally, cycloheximide+DEX treatment inhibits translation of mRNA for TF2s. Therefore, only the target genes of the over-expressed TF are identified, when GR-TF samples are compared to the empty vector (GR-no TF) control (Brooks et al., 2019).

The maize cells expressing the TF-GR fusion (RFP-positive FACs sorted cells) were collected for RNA-Seq analysis and compared to cells transfected with an empty vector (EV) lacking the TF. We performed the TARGET assay on the 23 maize TFs which span early to late responders, and included a handful of NxTime TFs that did not pass a two-fold change cutoff for the JIT analysis (FIG. 2A). On average, for the 23 maize TFs tested, each was validated to regulate ~4,000 direct target genes in the TARGET assay.

We next sought to validate the relevance of the direct regulated TF targets identified in the cell-based TARGET TF assay performed in maize cells to the N-response temporal cascade in maize in planta. To this end, we calculated the significance of the overlap between the direct TF targets identified in the cell-based TARGET assay in isolated maize cells with the NxTime genes identified in shoots and roots of whole maize seedlings (FIG. 2). Collectively, the directly regulated genes of the 23 TFs validated in the cell-based TARGET assay encompass over 85.3% of NxTime genes in shoots and 89.9% in roots of maize plants (FIG. 2D). Using the Fisher exact test, we identified significant overlap of each of the targets identified for the 23 TFs in the cell-based assay, with the N-response genes identified in shoots or roots of whole maize plants, as −log 10 (padjₒᵥₑᵣₗₐₚ) (FIG. 2B-C). Additionally, we determined the specificity of the TF to regulated nitrogen responsive genes in each organ of maize. To do this, we calculated the percent overlap of the TF-regulated genes in the cell-based TARGET assay, with the NxTime genes within each tissue of maize seedlings, as done previously for Arabidopsis (Brooks et al. 2019). This overlap analysis identified MYB34 (Zm00001d042830) and MYBR3 (Zm00001d038270) as the top-ranked TFs controlling N-response in maize shoots. In support of this, in a many-to-one comparison, the maize MYB ortholog in Arabidopsis, AtDIV1 (AT5G58900), shares a role in nitrogen signaling (Varala et al. 2018). Moreover, supporting is role in nitrogen use vivo, an Arabidopsis Atdiv1 mutant has a higher NUE, compared to wild type (Cheng et al. 2021). In maize roots, the highest-ranked TF for N-specificity in planta was NLP3, whose ortholog in Arabidopsis, AtNLP8, is known as a master regulator of nitrate-dependent seed germination (Yan et al. 2016). We also found TFs that are highly ranked for target gene overlap in one maize tissue and low in another, for example MYB21 and HB54 (FIG. 2B-C).

To make this large TF-target validation set a resource available to the maize community, we integrated the validated regulated targets for these 23 maize TFs into the ConnecTF platform, as described in (Brooks et al. 2021). In the next section, a description of how we used next this validated TF-target dataset for 23 maize TFs for gene regulatory network validation and refinement of the time-inferred nitrogen signaling network.

### Time-Based Network Inference and Validation of TFs Controlling the Temporal Nitrogen Response Cascade in Maize

Inferring gene regulatory networks (GRNs) from time-course RNA-Seq experiments offers the advantage of identifying potential causal relationships between TFs and genes. Unlike analyses based on steady-state data, time-course experiments provide a dynamic perspective. Using time series data to infer causality in TF-gene regulation enhances the ability to predict GRN responses at future time-points, a goal of systems biology (Krouk et al. 2010, 2013; Bar-Joseph et al. 2012; Marku and Pancaldi 2023).

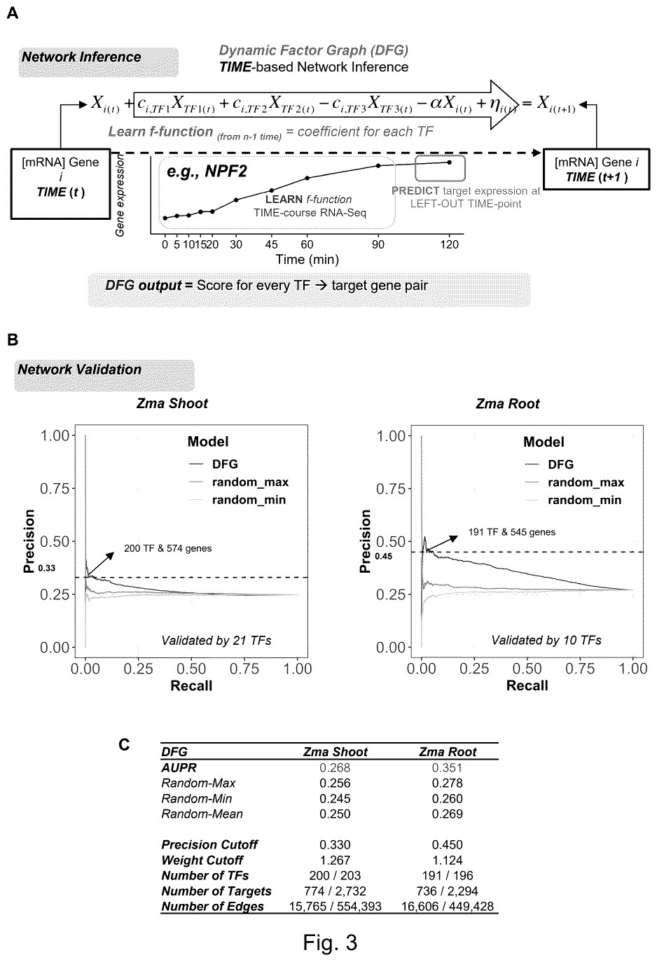

Previously, we constructed and validated nitrogen signaling GRNs (N-GRNs) in Arabidopsis shoots and roots using Dynamic Factor Graphs (DFG), a time-based machine learning GRN inference method (Krouk et al. 2010, 2013; Varala et al. 2018; Brooks et al. 2019). The DFG method uses fine scale time-series RNA-seq data to predict the future expression of a target gene. DFG uses the target gene's current state and the expression of TFs at time (t) to estimate expression at the next time point (t+1) (Krouk et al. 2010, 2013) (FIG. 3A).

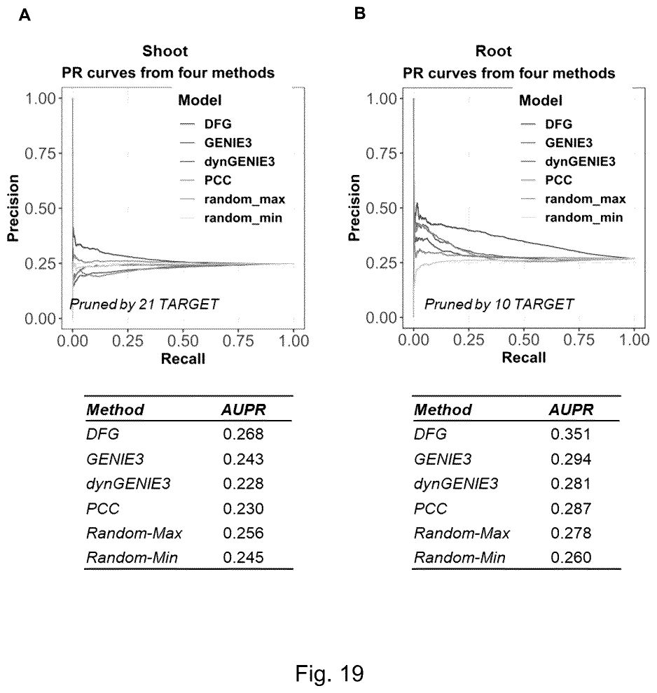

In this disclosure, we applied this time-based DFG method to the maize temporal N-response RNA-seq data which identified 2,732 NxTime responsive genes in shoots and 2,294 NxTime responsive genes in roots). Next, we used our validated TF-target data for 23 maize TFs from TARGET (FIG. 2) to evaluate the performance of the DFG-inferred maize networks in predicting TF-target regulation using an Area Under the Precision-Recall Curve (AUPR) analysis. We also compared the performance of our DFG predictions of TF-target relationships, with networks inferred from two popular methods, GENIE3 (Huynh-Thu et al. 2010), dynGENIE3 (Huynh-Thu and Geurts 2018), or by correlation methods such as Pearson Correlation Coefficient (PCC). We compared the performance of the above GRN methods using AUPR, which measures the accuracy of a TF-target prediction model by calculating the area under a curve that plots the precision (the ratio of true positive predictions to the total number of positive predictions) against recall (the ratio of true positive predictions to the total number of actual positives). AUPR analysis provides a single value that represents the model's overall performance. This analysis shows that our time-based DFG-inferred networks for both maize shoots (FIG. 19A) and roots (FIG. 19B) have the highest AUPRs, significantly outperforming networks derived from other network inference methods or random network models (p-value <0.001, permutation test). Therefore, the time-based DFG-inferred networks were selected for further downstream analysis in our model-to-crop N-GRN analysis.

In systems biology, networks with high precision offer a more focused selection of high-confidence true TF-gene interactions, thereby facilitating more targeted experimental validation. Thus, to further refine the accuracy of our time-based N-GRNs, we employed a Precision-Recall (PR) curve-based “pruning” approach-which prunes the network to include only high-confidence TF-target edges (Marbach et al. 2012; Chen and March 2018). For the AUPR analysis, we used TF-target interactions validated in the maize cell-based TARGET assay for the 23 TFs of maize whose N-responsive span the temporal N-response RNA-seq dataset (FIG. 2). This analysis enabled us to set precision cutoffs at 0.33 for the maize shoot GRN and 0.45 for the maize root GRN (FIG. 3B). These thresholds to identify high confidence edges in all the TFs in the N-GRN were chosen based on the point at which the PR curve plateaued on the recall x-axis. The TF-target network edges that passed these cut-off thresholds were deemed high-confidence TF-targets in the nitrogen-signaling GRNs (N-GRNs). This GRN pruning for high-confidence TF-targets retained nearly all maize NxTime responsive TFs (~200/203), while only ~30% of target N-response genes (~700/2,500) were retained (FIG. 3B). Several N-related GO terms are still enriched in the pruned genes, including nitrate assimilation (GO: 0042128), glutamine family amino acid biosynthetic process (GO: 0009084) and glutamate metabolic process (GO: 0006536), as well as energy-related GO terms, such as translation (GO: 0006412), regulation of proton transport (GO: 0010155) and electron transport chain (GO: 0022900).

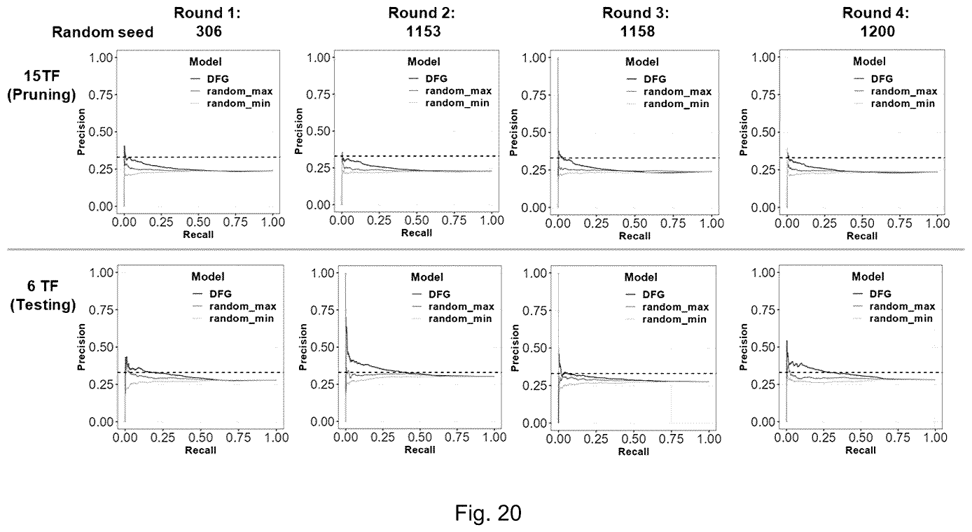

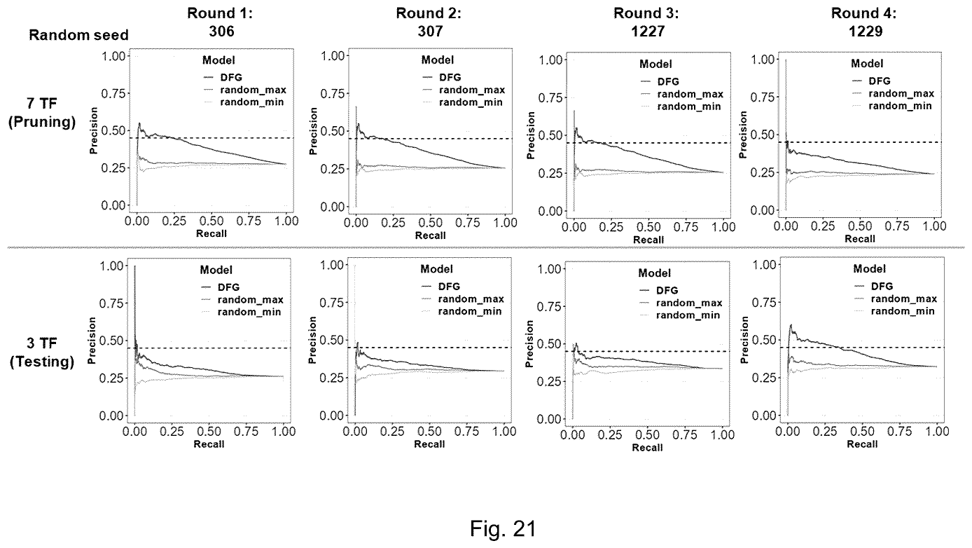

We next sought to ensure that our GRN pruning step was not overly influenced by the choice of TF validation datasets for the 23 maize TFs tested. To this end, we allocated 70% of the 23 maize TFs whose regulated targets were validated in TARGET assay to the pruning set, and the remaining 30% TS for testing, with the division process randomized and repeated four times. Despite slightly different AUPR scores depending on the subset of TFs selected, both pruning and testing phases returned PR curves significantly superior to those of random networks (FIG. 20; FIG. 21), indicating that our GRN pruning method was not biased by a few TFs amongst the 23 maize TFs whose targets we validated in the TARGET assay.

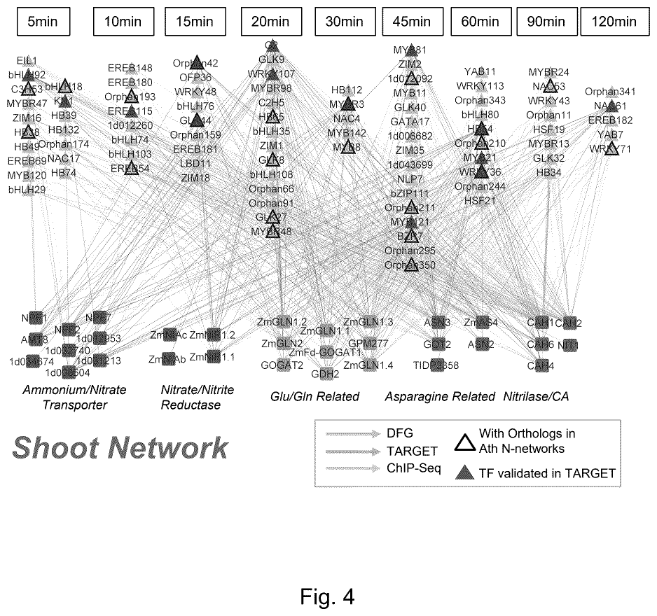

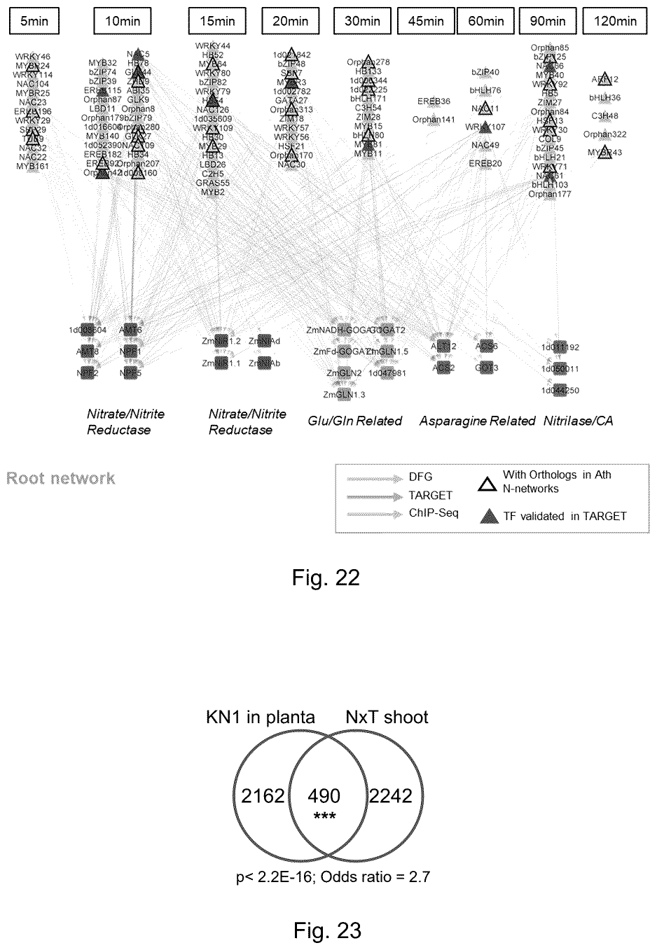

Next, to elucidate a high-confidence temporal maize N-GRN, we built ensemble N-GRNs by integrating TF-targets identified from; (i) our pruned DFG time-based nitrogen response network, (ii) validated TF-target regulation data from the 23 TF TARGET assays, and (iii) TF-target binding data from the published ChIP-Seq dataset for 104 maize TFs (22 TFs in the shoot, and 18 TFs in the root NxTime genes) (Tu et al. 2020). In our temporal N-response network in maize, the temporal action of 92 TFs in shoot and 111 TFs in root are based on their >2-fold N-induction in JIT time bins spanning from early (5 min) to late (120 min) N-responders. As a GRN visual, we highlighted the sub-network of TFs regulating the nitrogen uptake/assimilation pathway genes in maize shoots (FIG. 4) and roots (FIG. 22). This ensemble approach reveals the temporal nature of the GRN controlling nitrogen uptake/assimilation in maize.

### “Network Walking” Uncovers a New Role for KN1 in the Maize Nitrogen Response

Our temporal response N-network uncovered a set of novel early N-response genes (1,047) including 70 TFs (See Table Y) whose relevance to the N-response was not previously known. This includes a set of six TFs also listed in Table X: CH3H53, BES1, C3H26, WHY1, HB65 and Zm00001d005323. #Surprisingly one such TF is the well-studied transcription factor Knotted1 (KN1) known to be involved in maize leaf development (Greene et al. 1994; Kerstetter et al. 1997) which we identified as a “first responder” in the temporal nitrogen signaling cascade. This finding was based on our analysis of our temporal N-response GRN and time-based JIT responses of its genes. KN1 expression was significantly altered within 5 minutes of N-treatment (FIG. 2A). Previous studies have shown that KN1 is essential for shoot apical meristem function, orchestrating key developmental processes in maize leaves, tassels, and ears (Greene et al. 1994; Bolduc et al. 2012). A new role for KN1 in the temporal N-response in maize is supported by our discovery of a significant overlap between maize shoot NxTime genes uncovered in this disclosure and validated KN1 target genes in planta identified as mis-regulated in kn1 mutants (Bolduc et al. 2012) (p-value <2.2E-16, Fisher's exact test) (FIG. 23). These findings support a potential new role for KN1 in mediating the nitrogen response in maize shoots in planta.

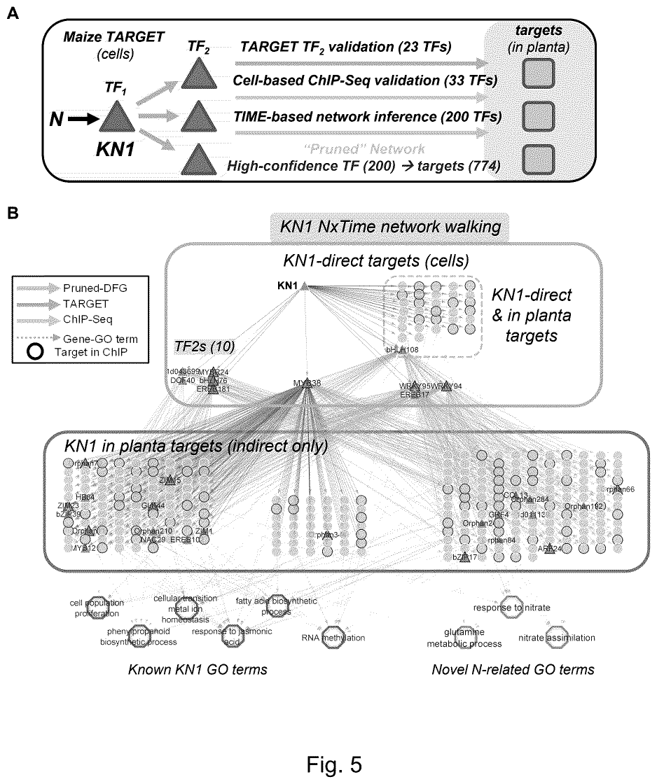

To validate the hypothesis that KN1 is an early N-responder that mediates the N-GRN response in maize, we sought to decipher the network path that KN1 might mediate to regulate these N-response target genes in planta. To this end, we first identified the direct regulated KN1 targets in maize cells by conducting a TARGET TF-perturbation experiment (FIG. 2A). Interestingly, despite KN1's early response to nitrogen, its direct targets identified in the TARGET assay rank 15th out of 23 TFs significance of overlap with NxTime genes in the shoot (FIG. 2B). This result suggests that KN1's regulatory effect on NxTime genes might be mediated by secondary transcription factors (TF2s) regulated directly by KN1. To test this hypothesis, we used “Network Walking” approach described in (Brooks et al. 2019, 2021) to trace the connections from KN1 to direct regulated TF2 genes, which would link KN1 to its indirect targets in planta (FIG. 5A). For this Network Walking approach, we used the ensemble N-GRN in shoots including edges from pruned high-confidence DFG, TF-targets validated in the cell-based TARGET assay, and cell-based ChIP-Seq data. The resulting “Network Walk” enabled us to connect KN1 indirectly to 63.1% of it's in planta N-responsive targets (309/490), via 10 N-responsive TF2s that are direct regulated targets of KN1 (FIG. 5B). The targets in this KN1 influenced network of N-response genes share GO terms with targets identified using kn1 mutants in planta (Bolduc et al. 2012), such as “cell population proliferation”. Interestingly, GO term analysis reveals a novel involvement of KN1 in nitrogen metabolism (FIG. 5B), extending its known role as a master regulator in shoot meristems (Smith et al. 1992; Kerstetter et al. 1997). Finally, to compare the KN1 binding data in maize cells and in planta, we performed additional TF-target validation of KN1. We did this by conducting ChIP-Seq on KN1 TF tagged with a small biotin ligase recognition peptide (BLRP) overexpressed in maize protoplasts (Lau and Bergmann 2015; Matsuda et al. 2017) (See Methods). These new ChIP data showed a significant overlap between the KN1 binding genes with the most significant cis-binding motifs similarities detected both in maize leaf protoplasts (this disclosure) and in ear tissue (Bolduc et al. 2012) (FIG. 24). Overall, these results suggest that protoplast-based overexpression of KN1 accurately reflects the gene binding and regulatory activities occurring in maize in planta.

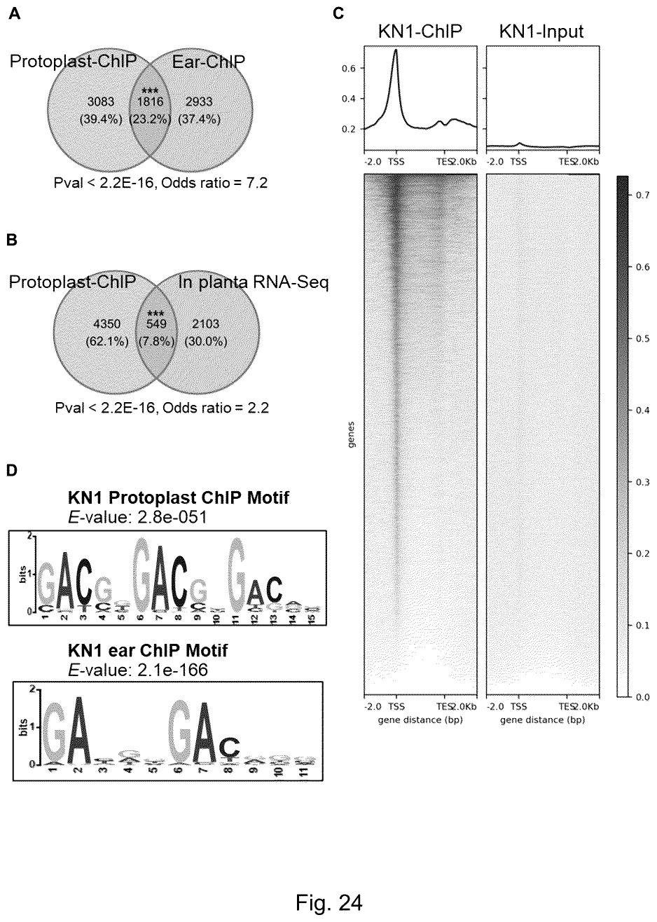

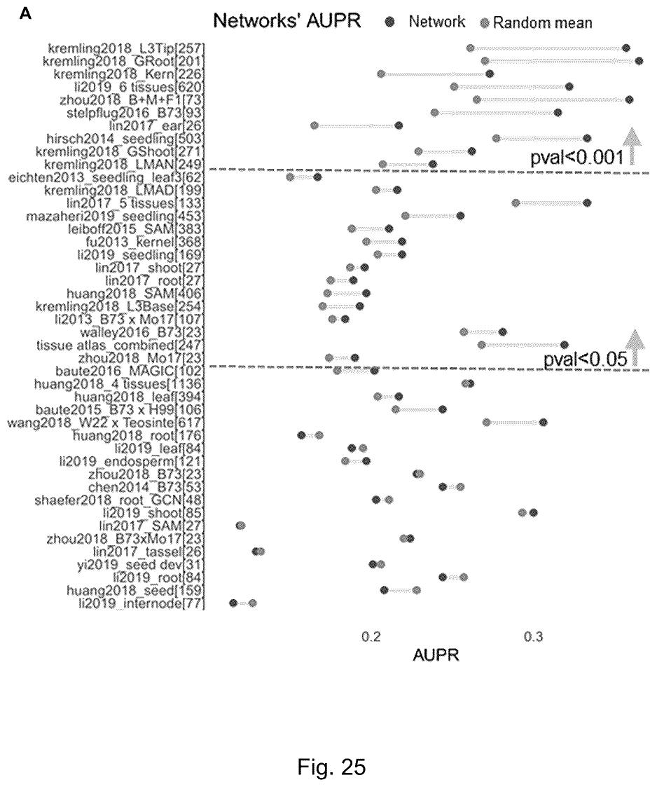

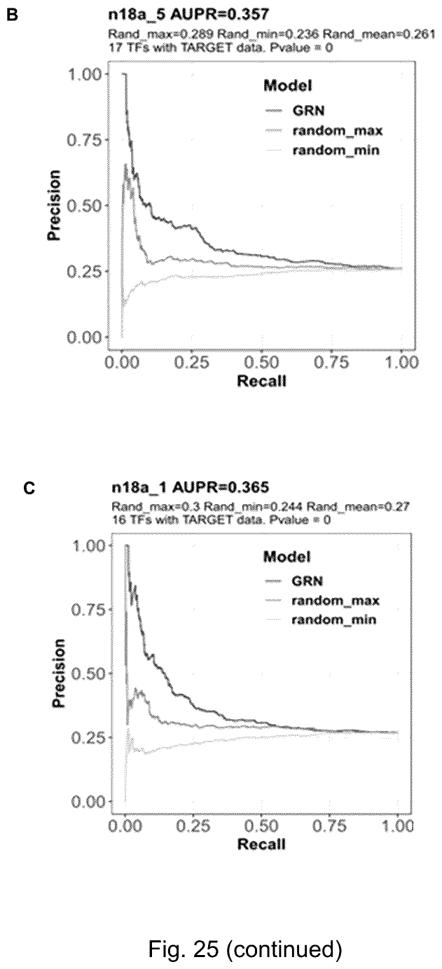

The 23 TF TARGET dataset of maize can also be used for validating various other networks. Utilizing AUPR analysis, we used the TF-target data for these 23 maize TFs to perform AUPR analysis of 45 GRNs from previously published research (Zhou et al. 2020). Our analysis identified 25 maize GRNs whose performance metrics surpassed random networks (p<0.05, permutation test with 1000 repeats), including ten GRNs with p-values smaller than 0.001 (FIG. 25). These ten maize GRNs include various tissues and genotypes are encompassed by the present disclosure.

### Model-to-Crop Conserved N-Response Genes Enhance Machine Learning of Genes-of-Importance to NUE

In the examples, we focused on maize to infer and validate a temporal GRNs involved in N-responses in this major crop. In example, we describe how our model-to-crop analysis showed that use of orthologous NxTime genes in machine learning significantly enhanced the predictive power of gene models for NUE, as in Cheng 2021 (Cheng et al. 2021). We first set out to identify the set of orthologous NxTime genes shared between maize and Arabidopsis. In our proof-of-principle example, we focused on the N-GRN subnetwork of TF predicted to regulate multiple genes in the N-uptake/assimilation pathway. We found that the shoot ensemble N-GRN for maize (FIG. 4) includes 92 TFs, 22 of which have orthologous TFs found in the Arabidopsis predicted networks (Gaudinier et al. 2018; Varala et al. 2018) (FIG. 4, not 22 validated TFs are denoted as triangles with black boarder). Note, for orthology inference between model-to-crop, we used the stringent orthology method OrthoFinder2 (Emms and Kelly 2019) (see Material and Methods section). With the discovery of orthologous TFs targeting genes in the N-pathway model-to-crop, we expanded our cross-species analysis to all the NxTime genes shared between maize and Arabidopsis. Through gene orthology, we found 353 shoot NxTime genes (34 TFs) and 406 root NxTime genes (41 TFs) in maize have orthologs in Arabidopsis that are also NxTime genes, which we refer to hereafter as conserved NxTime genes.

We next investigated whether using the conserved NxTime gene sets from our current N-response time-course experiments in maize and Arabidopsis could enhance machine predictions of NUE phenotypes in the field.

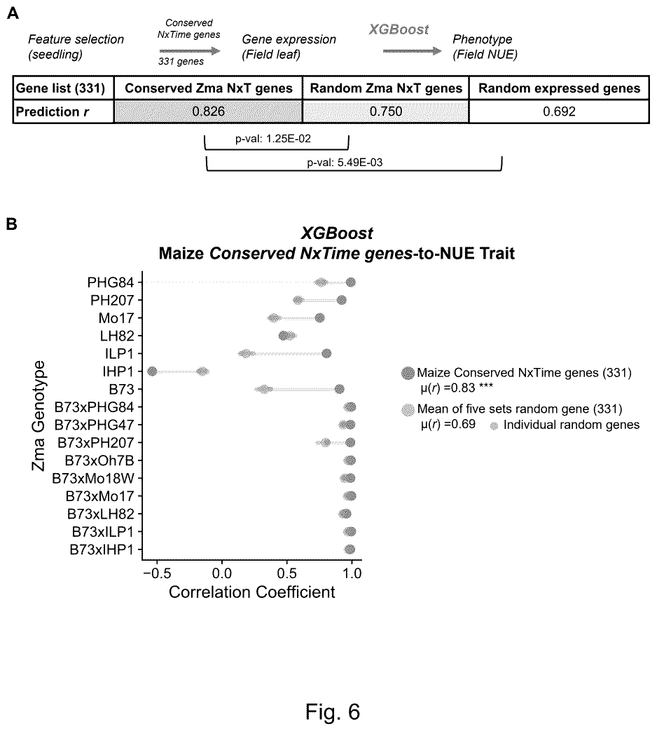

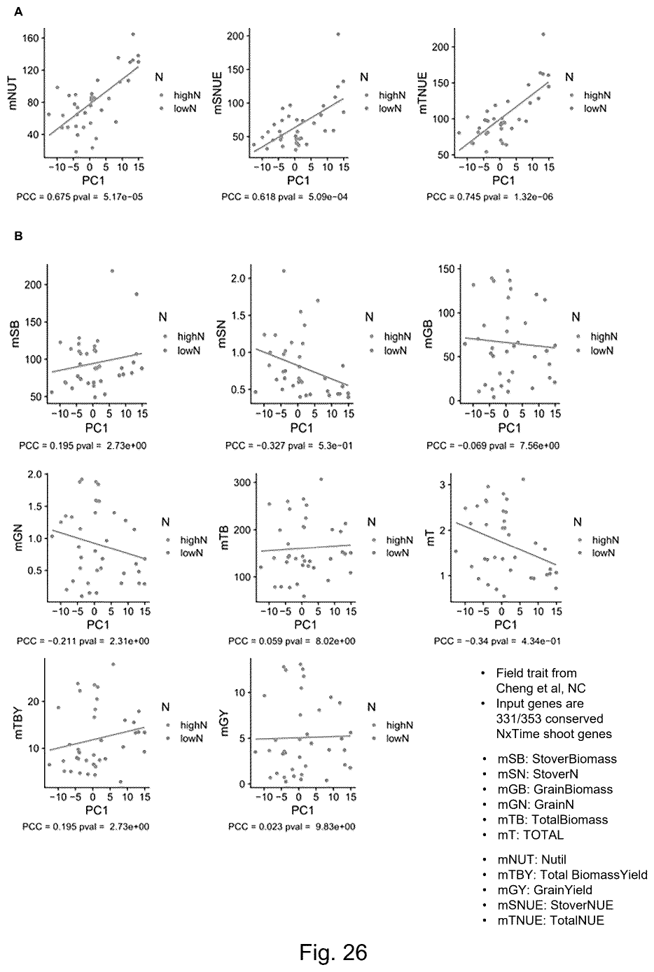

For machine learning, we used 331 out of 353 conserved maize NxTime genes identified in this disclosure which were expressed in the Cheng et al. maize dataset (Cheng et al. 2021) along with the phenotypes. To reduce dimensionality and avoid overfitting, we tested whether using the 331 conserved NxTime genes was a biologically principled way to enhance prediction. For our machine learning, we employed the XGBoost algorithm (Chen and Guestrin 2016), and used the field expression data of these selected maize genes to predict NUE phenotypes determined in the field from (Cheng et al. 2021). We found that the 331 model-to-crop conserved NxTime genes significantly outperformed the same number of randomly selected NxTime genes (pval: 1.25E-02) or expressed genes (pval: 5.49E-03) in predicting field NUE phenotypes (FIG. 6). Each gene within this conserved NxTime set of 331 maize genes was assigned a feature importance score, termed NUEscore, to quantify its predictive value of the traits across the 16 maize genotypes. Further, we examined the correlation between the eigengenes of these 331 conserved NxTime maize genes and numerous phenotypic field traits (Cheng et al. 2021). We found significant associations exclusively with NUE traits, rather than grain yield or biomass (FIG. 26). These findings demonstrate the use of the model-to-crop conserved NxTime genes identified in seedlings as reliable features for predicting NUE in mature maize grown under distinct N-field conditions.

NUE Regulon: Integration of Gene Networks with Machine Learning Scores for NUE Trait Predictions

To further enhance our model-to-crop GRN approach, in the examples below, we describe the NUE Regulon pipeline, which integrates; i) high-confidence GRNs, ii) gene orthology and iii) machine learning to learn network modules-of-importance to a trait. This disclosure relates to NUE traits, but the approach and pipeline can be applied to any species or trait of interest.

### Step 1: NUE Regulons: Integration of GRN and Machine Learning Knowledge:

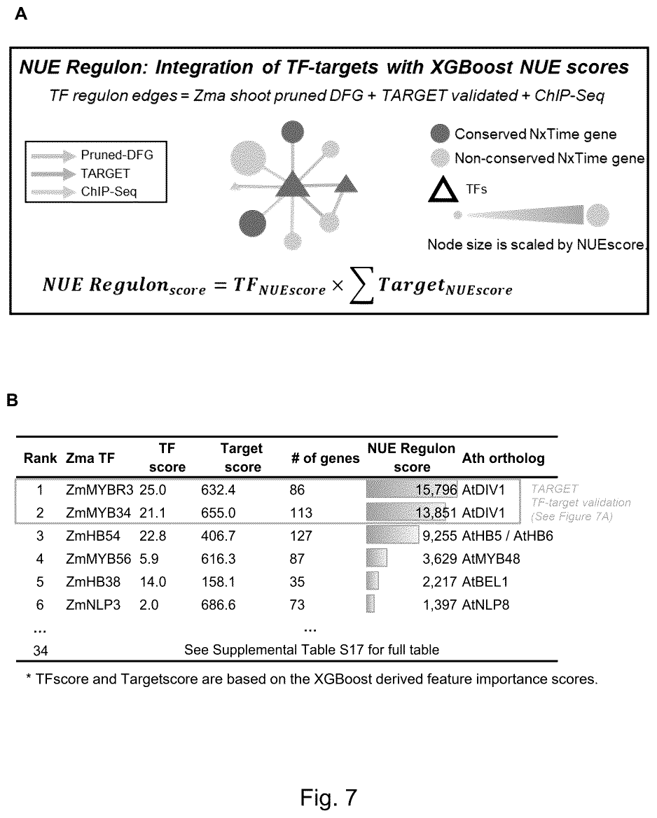

This first step aims to integrate network knowledge from the GRNs with machine learning of genes of importance to NUE. The goal is to identify the TFs whose target genes are collectively ranked the highest for importance to NUE. To this end, we used the ensemble N-GRN for maize shoots and incorporated the XGBoost NUE scores of the 331 NxTime genes conserved model-to-crop. We initiated this process by isolating a subnetwork within the ensemble N-GRN (FIG. 7A). The NUE-centric subnetwork includes the TF and their target genes (including TF2 targets) that are NxTime genes conserved across model-to-crop (green nodes, respectively in FIG. 7A).

### Step 2: NUE Regulon Score: Ranking Network Modules-of-Importance to NUE.

Next, we calculated the NUE Regulon score for each TF in the subnetwork. This NUE Regulon score represents a weighted importance to NUE (from XGBoost) of each TF and their target genes from the ensemble GRN (FIG. 7A). This NUE Regulon score is calculated from the NUE score for the TF times the aggregated NUE score of its NxTime target genes in the ensemble N-GRN (FIG. 7A). Ranking the NUE Regulon scores this way allowed us to identify TF hubs within NUE-centric subnetwork, distinguished not just by their network connectivity, but also by their high predictive value for targeting genes affecting NUE traits. MYBR3 and MYB34 emerged as the top two TFs relevant to maize NUE based on NUE Regulon score (FIG. 7B). This ranking is also supported by experimental data: 1) The direct targets of MYBR3 and MYB34 validated in the cell-based TARGET assay were the top-ranked TFs for specificity to regulating NxTime genes in maize shoots (FIG. 2B), and 2) In our gene orthology analysis, each of these maize MYB TFs are orthologous to Arabidopsis AtDIV1, for which loss-of-function mutants have been shown to have enhanced NUE, specifically under low nitrogen conditions (Cheng et al. 2021).

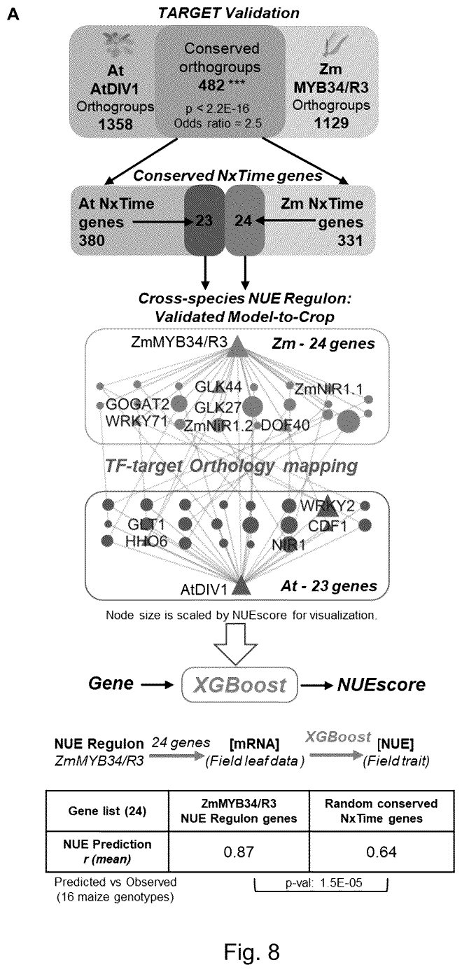

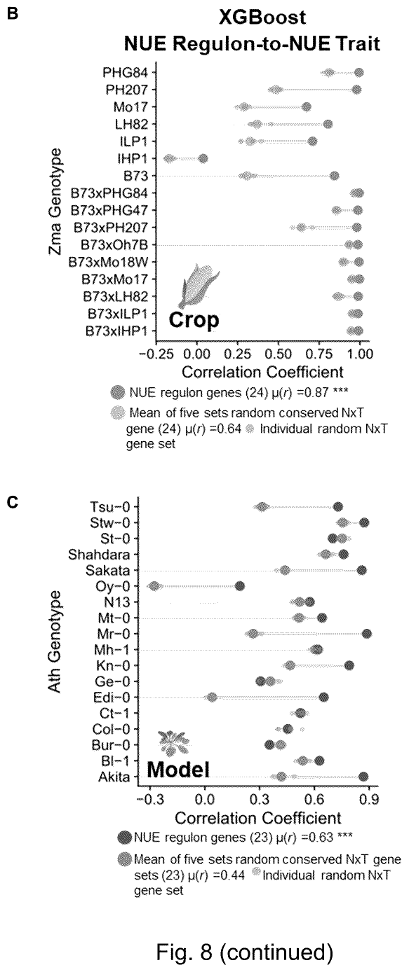

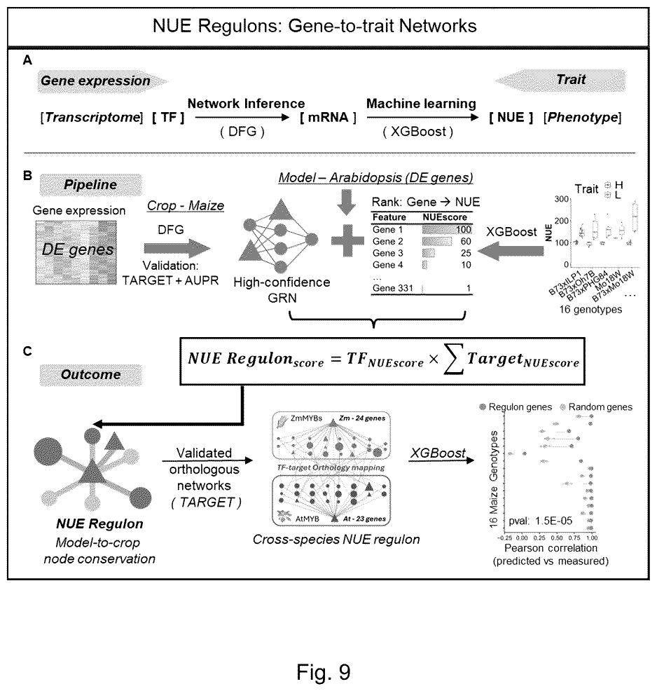

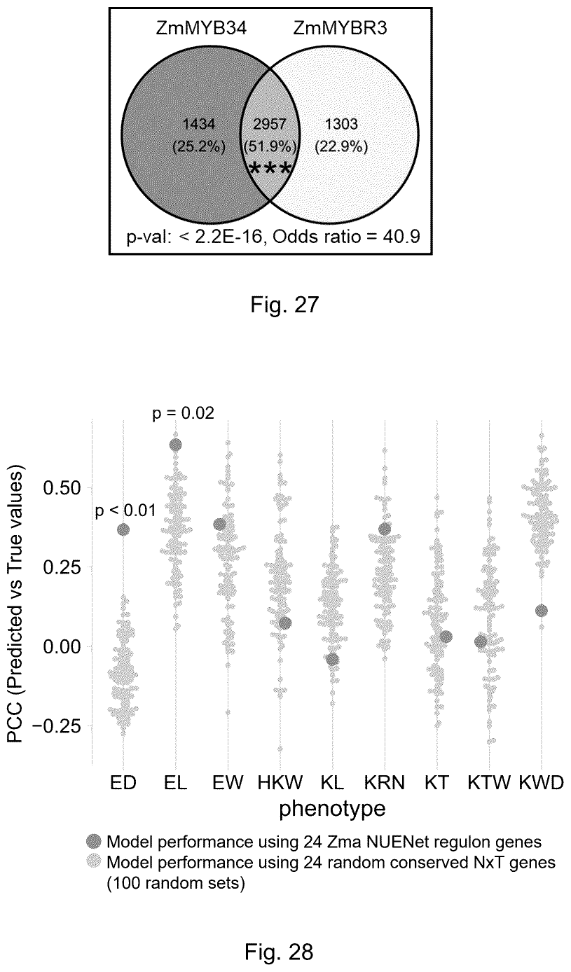

Step 3: Cross-species NUE Regulons: TF-target modules conserved model-to-crop. Given the many-to-one orthologous relationship between maize MYB34, MYBR3 and Arabidopsis AtDIV1, we first tested the potential redundancy of these two maize MYBs functions. We did this experimentally by comparing the direct regulated target genes of ZmMYB34 and ZmMYBR3 derived from TARGET TF perturbation assays performed in TF-transfected maize cells. This analysis revealed a significant overlap of the ZmMYB34 and ZmMYBR3 validated target genes (p-val<2.2E-16, Fisher's exact test) (FIG. 27), suggesting a functional redundancy between these two MYB TFs in maize. The intersection of target genes for ZmMYB34 and ZmMYBR3 (n=2,957) called ZmMYB34/R3, was used as their validated targets for the ensuing comparison to targets of their orthologous Arabidopsis TF AtDIV1 targets validated in the TARGET assay in Arabidopsis leaf protoplasts. We found a significant overlap of orthogroups targets (p<2.2E-16, Fisher's exact test) regulated by maize ZmMYB34/R3 and Arabidopsis ortholog AtDIV1 (FIG. 8A). Importantly, this finding indicates a conservation of orthologous TF-target regulatory function across 160 million years of evolutionary divergence between maize and Arabidopsis.

Not every orthologous validated target shared between ZmMYB34/R3 and AtDIV1 is essential for the NUE trait. By overlapping the orthologous targets shared between ZmMYB34/R3 and AtDIV1 with the conserved NxTime genes whose expression effectively predicted NUE outcomes in the field, we identified a specific subset of 23 Arabidopsis and 24 maize genes within a cross-species NUE regulon (FIG. 8A). This conserved NUE regulon includes key nitrogen pathway metabolic enzymes, such as nitrite reductase 1 (NIR1) and NADH-dependent glutamate synthase 1 (GLT1), as well as 3 TF2s including validated TF regulators of the N-response in Arabidopsis such as HHO6 (Varala et al. 2018; Ueda et al. 2020) and CDF1 (Varala et al. 2018) (FIG. 8A).

### Step 4: Evaluation of Model-to-Crop NUE Regulon in Predicting NUE in Field.

Given the conservation of this NUE regulon from model-to-crop (ZmMYB34/R3 & AtDIV1), we evaluated its potential to predict NUE traits in field grown maize using the XGBoost pipeline. Impressively, the predictive performance of the cross-species NUE Regulon (ZmMYB34/R3 & AtDIV1) target genes significantly surpassed that of an equivalent number of randomly conserved NxTime genes in both maize (pval: 1.5E-05-Wilcoxon signed-rank test) (FIG. 8B) and Arabidopsis (pval: 7.9E-04-Wilcoxon signed-rank test) (FIG. 8C). From the XGBoost models, we retrieved the NUEscore for the conserved regulon genes and visualized it on an orthology mapping plot (FIG. 8A). To further confirm the predictive power of the ZmMYB34/R3 regulon genes, we tested them on a different maize dataset which including expression and field phenotypes from 137 Chinese elite inbred lines (Li et al. 2023). It's known that maize ear development is significantly affected by nitrogen availability (Liao et al. 2012; Gheith et al. 2022). Using random forest models, we found the 24 genes in the ZmMYB34/R3 NUE regulon show significantly better prediction performance for maize ear diameter and ear length, compared to an equal number of random conserved NxTime genes (FIG. 28).

The disclosure demonstrates that the described cross-species NUE Regulon analysis pipeline identified, a precise set of orthologous TF-target genes conserved model-to-crop whose expression levels significantly improved machine learning of NUE trait prediction in Arabidopsis and maize grown in the field. This reveals that bridging 1 model-to-crop regulatory links between model-to-crop can enhance the ability to identify genes-of-importance to NUE. Information gained from this NUE regulon can be applied in molecular breeding or transgenesis to improve NUE in crops.

### Discussion of Examples

Without intending to be constrained by any particular theory, it is considered this disclosure provides a description of a combined computational and experimental strategy to identify genes-of-importance to an agronomic trait. In a systems biology approach, the NUE Regulon analysis pipeline integrates transcriptomics & traits by combining network inference, machine learning and model-to-crop translation. Specifically, we show how a cross-species NUE Regulon significantly enhances machine learning of gene network regulons-of-importance to NUE in a model, Arabidopsis and also in field grown mature maize plants. Impressively, this disclosure, the described model-to-crop study indicates a conservation of TF-target regulatory module function across 160 million years of evolutionary divergence between maize and Arabidopsis. More broadly, this model-to-crop analysis approach can be applied to any species or trait-of-interest. Below, we discuss how transferring integrated knowledge from data rich experimental models-to-crops presents significant opportunities, as well as future challenges.

### Model-to-Crop Translation of Temporal Regulatory Networks Controlling NUE.

This disclosure uses time, a largely unexplored dimension of the N-GRN response, especially in maize. As causality moves forward in time, fine-scale time-series RNA-seq data is a valuable source to derive predictive networks, as we have previously shown in Arabidopsis (Krouk et al. 2010, 2013; Varala et al. 2018). In this disclosure, we implement and validate a time-inferred causal gene regulatory network (GRN) in maize. We then compare it to a parallel study in the model Arabidopsis (Varala et al. 2018) to reveal N-regulatory temporal networks and network regulons conserved model-to-crop.

To do this, we first deciphered the temporal control of nitrogen responsive gene regulatory networks (N-GRNs) in maize using fine-scale time-course RNA-Seq experiments consisting of 10 fine-scale time-points (0-120 min) (see Methods). Using this data, we identified the maize NxTime response genes using spline analysis (Storey et al. 2005; Varala et al. 2018), and identified their temporal cascade of regulation using Just-In-Time (JIT) analysis (see Methods), as we previously did in Arabidopsis (Varala et al. 2018). By comparing the N-responsive JIT genes in maize with their counterparts in Arabidopsis from a parallel experiment (Varala et al. 2018), we uncovered a temporally resolved nitrogen signaling cascade of gene functions conserved between model-to-crop (FIG. 1).

Further leveraging our time-course experimental design of N-responsive genes, we employed a time-based network inference method called Dynamic Factor Graph (DFG) (Krouk et al. 2010, 2013; Varala et al. 2018; Brooks et al. 2019) to construct causal N-GRNs in maize shoots and roots. To experimentally validate these inferred networks, we used TARGET, a medium-throughput plant cell-based TF perturbation assay (FIG. 2). Results from the TARGET assay validated TF-target direct regulatory edges for 23 maize TFs whose targets encompass ~85-90% of the early-to-late temporally N-responsive genes in planta (FIG. 2B). The validated target data for these 23 maize TFs also were used precision/recall AUPR analysis to prune the accuracy of the predicted TF-target edges for ~200 TFs and 700 target genes (~16,000 high confidence TF-target edges) in our N-GRNs (FIG. 3). As an example, we showed that this pruned N-GRN includes the validated and predicted TF regulators of 32 genes in the N-uptake/assimilation pathway in maize shoots (FIG. 4). Our integration of the high-confidence TF-target edges identified in maize cells (via TARGET Assau) with in planta data of a maize TF mutant in leaf development (Bolduc et al. 2012), led to the discovery of a novel role for the well-studied maize Knotted1 TF in nitrogen signaling, using an approach called “Network Walking”, described in detail in (Brooks et al. 2019, 2021) (FIG. 5).

Next, the disclosure demonstrates how TFs network regulons in the pruned GRN might influence NUE outcomes. To this end, we integrated our temporal N-response GRNs with NUE phenotype data using XGBoost machine learning analysis (Chen and Guestrin 2016), applied to maize as described in Cheng et al., 2021. In our model-to-crop analysis, we demonstrated that conserved NxTime genes between maize and Arabidopsis can reliably predict NUE in maize. Moreover, by integrating network structures with NUE scores, we identified and validated a conserved Maize-Arabidopsis NUE network regulon (ZmMYB34/R3-AtDIV1) of 24/23 genes whose expression levels significantly enhances the ability to predict NUE traits from RNA in left out samples for both model-and-crop.

### Exploiting Time-Series Data to Enhance Regulatory Network Inference and Learn Early N-Signaling Events in Maize.

Our analysis takes advantage of time-series data to infer regulatory networks. Unlike steady-state experiments that take a snapshot the transcriptome at a single point, time-course experiments find changes in gene expression throughout development or treatment, providing a deeper insight into gene regulatory dynamics (Storey et al. 2005; Aryee et al. 2009; Krouk et al. 2013). In addition to enhancing predictions of TF-target causality, measurements across multiple time-points act as replicates, significantly boosting the statistical power for differential expressed gene analysis (Tai and Speed 2009; Sun et al. 2016). In this disclosure, we used a spline model to identify NxTime genes (FIG. 1C). The spline model is known for its flexibility and efficiency in detecting non-linear gene expression changes (Coffey et al. 2014; Spies et al. 2017). Since all time-points are used in the DE testing, instead of treated versus control at individual time points, spline modeling handles noisy data and increases confidence in detecting DE genes (Luan and Li 2003; Krouk et al. 2010; Varala et al. 2018; Alvarez et al. 2021; Shanks et al. 2024). Recently, spline modeling has also been applied in detecting DE genes from temporal single-cell transcriptomic data in animals (Van den Berge et al. 2020; Fan et al. 2024). Our time-course experiment enabled us to pinpoint the first time-point that maize just-in-time (JIT) genes that respond to nitrogen above a two-fold threshold (FIG. 1C). These JIT gene sets, were also observed in a parallel study in Arabidopsis (Varala et al. 2018) and by comparison, we determined that share biological processes conserved across model-to-crop (FIGS. 1D & E). This finding suggest a conserved plant mechanism for temporal transcriptional control of metabolic genes in plants in response to nutrient sensing, analogous to systems seen in E. coli and yeast (McAdams and Shapiro 2003; Zaslaver et al. 2004; Chechik et al. 2008). In other studies, in addition to the spline model, other methods for time-course transcriptome analysis include Granger-causality (Heerah et al. 2021), hidden Markov model (Leng et al. 2015), and deep learning (Yuan and Bar-Joseph 2021).

Prior studies of nitrogen signaling responses in maize have primarily focused on single time-points (Ge et al. 2020, 5; Wang et al. 2020b; Buoso et al. 2021a; Cheng et al. 2021). This disclosure expands this knowledge-base to gene expression dynamics in both maize shoots and roots, by capturing previously undetected early response genes and has nearly doubled the N-responsive gene knowledge from previous studies (FIG. 12). By comparing our new time-series N-response data in maize with a parallel time-course experiment in Arabidopsis, we uncovered a conserved N signaling cascade between two species, despite their differences as a C3 dicot (Arabidopsis) and a C4 monocot (maize). In addition to identifying a large number of early- and dynamic N-response genes that were missed in steady state studies of maize, we identified 353 NxTime genes in the shoot and 406 NxTime genes in the root conserved across model-to-crop. Our approach to identify NUE Regulon provides a systematic way for identifying temporal response networks and enhances the transfer of knowledge from model organisms to crops.

### Gene Regulatory Network Validation & Refinement: Precision/Recall Analysis and Network Pruning.

One bottleneck in transferring knowledge from model-to-crop is the validation of TF-target edges in the GRNs, especially in crops. An aspect of network inference studies is the incorporation of validated TF-to-target data which enables network refinement and “pruning” of high confidence regulatory edges in a GRN. To enable such GRN validations in maize, we validated maize TF-targets in by adapting a medium through-put cell-based TF perturbation assay called TARGET to maize, from the protocols initially developed in Arabidopsis (Bargmann et al. 2013; Brooks et al. 2019). The TARGET assay enabled us to rapidly validate direct regulated TF-targets in isolated maize cells. The TF-target validation in the TARGET assay in maize can be completed in two days for four to eight TFs (in triplicate) from protoplast isolation to RNA extraction. This TARGET system has been successfully used in Arabidopsis and rice, which have demonstrated its ability to capture TF-target regulatory edges in isolated cells with relevance to in planta studies (Bargmann et al. 2013; Varala et al. 2018; Brooks et al. 2019; Shanks et al. 2022). Herein, we show that the genome-wide targets of 23 maize TFs validated in the maize cell-based TARGET system capture ~90% of the NxTime-responsive genes in maize shoots and roots (FIG. 2). Compared to ChIP-Seq, and other cell-based TF perturbation assays (Zhu et al. 2024), the TARGET assay identifies directly regulated TF-targets. This is accomplished by controlling entry of the TF into the nucleus, using a TF-GR glucocorticoid receptor (GR) domain fusion. TF nuclear import is thus controlled by addition of dexamethasone (DEX). When done in the presence of cycloheximide (CHX) treatment, the regulation of direct targets ensures, without triggering the translation of secondary TFs (Bargmann et al. 2013). This approach to identify direct regulated targets of a TF (e.g. TF-GR fusion+CHX) has also been used extensively in planta (Yamaguchi et al. 2015).

The TARGET assay also provides an evidence-based method to validate and prune the GRNs. Instead of arbitrarily selecting the top 1% or 10% as the high-confidence TF-targets in GRNs, we used Area Under the Precision-Recall (AUPR) to guide where to prune the network (FIG. 3). AUPR is suggested to be the optimal performance measurement of a network, as it emphasizes true positive predictions and ignores true negatives, which could overwhelm the GRN prediction (Aalto et al. 2020; Pratapa et al. 2020). In this disclosure, we used the genome-wide targets of 23 maize TFs that we validated in the cell-base TARGET assay to perform AUPR analysis. This enabled us to prune our maize GRN for high-confidence edges connecting ~200 TFs to ~700 target genes via ~16,000 edges (FIG. 3). Given that many agronomic traits are governed by such complex networks, having a high-quality GRN is important for identifying key genes that control traits, including NUE (Hirel et al. 2007; Gao et al. 2022). The recently proposed “omnigenic” model hypothesized that all genes expressed in a cell contribute to controlling a complex trait (Boyle et al. 2017; Mathieson 2021). GRNs are highly interconnected, with many TFs influencing the expression of core genes. For instance, we observed that the temporal nitrogen response GRN controlling nitrogen uptake/metabolism genes in maize is highly interconnected and regulated by hundreds of TFs (FIG. 4; FIG. 22).

### Model-to-Crop Conservation of GRN Network Regulons: NUE Phenotype as a Prototype Working Example.

To untangle the GRNs, this disclosure provides and validates a framework to rank TFs within these networks to identify core regulators for complex traits such as NUE. Although previous studies in both plant (Obertello et al. 2015; Du et al. 2020) and animal research (Yu et al. 2012; Mueller et al. 2017; Möller et al. 2022) have highlighted the gene module conservation between model and non-model organism, the link between network module-to-phenotype remains largely unexplored (Langfelder and Horvath 2008). Our framework to rank regulatory genes relative to a trait combines cross-species comparison and machine learning. Our rationale is if a conserved GRN network regulon is validated in diverse species, such as Arabidopsis and maize, which are separated by 160 million years (Kumar et al. 2017), then this regulon is highly likely to be important to a trait. Moreover, the TARGET assay opens up higher throughput initial testing in the model.

To test this hypothesis, we focused on a sub-network regulon (NUE Regulon) for the NUE trait. To enhance our machine learning of genes-of-importance to NUE, we first reduced the dimensionality of genes used in our models to the 353 evolutionarily conserved maize-Arabidopsis NxTime gene sets (FIG. 6), as in Cheng et al 2021. Using this evolutionarily conserved NxTime gene set, we showed the model-to-crop N-response genes selected at the seedling stage (FIG. 6A) can predict the NUE phenotype at adult stages in the field (FIG. 6B). For the NUE Regulon approach, we calculated the “NUE Regulon score” by integrating the machine learning-derived gene importance scores of the TF and its targets within the network context (FIG. 7). Moreover, we validated the top TFs regulation by TARGET in model (AtDIV1) and crop (ZmMYB34/R3) using the cell-based TARGET assay (FIG. 8). This validated that a model-to-crop conserved ZmMYB34/R3 regulon for NUE which comprises only 24 genes, can accurately predict field NUE and ear phenotypes in maize (FIG. 8; FIG. 28). Using this precise gene set of TF regulon genes-of-importance to predict field outcomes in maize seedlings, can facilitate improvements in NUE through molecular breeding programs, transgenics or genome-editing, as described herein.

Overall, this disclosure integrates GRN analysis with machine learning and gene orthology to identify regulons conserved model-to-crop species. Moreover, we validated the orthologous TFs regulons across species in plant cells. While we focused on the NUE trait, this strategy can be applied to any other crop or traits by utilizing information from any model species. The selection for or modification of such NUE regulons can be used to enhance NUE in crops, improving yield and mitigating N-fertilizer contribution to greenhouse gas emission.

### Methods

### Plant Material and Nitrogen Treatment

Zea mays (B73) seeds were obtained from U.S. National Plant Germplasm System (npgsweb.ars-grin.gov/gringlobal/accessiondetail?accid=PI+550473). On day one, seeds were soaked in sterilized water overnight. On the next day, seeds were sterilized in a glass beaker with 50% commercial bleach (CLOROX) for 20 min. Then the seeds were rinsed five times with sterilized water, each time for 20 mins. Six sterilized seeds were paper rolled on a double-layered, pre-rinsed 60 cm×10 cm paper towel. Seeds were placed 2 cm below the paper edge. The paper rolls with seeds were covered with foil paper and stored in a plastic container filled with 200 ml water. Plants were checked daily. All plants were grown in a PERCIVAL growth chamber at New York University (16 h light-8 h dark, 27 °C at light-24 °C at dark cycle, 70% relative humidity). Water in each container was replaced by nitrate-free MS hydroponic medium (Caisson Labs-MSP10-50LT) on day six. On day eight, the germinated seedlings were taken out of the paper rolls and endosperms were removed with a razor blade by hand. After the removal of the endosperms, seedlings were grown in the fresh nitrate-free MS hydroponic medium. All plants were handled between 10 am and 12 μm Eastern Standard Time. The seedlings were starved from nitrogen for two days. On the day of the sampling (day ten), two hours after the start of the light period (10 am), seedlings were transferred to the fresh hydroponic medium containing either the Nitrogen in MS medium (20 mM KNO₃+20 mM NH₄NO₃) or 20 mM KCl and harvested at time intervals 0, 5, 10, 15, 20, 30, 45, 60, 90, and 120 minutes. Ten percent of nitrogen in the MS medium was labelled with the ¹⁵N isotope (Cambridge Isotope lab-NLM-390-1). Shoots and roots from three seedlings were combined as one biological replicate. Three biological replicates were collected per time point per treatment. They were immediately flash-frozen in liquid nitrogen and stored in an −80 °C freezer. The ground tissues were sent to UC Davis Stable Isotope Facility for ¹⁵N measurement. The ¹⁵N measurements in shoots and roots were plotted over time, and a simple linear regression model was applied using the ‘lm( )’ function in R. To calculate the correlation between gene expression and ¹⁵N levels, Pearson's correlation coefficients were calculated using the ‘cor.test( )’ function in R. P-values were adjusted for multiple testing using the ‘p.adjust (method=“BH”)’ function.

### RNA Extraction and Library Preparation for RNA-Sequencing

About 100 mg finely ground tissues were used for RNA extraction using the Zymo Direct-zol RNA Miniprep Kits (R2052). The RNA quality was checked on a Nanodrop and Agilent TapeStation (5067-5576). The Lexogen QuantSeq 3′ mRNA-Seq Library Prep Kit (015.2×96) with UMI (081.96) was used for the mRNA library preparation. The libraries were pooled and sequenced on the Illumina NextSeq 500 platform at NYU-CGSB Genomics Core using the HighOutput 1×75 bp mode.

The UMI-incorporated RNA-Seq libraries were analyzed following Lexogen's guidance (www.lexogen.com/quantseq-3mrna-sequencing/). The reads' UMI were extracted from raw fastq files using ‘extract’ command from UMI-tools v1.1.1 (Smith et al., 2017). The fastq files were trimmed by fastp 0.21.0 (Chen et al. 2018). The clean fastq files were aligned to maize genome version 4 downloaded from Ensembl Plant (Kersey et al. 2018) using STAR 2.7.6a (Dobin et al. 2013). The identical UMI reads were deduplicated using dedup command from UMI-tools v1.1.1 (Smith et al. 2017). The read counts matrix was generated by featureCounts v2.0.1 (Liao et al. 2014) from the deduplicated bam files.

### Identifying NxTime Genes Fitting a Spline Model

To identify the high confidence Nitrogen-responsive genes (referred to as NxTime genes), we fit the transcriptome data (+N vs −N) into a cubic spline model as previously described (Varala et al. 2018) with modifications (FIG. 1C). The data was modeled with a degree of freedom equal to five. Then we used the generalized likelihood ratio test (lrt) in edge package (Storey et al. 2005) in R to test the NxTime genes whose N-response curve of mRNA levels differs significantly from −N controls (FIG. 1C). The bootstrap method with 1000 iterations was applied in the lrt test. We set the cutoff for significant NxTime genes in shoot and root to include approximately 200 TFs. This cutoff prevents overfitting in the network inference step (avoiding too many regulators) and maintains consistency with the Arabidopsis time-course data. We identified 2,732 NxTime genes in shoot (q-value <0.05) and 2,294 NxTime genes in root (q-value <1E-5).

### Identifying Nitrogen Pathway Genes and Cross Annotation Model-to-Crop.

We conducted a comprehensive search across three databases, including MaizeGDB (Andorf et al. 2015), UniProt (The UniProt Consortium 2019) and KEGG (Kanehisa et al. 2023), for key enzymes in the nitrogen metabolism pathways. To avoid missing maize genes due to the lack of annotation, we performed reverse BLASTP search (Obertello et al. 2015) using Diamond (Buchfink et al. 2021) between well-annotated Arabidopsis genes and maize genes. Maize genes with e-value<1E-20 in both queries (maize to Arabidopsis, and Arabidopsis to maize) were annotated with Arabidopsis functions. The gene nomenclature adheres to MaizeGDB and TAIR conventions, except for those cited from published sources (Liu et al. 2022).

### Just-In-Time Analysis, GO Enrichment and Motif Enrichment

For the Just-in-time (JIT) analysis (FIG. 1C), we normalized the expression matrix using ‘betweenLaneNormalization( )’ function in EDASeq (Risso et al. 2011). From the normalized expression matrix, we calculated the gene expression fold-change (FC) of +N/−N at each time point. Genes were binned to the JIT categories base on their first-time showing |log 2FC|>1. The previously published Arabidopsis NxTime data (Varala et al. 2018) collected from identically treated Arabidopsis seedlings was re-processed following the same criteria for consistency. For all the GO enrichment analyses, the topGO package (Alexa et al. 2006) with the Maize-GAMER annotation (Wimalanathan et al. 2018) was used for maize and the org. At.tair.db (Carlson et al. 2019) was used for Arabidopsis. The Fisher exact test using ‘weight01’ algorithm (default in topGO) were applied to all the GO term enrichment tests. Motif enrichment was performed by SEA from MEME suite (Bailey and Grant 2021) using the default setting with the order2 background model calculated from all promoter regions (—bfile). Two cis-motif sources were used. The first was the consensus cis-motif clusters from Brooks et al (Brooks et al. 2019), containing eighty (sub) family level TF motifs. The second was the JASPAR2022 Plant Core motifs (Castro-Mondragon et al. 2022) which includes 656 high-quality plant TF motifs. Motifs with QVALUE<0.05 and ENR_RATIO>3 from the SEA outputs were considered as enriched for the gene sets.

### Maize TARGET Experiments for Medium Thru-Put Validation of TF-Targets in Cells.

All maize TARGET experiments listed in FIG. 2 were performed using the p1107 plasmid which contains a TF-GR fusion protein and selectable marker gene for FACS was described previously (Shanks et al. 2022).

The maize transfection protocol was adapted from our Arabidopsis TARGET protocol (Bargmann et al. 2013; Brooks et al. 2019) and Dr. Jen Sheen's protocol (molbio.mgh.harvard.edu/sheenweb/protocols.html) with some modifications. The maize seeds were soaked overnight and planted in pots filled with Turface MVP. The pots were placed at the growth chamber, under 16 h light/8 h dark diurnal cycle, at temperatures 27 and 24 °C respectively and 70% humidity for three days. After seeds were germinated and about 5 cm above ground, the pots were moved to the dark room at temperature 27 °C and 70% humidity for etiolation for seven days. On the day of the TARGET experiment, etiolated middle section (~4 cm) of the second leaves were cut into 1 mm pieces and soaked in the cell-wall digestion solution (1.5% cellulase RS, 0.3% macerozyme R10 (Yakult Honsha), 0.6M mannitol, 10 mM MES (pH 5.7), 1 mM CaCl2, 5 mM b-mercaptoethanol, and 0.1% BSA) in a petri dish. The petri dish was vacuum infiltrated for 30 minutes and shaken for 95 min at 50 rpm in the dark, followed by 10 min at 70 rpm to fully release cells. Maize protoplast cells were filtered through a 40 μm cell strainer (BD Falcon, USA) and spun down for 2 min at 280 g. The cells were washed with 10 mL Buffer (10 mM MES, pH 5.7, 0.6M mannitol, 10 mM KCl) three times. After the final wash, cells were resuspended in the Buffer to 1.0×10⁶ cells/mL and were ready for transfection. For protoplast transfection, we mixed 150 μL protoplasts (1.5×10⁵ cells), 40 ug plasmid and 120 μL Buffer in a 2 mm electroporator cuvette (BioRad #1652086). The BioRad Gene Pulser was used for the electroporation with one shot and the following condition: 150V, 200 OHMs, 125 uF. After electroporation, 700 μL cold Buffer was added to the cuvette. Then the protoplast mix was transferred to a new round bottom 2 mL tube and stored at room temperature in the dark overnight. On the next day, protoplasts were treated with either +Nitrogen (20 mM KNO₃ and 20 mM NH₄NO₃) or −Nitrogen (20 mM KCl) for two hours. The cells were treated with 30 μM cycloheximide (CHX) for 20 minutes (to block translation of secondary TF targets genes), then a three-hour 10 mM dexamethasone (DEX) treatment (to induce TF-GR nuclear import). After three hours, TF vector and control empty vector transfected protoplasts were FACS sorted for RFP signals into 150 μL TRI regent for RNA extraction (Zymo, R2061). The RNA quality was checked on the Agilent TapeStation (5067-5576). We used Lexogen QuantSeq 3′ mRNA-Seq Library Prep Kit (Lexogen, 015.2×96) for RNASeq library preparation.

### Arabidopsis TARGET Experiment to Identify Direct Regulated TF-Targets.

The Arabidopsis TARGET experiments for TFs listed in FIG. 2 were performed using the pBOB11 plasmid, as described previously (Brooks et al. 2019). An empty vector plasmid (with no TF of interest) was also transfected to be used as a control for identifying DEGs. The protocol for protoplasting and transfection leaf cell was adapted from the Sheen Lab (Yoo et al. 2007). In brief, rosette leaves from ~4-week-old soil-grown plants were harvested, sliced with a razor, and incubated in enzyme solution (1% cellulase R10, 0.2% macerozyme R10 (Yakult Honsha), 0.4 M mannitol, 20 mM MES pH 5.7, 20 mM KCl, 10 mM CaCl₂), and 0.1% BSA) for 90 min. Cells were strained through a 70 μM filter, collected by centrifugation at 300 g, and washed with W5 buffer (150 mM NaCl, 125 mM CaCl2, 5 mM KCl, 2 mM MES pH 5.7, 5 mM glucose). Cells were collected by centrifugation and transferred to MMg solution (400 mM mannitol, 10 mM MgCl₂, 4 mM MES pH 5.7) for transfection. Cells were diluted to a cell density of 6×10⁵ cells/ml with MMg and mixed with 20 μl plasmid DNA (20-50 μg DNA) in a 2 mL microcentrifuge tube. 120 μl PEG solution (40% PEG4000, 200 mM mannitol, 100 mM CaCl₂)) was added to the cells and mixed by inversion. After incubating for 2-5 min, W5 buffer was added, and the cells were washed twice in W5 buffer. Cells were then transferred to a 48-well plate and incubated overnight with shaking. To start the TARGET experiment, cycloheximide was added to a final concentration of 35 μM for 20 minutes, before addition of dexamethasone to a final concentration of 10 μM for 3 hours. After the incubation, cells were analyzed with a FACSAria II (BD Biosciences) and sorted directly into RNA Lysis Buffer from the Quick-RNA Miniprep Kit (Zymo Research), which was then used for RNA isolation following the manufacturer's instructions. Illumina library preparation was performed using the NEBNext Ultra II RNA Library Prep Kit for Illumina (NEB #E7770L) with NEBNext Multiplex Oligos for Illumina (NEB #E6609S) according to the manufacturer's instructions.

### Maize KN1 ChIP-Seq Experiment in Isolated Leaf Cells.

The maize protoplast ChIP protocol for KN1 targets (FIG. 5B) was adapted from the previous uChIP-Seq protocol (Lau and Bergmann 2015; Para et al. 2018) with modifications. In the p1107 vector, the biotin ligase recognition peptide (BLRP) tag was placed at the 5′ end of the inserted TF. The p1107-KN1 plasmid was transfected into the etiolated maize leaf protoplasts as described above. After transfection, a final concentration of 50 μM biotin (Sigma B4639) was added to the buffer (10 mM MES, pH 5.7, 0.6M mannitol, 10 mM KCl). Protoplasts were collected by centrifugation at 500 g for 2 min and resuspended in 500 μL buffer. About 60,000 transfected cells are sufficient for one ChIP reaction. For cross-linking, 13.7 μL of 36.5% formaldehyde was added and incubated at room temperature for 7 min. The reaction was quenched with 32 μL of 2M glycine and incubated at room temperature for an additional 7 min. The cells were then centrifuged at 600 g for 2 min at 4 °C using a swing-bucket rotor. After removing the supernatant, the pellet was rinsed twice with 500 μL of ice-cold buffer, centrifuging at 600 g for 2 min at 4 °C between washes. The final pellet was snap-frozen in liquid nitrogen and stored at −80 °C.

Chromatin was isolated by adding 100 μL of pre-chilled lysis buffer (50 mM Tris-HCl, pH 8, 10 mM EDTA, 1% SDS, and complete mini protease inhibitor) to the protoplasts, incubating on ice for 10 min, and resuspending by gentle pipetting. The lysate was transferred to a tube for sonication using a Bioruptor Pico Sonicator with 5 cycles of 30s on, 30s off in ice water. Subsequently, 500 μL of ChIP dilution buffer (10 mM Tris-HCl, pH 8, 150 mM NaCl, 1 mM EDTA, 1.1% Triton X-100, and complete mini protease inhibitor-Roche 11836153001) was added to the lysate, and the mixture was centrifuged at 12,000 g for 10 min at 4 °C. The supernatant (chromatin) was transferred to a new pre-chilled tube. This was repeated by adding 400 μL of ChIP dilution buffer to the pellet, centrifuging again, and pooling the supernatants to dilute SDS from 1% to 0.1%.

During centrifugation, Dynabeads® Protein A magnetic beads (Invitrogen 10001D) were washed by ensuring a homogeneous suspension and resuspending 20 μL of beads per sample in 1 mL of ChIP dilution buffer. The beads were placed on a magnetic stand for 30 s on ice, and the supernatant was discarded. This wash was repeated. The beads were then resuspended in 20*n μL of ChIP dilution buffer (n=number of reactions). Pre-washed Protein A (20 μL) was added to the chromatin mixture and incubated for 1 h at room temperature on a rotator. Beads were captured on a magnetic stand, and the supernatant was transferred to a new tube, setting aside 30-50 μL of chromatin as input for ChIP.

For immunoprecipitation, 50 μL of Streptavidin-beads (Invitrogen 65801D) were washed three times with 1 mL of ChIP dilution buffer and resuspended in 100*n μL of ChIP dilution buffer. The beads were added to the pre-cleared chromatin and incubated overnight with gentle agitation at 4 °C or for 1 h at room temperature. Beads were captured on a magnetic stand, the supernatant was discarded, and the beads were sequentially washed with 1 mL each of Wash buffers 1 (2% SDS), 2 (20 mM Tris-HCl (pH 8), 500 mM NaCl, 2 mM EDTA, 0.1% Deoxycholate, 0.1% (wt/vol) SDS and 1% (vol/vol) Triton X-100), 3 (10 mM Tris-HCl (pH 8), 250 mM LiCl, 1 mM EDTA, 1% (vol/vol) NP-40 and 0.5% (wt/vol) sodium deoxycholate), and 4 (10 mm Tris-HCl (pH 8), 1 mM EDTA), with each wash lasting 8 min at room temperature. Immune complexes were eluted by adding 95 μL of freshly prepared ChIP elution buffer (100 mM NaHCO₃, 1% SDS), and incubating at 65 °C for 40 min with a mixing frequency of 1200 rpm. Tubes were briefly centrifuged (3600 g for 10 s), placed on a magnetic stand for 20-30 s, and 100 μL of eluates were transferred to new tubes.

For reverse cross-linking, 4 μL of 5M NaCl was added to the 100 μL ChIPed DNA, and input samples were thawed, adjusted to a final volume of 100 μL with ChIP elution buffer, and supplemented with 4 μL of 5M NaCl. Samples were incubated overnight at 65 °C. RNase A (1 μL, Thermo Scientific EN0531) was added to the eluates, mixed, and incubated at 60 °C for 30 min. Subsequently, 2.5 μL of protease solution (ten-fold diluted Proteinase K, Roche 3115887001) was added to each sample and incubated for 1 h at 63 °C. DNA was purified using the ChIP DNA Clean & Concentrator™ kit (Zymo D5205). DNA concentration was measured using Qubit. The cells were incubated in dark overnight. We used NEBNext® Ultra™ II DNA Library Prep Kit (NEB E7645S) for sequencing libraries. The libraries were sequenced on the Illumina NextSeq 500 platform at NYU-CGSB Genomics Core using the HighOutput 1×75 bp mode.

### Maize KN1 Protoplast ChIP-Seq Data Analysis

The sequenced raw fastq reads were trimmed by fastp 0.21.0 (Chen et al. 2018). Clean reads were aligned to the maize genome version 4 downloaded from Ensembl Plant (Kersey et al. 2018) using bowtie 2.4.2 with default parameters (Langmead and Salzberg 2012). Aligned reads with MAPQ values smaller than 10 were discarded using samtools 1.11 (Li et al. 2009). The aligned bam files were converted int bigwig files using ‘bamCoverage’ command from deepTools 3.5.0 (Ramírez et al. 2016). The peaks were called using macs 2.2.7.1 (Zhang et al. 2008) with non-ChIP DNA as control. The closet genes to the peaks were determined using bedtools 2.29.2 (Quinlan and Hall 2010). Top 600 peaks within +/1 kb of genes were kept for the motif analysis. The +/−250 bp around the peak submits were used as input for MEME-ChIP (Bailey et al. 2015). The heatmap of the ChIP signal was plotted by ‘plotHeatmap’ command from deepTools 3.5.0 (Ramírez et al. 2016). This data was used in FIG. 5 and FIG. 24.

### Maize and Arabidopsis TARGET Data RNA-Seq Analysis

All RNA-seq raw reads for maize TARGET experiments were first processed to remove optical duplicates (Clumpify 37.50) and adapters (BBDuk 37.50). The trimmed reads were aligned to the maize genome version 4 using BBMap (37.50) (sourceforge.net/projects/bbmap/). The mapped reads were assigned to gene loci using featureCounts (Liao et al. 2014) with the latest annotation AGPv4.32. Genes passing the expression threshold (>1 CPM per sample) were normalized using upper-quantile normalization in EDASeq 2.18.0 (Risso et al. 2011) and replicate samples RUVSeq 1.18.0 (Risso et al. 2014). Differential gene expression analysis was conducted using edgeR (4.32) (McCarthy et al. 2012) to identify target genes for each TF, comparing empty vector- and TF-transfected samples with and without N treatment in parallel. The union of DEGs (FDR<0.05) from minus- and plus-N conditions was considered the target genes for a given TF. For the Arabidopsis AtDIV1 TARGET assay, raw fastq files were trimmed by fastp 0.21.0 (Chen et al. 2018). The clean fastq files were aligned to TAIR10 genome using hisat2.2.1 with default parameters (Kim et al. 2015). The read counts matrix was generated by featureCounts v2.0.1 (Liao et al. 2014) from the aligned bam files. The DESeq2 (Love et al. 2014) was used to identify differentially expressed genes between AtDIV1 and empty vector with False Discovery Rate (FDR) less than 0.05. This data was used in FIG. 8.

### DFG Network Construction and Network Pruning

The Dynamic Factor Graphs (DFG) method was used for the network inference as described previously (Varala et al. 2018; Brooks et al. 2019) as depicted in FIG. 3. The DFG output is a table with all possible TF-target edges. Every edge has a score indicating the confidence of that edge. In order to get the high-confidence N-signaling network, we applied network pruning based on Area Under the Precision-Recall (AUPR) curve (Varala et al. 2018; Brooks et al. 2019). We plotted the AUPR curve using maize TFs' TARGET results as gold standard. Then we chose the cutoff of the DFG network where the precision score flats on the AUPR curve. Only edges with score higher than the cutoff were retained in the final high-confidence DFG networks. The pvalue of the DFG network was computed from the permutation test. For this, we generated 1000 random networks and calculated the AUPR values by permutating edge scores. The highest, lowest and mean values of these 1000 random networks were recorded and compared with the DFG network. The network AUPR values were calculated by the precrec package in R (Saito and Rehmsmeier 2017). We used the same input for networks that were constructed by GENIE3 (Huynh-Thu et al. 2010), dyGENIE3 (Geurts 2018) and Pearson's correlation (cor(method=“p”) function in R).

### Gene Orthology and N-Response Between Orthologs

To infer the orthology between maize and Arabidopsis, we ran OrthoFinder2 (Emms and Kelly 2015) with the following command ‘orthofinder-a 8-t 16-M msa-A mafft’. Protein sequences of the longest transcripts were downloaded from Phytozome 12 (Goodstein et al. 2012) for eight species, including A. thaliana, B. distachyon, M. sinensis, O. sativa, P. virgatum, S. bicolor, S. italica and Z. mays. The protein sequences were used in OrthoFinder2 and the output ortholog table between maize and Arabidopsis were retrieved.

XGBoost Machine Learning Prediction Models of NUE Trait from Gene Expression

To predict NUE phenotypes from gene expression levels, we used XGBoost (Chen and Guestrin 2016) the machine learning pipeline described previously (Cheng et al. 2021). The R version of XGBoost (cran.r-project.org/web/packages/xgboost) was used for the analysis. Gene features were selected from either the conserved NxTime genes or conserved NUE Regulon genes. There are 16 genotypes in the maize field data with gene expression and NUE phenotype (Cheng et al. 2021). We trained the XGBoost model on the 15 genotypes and tested the model on the left-out genotype. A grid search was conducted to finetune the following hyperparameters, including ‘nrounds’, ‘col-colsample_bytree’, ‘eta’, ‘subsample’ and ‘max_depth’. Model performance was evaluated by calculating Pearson's correlation coefficients between the predicted and actual values and reported the r from 100 iterations. The combined gene importance scores from all genotypes were named NUEscore. The same workflow was applied to the Arabidopsis dataset with 18 genotypes (from Cheng et al 2021).

Network Visualization and Machine Learning Integration into NUE Regulons.

To connect the ensemble N-GRN structure with the NUEscore, we computed the NUE Regulon score for each model-to-crop conserved N-responsive TF by aggregating the NUEscore of their target genes times the TF's own NUEscore (FIG. 7). The NUE Regulon score provides a weighted importance of TFs and their associated target genes, linking network connectivity with the gene importance. The ensemble N-GRN in shoot contains edges from pruned DFG network, alongside validated TARGET and validated ChIP-Seq regulations.

The AtDIV1 and MYB34/R3 TARGET results were first mapped to their respective orthogroups based on the orthology from OrthoFinder2. The intersection of these orthogroups were tested using Fisher exact test implemented in GeneOverlap R package (Shen 2023). All network visualizations were done in Cytoscape 3 (Shannon et al. 2003).

### Analysis of External Published Datasets

For maize gene expression across developmental time in leaf and root, the FPKM (Fragments Per Kilobase of transcript per Million mapped reads) values for NxTime genes were retrieved from qTeller (Woodhouse et al. 2021). The expression matrices were normalized using z-scores and plotted using ComplexHeatmap (Gu 2022). For “NGWAS” dataset in FIG. 12, we retrieved genes mentioned in the published manuscript on GWAS meta-analysis for NUE (Xing et al. 2024). The maize v5 IDs were converted to v4 IDs according to MaizeGDB (Andorf et al. 2015).

For the Monsanto leaf dataset (Yang et al. 2011), the gene expression was measured by Affymetrix microarray. The probe sequences were obtained from NCBI GEO (GPL14616). These sequences were aligned to the maize v4 cDNA sequence using the BLAST with the following parameters ‘blastn-task megablast-query probe.fa-db Zea_mays.AGPv4.cdna.all.fa-evalue 0.0001-out monsanto_probe_raw BLAST_result.txt-outfit “7 qseqid sseqid evalue pident means”-best_hit_overhang 0.1-max_target_seqs 1’. Probed mapped to multiple genes were removed. To identify the N-responsive genes, we downloaded the raw array data from NCBI GEO (GSE32361) and used limma to identify probes that are differentially expressed between rescue nitrogen and low nitrogen conditions.

For the other published maize N-response RNA-Seq datasets (Ge et al. 2020; Wang et al. 2020b; Buoso et al. 2021a; Li et al. 2023), we used the same pipeline as our maize NxTime dataset, except no UMI steps. We re-process the raw fastq reads, downloaded from NCBI SRA to minimize the differences between various processing methods as reported in the original publication. In general, raw fastq reads were downloaded from NCBI-SRA and trimmed by fastp 0.21.0 (Chen et al. 2018). The clean reads were aligned to the maize genome version 4 using STAR 2.7.6a (Dobin et al. 2013). The featureCounts v2.0.1 (Liao et al. 2014) was used to generate gene count matrices. The N-responsive genes were called using DESeq2 (Love et al. 2014) fitting model ‘~ Ntreatment’ which compares nitrogen-treated with non-treated (or low concentration) samples.

References—no reference cited in this disclosure is an indication that the reference is material to patentability
Aalto A, Viitasaari L, Ilmonen P, Mombaerts L, and Gonçalves J. Gene regulatory network inference from sparsely sampled noisy data. Nat Commun. 2020: 11 (1): 3493. doi.org/10.1038/s41467-020-17217-1
Aibar S, González-Blas C B, Moerman T, Imrichova H, Hulselmans G, Rambow F, Marine J-C, Geurts P, Aerts J, and van den Oord J. SCENIC: single-cell regulatory network inference and clustering. Nature methods. 2017: 14 (11): 1083. doi.org/10.1038/nmeth.4463
Alexa A, Rahnenführer J, and Lengauer T. Improved scoring of functional groups from gene expression data by decorrelating G O graph structure. Bioinformatics. 2006: 22 (13): 1600-1607. doi.org/10.1093/bioinformatics/btl140
Altenhoff A M, Warwick Vesztrocy A, Bernard C, Train C-M, Nicheperovich A, Prieto Baños S, Julca I, Moi D, Nevers Y, Majidian S, et al. OMA orthology in 2024: improved prokaryote coverage, ancestral and extant G O enrichment, a revamped synteny viewer and more in the OMA Ecosystem. Nucleic Acids Research. 2024: 52 (D1): D513-D521. doi.org/10.1093/nar/gkad1020
Alvarez J M, Brooks M D, Swift J, and Coruzzi G M. Time-Based Systems Biology Approaches to Capture and Model Dynamic Gene Regulatory Networks. Annual Review of Plant Biology. 2021: 72 (Volume 72, 2021): 105-131. doi.org/10.1146/annurev-arplant-081320-090914
Alvarez J M, Schinke A-L, Brooks M D, Pasquino A, Leonelli L, Varala K, Safi A, Krouk G, Krapp A, and Coruzzi G M. Transient genome-wide interactions of the master transcription factor NLP7 initiate a rapid nitrogen-response cascade. Nature communications. 2020: 11 (1): 1-13. doi.org/10.1038/s41467-020-14979-6
Andorf C M, Cannon E K, Portwood J L, Gardiner J M, Harper L C, Schaeffer M L, Braun B L, Campbell D A, Vinnakota A G, and Sribalusu V V. MaizeGDB update: new tools, data and interface for the maize model organism database. Nucleic acids research. 2015: 44 (D1): D1195-D1201. doi.org/10.1093/nar/gkv1007
Aryee M J, Gutiérrez-Pabello J A, Kramnik I, Maiti T, and Quackenbush J. An improved empirical bayes approach to estimating differential gene expression in microarray time-course data: BETR (Bayesian Estimation of Temporal Regulation). BMC Bioinformatics. 2009: 10 (1): 409. doi.org/10.1186/1471-2105-10-409
Bailey T L and Grant C E. SEA: Simple Enrichment Analysis of motifs. 2021:2021.08.23.457422. doi.org/10.1101/2021.08.23.457422
Bailey T L, Johnson J, Grant C E, and Noble W S. The MEME Suite. Nucleic Acids Research. 2015: 43 (W1): W39-W49. doi.org/10.1093/nar/gkv416
Bargmann BOR, Marshall-Colon A, Efroni I, Ruffel S, Birnbaum K D, Coruzzi G M, and Krouk G. TARGET: A transient transformation system for genome-wide transcription factor target discovery. Molecular Plant. 2013: 6 (3): 978-980. doi.org/10.1093/mp/sst010
Bar-Joseph Z, Gitter A, and Simon I. Studying and modelling dynamic biological processes using time-series gene expression data. Nat Rev Genet. 2012: 13 (8): 552-564. doi.org/10.1038/nrg3244
Bolduc N, Yilmaz A, Mejia-Guerra M K, Morohashi K, O'Connor D, Grotewold E, and Hake S. Unraveling the KNOTTED1 regulatory network in maize meristems. Genes & development. 2012: 26 (15): 1685-90. doi.org/10.1101/gad.193433.112
Boyle E A, Li Y I, and Pritchard J K. An expanded view of complex traits: from polygenic to omnigenic. Cell. 2017: 169 (7): 1177-1186. doi.org/10.1016/j.cell.2017.05.038
Brooks M D, Cirrone J, Pasquino A V, Alvarez J M, Swift J, Mittal S, Juang C-L, Varala K, Gutiérrez R A, Krouk G, et al. Network Walking charts transcriptional dynamics of nitrogen signaling by integrating validated and predicted genome-wide interactions. Nature Communications. 2019: 10 (1): 1569. doi.org/10.1038/s41467-019-09522-1
Brooks M D, Juang C L, Katari M S, Alvarez J M, Pasquino A, Shih H J, Huang J, Shanks C, Cirrone J, and Coruzzi G M. ConnecTF: A platform to integrate transcription factor-gene interactions and validate regulatory networks. Plant Physiology. 2021: 185 (1): 49-66. doi.org/10.1093/PLPHYS/KIAA012
Buchfink B, Reuter K, and Drost H-G. Sensitive protein alignments at tree-of-life scale using DIAMOND. Nat Methods. 2021: 18 (4): 366-368. doi.org/10.1038/s41592-021-01101-x
Buoso S, Tomasi N, Arkoun M, Maillard A, Jing L, Marroni F, Pluchon S, Pinton R, and Zanin L. Transcriptomic and metabolomic profiles of Zea mays fed with urea and ammonium. Physiologia Plantarum. 2021a: 173 (3): 935-953. doi.org/10.1111/ppl.13493
Buoso S, Tomasi N, Said-Pullicino D, Arkoun M, Yvin J-C, Pinton R, and Zanin L. Characterization of physiological and molecular responses of Zea mays seedlings to different urea-ammonium ratios. Plant Physiology and Biochemistry. 2021b: 162:613-623. doi.org/10.1016/j.plaphy.2021.03.037
Cao H, Liu Z, Guo J, Jia Z, Shi Y, Kang K, Peng W, Wang Z, Chen L, Neuhaeuser B, et al. ZmNRT1.1B (ZmNPF6.6) determines nitrogen use efficiency via regulation of nitrate transport and signalling in maize. Plant Biotechnol J. 2023. doi.org/10.1111/pbi.14185
Carlson M, Falcon S, Pages H, and Li N. org. At. tair. db: Genome wide annotation for Arabidopsis. Preprint at doi org/1018129 B. 2019:9.
Castaings L, Camargo A, Pocholle D, Gaudon V, Texier Y, Boutet-Mercey S, Taconnat L, Renou J-P, Daniel-Vedele F, Fernandez E, et al. The nodule inception-like protein 7 modulates nitrate sensing and metabolism in Arabidopsis. The Plant Journal. 2009: 57 (3): 426-435. doi.org/10.1111/j.1365-313X.2008.03695.x
Castro-Mondragon J A, Riudavets-Puig R, Rauluseviciute I, Berhanu Lemma R, Turchi L, Blanc-Mathieu R, Lucas J, Boddie P, Khan A, Manosalva Pérez N, et al. JASPAR 2022: the 9th release of the open-access database of transcription factor binding profiles. Nucleic Acids Research. 2022: 50 (D1): D165-D173. doi.org/10.1093/nar/gkab1113
Chechik G, Oh E, Rando O, Weissman J, Regev A, and Koller D. Activity motifs reveal principles of timing in transcriptional control of the yeast metabolic network. Nat Biotechnol. 2008: 26 (11): 1251-1259. doi.org/10.1038/nbt.1499
Chen F, Mackey A J, Vermunt J K, and Roos D S. Assessing Performance of Orthology Detection Strategies Applied to Eukaryotic Genomes. PLOS ONE. 2007: 2 (4): e383. doi.org/10.1371/journal.pone.0000383
Chen S and Mar J C. Evaluating methods of inferring gene regulatory networks highlights their lack of performance for single cell gene expression data. BMC Bioinformatics. 2018: 19 (1): 232. doi.org/10.1186/s12859-018-2217-z
Chen S, Zhou Y, Chen Y, and Gu J. fastp: an ultra-fast all-in-one FASTQ preprocessor. Bioinformatics. 2018: 34 (17): 1884-1890. doi.org/10.1093/bioinformatics/bty560
Chen T and Guestrin C. XGBoost: A Scalable Tree Boosting System. In. Proceedings of the 22nd ACM SIGKDD International Conference on Knowledge Discovery and Data Mining, KDD ‘16. (Association for Computing Machinery: New York, N Y, USA), pp. 785-794. doi.org/10.1145/2939672.2939785
Cheng C-Y, Li Y, Varala K, Bubert J, Huang J, Kim G J, Halim J, Arp J, Shih H-J S, Levinson G, et al. Evolutionarily informed machine learning enhances the power of predictive gene-to-phenotype relationships. Nature Communications. 2021: 12 (1): 5627. doi.org/10.1038/s41467-021-25893-w
Coffey N, Hinde J, and Holian E. Clustering longitudinal profiles using P-splines and mixed effects models applied to time-course gene expression data. Computational Statistics & Data Analysis. 2014:71:14-29. doi.org/10.1016/j.csda.2013.04.001
Contreras-López O, Vidal E A, Riveras E, Alvarez J M, Moyano T C, Sparks E E, Medina J, Pasquino A, Benfey P N, and Coruzzi G M. Spatiotemporal analysis identifies ABF2 and ABF3 as key hubs of endodermal response to nitrate. Proceedings of the National Academy of Sciences. 2022: 119 (4). doi.org/10.1073/pnas.2107879119
Cui H. Challenges and Approaches to Crop Improvement Through C3-to-C4 Engineering. Front Plant Sci. 2021:12. doi.org/10.3389/fpls.2021.715391
Curci P L, Zhang J, Mahler N, Seyfferth C, Mannapperuma C, Diels T, Van Hautegem T, Jonsen D, Street N, Hvidsten T R, et al. Identification of growth regulators using cross-species network analysis in plants. Plant Physiology. 2022: 190 (4): 2350-2365. doi.org/10.1093/plphys/kiac374
Das M, Haberer G, Panda A, Das Laha S, Ghosh T C, and Schaffner A R. Expression Pattern Similarities Support the Prediction of Orthologs Retaining Common Functions after Gene Duplication Events. Plant Physiology. 2016: 171 (4): 2343-2357. doi.org/10.1104/pp. 15.01207
Dobin A, Davis C A, Schlesinger F, Drenkow J, Zaleski C, Jha S, Batut P, Chaisson M, and Gingeras T R. STAR: ultrafast universal RNA-seq aligner. Bioinformatics. 2013: 29 (1): 15-21. doi.org/10.1093/bioinformatics/bts635
Du H, Ning L, He B, Wang Y, Ge M, Xu J, and Zhao H. Cross-Species Root Transcriptional Network Analysis Highlights Conserved Modules in Response to Nitrate between Maize and Sorghum. IJMS. 2020: 21 (4): 1445. doi.org/10.3390/ijms21041445
Emms D M and Kelly S. OrthoFinder: solving fundamental biases in whole genome comparisons dramatically improves orthogroup inference accuracy. Genome Biology. 2015: 16 (1): 157. doi.org/10.1186/s13059-015-0721-2
Emms D M and Kelly S. OrthoFinder: phylogenetic orthology inference for comparative genomics. Genome Biology. 2019: 20 (1): 238. doi.org/10.1186/s13059-019-1832-y
Fan Y, Li L, and Sun S. Powerful and accurate detection of temporal gene expression patterns from multi-sample multi-stage single-cell transcriptomics data with TDEseq. Genome Biology. 2024: 25 (1): 96. doi.org/10.1186/s13059-024-03237-3
FAO. Agricultural production statistics 2000-2022. FAOSTAT Analytical Briefs. 2023: (No. 79).
Ferrier T, Matus J T, Jin J, and Riechmann J L. Arabidopsis paves the way: genomic and network analyses in crops. Current Opinion in Biotechnology. 2011: 22 (2): 260-270. doi.org/10.1016/j.copbio.2010.11.010
Gao Y and Cabrera Serrenho A. Greenhouse gas emissions from nitrogen fertilizers could be reduced by up to one-fifth of current levels by 2050 with combined interventions. Nat Food. 2023: 4 (2): 170-178. doi.org/10.1038/s43016-023-00698-w
Gao Y, Qi S, and Wang Y. Nitrate signaling and use efficiency in crops. Plant Comm. 2022: 3 (5). doi.org/10.1016/j.xplc.2022.100353
Gaudinier A, Rodriguez-Medina J, Zhang L, Olson A, Liseron-Monfils C, Bågman A, Foret J, Abbitt S, Tang M, Li B, et al. Transcriptional regulation of nitrogen-associated metabolism and growth. Nature. 2018: In press. doi.org/10.1038/s41586-018-0656-3
Ge M, Wang Y, Liu Y, Jiang L, He B, Ning L, Du H, Lv Y, Zhou L, Lin F, et al. The NIN-like protein 5 (ZmNLP5) transcription factor is involved in modulating the nitrogen response in maize. The Plant Journal. 2020: 102 (2): 353-368. doi.org/10.1111/tpj.14628
Geurts P. dynGENIE3: dynamical GENIE3 for the inference of gene networks from time series expression data. Scientific reports. 2018: 8 (1): 3384.
Gheith EMS, El-Badry O Z, Lamlom S F, Ali H M, Siddiqui M H, Ghareeb R Y, El-Sheikh M H, Jebril J, Abdelsalam N R, and Kandil E E. Maize (Zea mays L.) Productivity and Nitrogen Use Efficiency in Response to Nitrogen Application Levels and Time. Front Plant Sci. 2022:13. doi.org/10.3389/fpls.2022.941343
Goodstein D M, Shu S, Howson R, Neupane R, Hayes R D, Fazo J, Mitros T, Dirks W, Hellsten U, Putnam N, et al. Phytozome: a comparative platform for green plant genomics. Nucleic Acids Research. 2012: 40 (D1): D1178-D1186. doi.org/10.1093/nar/gkr944
Greene B, Walko R, and Hake S. Mutator insertions in an intron of the maize knotted1 gene result in dominant suppressible mutations. Genetics. 1994: 138 (4): 1275-1285. doi.org/10.1093/genetics/138.4.1275
Gu Z. Complex heatmap visualization. iMeta. 2022: 1 (3): e43. doi.org/10.1002/imt2.43
Guan M, Møller I S, and Schjoerring J K. Two cytosolic glutamine synthetase isoforms play specific roles for seed germination and seed yield structure in Arabidopsis. Journal of Experimental Botany. 2015: 66 (1): 203-212. doi.org/10.1093/jxb/eru411
Guan P, Wang R, Nacry P, Breton G, Kay S A, Pruneda-Paz J L, Davani A, and Crawford N M. Nitrate foraging by Arabidopsis roots is mediated by the transcription factor TCP20 through the systemic signaling pathway. Proceedings of the National Academy of Sciences. 2014: 111 (42): 15267-15272. doi.org/10.1073/pnas.1411375111

Guillotin B, Rahni R, Passalacqua M, Mohammed M A, Xu X, Raju S K, Ramírez C O, Jackson D, Groen S C, Gillis J, et al. A pan-grass transcriptome reveals patterns of cellular divergence in crops. Nature. 2023: 617 (7962): 785-791. doi.org/10.1038/s41586-023-06053-0
Gutierrez R A, Stokes T L, Thum K, Xu X, Obertello M, Katari M S, Tanurdzic M, Dean A, Nero D C, McClung C R, et al. Systems approach identifies an organic nitrogen-responsive gene network that is regulated by the master clock control gene CCA1. Proceedings of the National Academy of Sciences. 2008: 105 (12): 4939-4944. doi.org/10.1073/pnas.0800211105
Hartmann A, Berkowitz O, Whelan J, and Narsai R. Cross-species transcriptomic analyses reveals common and opposite responses in Arabidopsis, rice and barley following oxidative stress and hormone treatment. BMC Plant Biology. 2022: 22 (1): 62. doi.org/10.1186/s12870-021-03406-7 Heerah S, Molinari R, Guerrier S, and Marshall-Colon A. Granger-causal testing for irregularly sampled time series with application to nitrogen signalling in Arabidopsis. Bioinformatics. 2021: 37 (16): 2450-2460. doi.org/10.1093/bioinformatics/btab126
Hirel B, Le Gouis J, Ney B, and Gallais A. The challenge of improving nitrogen use efficiency in crop plants: towards a more central role for genetic variability and quantitative genetics within integrated approaches. J Exp Bot. 2007: 58 (9): 2369-2387. doi.org/10.1093/jxb/erm097
Huynh-Thu V A and Geurts P. dynGENIE3: dynamical GENIE3 for the inference of gene networks from time series expression data. Sci Rep. 2018: 8 (1): 3384. doi.org/10.1038/s41598-018-21715-0
Huynh-Thu V A, Irrthum A, Wehenkel L, and Geurts P. Inferring regulatory networks from expression data using tree-based methods. PLOS ONE. 2010: 5 (9): 1-10. doi.org/10.1371/journal.pone.0012776
Jiang L, Ball G, Hodgman C, Coules A, Zhao H, and Lu C. Analysis of Gene Regulatory Networks of Maize in Response to Nitrogen. Genes (Basel). 2018: 9 (3): 151. doi.org/10.3390/genes9030151
Kanehisa M, Furumichi M, Sato Y, Kawashima M, and Ishiguro-Watanabe M. KEGG for taxonomy-based analysis of pathways and genomes. Nucleic Acids Research. 2023: 51 (D1): D587-D592. doi.org/10.1093/nar/gkac963
Kersey P J, Allen J E, Allot A, Barba M, Boddu S, Bolt B J, Carvalho-Silva D, Christensen M, Davis P, Grabmueller C, et al. Ensembl Genomes 2018: an integrated omics infrastructure for non-vertebrate species. Nucleic Acids Research. 2018: 46 (D1): D802-D808. doi.org/10.1093/nar/gkx1011
Kerstetter R A, Laudencia-Chingcuanco D, Smith L G, and Hake S. Loss-of-function mutations in the maize homeobox gene, knotted1, are defective in shoot meristem maintenance. Development. 1997: 124 (16): 3045-3054. doi.org/10.1242/dev.124.16.3045
Kim D, Langmead B, and Salzberg S L. HISAT: a fast spliced aligner with low memory requirements. Nature methods. 2015: 12 (4): 357-360. doi.org/10.1038/nmeth.3317
Koonin E V. Orthologs, paralogs, and evolutionary genomics. Annu Rev Genet. 2005:39:309-338. doi.org/10.1146/annurev.genet.39.073003.114725
Krouk G, Lingeman J, Colon A, Coruzzi G, and Shasha D. Gene regulatory networks in plants: learning causality from time and perturbation. Genome Biology. 2013: 14 (6): 123. doi.org/10.1186/gb-2013-14-6-123
Krouk G, Mirowski P, LeCun Y, Shasha D E, and Coruzzi G M. Predictive network modeling of the high-resolution dynamic plant transcriptome in response to nitrate. Genome Biology. 2010: 11 (12): R123. doi.org/10.1186/gb-2010-11-12-r123
Kumar S, Stecher G, Suleski M, and Hedges S B. TimeTree: a resource for timelines, timetrees, and divergence times. Molecular Biology and Evolution. 2017: 34 (7): 1812-1819. doi.org/10.1093/molbev/msx116
Langfelder P and Horvath S. WGCNA: an R package for weighted correlation network analysis. BMC bioinformatics. 2008:9:559. doi.org/10.1186/1471-2105-9-559
Langmead B and Salzberg S L. Fast gapped-read alignment with Bowtie 2. Nature methods. 2012: 9 (4): 357. doi.org/10.1038/nmeth.1923
Lassaletta L, Billen G, Grizzetti B, Anglade J, and Garnier J. 50 year trends in nitrogen use efficiency of world cropping systems: the relationship between yield and nitrogen input to cropland. Environ Res Lett. 2014: 9 (10): 105011. doi.org/2020031315111836
Lau O S and Bergmann D C. MOBE-ChIP: a large-scale chromatin immunoprecipitation assay for cell type-specific studies. The Plant Journal. 2015: 84 (2): 443-450. doi.org/10.1111/tpj.13010
Lee Y-C, Tsai P-T, Huang X-X, and Tsai H-L. Family Members Additively Repress the Ectopic Expression of BASIC PENTACYSTEINE3 to Prevent Disorders in Arabidopsis Circadian Vegetative Development. Front Plant Sci. 2022:13:919946. doi.org/10.3389/fpls.2022.919946
Leng N, Li Y, McIntosh B E, Nguyen B K, Duffin B, Tian S, Thomson J A, Dewey C N, Stewart R, and Kendziorski C. EBSeq-HMM: a Bayesian approach for identifying gene-expression changes in ordered RNA-seq experiments. Bioinformatics. 2015: 31 (16): 2614-2622. doi.org/10.1093/bioinformatics/btv193
Li C, Li Y, Song G, Yang D, Xia Z, Sun C, Zhao Y, Hou M, Zhang M, Qi Z, et al. Gene expression and expression quantitative trait loci analyses uncover natural variations underlying the improvement of important agronomic traits during modern maize breeding. The Plant Journal. 2023: 115 (3): 772-787. doi.org/10.1111/tpj.16260
Li H, Handsaker B, Wysoker A, Fennell T, Ruan J, Homer N, Marth G, Abecasis G, and Durbin R. The sequence alignment/map format and SAMtools. Bioinformatics. 2009: 25 (16): 2078-2079. doi.org/10.1093/bioinformatics/btp352
Li S, Tian Y, Wu K, Ye Y, Yu J, Zhang J, Liu Q, Hu M, Li H, Tong Y, et al. Modulating plant growth-metabolism coordination for sustainable agriculture. Nature. 2018. doi.org/10.1038/s41586-018-0415-5
Liao C, Peng Y, Ma W, Liu R, Li C, and Li X. Proteomic Analysis Revealed Nitrogen-mediated Metabolic, Developmental, and Hormonal Regulation of Maize (Zea mays L.) Ear Growth. Journal of Experimental Botany. 2012: 63 (14): 5275-5288. doi.org/10.1093/jxb/ers187
Liao Y, Smyth G K, and Shi W. featureCounts: an efficient general purpose program for assigning sequence reads to genomic features. Bioinformatics. 2014: 30 (7): 923-930. doi.org/10.1093/bioinformatics/btt656
Liu M, Guo C, Xie K, Chen K, Chen J, Wang Y, and Wang X. A cross-species co-functional gene network underlying leaf senescence. Hortic Res. 2023: 10 (1): uhac251. doi.org/10.1093/hr/uhac251
Liu X, Hu B, and Chu C. Nitrogen assimilation in plants: current status and future prospects. Journal of Genetics and Genomics. 2022: 49 (5): 394-404. doi.org/10.1016/j.jgg.2021.12.006
Lothier J, Gaufichon L, Sormani R, Lemaître T, Azzopardi M, Morin H, Chardon F, Reisdorf-Cren M, Avice J-C, and Masclaux-Daubresse C. The cytosolic glutamine synthetase GLN1; 2 plays a role in the control of plant growth and ammonium homeostasis in Arabidopsis rosettes when nitrate supply is not limiting. Journal of Experimental Botany. 2011: 62 (4): 1375-1390. doi.org/10.1093/jxb/erq299
Love M I, Huber W, and Anders S. Moderated estimation of fold change and dispersion for RNA-seq data with DESeq2. Genome biology. 2014: 15 (12): 1. doi.org/10.1186/s13059-014-0550-8
Luan Y and Li H. Clustering of time-course gene expression data using a mixed-effects model with B-splines. Bioinformatics. 2003: 19 (4): 474-482. doi.org/10.1093/bioinformatics/btg014
Ma C, Zhang H H, and Wang X. Machine learning for Big Data analytics in plants. Trends in Plant Science. 2014: 19 (12): 798-808. doi.org/10.1016/j.tplants.2014.08.004
Marbach D, Costello J C, Kuffner R, Vega NNM, Prill R J, Camacho D M, Allison K R, Aderhold A, Allison K R, Bonneau R, et al. Wisdom of crowds for robust gene network inference. Nat Methods. 2012: 9 (8): 796-804. doi.org/10.1038/nmeth.2016
Marku M and Pancaldi V. From time-series transcriptomics to gene regulatory networks: A review on inference methods. PLOS Computational Biology. 2023: 19 (8): e 1011254. doi.org/10.1371/journal.pcbi. 1011254
Martin A, Lee J, Kichey T, Gerentes D, Zivy M, Tatout C, Dubois F, Balliau T, Valot B, Davanture M, et al. Two Cytosolic Glutamine Synthetase Isoforms of Maize Are Specifically Involved in the Control of Grain Production. The Plant Cell. 2006: 18 (11): 3252-3274. doi.org/10.1105/tpc.106.042689
Mathieson I. The omnigenic model and polygenic prediction of complex traits. The American Journal of Human Genetics. 2021: 108 (9): 1558-1563. doi.org/10.1016/j.ajhg.2021.07.003
Matsuda K, Mikami T, Oki S, Iida H, Andrabi M, Boss J M, Yamaguchi K, Shigenobu S, and Kondoh H. ChIP-seq analysis of genomic binding regions of five major transcription factors highlights a central role for ZIC2 in the mouse epiblast stem cell gene regulatory network. Development. 2017: 144 (11): 1948-1958. doi.org/10.1242/dev.143479
McAdams H H and Shapiro L. A Bacterial Cell-Cycle Regulatory Network Operating in Time and Space. Science. 2003: 301 (5641): 1874-1877. doi.org/10.1126/science.1087694
McCarthy D J, Chen Y, and Smyth G K. Differential expression analysis of multifactor RNA-Seq experiments with respect to biological variation. Nucleic acids research. 2012: 40 (10): 4288-4297. doi.org/10.1093/nar/gks042
Möller S, Saul N, Projahn E, Barrantes I, Gezsi A, Walter M, Antal P, and Fuellen G. Gene co-expression analyses of health (span) across multiple species. NAR Genomics and Bioinformatics. 2022: 4 (4): lqac083. doi.org/10.1093/nargab/lqac083
Moreno-Hagelsieb G and Latimer K. Choosing BLAST options for better detection of orthologs as reciprocal best hits. Bioinformatics. 2008: 24 (3): 319-324. doi.org/10.1093/bioinformatics/btm585
Mu Y, Zou M, Sun X, He B, Xu X, Liu Y, Zhang L, and Chi W. BASIC PENTACYSTEINE Proteins Repress ABSCISIC ACID INSENSITIVE4 Expression via Direct Recruitment of the Polycomb-Repressive Complex 2 in Arabidopsis Root Development. Plant and Cell Physiology. 2017: 58 (3): 607-621. doi.org/10.1093/pcp/pcx006
Mueller A J, Canty-Laird E G, Clegg P D, and Tew S R. Cross-species gene modules emerge from a systems biology approach to osteoarthritis. npj Syst Biol Appl. 2017: 3 (1): 1-15. doi.org/10.1038/s41540-017-0014-3
Naulin P A, Armijo G I, Vega A S, Tamayo K P, Gras D E, de la Cruz J, and Gutiérrez R A. Nitrate Induction of Primary Root Growth Requires Cytokinin Signaling in Arabidopsis thaliana. Plant Cell Physiol. 2020: 61 (2): 342-352. doi.org/10.1093/pcp/pcz199
Nevers Y, Jones TEM, Jyothi D, Yates B, Ferret M, Portell-Silva L, Codo L, Cosentino S, Marcet-Houben M, and Vlasova A. The Quest for Orthologs orthology benchmark service in 2022. Nucleic Acids Research. 2022. doi.org/10.1093/nar/gkac330
Obertello M, Shrivastava S, Katari M S, and Coruzzi G M. Cross-Species Network Analysis Uncovers Conserved Nitrogen-Regulated Network Modules in Rice. Plant Physiology. 2015: 168 (4): 1830-1843. doi.org/10.1104/U.S. Plant Pat. No. 114,255877
Para A, Li Y, and Coruzzi G M. u ChIP-seq for genome-wide mapping of in vivo T F-DNA interactions in Arabidopsis root protoplasts. Root development: methods and protocols. 2018:249-261. doi.org/10.1007/978-1-4939-7747-5 19
Patel R V, Nahal H K, Breit R, and Provart N J. BAR expressolog identification: expression profile similarity ranking of homologous genes in plant species. The Plant Journal. 2012: 71 (6): 1038-1050. doi.org/10.1111/j.1365-313X.2012.05055.x
Persson E and Sonnhammer ELL. InParanoiDB 9: Ortholog Groups for Protein Domains and Full-Length Proteins. Journal of Molecular Biology. 2023: 435 (14): 168001. doi.org/10.1016/j.jmb.2023.168001
Pratapa A, Jalihal A P, Law J N, Bharadwaj A, and Murali T. Benchmarking algorithms for gene regulatory network inference from single-cell transcriptomic data. Nature methods. 2020: 17 (2): 147-154. doi.org/10.1038/s41592-019-0690-6
Quinlan A R and Hall I M. BEDTools: a flexible suite of utilities for comparing genomic features. Bioinformatics. 2010: 26 (6): 841-842. doi.org/10.1093/bioinformatics/btq033
Ramírez F, Ryan D P, Grüning B, Bhardwaj V, Kilpert F, Richter A S, Heyne S, Dündar F, and Manke T. deep Tools2: a next generation web server for deep-sequencing data analysis. Nucleic Acids Res. 2016: 44 (W1): W160-165. doi.org/10.1093/nar/gkw257
Risso D, Ngai J, Speed T P, and Dudoit S. Normalization of RNA-seq data using factor analysis of control genes or samples. Nature Biotechnology. 2014: 32 (9): 896-902. doi.org/10.1038/nbt.2931
Risso D, Schwartz K, Sherlock G, and Dudoit S. G C-Content Normalization for RNA-Seq Data. BMC Bioinformatics. 2011: 12 (1): 480. doi.org/10.1186/1471-2105-12-480
Ruffel S, Krouk G, Ristova D, Shasha D, Birnbaum K D, and Coruzzi G M. Nitrogen economics of root foraging: Transitive closure of the nitrate-cytokinin relay and distinct systemic signaling for N supply vs. demand. Proceedings of the National Academy of Sciences. 2011: 108 (45): 18524-18529. doi.org/10.1073/pnas. 1108684108
Saito T and Rehmsmeier M. Precrec: fast and accurate precision-recall and ROC curve calculations in R. Bioinformatics. 2017: 33 (1): 145-147. doi.org/10.1093/bioinformatics/btw570
Shanks C M, Hecker A, Cheng C-Y, Brand L, Collani S, Schmid M, Schaller G E, Wanke D, Harter K, and Kieber J J. Role of BASIC PENTACYSTEINE transcription factors in a subset of cytokinin signaling responses. The Plant Journal. 2018: 95 (3): 458-473. doi.org/10.1111/tpj.13962
Shanks C M, Huang J, Cheng C-Y, Shih H-J S, Brooks M D, Alvarez J M, Araus V, Swift J, Henry A, and Coruzzi G M. Validation of a high-confidence regulatory network for gene-to-NUE phenotype in field-grown rice. Frontiers in Plant Science. 2022:13. doi.org/10.3389/fpls.2022.1006044
Shanks C M, Rothkegel K, Brooks M D, Cheng C-Y, Alvarez J M, Ruffel S, Krouk G, Gutiérrez R A, and Coruzzi G M. Nitrogen sensing and regulatory networks: it's about time and space. The Plant Cell. 2024: 36 (5): 1482-1503. doi.org/10.1093/plcell/koae038
Shannon P, Markiel A, Ozier O, Baliga N S, Wang J T, Ramage D, Amin N, Schwikowski B, and Ideker T. Cytoscape: a software environment for integrated models of biomolecular interaction networks. Genome research. 2003: 13 (11): 2498-2504. doi.org/10.1101/gr.1239303
Shen B, Coruzzi G M, Shasha D S. Bipartite networks represent causality better than simple networks: evidence, algorithms and applications. Frontiers in Genetics, 8 May 2024 Sec. Computational Genomics; Volume 15-2024
Shen L. GeneOverlap: An R package to test and visualize gene overlaps. R Package. 2023:3. doi.org/10.18129/B9.bioc.GeneOverlap
Smith L G, Greene B, Veit B, and Hake S. A dominant mutation in the maize homeobox gene, Knotted-1, causes its ectopic expression in leaf cells with altered fates. Development. 1992: 116 (1): 21-30. doi.org/10.1242/dev.116.1.21
Smith T, Heger A, and Sudbery I. UMI-tools: modeling sequencing errors in Unique Molecular Identifiers to improve quantification accuracy. Genome research. 2017: 27 (3): 491-499. doi.org/10.1101/gr.209601.116
Spies D, Renz P F, Beyer T A, and Ciaudo C. Comparative analysis of differential gene expression tools for RNA sequencing time course data. Briefings in Bioinformatics. 2017: (July): 1-11. doi.org/10.1093/bib/bbx115
Springer N, León N de, and Grotewold E. Challenges of Translating Gene Regulatory Information into Agronomic Improvements. Trends in Plant Science. 2019: 24 (12): 1075-1082. doi.org/10.1016/j.tplants.2019.07.004
Storey J D, Xiao W, Leek J T, Tompkins R G, and Davis R W. Significance analysis of time course microarray experiments. Proceedings of the National Academy of Sciences of the United States of America. 2005: 102 (36): 12837-12842. doi.org/10.1073/pnas.0504609102
Sun X, Dalpiaz D, Wu D, S. Liu J, Zhong W, and Ma P. Statistical inference for time course RNA-Seq data using a negative binomial mixed-effect model. BMC Bioinformatics. 2016: 17 (1): 324. doi.org/10.1186/s12859-016-1180-9
Tai Y C and Speed T P. On Gene Ranking Using Replicated Microarray Time Course Data. Biometrics. 2009: 65 (1): 40-51. doi.org/10.1111/j.1541-0420.2008.01057.x
The UniProt Consortium. UniProt: a worldwide hub of protein knowledge. Nucleic Acids Research. 2019: 47 (D1): D506-D515. doi.org/10.1093/nar/gkyl049
Tu X, Mejía-Guerra M K, Franco JAV, Tzeng D, Chu P-Y, Shen W, Wei Y, Dai X, Li P, and Buckler E S. Reconstructing the maize leaf regulatory network using ChIP-seq data of 104 transcription factors. Nature communications. 2020: 11 (1): 1-13. doi.org/10.1038/s41467-020-18832-8
Ueda Y, Nosaki S, Sakuraba Y, Miyakawa T, Kiba T, Tanokura M, and Yanagisawa S. NIGT1 family proteins exhibit dual mode DNA recognition to regulate nutrient response-associated genes in Arabidopsis. PLOS genetics. 2020: 16 (11): e1009197. doi.org/10.1371/journal.pgen.1009197
Van den Berge K, Roux de Bézieux H, Street K, Saelens W, Cannoodt R, Saeys Y, Dudoit S, and Clement L. Trajectory-based differential expression analysis for single-cell sequencing data. Nat Commun. 2020: 11 (1): 1201. doi.org/10.1038/s41467-020-14766-3
Varala K, Marshall-Colón A, Cirrone J, Brooks M D, Pasquino A V, Léran S, Mittal S, Rock™, Edwards M B, Kim G J, et al. Temporal transcriptional logic of dynamic regulatory networks underlying nitrogen signaling and use in plants. Proceedings of the National Academy of Sciences. 2018: 115 (25): 6494-6499. doi.org/10.1073/pnas.1721487115
Wang W, Hu B, Li A, and Chu C. NRT1.Is in plants: functions beyond nitrate transport. J Exp Bot. 2020a: 71 (15): 4373-4379. doi.org/10.1093/jxb/erz554
Wang Y, Xu J, Ge M, Ning L, Hu M, and Zhao H. High-resolution profile of transcriptomes reveals a role of alternative splicing for modulating response to nitrogen in maize. BMC Genomics. 2020b: 21 (1): 353. doi.org/10.1186/s12864-020-6769-8
Wimalanathan K, Friedberg I, Andorf C M, and Lawrence-Dill C J. Maize G O Annotation-Methods, Evaluation, and Review (maize-GAMER). Plant Direct. 2018: 2 (4): e00052. doi.org/10.1002/pld3.52
Woodhouse M R, Sen S, Schott D, Portwood J L I I, Freeling M, Walley J W, Andorf C M, and Schnable J C. qTeller: a tool for comparative multi-genomic gene expression analysis. Bioinformatics. 2021: 38 (1): 236-242. doi.org/10.1093/bioinformatics/btab604
Wu T-Y, Goh H, Azodi C B, Krishnamoorthi S, Liu M-J, and Urano D. Evolutionarily conserved hierarchical gene regulatory networks for plant salt stress response. Nat Plants. 2021: 7 (6): 787-799. doi.org/10.1038/s41477-021-00929-7
Xing J, Zhang J, Wang Y, Wei X, Yin Z, Zhang Y, Pu A, Dong Z, Long Y, and Wan X. Mining genic resources regulating nitrogen-use efficiency based on integrative biological analyses and their breeding applications in maize and other crops. The Plant Journal. 2024: 117 (4): 1148-1164. doi.org/10.1111/tpj.16550
Yamaguchi N, Winter C M, Wellmer F, and Wagner D. Identification of Direct Targets of Plant Transcription Factors Using the G R Fusion Technique. Plant Functional Genomics: Methods and Protocols: Second Edition. 2015:123-138. doi.org/10.1007/978-1-4939-2444-8
Yan D, Easwaran V, Chau V, Okamoto M, Ierullo M, Kimura M, Endo A, Yano R, Pasha A, Gong Y, et al. NIN-like protein 8 is a master regulator of nitrate-promoted seed germination in Arabidopsis. Nat Commun. 2016: 7 (1): 13179. doi.org/10.1038/ncomms13179
Yang X S, Wu J, Ziegler T E, Yang X, Zayed A, Rajani M S, Zhou D, Basra A S, Schachtman D P, Peng M, et al. Gene Expression Biomarkers Provide Sensitive Indicators of in Planta Nitrogen Status in Maize. Plant Physiology. 2011: 157 (4): 1841-1852. doi.org/10.1104/U.S. Plant Pat. No. 111,187898
Yates A D, Allen J, Amode R M, Azov A G, Barba M, Becerra A, Bhai J, Campbell L I, Carbajo Martinez M, Chakiachvili M, et al. Ensembl Genomes 2022: an expanding genome resource for non-vertebrates. Nucleic Acids Research. 2022: 50 (D1): D996-D1003. doi.org/10.1093/nar/gkab1007
Yoo S-D, Cho Y-H, and Sheen J. Arabidopsis mesophyll protoplasts: a versatile cell system for transient gene expression analysis. Nature protocols. 2007: 2 (7): 1565-1572. doi.org/10.1038/nprot.2007.199
Yu S, Zheng L, Li Y, Li C, Ma C, Li Y, Li X, and Hao P. A cross-species analysis method to analyze animal models’ similarity to human's disease state. BMC Systems Biology. 2012: 6 (3): S18. doi.org/10.1186/1752-0509-6-S3-S18
Yuan Y and Bar-Joseph Z. Deep learning of gene relationships from single cell time-course expression data. Brief Bioinform. 2021: 22 (5): bbab142. doi.org/10.1093/bib/bbab142
Zaslaver A, Mayo A E, Rosenberg R, Bashkin P, Sberro H, Tsalyuk M, Surette M G, and Alon U. Just-in-time transcription program in metabolic pathways. Nat Genet. 2004: 36 (5): 486-491. doi.org/10.1038/ng1348
Zhang S, Zhang Y, Li K, Yan M, Zhang J, Yu M, Tang S, Wang L, Qu H, Luo L, et al. Nitrogen Mediates Flowering Time and Nitrogen Use Efficiency via Floral Regulators in Rice. Current Biology. 2021: 31 (4): 671-683.e5. doi.org/10.1016/j.cub.2020.10.095
Zhang X, Davidson E A, Mauzerall D L, Searchinger T D, Dumas P, and Shen Y. Managing nitrogen for sustainable development. Nature. 2015: 528 (7580): 51-59. doi.org/10.1038/nature15743
Zhang Y, Liu T, Meyer C A, Eeckhoute J, Johnson D S, Bernstein B E, Nussbaum C, Myers R M, Brown M, Li W, et al. Model-based analysis of ChIP-Seq (MACS). Genome Biology. 2008: 9 (9). doi.org/10.1186/gb-2008-9-9-r137
Zhang Y, Ngu D W, Carvalho D, Liang Z, Qiu Y, Roston R L, and Schnable J C. Differentially Regulated Orthologs in Sorghum and the Subgenomes of Maize. The Plant Cell. 2017: 29 (8): 1938-1951. doi.org/10.1105/tpc.17.00354
Zhou P, Li Z, Magnusson E, Cano F G, Crisp P A, Noshay J M, Grotewold E, Hirsch C N, Briggs S P, and Springer N M. Meta gene regulatory networks in maize highlight functionally relevant regulatory interactions. The Plant Cell. 2020: 32 (5): 1377-1396. doi.org/10.1105/tpc.20.00080
Zhu J, Li S, Jiang H, Lv D, Ma S, Wang B, Lu X, Yang W, Chen R, and Zhou X. Protoplast transient expression-based RNA-sequencing: A simple method to screen transcriptional regulation in plants. Plant Physiology. 2024: 194 (1): 408-411. doi.org/10.1093/plphys/kiad495

TABLE 1
XGBoost—NUE scores for 34 maize TFs (FIGS. 6 and 7).

| | | XGBoost | Maize | |
|---|---|---|---|---|
| Row | Maize geneID | NUE score | Symbol | Maize gene description |
| 1 | Zm00001d038270 | 24.98 | MYBR3 * | MYB-related-transcription factor 3 |
| 2 | Zm00001d005951 | 22.75 | HB54 | Homeobox-transcription factor 54 |
| 3 | Zm00001d042830 | 21.15 | MYB34 * | MYB-transcription factor 34 |
| 4 | Zm00001d035907 | 14.02 | HB38 | Homeobox-transcription factor 38 |
| 5 | Zm00001d013202 | 8.92 | GLK8 | G2-like-transcription factor 8 |
| 6 | Zm00001d030678 | 5.89 | MYB56 | MYB-transcription factor 56 |
| 7 | Zm00001d016332 | 4.37 | NA | Probable protein phosphatase 2C 72 |
| 8 | Zm00001d028752 | 4.29 | NA | Probable protein phosphatase 2C 30 |
| 9 | Zm00001d032999 | 2.85 | DOF40 | C2C2-Dof-transcription factor 40 |
| 10 | Zm00001d053566 | 2.38 | NA | Probable protein phosphatase 2C 72 |
| 11 | Zm00001d003176 | 2.03 | NLP3 | NLP-transcription factor 3 |
| 12 | Zm00001d034160 | 1.09 | GLK44 | G2-like-transcription factor 44 |
| 13 | Zm00001d012749 | 1.00 | HSTF2 | heat shock transcription factor2 |
| 14 | Zm00001d039014 | 0.79 | C3H53 | C3H-transcription factor 353 |
| 15 | Zm00001d047732 | 0.77 | NA | Probable protein phosphatase 2C 30 |
| 16 | Zm00001d012337 | 0.51 | MYB79 | MYB-transcription factor 79 |
| 17 | Zm00001d011139 | 0.41 | HDT102 | histone deacetylase 102 |
| 18 | Zm00001d027530 | 0.14 | NACTF53 | NAC-transcription factor 53 |
| 19 | Zm00001d021927 | 0.12 | BES1 | brassinosteroid insensitive EMS-suppressor |
| | | | | homolog 1 |
| 20 | Zm00001d024547 | 0.09 | MYBR48 | MYB-related-transcription factor 48 |
| 21 | Zm00001d036796 | 0.09 | C3H26 | C3H-transcription factor 326 |
| 22 | Zm00001d012092 | 0.09 | HDT104 | histone deacetylase 104 |
| 23 | Zm00001d053259 | 0.06 | NA | Probable protein phosphatase 2C 74 |
| 24 | Zm00001d006677 | 0.06 | BZR7 | BZR-transcription factor 7 |
| 25 | Zm00001d036148 | 0.05 | WHY1 | whirly 1 |
| 26 | Zm00001d018178 | 0.03 | BZIP4 | bZIP-transcription factor 4 |
| 27 | Zm00001d007962 | 0.02 | GLK27 | G2-like-transcription factor 27 |
| 28 | Zm00001d005323 | 0.02 | NA | C2H2-like zinc finger protein |
| 29 | Zm00001d025323 | 0.01 | GRMZM2 | Probable protein phosphatase 2C 40 |
| | | | G443509 | |
| 30 | Zm00001d034447 | 0.01 | BZIP41 | bZIP-transcription factor 41 |
| 31 | Zm00001d027852 | 0.01 | HB102 | Homeobox-transcription factor 102 |
| 32 | Zm00001d034515 | 0.00 | HB65 | Homeobox-transcription factor 65 |
| 33 | Zm00001d006001 | 0.00 | WRKY71 | WRKY-transcription factor 71 |
| 34 | Zm00001d042965 | 0.00 | NA | Nucleic acid binding protein |

TABLE 2
XGBoost—NUE scores for 319 maize non-TFs (FIGS. 6 and 7).

| | | XGBoost | Maize | |
|---|---|---|---|---|
| Row | Maize geneID | NUE score | Symbol | Maize gene description |
| 1 | Zm00001d024633 | 166.07 | IDP52 | Monodehydroascorbate reductase 5 |
| | | | | mitochondrial |
| 2 | Zm00001d033543 | 155.43 | GRMZM2 | Integral membrane HPP family protein |
| | | | G133684 | |
| 3 | Zm00001d032311 | 121.08 | TRPS4 | trehalose-6-phosphate synthase4 |
| 4 | Zm00001d005925 | 114.39 | NA | Glucose-6-phosphate isomerase 1 |
| | | | | chloroplastic |
| 5 | Zm00001d040567 | 73.05 | NA | CBL-interacting serine/threonine-protein |
| | | | | kinase 1 |
| 6 | Zm00001d054044 | 57.72 | CAT3 | catalase3 |
| 7 | Zm00001d042453 | 56.34 | ALD1 | aldolase 1 |
| 8 | Zm00001d049995 | 51.74 | NNR1 | nitrate reductase(NADH)1 |
| 9 | Zm00001d011454 | 47.92 | CAH6 | carbonic anhydrase6 |
| 10 | Zm00001d018161 | 47.23 | NII1 | nitrate reductase l |
| 11 | Zm00001d025015 | 37.59 | PGD4 | phosphogluconate dehydrogenase4 |
| 12 | Zm00001d034914 | 35.78 | NA | Protein EXPORTIN 1A |
| 13 | Zm00001d046919 | 34.86 | NA | Chloride channel protein CLC-b |
| 14 | Zm00001d015662 | 34.66 | NA | Probable rRNA-processing protein EBP2 |
| | | | | homolog |
| 15 | Zm00001d018031 | 34.27 | EXPA2 | alpha expansin2 |
| 16 | Zm00001d021995 | 29.64 | NA | LOB domain-containing protein 38 |
| 17 | Zm00001d010128 | 25.88 | ARPP2B | acidic ribosomal protein P2b (rpp2b) |
| 18 | Zm00001d043757 | 18.78 | NA | Nuclear pore complex protein NUP50A |
| 19 | Zm00001d041945 | 18.40 | NA | transducin family protein/WD-40 repeat |
| | | | | family protein |
| 20 | Zm00001d008594 | 17.18 | NA | 60S ribosomal protein L26-1 |
| 21 | Zm00001d044138 | 15.45 | SUM1 | siroheme uroporphyrinogen |
| | | | | methyltransferase 1 |
| 22 | Zm00001d043444 | 15.12 | UMC2272 | plant-specific domain TIGR01615 family |
| | | | | protein expressed |
| 23 | Zm00001d044495 | 13.91 | SEE2B | senescence enhanced2b |
| 24 | Zm00001d005329 | 12.60 | CL39673_1A | Haloacid dehalogenase-like hydrolase |
| | | | | (HAD) superfamily protein |
| 25 | Zm00001d038146 | 10.82 | NA | Oxidoreductase%2C 20G-Fe oxygenase |
| | | | | family protein |
| 26 | Zm00001d038891 | 10.22 | PEAMT2 | Phosphoethanolamine N-methyltransferase |
| | | | | 3 |
| 27 | Zm00001d001947 | 10.16 | NA | TLD-domain containing nucleolar protein |
| 28 | Zm00001d016407 | 8.58 | NA | SPOC domain/Transcription elongation |
| | | | | factor S-II protein |
| 29 | Zm00001d008387 | 8.05 | RPS5 | ribosomal protein S5 |
| 30 | Zm00001d009631 | 8.01 | UMC1385 | CTP synthase family protein |
| 31 | Zm00001d007937 | 7.53 | ALT8 | alanine amino transferase8 |
| 32 | Zm00001d016719 | 7.48 | NA | U-box domain-containing protein 29 |
| 33 | Zm00001d031659 | 7.41 | PDH2 | pyruvate dehydrogenase2 |
| 34 | Zm00001d052165 | 7.25 | NII2 | nitrite reductase2 |
| 35 | Zm00001d037708 | 7.25 | NA | UDP-glycosyltransferase 75B1 |
| 36 | Zm00001d010282 | 6.86 | NA | Single-stranded nucleic acid binding R3H |
| | | | | protein |
| 37 | Zm00001d022557 | 6.70 | NAS5 | nicotianamine synthase5 |
| 38 | Zm00001d037620 | 6.28 | RPL13 | 60S ribosomal protein L13 |
| 39 | Zm00001d026137 | 6.24 | NA | protein; OSJNBa0084K11.10 protein |
| 40 | Zm00001d045069 | 5.96 | NA | Ribosomal protein L19 |
| 41 | Zm00001d018206 | 5.75 | NNR2 | nitrate reductase2 |
| 42 | Zm00001d041072 | 5.62 | NA | ABC transporter F family member 1 |
| 43 | Zm00001d038346 | 4.34 | NA | NA |
| 44 | Zm00001d052941 | 3.95 | NA | zinc knuckle (CCHC-type) family protein |
| 45 | Zm00001d011130 | 3.88 | IDP2365 | Nucleosome assembly protein 1; 2 |
| 46 | Zm00001d038190 | 3.81 | DIP1 | DBF1-interactor protein 1 |
| 47 | Zm00001d024587 | 3.62 | NPF1 | nitrate transporter/peptide transporter |
| | | | | family 1 |
| 48 | Zm00001d044056 | 3.62 | KCH1 | potassium channel 1 |
| 49 | Zm00001d040515 | 3.60 | NA | Gamma carbonic anhydrase 1 |
| | | | | mitochondrial |
| 50 | Zm00001d031833 | 3.36 | NA | Mitochondrial 2-oxoglutarate/malate |
| | | | | carrier protein |
| 51 | Zm00001d043632 | 3.16 | NA | Hyaluronan/mRNA binding family |
| 52 | Zm00001d040735 | 3.10 | INVAN10 | invertase alkaline neutral10 |
| 53 | Zm00001d005023 | 3.00 | NA | 40S ribosomal protein S16 |
| 54 | Zm00001d006704 | 2.39 | NA | RNA-binding (RRM/RBD/RNP motifs) |
| | | | | family protein |
| 55 | Zm00001d012598 | 2.38 | NA | 40S ribosomal protein S25-2 |
| 56 | Zm00001d028426 | 2.34 | NA | 40S ribosomal protein S20-1 |
| 57 | Zm00001d028622 | 2.11 | NPI242B | 60S ribosomal protein L3-1 |
| 58 | Zm00001d050920 | 2.09 | NA | 60S ribosomal protein L6 |
| 59 | Zm00001d005421 | 1.73 | PIP2B | plasma membrane intrinsic protein2 |
| 60 | Zm00001d047290 | 1.64 | NCP3 | nine complex protein3 |
| 61 | Zm00001d046682 | 1.58 | NA | Peroxiredoxin-5 |
| 62 | Zm00001d027512 | 1.57 | NA | 60S ribosomal protein L5-1 |
| 63 | Zm00001d039908 | 1.54 | NA | Ribosomal protein L26 |
| 64 | Zm00001d006822 | 1.46 | NA | Pentatricopeptide repeat-containing protein |
| | | | | PNM1 mitochondrial |
| 65 | Zm00001d034760 | 1.41 | FDX3 | ferredoxin3 |
| 66 | Zm00001d017691 | 1.39 | HAGTF25 | GNAT-transcription factor 25 |
| 67 | Zm00001d051543 | 1.37 | NA | 60S ribosomal protein L12-3 |
| 68 | Zm00001d053635 | 1.35 | NA | 40S ribosomal protein S8 |
| 69 | Zm00001d042525 | 1.33 | RPL5A | 60S ribosomal protein L5-1 homolog a |
| 70 | Zm00001d013940 | 1.23 | NA | Nucleolar complex protein 2 homolog |
| 71 | Zm00001d019473 | 1.16 | NA | 60S ribosomal protein L8-3 |
| 72 | Zm00001d026635 | 1.16 | ALLA1 | allantoinase 1 |
| 73 | Zm00001d042183 | 1.10 | NA | alpha/beta-Hydrolases superfamily protein |
| 74 | Zm00001d012391 | 1.06 | PCO085448 | Delta 1-pyrroline-5-carboxylate synthetase |
| | | | | isoform 1%3B Delta 1-pyrroline-5- |
| | | | | carboxylate synthetase isoform 2 |
| 75 | Zm00001d007836 | 1.04 | NA | NA |
| 76 | Zm00001d042764 | 1.02 | NA | hemerythrin HHE cation-binding domain |
| | | | | protein |
| 77 | Zm00001d040056 | 1.01 | NA | NA |
| 78 | Zm00001d037748 | 1.01 | NA | Thioredoxin-like 2-2 chloroplastic |
| 79 | Zm00001d018957 | 0.99 | NA | 60S ribosomal protein L4-1 |
| 80 | Zm00001d014969 | 0.96 | NA | Ribosomal protein L37 |
| 81 | Zm00001d002483 | 0.96 | NA | Transducin family protein/WD-40 repeat |
| | | | | family protein |
| 82 | Zm00001d040309 | 0.95 | NA | Mediator-associated protein 2 |
| 83 | Zm00001d047340 | 0.83 | NA | Allene oxide cyclase 3 chloroplastic |
| 84 | Zm00001d011965 | 0.80 | IDP2350 | Aldolase-type TIM barrel family protein |
| 85 | Zm00001d038022 | 0.75 | NA | Pentatricopeptide repeat-containing protein |
| | | | | chloroplastic |
| 86 | Zm00001d037992 | 0.74 | NA | ERBB-3 BINDING PROTEIN 1 |
| 87 | Zm00001d040659 | 0.73 | NA | Late embryogenesis abundant protein Lea5 |
| 88 | Zm00001d010710 | 0.68 | NA | Ubiquitin carboxyl-terminal hydrolase 25 |
| 89 | Zm00001d032873 | 0.67 | UMC2181 | Metal ion binding protein |
| 90 | Zm00001d050043 | 0.67 | NA | Putative wound responsive protein%3B |
| | | | | Putative wound responsive protein isoform |
| | | | | 1%3B Putative wound responsive protein |
| | | | | isoform 2%3B Wound responsive protein |
| 91 | Zm00001d043625 | 0.65 | NA | Plasminogen activator inhibitor 1 RNA- |
| | | | | binding protein |
| 92 | Zm00001d034550 | 0.60 | NA | 40S ribosomal protein S2-1 |
| 93 | Zm00001d021936 | 0.60 | NA | Putative polyol transporter 1 |
| 94 | Zm00001d016841 | 0.56 | NA | monooxygenase 1 |
| 95 | Zm00001d042443 | 0.49 | NA | BTB/POZ and TAZ domain-containing |
| | | | | protein 2 |
| 96 | Zm00001d040204 | 0.47 | NA | NA |
| 97 | Zm00001d021379 | 0.47 | NA | S15/NS1 RNA-binding protein |
| 98 | Zm00001d018383 | 0.47 | RPL39 | ribosomal protein L39 |
| 99 | Zm00001d013326 | 0.46 | NA | NA |
| 100 | Zm00001d034345 | 0.44 | FNR1 | ferredoxin NADP reductase l |
| 101 | Zm00001d052136 | 0.44 | ACCO20 | 1-aminocyclopropane-1-carboxylate |
| | | | | oxidase20 |
| 102 | Zm00001d009146 | 0.43 | SAMS2 | S-adenosylmethionine synthetase2 |
| 103 | Zm00001d028750 | 0.43 | ASN3 | asparagine synthetase3 |
| 104 | Zm00001d007508 | 0.41 | NA | T-complex protein 1 subunit theta |
| 105 | Zm00001d038541 | 0.41 | SDG111 | set domain gene111 |
| 106 | Zm00001d002880 | 0.40 | IMD2 | isopropylmalate dehydrogenase2 |
| 107 | Zm00001d023379 | 0.40 | PYK2 | pyruvate kinase2 |
| 108 | Zm00001d035595 | 0.38 | DHAR3 | dehydroascorbate reductase like3 |
| 109 | Zm00001d020852 | 0.38 | NA | Cellulose synthase-like protein E1 |
| 110 | Zm00001d010606 | 0.35 | NA | Protein RETICULATA-RELATED 6 |
| | | | | chloroplastic |
| 111 | Zm00001d045889 | 0.35 | MAGI104697 | 60S ribosomal protein L31 |
| 112 | Zm00001d008257 | 0.33 | NA | NA |
| 113 | Zm00001d054089 | 0.31 | NA | Putative TCP-1/cpn60 chaperonin family |
| | | | | protein isoform 1%3B Putative TCP-1/ |
| | | | | cpn60 chaperonin family protein isoform |
| | | | | 2 |
| 114 | Zm00001d035578 | 0.31 | NA | P-loop containing nucleoside triphosphate |
| | | | | hydrolases superfamily protein |
| 115 | Zm00001d031870 | 0.31 | NA | Zinc finger protein 593 |
| 116 | Zm00001d033924 | 0.30 | NA | NA |
| 117 | Zm00001d016897 | 0.30 | NA | Polyadenylate-binding protein RBP47B |
| 118 | Zm00001d045352 | 0.30 | NFA101A | nucleosome/chromatin asssembly factor A |
| 119 | Zm00001d048953 | 0.30 | KCS3 | 3-ketoacyl-CoA synthase3 |
| 120 | Zm00001d009870 | 0.29 | NA | Elongation factor 1-alpha |
| 121 | Zm00001d031120 | 0.27 | NA | Cationic amino acid transporter 4 vacuolar |
| 122 | Zm00001d049187 | 0.27 | PGD3 | phosphogluconate dehydrogenase3 |
| 123 | Zm00001d012771 | 0.26 | NA | tRNA/rRNA methyltransferase (SpoU) |
| | | | | family protein |
| 124 | Zm00001d017398 | 0.23 | NA | Protein OBERON 2 |
| 125 | Zm00001d041511 | 0.23 | NA | Heparanase-like protein 1 |
| 126 | Zm00001d031058 | 0.23 | NA | Receptor homology region transmembrane |
| | | | | domain- and RING domain-containing |
| | | | | protein 2 |
| 127 | Zm00001d018406 | 0.22 | NA | MGD2 |
| 128 | Zm00001d029628 | 0.20 | PSEI7 | cystatin 7 |
| 129 | Zm00001d045740 | 0.20 | NA | Transmembrane amino acid transporter |
| | | | | family protein |
| 130 | Zm00001d034606 | 0.19 | IWS1 | IWS1/SPN1-transcription factor 1 |
| 131 | Zm00001d050526 | 0.19 | NA | Importin beta-like SAD2 |
| 132 | Zm00001d043047 | 0.19 | EXPA1 | alpha expansin 1 |
| 133 | Zm00001d032046 | 0.18 | CKX10 | cytokinin oxidase 10 |
| 134 | Zm00001d005978 | 0.18 | XER1 | xerico1 |
| 135 | Zm00001d047859 | 0.17 | MS45 | male sterile45 |
| 136 | Zm00001d036570 | 0.17 | NA | 60S ribosomal protein L27 |
| 137 | Zm00001d013392 | 0.17 | NA | Histone H2A |
| 138 | Zm00001d039865 | 0.17 | TPI4 | triose phosphate isomerase4 |
| 139 | Zm00001d045611 | 0.15 | NA | Protein MEI2-like 5 |
| 140 | Zm00001d049409 | 0.15 | ACO1 | aconitase1 |
| 141 | Zm00001d003112 | 0.15 | PCO142939 | Leucine-rich repeat family protein |
| 142 | Zm00001d031332 | 0.15 | PZA03531 | Heat shock protein 90-2 |
| 143 | Zm00001d050838 | 0.15 | NA | Polyadenylate-binding protein RBP47B |
| 144 | Zm00001d036359 | 0.14 | NA | Transducin/WD40 repeat-like superfamily |
| | | | | protein |
| 145 | Zm00001d040390 | 0.14 | NA | NA |
| 146 | Zm00001d008708 | 0.14 | NA | Putative DUF1421 domain family protein |
| 147 | Zm00001d015300 | 0.14 | NA | 60S ribosomal protein L19 |
| 148 | Zm00001d032272 | 0.14 | NA | NA |
| 149 | Zm00001d053957 | 0.13 | NA | Phosphatidate cytidylyltransferase |
| 150 | Zm00001d004839 | 0.13 | NA | 60S ribosomal protein L3-1 |
| 151 | Zm00001d032894 | 0.12 | PZA00381 | Protein RRP6-like 2 |
| 152 | Zm00001d005647 | 0.12 | NA | Probable receptor-like protein kinase |
| 153 | Zm00001d042388 | 0.12 | NA | DUF538 family protein |
| 154 | Zm00001d011758 | 0.12 | NA | NA |
| 155 | Zm00001d021858 | 0.11 | NPI263 | Protein TIME FOR COFFEE |
| 156 | Zm00001d038671 | 0.11 | NA | Topoisomerase II-associated protein PAT1 |
| 157 | Zm00001d010744 | 0.11 | CL4945_1 | Gamma-glutamyl hydrolase 1 |
| 158 | Zm00001d001798 | 0.11 | MIS12B | minichromosome instability 12b |
| 159 | Zm00001d013758 | 0.11 | NA | 60S ribosomal protein L24 |
| 160 | Zm00001d043845 | 0.11 | NA | Glutamate synthase 1 [NADH] |
| | | | | chloroplastic |
| 161 | Zm00001d048238 | 0.10 | NA | EMB514 |
| 162 | Zm00001d052461 | 0.10 | NA | NA |
| 163 | Zm00001d021196 | 0.10 | NA | DEAD-box ATP-dependent RNA helicase |
| | | | | 7 |
| 164 | Zm00001d011174 | 0.10 | SEE2A | senescence enhanced2a |
| 165 | Zm00001d017085 | 0.10 | NA | beta-14-N-acetylglucosaminyltransferase |
| | | | | family protein |
| 166 | Zm00001d008289 | 0.09 | NA | La-related protein 1A |
| 167 | Zm00001d049641 | 0.09 | GPC1 | glyceraldehyde-3-phosphate |
| | | | | dehydrogenase1 |
| 168 | Zm00001d002091 | 0.09 | NA | Putative chloride channel-like protein |
| | | | | CLC-g |
| 169 | Zm00001d053834 | 0.09 | NA | Putative ubiquitin family protein%3B |
| | | | | Ubiquitin2 |
| 170 | Zm00001d013544 | 0.09 | NA | Serine/threonine protein phosphatase 2A 57 |
| | | | | kDa regulatory subunit B' beta isoform |
| 171 | Zm00001d012052 | 0.09 | PCO106379 | RNA-binding (RRM/RBD/RNP motifs) |
| | | | | family protein |
| 172 | Zm00001d037111 | 0.09 | NA | 40S ribosomal protein S17-1 |
| 173 | Zm00001d029594 | 0.09 | AOC1 | allene oxide cyclase1 |
| 174 | Zm00001d035937 | 0.08 | NA | Chaperonin 60 subunit beta 2 chloroplastic |
| 175 | Zm00001d051754 | 0.08 | ALDH2 | aldehyde dehydrogenase2 |
| 176 | Zm00001d018542 | 0.08 | NA | DEAD-box ATP-dependent RNA helicase |
| | | | | 34 |
| 177 | Zm00001d051806 | 0.07 | NA | Protein binding protein |
| 178 | Zm00001d011034 | 0.07 | DHAR1 | dehydroascorbate reductase like1 |
| 179 | Zm00001d051666 | 0.07 | INVAN1 | invertase alkaline neutral1 |
| 180 | Zm00001d044664 | 0.07 | NA | 40S ribosomal protein S16-3 |
| 181 | Zm00001d020647 | 0.07 | TIDP3675 | Phospholipid:diacylglycerol acyltransferase |
| | | | | 1 |
| 182 | Zm00001d044096 | 0.07 | CAH2 | carbonic anhydrase2 |
| 183 | Zm00001d014701 | 0.07 | GLK10 | G2-like-transcription factor 10 |
| 184 | Zm00001d033983 | 0.07 | MAGI87281 | Aluminium induced protein with YGL and |
| | | | | LRDR motifs |
| 185 | Zm00001d010646 | 0.06 | SDG105 | set domain gene105 |
| 186 | Zm00001d015196 | 0.06 | NA | DNA polymerase V family |
| 187 | Zm00001d008327 | 0.06 | PZA01623 | H/ACA ribonucleoprotein complex subunit |
| | | | | 4 |
| 188 | Zm00001d034422 | 0.06 | NA | 40S ribosomal protein S18 |
| 189 | Zm00001d010944 | 0.05 | NA | DUF724 domain-containing protein 2 |
| 190 | Zm00001d007154 | 0.05 | IDP469 | 60S ribosomal protein L22-2 |
| 191 | Zm00001d052194 | 0.05 | HSP22 | heat shock protein22 |
| 192 | Zm00001d017927 | 0.05 | NA | Fcf2 pre-rRNA processing protein |
| 193 | Zm00001d042921 | 0.05 | NA | SLT1 protein |
| 194 | Zm00001d046170 | 0.05 | PEP1 | phosphoenolpyruvate carboxylase1 |
| 195 | Zm00001d013411 | 0.05 | NA | UV-B-induced protein chloroplastic |
| 196 | Zm00001d045000 | 0.05 | NA | 40S ribosomal protein S17-4 |
| 197 | Zm00001d018107 | 0.05 | NA | Protein EXORDIUM |
| 198 | Zm00001d053022 | 0.05 | NA | Transcription factor GTE12 |
| 199 | Zm00001d001899 | 0.04 | ARPP2A-3 | acidic ribosomal protein P2a-3 |
| 200 | Zm00001d015449 | 0.04 | NA | Dimethyladenosine transferase |
| 201 | Zm00001d051885 | 0.04 | NA | ATG8-interacting protein 1 |
| 202 | Zm00001d052945 | 0.04 | NA | Probable ribonuclease P/MRP protein |
| | | | | subunit POP5 |
| 203 | Zm00001d010220 | 0.04 | NA | 40S ribosomal protein S12 |
| 204 | Zm00001d047758 | 0.04 | MPK1 | MAP kinase 1 |
| 205 | Zm00001d004033 | 0.04 | NA | DnaJ domain ;Myb-like DNA-binding |
| | | | | domain |
| 206 | Zm00001d018295 | 0.04 | NA | ARM repeat superfamily protein |
| 207 | Zm00001d032555 | 0.04 | BNLG1564 | bnlg1564; : |
| 208 | Zm00001d053580 | 0.04 | NA | Dihydrolipoyllysine-residue |
| | | | | acetyltransferase component 2 of pyruvate |
| | | | | dehydrogenase complex mitochondrial |
| 209 | Zm00001d044529 | 0.04 | NPF7 | nitrate transporter/peptide transporter |
| | | | | family7 |
| 210 | Zm00001d053887 | 0.03 | NA | Molybdopterin synthase catalytic subunit |
| 211 | Zm00001d047566 | 0.03 | NA | Mitochondrial import receptor subunit |
| | | | | TOM40-1 |
| 212 | Zm00001d006250 | 0.03 | NA | Alba DNA/RNA-binding protein |
| 213 | Zm00001d041994 | 0.03 | NA | Leucine-rich repeat (LRR) family protein |
| 214 | Zm00001d021313 | 0.03 | NA | PIN domain-like family protein |
| 215 | Zm00001d048924 | 0.03 | NA | Putative DEAD-box ATP-dependent RNA |
| | | | | helicase family protein |
| 216 | Zm00001d006613 | 0.02 | NA | eukaryotic translation initiation factor 2 |
| | | | | gamma subunit |
| 217 | Zm00001d050961 | 0.02 | NA | N-acetylglucosaminyltransferase III |
| 218 | Zm00001d002301 | 0.02 | NA | NA |
| 219 | Zm00001d022504 | 0.02 | NHX5 | Na+/H+ antiporter 5 |
| 220 | Zm00001d053564 | 0.02 | NA | KH domain-containing protein |
| 221 | Zm00001d006507 | 0.02 | NA | Phospholipase A I |
| 222 | Zm00001d007772 | 0.02 | NA | RNA-binding (RRM/RBD/RNP motifs) |
| | | | | family protein |
| 223 | Zm00001d009776 | 0.02 | NA | Translation initiation factor IF-2 |
| 224 | Zm00001d015375 | 0.02 | UMC2298 | 60S ribosomal protein L27a-3 |
| 225 | Zm00001d014781 | 0.02 | NA | NA |
| 226 | Zm00001d038267 | 0.02 | UMC2524 | maternal effect embryo arrest 59 |
| 227 | Zm00001d029973 | 0.02 | NA | Nucleic acid binding protein |
| 228 | Zm00001d010850 | 0.02 | NA | Transducin family protein/WD-40 repeat |
| | | | | family protein |
| 229 | Zm00001d021420 | 0.02 | NA | Nudix hydrolase 23 chloroplastic |
| 230 | Zm00001d012485 | 0.02 | MMP64 | Transmembrane amino acid transporter |
| | | | | family protein |
| 231 | Zm00001d012633 | 0.02 | NA | Eukaryotic aspartyl protease family protein |
| 232 | Zm00001d008604 | 0.02 | NA | Protein NRT1/PTR FAMILY 8.1 |
| 233 | Zm00001d021070 | 0.02 | PDH4 | pyruvate dehydrogenase4 |
| 234 | Zm00001d012549 | 0.02 | NA | Probable U3 small nucleolar RNA- |
| | | | | associated protein 11 |
| 235 | Zm00001d025132 | 0.02 | NA | Nucleic acid-binding OB-fold-like protein |
| 236 | Zm00001d051001 | 0.02 | GPC3 | glyceraldehyde-3-phosphate |
| | | | | dehydrogenase3 |
| 237 | Zm00001d010075 | 0.02 | NA | Tetratricopeptide repeat (TPR)-like |
| | | | | superfamily protein |
| 238 | Zm00001d002591 | 0.02 | NA | Nuclear transport factor 2 (NTF2) family |
| | | | | protein |
| 239 | Zm00001d014585 | 0.02 | NA | Cytoplasmic 60S subunit biogenesis factor |
| | | | | REI1 homolog 1 |
| 240 | Zm00001d020463 | 0.02 | NA | Sugar transport protein 7 |
| 241 | Zm00001d050340 | 0.02 | NA | NA |
| 242 | Zm00001d048358 | 0.02 | ACO3 | aconitase3 |
| 243 | Zm00001d029529 | 0.02 | BNL9.11B | bn19.11b(lts); : |
| | | | (LTS) | |
| 244 | Zm00001d022513 | 0.02 | NA | Elongation factor 1-beta 2 |
| 245 | Zm00001d031660 | 0.02 | NA | NA |
| 246 | Zm00001d017790 | 0.01 | NA | Carbamoyl-phosphate synthase small chain |
| | | | | chloroplastic |
| 247 | Zm00001d051062 | 0.01 | NA | GrpE protein homolog |
| 248 | Zm00001d053547 | 0.01 | NA | 60S ribosomal protein L11-1 |
| 249 | Zm00001d049399 | 0.01 | NA | Putative nucleolin-like family protein |
| 250 | Zm00001d005089 | 0.01 | NA | mRNA turnover protein 4 |
| 251 | Zm00001d019182 | 0.01 | NA | Eukaryotic translation initiation factor 3 |
| | | | | subunit E |
| 252 | Zm00001d046530 | 0.01 | NA | Tetratricopeptide repeat (TPR)-like |
| | | | | superfamily protein |
| 253 | Zm00001d021511 | 0.01 | NA | Ribosomal protein L15 |
| 254 | Zm00001d021935 | 0.01 | NA | Putative polyol transporter 1 |
| 255 | Zm00001d017168 | 0.01 | NA | Acetolactate synthase small subunit 1 |
| | | | | chloroplastic |
| 256 | Zm00001d052062 | 0.01 | NA | Splicing factor%2C arginine/serine-rich |
| | | | | 7%3B Splicing factor%2C arginine/serine- |
| | | | | rich 7 isoform 1%3B Splicing factor%2C |
| | | | | arginine/serine-rich 7 isoform 2 |
| 257 | Zm00001d027619 | 0.01 | AMYB2 | beta amylase2 |
| 258 | Zm00001d018896 | 0.01 | UMC1159 | evolutionarily conserved C-terminal region |
| | | | | 2 |
| 259 | Zm00001d010248 | 0.01 | IDP602 | Electron transporter |
| 260 | Zm00001d008394 | 0.01 | NA | Thylakoid lumenal 17.9 kDa protein |
| | | | | chloroplastic |
| 261 | Zm00001d034856 | 0.01 | TUFM1 | elongation factor TU mitochondriall |
| 262 | Zm00001d011377 | 0.01 | AAS13 | auxin amido synthetase 1 3 |
| 263 | Zm00001d013056 | 0.01 | NA | DEAD-box ATP-dependent RNA helicase |
| | | | | 51 |
| 264 | Zm00001d026254 | 0.01 | NA | 60S ribosomal protein L7-2 |
| 265 | Zm00001d016720 | 0.01 | PCO136495A | Serine decarboxylase |
| 266 | Zm00001d010445 | 0.01 | PYL8 | pyrabactin resistance-like protein8 |
| 267 | Zm00001d041719 | 0.01 | NA | Heat shock protein 90-6 mitochondrial |
| 268 | Zm00001d025777 | 0.01 | UMC1280 | Eukaryotic translation initiation factor |
| | | | | isoform 4G-2 |
| 269 | Zm00001d016916 | 0.01 | UMC1264 | Nucleotide-sugar transporter/ sugar porter |
| 270 | Zm00001d010431 | 0.01 | NA | Elongation factor 1-beta |
| 271 | Zm00001d029530 | 0.01 | NA | binding |
| 272 | Zm00001d019108 | 0.01 | RPS1 | Ribosomal protein S1-like1 |
| 273 | Zm00001d017242 | 0.01 | NA | H/ACA ribonucleoprotein complex subunit |
| | | | | 3-like protein |
| 274 | Zm00001d048157 | 0.01 | NA | 40S ribosomal protein S3a |
| 275 | Zm00001d022406 | 0.01 | NA | Probable mitochondrial adenine nucleotide |
| | | | | transporter BTL1 |
| 276 | Zm00001d048834 | 0.01 | NA | DNA binding;DNA-directed RNA |
| | | | | polymerases |
| 277 | Zm00001d015356 | 0.00 | HPPD1 | 4-hydroxyphenylpyruvate dioxygenase 1 |
| 278 | Zm00001d008700 | 0.00 | VT2 | vanishing tassel2 |
| 279 | Zm00001d031423 | 0.00 | UMC2234 | Zinc finger A20 and AN1 domain- |
| | | | | containing stress-associated protein 5 |
| 280 | Zm00001d052820 | 0.00 | NA | RNA-binding (RRM/RBD/RNP motifs) |
| | | | | family protein |
| 281 | Zm00001d013252 | 0.00 | MTA2 | mouse transplantation antigen homolog2 |
| 282 | Zm00001d053396 | 0.00 | NA | Abscisic acid receptor PYL9 |
| 283 | Zm00001d029142 | 0.00 | NA | Nuclear pore complex protein NUP93A |
| 284 | Zm00001d017185 | 0.00 | NA | Protein NUCLEAR FUSION DEFECTIVE |
| | | | | 5 mitochondrial |
| 285 | Zm00001d038807 | 0.00 | NA | periodic tryptophan protein 2 |
| 286 | Zm00001d020188 | 0.00 | NA | Protein arginine N-methyltransferase 1.1 |
| 287 | Zm00001d025025 | 0.00 | NA | Electron transfer flavoprotein subunit beta |
| | | | | mitochondrial |
| 288 | Zm00001d021938 | 0.00 | NA | Putative polyol transporter 1 |
| 289 | Zm00001d005200 | 0.00 | NA | 40S ribosomal protein S12 |
| 290 | Zm00001d038021 | 0.00 | NA | ARM repeat superfamily protein |
| 291 | Zm00001d003031 | 0.00 | AY104214 | DEAD-box ATP-dependent RNA helicase |
| | | | | 13 |
| 292 | Zm00001d037700 | 0.00 | NA | Heat shock protein 4 |
| 293 | Zm00001d014090 | 0.00 | CPN1 | chaperonin 1 |
| 294 | Zm00001d039321 | 0.00 | NA | SMR domain-containing protein |
| 295 | Zm00001d039883 | 0.00 | NA | Mitochondrial glycoprotein family protein |
| 296 | Zm00001d007316 | 0.00 | CL10369_1 | Oligosaccharyltransferase |
| | | | | complex/magnesium transporter family |
| | | | | protein |
| 297 | Zm00001d048460 | 0.00 | NA | CBL-interacting serine/threonine-protein |
| | | | | kinase 9 |
| 298 | Zm00001d012069 | NotExpressed | NA | DUF506 family protein |
| | | InCheng | | |
| 299 | Zm00001d038476 | NotExpressed | EXPA5 | alpha expansin5 |
| | | InCheng | | |
| 300 | Zm00001d021705 | NotExpressed | NA | NA |
| | | InCheng | | |
| 301 | Zm00001d033284 | NotExpressed | NA | WAT1-related protein |
| | | InCheng | | |
| 302 | Zm00001d046998 | NotExpressed | NA | Protein LIGHT-DEPENDENT SHORT |
| | | InCheng | | HYPOCOTYLS 5 |
| 303 | Zm00001d044608 | NotExpressed | ASN2 | asparagine synthetase2 |
| | | InCheng | | |
| 304 | Zm00001d032181 | NotExpressed | IDP243 | 60S ribosomal protein L34-3 |
| | | InCheng | | |
| 305 | Zm00001d020681 | NotExpressed | NA | NA |
| | | InCheng | | |
| 306 | Zm00001d000313 | NotExpressed | NA | Ubiquitin carboxyl-terminal hydrolase 25 |
| | | InCheng | | |
| 307 | Zm00001d006680 | NotExpressed | NA | P-loop containing nucleoside triphosphate |
| | | InCheng | | hydrolases superfamily protein |
| 308 | Zm00001d020810 | NotExpressed | NA | S-adenosyl-L-methionine-dependent |
| | | InCheng | | methyltransferases superfamily protein |
| 309 | Zm00001d010512 | NotExpressed | NA | ARM repeat superfamily protein |
| | | InCheng | | |
| 310 | Zm00001d022124 | NotExpressed | MAGI89825 | 60S ribosomal protein L27a-3 |
| | | InCheng | | |
| 311 | Zm00001d044037 | NotExpressed | UMC1273 | Protein trichome birefringence-like 39 |
| | | InCheng | | |
| 312 | Zm00001d040257 | NotExpressed | NA | T-complex protein 1 subunit beta |
| | | InCheng | | |
| 313 | Zm00001d043962 | NotExpressed | NA | GBF-interacting protein 1 |
| | | InCheng | | |
| 314 | Zm00001d032035 | NotExpressed | NA | cytochrome P450 family 706 subfamily A |
| | | InCheng | | polypeptide 5 |
| 315 | Zm00001d048837 | NotExpressed | CKI8 | cyclin-dependent kinase inhibitor8 |
| | | InCheng | | |
| 316 | Zm00001d034795 | NotExpressed | PHB4 | prohibitin4 |
| | | InCheng | | |
| 317 | Zm00001d000439 | NotExpressed | NA | SNARE associated Golgi protein family |
| | | InCheng | | |
| 318 | Zm00001d002371 | NotExpressed | PCO135860 | Protein kinase superfamily protein |
| | | InCheng | | |
| 319 | Zm00001d051343 | NotExpressed | NA | Protein kinase superfamily protein |
| | | InCheng | | |

TABLE 3
NUE Regulon scores for 34 TFs in maize (FIG. 7).
Maize TFs within NUE-centric subnetwork is ranked by their
NUE Regulon scores. Targets for seven TFs were validated by
the TARGET assay (Bold). This table is an extension of FIG. 7B.

| | | | Total | NUE | Validated in |
|---|---|---|---|---|---|
| | | | regulated | Regulon | maize cells |
| Row | TFs | TF Symbol | genes | score | (TARGET assay)ª |
| 1 | Zm00001d038270 | ZmMYBR3 * | 740 | 15795.8 | Yes |
| 2 | Zm00001d042830 | ZmMYB34 * | 809 | 13850.7 | Yes |
| 3 | Zm00001d005951 | ZmHB54 | 1003 | 9254.9 | Yes |
| 4 | Zm00001d030678 | ZmMYB56 | 684 | 3628.7 | Yes |
| 5 | Zm00001d035907 | ZmHB38 | 292 | 2216.7 | — |
| 6 | Zm00001d003176 | ZmNLP3 | 389 | 1396.8 | Yes |
| 7 | Zm00001d028752 | ZmOrphan42 | 622 | 981.1 | Yes |
| 8 | Zm00001d053566 | ZmOrphan350 | 121 | 411.9 | — |
| 9 | Zm00001d034160 | ZmGLK44 | 353 | 342.4 | Yes |
| 10 | Zm00001d016332 | ZmOrphan210 | 46 | 321.4 | — |
| 11 | Zm00001d012749 | ZmHSF26 | 117 | 77.5 | — |
| 12 | Zm00001d047732 | ZmOrphan 193 | 81 | 67.1 | — |
| 13 | Zm00001d027530 | ZmNAC53 | 112 | 8.4 | — |
| 14 | Zm00001d012092 | 1d012092 | 109 | 7.5 | — |
| 15 | Zm00001d021927 | ZmBZR10 | 91 | 7.0 | — |
| 16 | Zm00001d024547 | ZmMYBR48 | 69 | 5.5 | — |
| 17 | Zm00001d032999 | ZmDOF40 | 36 | 4.7 | — |
| 18 | Zm00001d053259 | ZmOrphan211 | 53 | 3.8 | — |
| 19 | Zm00001d036796 | ZmC3H26 | 119 | 3.6 | — |
| 20 | Zm00001d013202 | ZmGLK8 | 59 | 2.9 | — |
| 21 | Zm00001d034447 | ZmbZIP41 | 150 | 0.9 | — |
| 22 | Zm00001d025323 | ZmOrphan170 | 82 | 0.9 | — |
| 23 | Zm00001d027852 | ZmHB102 | 15 | 0.5 | — |
| 24 | Zm00001d007962 | ZmGLK27 | 74 | 0.5 | — |
| 25 | Zm00001d039014 | ZmC3H53 | 30 | 0.4 | — |
| 26 | Zm00001d006001 | ZmWRKY71 | 117 | 0.4 | — |
| 27 | Zm00001d036148 | ZmWHIRLY1 | 62 | 0.2 | — |
| 28 | Zm00001d034515 | ZmHB65 | 58 | 0.1 | — |
| 29 | Zm00001d042965 | 1d042965 | 60 | 0.1 | — |
| 30 | Zm00001d011139 | 1d011139 | 8 | 0.0 | — |
| 31 | Zm00001d018178 | ZmbZIP4 | 28 | 0.0 | — |
| 32 | Zm00001d006677 | ZmBZR7 | 29 | 0.0 | — |
| 33 | Zm00001d005323 | 1d005323 | 43 | 0.0 | — |
| 34 | Zm00001d012337 | ZmMYB79 | 5 | 0.0 | — |

TABLE 4
Validated TF-NUE Regulon genes: Maize TFs ZmMYB34/R3 and Arabidopsis
TF DIV1 (FIG. 8).
XGBoost NUE scores for the 25 genes in the ZmMYB34/R3 NUE Regulon and
their orthologs in Arabidopsis DIV1 Regulon validated in the TARGET
TF assay. Transcription factors (TFs) in the regulon are in bold.

| | | Maize | | | | Arabidopsis | | |
|---|---|---|---|---|---|---|---|---|
| | | XGBoost | | | | XGBoost | | |
| | | NUE | Maize | Maize | Arabidopsis | NUE | Arabidopsis | Arabidopsis gene |
| Row | Maize geneID | score | Symbol | gene description | ortholog | score | Symbol | description |
| 1 | Zm00001d038270 | 320.53 | MYBR3* | MYB-related- | AT5G58900 | 167.20 | DIV1 | Homeodomain-like |
| | | | (TF) | transcription factor 3 | | | (TF) | transcriptional |
| | | | | | | | | regulator |
| 2 | Zm00001d042830 | — | MYB34* | MYB-transcription | AT5G58900 | 167.20 | DIV1 | Homeodomain-like |
| | | | (TF) | factor 34 | | | (TF) | transcriptional |
| | | | | | | | | regulator |

Target genes - Transcription Factors

| | | | | | | | | |
|---|---|---|---|---|---|---|---|---|
| 3 | Zm00001d034160 | 85.92 | GLK44 | G2-like-transcription | AT1G49560 | 52.76 | HHO6 | Homeodomain-like |
| | | | (TF) | factor 44 | | | (TF) | superfamily protein |
| 4 | Zm00001d007962 | 11.64 | GLK27 | G2-like-transcription | AT1G49560 | 52.76 | HHO6 | Homeodomain-like |
| | | | (TF) | | | | (TF) | superfamily protein |
| | | | | factor 27 | | | | |
| 5 | Zm00001d032999 | 86.57 | DOF40 | C2C2-Dof- | AT5G62430 | 59.55 | CDF1 | cycling DOF |
| | | | (TF) | transcription factor 40 | | | (TF) | factor 1 |
| 6 | Zm00001d006001 | 28.78 | WRKY71 | WRKY-transcription | AT5G56270 | 211.53 | WRKY2 | WRKY DNA-binding |
| | | | (TF) | factor 71 | | | (TF) | protein 2 |

Target genes - Nitrogen assimilation genes

| | | | | | | | | |
|---|---|---|---|---|---|---|---|---|
| 7 | Zm00001d052165 | 9.36 | ZmNiR1.1 | nitrite reductase2 | AT2G15620 | 106.62 | NIR1 | nitrite reductase 1 |
| 8 | Zm00001d018161 | 97.80 | ZmNiR1.2 | nitrate reductase1 | AT2G15620 | 106.62 | NIR1 | nitrite reductase 1 |
| 9 | Zm00001d043845 | 1.79 | GOGAT2 | Glutamate synthase 1 | AT5G53460 | 73.63 | GLT1 | NADH-dependent |
| | | | | [NADH] chloroplastic | | | | glutamate synthase 1 |

Target genes - Other processes

| | | | | | | | | |
|---|---|---|---|---|---|---|---|---|
| 10 | Zm00001d018031 | 69.77 | EXPA2 | alpha expansin2 | AT1G69530 | 42.02 | EXPA1 | expansin A1 |
| 11 | Zm00001d031332 | 1.30 | PZA03531 | Heat shock | AT5G56030 | 74.34 | HSP81-2 | heat shock |
| | | | | protein 90-2 | | | | protein 81-2 |
| 12 | Zm00001d031332 | 1.30 | PZA03531 | Heat shock | AT5G56010 | 60.37 | HSP81-3 | heat shock |
| | | | | protein 90-2 | | | | protein 81-3 |
| 13 | Zm00001d009146 | 5.24 | SAMS2 | S-adenosylmethionine | AT1G02500 | 33.68 | SAM1 | S-adenosylmethionine |
| | | | | synthetase2 | | | | synthetase 1 |
| 14 | Zm00001d048157 | 328.35 | NA | 40S ribosomal | AT4G34670 | 86.07 | NA | Ribosomal |
| | | | | protein S3a | | | | protein S3Ae |
| 15 | Zm00001d028622 | 266.27 | NPI242B | 60S ribosomal | AT1G61580 | 110.18 | RPL3B | R-protein L3 B |
| | | | | protein L3-1 | | | | |
| 16 | Zm00001d035937 | 5.62 | NA | Chaperonin 60 subunit | AT3G13470 | 121.33 | Cpn60beta2 | TCP-1/cpn60 |
| | | | | beta 2 chloroplastic | | | | chaperonin family |
| | | | | | | | | protein |
| 17 | Zm00001d020852 | 19.03 | NA | Cellulose | AT1G55850 | 73.00 | CSLE1 | cellulose synthase |
| | | | | synthase-like | | | | like E1 |
| | | | | protein E1 | | | | |
| 18 | Zm00001d033983 | 48.10 | MAGI87281 | Aluminium induced | AT3G22850 | 50.43 | NA | Aluminium induced |
| | | | | protein with YGL | | | | protein with YGL |
| | | | | and LRDR motifs | | | | and LRDR motifs |
| 19 | Zm00001d023379 | 1.53 | PYK2 | pyruvate kinase2 | AT2G36580 | 80.87 | NA | Pyruvate kinase |
| | | | | | | | | family protein |
| 20 | Zm00001d043444 | 151.60 | UMC2272 | plant-specific domain | AT2G20670 | 67.34 | NA | Protein of unknown |
| | | | | TIGR01615 family | | | | function (DUF506) |
| | | | | protein expressed | | | | |
| 21 | Zm00001d031833 | 7.28 | NA | Mitochondrial | AT2G22500 | 65.18 | UCP5 | uncoupling protein 5 |
| | | | | 2-oxoglutarate/malate | | | | |
| | | | | carrier protein | | | | |
| 22 | Zm00001d047340 | 4.19 | NA | Allene oxide cyclase | AT3G25760 | 50.36 | AOC1 | allene oxide cyclase 1 |
| | | | | 3 chloroplastic | | | | |
| 23 | Zm00001d042443 | 1.76 | NA | BTB/POZ and TAZ | AT3G48360 | 71.10 | BT2 | BTB and TAZ |
| | | | | domain-containing | | | | domain protein 2 |
| | | | | protein 2 | | | | |
| 24 | Zm00001d052194 | 0.39 | HSP22 | heat shock | AT5G51440 | 44.42 | NA | HSP20-like |
| | | | | protein22 | | | | chaperones |
| | | | | | | | | superfamily protein |
| 25 | Zm00001d020463 | 3.06 | NA | Sugar transport | AT4G02050 | 32.11 | STP7 | sugar transporter |
| | | | | protein 7 | | | | protein 7 |
| 26 | Zm00001d001947 | 44.11 | NA | TLD-domain | AT5G39590 | 65.90 | NA | TLD-domain |
| | | | | containing | | | | containing |
| | | | | nucleolar protein | | | | nucleolar protein |

TABLE 5
Maize ZmMYB34/R3 TF -NUE Regulon genes and the in planta
validation if available (FIG. 8).
The 25 genes in the ZmMYB34/R3 Regulon and the in planta validated
functions if available. Transcription factors (TFs) in the regulon are in bold.

| | | | | Validated in | | | |
|---|---|---|---|---|---|---|---|
| | | Maize | | MYB34/R3 | | Nitrogen- | |
| | | NUE | Maize | TARGET | In planta | related | In planta |
| Row | Maize geneID | score | Symbol | assay | functions | functions | evidence |
| 1 | Zm00001d038270 | 320.53 | MYBR3 * | — | NA | NA | NA |
| | | | (TF) | | | | |
| 2 | Zm00001d042830 | — | MYB34 * | — | NA | NA | NA |
| | | | (TF) | | | | |

Target genes - Transcription Factors

| | | | | | | | |
|---|---|---|---|---|---|---|---|
| 3 | Zm00001d034160 | 85.92 | GLK44 | Yes | GLK44 | NA | Over-expression |
| | | | (TF) | | regulate | | lines; RNA-Seq; |
| | | | | | tryptophan | | Metabolomics |
| | | | | | biosynthes is | | [1] |
| | | | | | and positively | | |
| | | | | | regulates | | |
| | | | | | drought | | |
| | | | | | tolerance [1] | | |
| 4 | Zm00001d007962 | 11.64 | GLK27 | Yes | NA | NA | NA |
| | | | (TF) | | | | |
| 5 | Zm00001d032999 | 86.57 | DOF40 | Yes | NA | NA | NA |
| | | | (TF) | | | | |
| 6 | Zm00001d006001 | 28.78 | WRKY71 | Yes | NA | NA | NA |
| | | | (TF) | | | | |

Target genes - Nitrogen assimilation genes

| | | | | | | | |
|---|---|---|---|---|---|---|---|
| 7 | Zm00001d052165 | 9.36 | Z | Yes | NA | NA | NA |
| | | | mNiR1.1 | | | | |
| 8 | Zm00001d018161 | 97.80 | Z | Yes | NA | NA | NA |
| | | | mNiR1.2 | | | | |
| 9 | Zm00001d043845 | 1.79 | GOGAT2 | Yes | NA | NA | NA |

Target genes - Other processes

| | | | | | | | |
|---|---|---|---|---|---|---|---|
| 10 | Zm00001d018031 | 69.77 | EXPA2 | Yes | NA | NA | NA |
| 11 | Zm00001d031332 | 1.30 | PZA03531 | Yes | NA | NA | NA |
| 12 | Zm00001d009146 | 5.24 | SAMS2 | Yes | NA | NA | NA |
| 13 | Zm00001d048157 | 328.35 | NA | Yes | NA | NA | NA |

TABLE 6
Number of genes-of-importance to NUE under low
N that are activated or repressed by each
TF based on TARGET TF Assay in maize cells.
Number of genes-of-importance to maize field NUE under
low N, learned from Bipartite method (Shen et al 2024),
that are regulated by maize TFs listed in Table 1 and FIG. 29.
Results of TARGET TF-assay conducted in maize cell
distinguish the directly activated vs. repressed genes of each TF.

| | | Activated | Repressed |
|---|---|---|---|
| | | Genes-of-NUE | Genes-of-NUE |
| Gene ID | TF Name | Importance | Importance |
| Zm00001d039267 | ARF7 | 274 | 313 |
| Zm00001d046937 | bZIP9 | 228 | 238 |
| Zm00001d014863 | RVE2 | 202 | 231 |
| Zm00001d044785 | GLK1 | 260 | 160 |
| Zm00001d024543 | NAC61 | 207 | 198 |
| Zm00001d005951 | HB6 | 217 | 145 |
| Zm00001d032694 | MYB121 | 168 | 174 |
| Zm00001d005208 | NAC5 | 184 | 134 |
| Zm00001d033965 | WRKY107 | 142 | 169 |
| Zm00001d042830 | MYB34 | 117 | 157 |
| Zm00001d049173 | WRKY36 | 107 | 157 |
| Zm00001d038270 | MYBR3 | 104 | 141 |
| Zm00001d028752 | Orphan42 | 96 | 141 |
| Zm00001d011187 | bHLH92 | 81 | 116 |
| Zm00001d028999 | NAC44 | 96 | 94 |
| Zm00001d039260 | G2 | 124 | 44 |
| Zm00001d039893 | MYB81 | 51 | 114 |
| Zm00001d021208 | EREB115 | 62 | 95 |
| Zm00001d029339 | bHLH47 | 56 | 100 |
| Zm00001d034160 | GLK44 | 34 | 90 |
| Zm00001d030678 | MYB56 | 64 | 60 |
| Zm00001d035605 | MYB21 | 48 | 72 |
| Zm00001d006835 | NFYA3 | 48 | 67 |
| Zm00001d003176 | ZmNLP3 | 70 | 37 |
| Zm00001d024160 | bZIP107 | 45 | 50 |
| Zm00001d037607 | WRKY125 | 36 | 46 |
| Zm00001d040318 | NACTF66 | 42 | 36 |
| Zm00001d033859 | KN1 | 11 | 26 |
| Zm00001d042609 | NAC109 | 17 | 17 |
| Zm00001d002143 | bZIP27 | 21 | 7 |
| Zm00001d046223 | HB21 | 12 | 4 |
| Zm00001d007436 | CA5P8 | 11 | 4 |
| Zm00001d005029 | GRAS37 | 5 | 4 |

TABLE 7
Ranked list of Transcription Factors-of-importance to NUE
to be overexpressed for increased NUE on Low N.
Top 10 transcription factors that can be overexpressed in maize for
increased NUE. The “Positive Influence to NUE Score” reflects the
likelihood that overexpression of the transcription factor will
positively influence NUE traits. The scoring incorporates Bipartite
learned genes-of-importance's positive or negative association to
maize field NUE with each TF's activation or repression of
those same learned genes-of-importance to NUE, which is
measured from the maize TF TARGET assay (see Table 6).

| | | Net Positive Influence |
|---|---|---|
| Gene ID | TF Name | of TF to NUE Score |
| Zm00001d032694 | MYB121 | 185.7 |
| Zm00001d038270 | MYBR3 | 164.3 |
| Zm00001d024543 | NAC61 | 133.5 |
| Zm00001d005951 | HB6 | 119.7 |
| Zm00001d044785 | GLK1 | 109.1 |
| Zm00001d030678 | MYB56 | 93.7 |
| Zm00001d049173 | WRKY36 | 90.1 |
| Zm00001d042830 | MYB34 | 88.2 |
| Zm00001d028999 | NAC44 | 83.2 |
| Zm00001d039267 | ARF7 | 73.0 |

TABLE 8
Ranked list of Transcription Factors for disruption or inhibition
of expression to influence increased NUE on low N.
Top 5 transcriptions factors to be disrupted or inhibited in expression
in maize for increased NUE. The “Negative Influence to NUE Score”
reflects the likelihood that overexpression of the transcription factor
will negatively influence NUE traits. The scoring incorporates
Bipartite learned genes-of-importance's positive or negative
association to maize field NUE with each TF's activation or
repression of those same learned genes-of-importance, which is
measured from the maize TF TARGET assay (see Table 6).

| | | Net Negative TF |
|---|---|---|
| Gene ID | TF Name | Influence to NUE Score |
| Zm00001d034160 | GLK44 | −69.5 |
| Zm00001d039260 | G2 | −52.6 |
| Zm00001d006835 | NFYA3 | −28.8 |
| Zm00001d007436 | CA5P8 | −8.7 |
| Zm00001d033859 | KN1 | −6.1 |

TABLE 9
TF pairs for complementary overexpression to improve NUE on low N.
Transcription factor pairs with complementary positive impacts on
maize field NUE based on regulation of genes-of-importance to NUE in
TF TARGET Assay in maize cells (see Table 6). These pairs are the
top 10 TF pairs for co-overexpression in maize to further improve NUE
I n low N, compared to single TF overexpression.

| | | | | TF pair complementary |
|---|---|---|---|---|
| | TF | | TF | Positive Influence |
| Gene 1 ID | Name 1 | Gene 2 ID | Name 2 | to NUE Score |
| Zm00001d032694 | MYB121 | Zm00001d038270 | MYBR3 | 350 |
| Zm00001d032694 | MYB121 | Zm00001d024543 | NAC61 | 319.2 |
| Zm00001d005951 | HB6 | Zm00001d032694 | MYB121 | 305.4 |
| Zm00001d038270 | MYBR3 | Zm00001d024543 | NAC61 | 297.8 |
| Zm00001d044785 | GLK1 | Zm00001d032694 | MYB121 | 294.8 |
| Zm00001d005951 | HB6 | Zm00001d038270 | MYBR3 | 284 |
| Zm00001d032694 | MYB121 | Zm00001d030678 | MYB56 | 279.4 |
| Zm00001d032694 | MYB121 | Zm00001d049173 | WRKY36 | 275.8 |
| Zm00001d032694 | MYB121 | Zm00001d042830 | MYB34 | 273.9 |
| Zm00001d044785 | GLK1 | Zm00001d038270 | MYBR3 | 273.4 |

TABLE 10
TF pairs for complementary disruption or inhibition of expression
to improve NUE on low N.
Transcription factor pairs with complementary negative impacts on
maize field NUE on low N based on regulation of genes-of-importance
to NUE (See Table 6). These TF pairs are the top 10 TF pairs for gene
disruption or inhibition of expression in maize to enhance NUE on
low N compared to single TF disruption or inhibition of expression.

| | | | | TF pair |
|---|---|---|---|---|
| | | | | Complementary |
| | TF | | TF | Negative Influence |
| Gene 1 ID | Name 1 | Gene 2 ID | Name 2 | to NUE Score |
| Zm00001d039260 | G2 | Zm00001d034160 | GLK44 | −122.1 |
| Zm00001d034160 | GLK44 | Zm00001d006835 | NFYA3 | −98.3 |
| Zm00001d039260 | G2 | Zm00001d006835 | NFYA3 | −81.4 |
| Zm00001d007436 | CA5P8 | Zm00001d034160 | GLK44 | −78.3 |
| Zm00001d034160 | GLK44 | Zm00001d033859 | KN1 | −75.6 |
| Zm00001d034160 | GLK44 | Zm00001d033965 | WRKY107 | −73.9 |
| Zm00001d034160 | GLK44 | Zm00001d005029 | GRAS37 | −65 |
| Zm00001d021208 | EREB115 | Zm00001d034160 | GLK44 | −63.2 |
| Zm00001d007436 | CA5P8 | Zm00001d039260 | G2 | −61.3 |
| Zm00001d029339 | bHLH47 | Zm00001d034160 | GLK44 | −60.9 |

TABLE 11
TF pairs where TF1 is overexpressed and TF2 is disrupted or
inhibited for expression to improve NUE on low N.
Top 10 transcription factor pairs where overexpressing TF1, while
simultaneously disrupting or inhibiting gene expression of TF2 are
predicted to have a combinatorial positive activation score to
improve maize field NUE on low N.

| | | | | TF pair |
|---|---|---|---|---|
| | | | Disruption or | Combinatorial |
| Overexpress Gene | Overexpress | Knockdown Gene | inhibition of | NUE Activation |
| ID | TF Name | ID | TF Name | Score |
| Zm00001d032694 | MYB121 | Zm00001d034160 | GLK44 | 255.2 |
| Zm00001d032694 | MYB121 | Zm00001d039260 | G2 | 238.3 |
| Zm00001d038270 | MYBR3 | Zm00001d034160 | GLK44 | 233.8 |
| Zm00001d038270 | MYBR3 | Zm00001d039260 | G2 | 216.9 |
| Zm00001d032694 | MYB121 | Zm00001d006835 | NFYA3 | 214.5 |
| Zm00001d024543 | NAC61 | Zm00001d034160 | GLK44 | 203 |
| Zm00001d032694 | MYB121 | Zm00001d007436 | CA5P8 | 194.4 |
| Zm00001d038270 | MYBR3 | Zm00001d006835 | NFYA3 | 193.1 |
| Zm00001d032694 | MYB121 | Zm00001d033859 | KN1 | 191.8 |
| Zm00001d032694 | MYB121 | Zm00001d033965 | WRKY107 | 190.1 |

TABLE 12
Cross-referenced list of TFs-of-importance to NUE from
Table 1 and FIG. 29.
The table is ordered by TF's co-appearance in the three analyses.
All tables' hereafter will follow this TF order.

| Gene ID | Gene Name | Table 1 | FIG. 29 |
|---|---|---|---|
| Zm00001d038270 | MYBR3 | 1 | 1 |
| Zm00001d042830 | MYB34 | 1 | 1 |
| Zm00001d002234 | HB75 | 0 | 1 |
| Zm00001d006293 | NLP17 | 0 | 1 |
| Zm00001d005029 | GRAS37 | 0 | 1 |
| Zm00001d042609 | NAC109 | 0 | 1 |
| Zm00001d024160 | BZIP107 | 0 | 1 |
| Zm00001d028999 | NAC44 | 0 | 1 |
| Zm00001d037607 | WRKY125 | 0 | 1 |
| Zm00001d030678 | MYB56 | 1 | 1 |
| Zm00001d003176 | NLP3 | 1 | 1 |
| Zm00001d028752 | ORPHAN42 | 1 | 1 |
| Zm00001d034160 | GLK44 | 1 | 1 |
| Zm00001d039267 | ARF7 | 0 | 1 |
| Zm00001d034298 | BHLH47 | 0 | 1 |
| Zm00001d011187 | BHLH92 | 0 | 1 |
| Zm00001d002143 | BZIP27 | 0 | 1 |
| Zm00001d018178 | BZIP4 | 1 | 0 |
| Zm00001d034447 | BZIP41 | 1 | 0 |
| Zm00001d020025 | BZIP9 | 0 | 1 |
| Zm00001d021927 | BZR10 | 1 | 0 |
| Zm00001d006677 | BZR7 | 1 | 0 |
| Zm00001d036796 | C3H26 | 1 | 0 |
| Zm00001d039014 | C3H53 | 1 | 0 |
| Zm00001d007436 | CA5P8 | 0 | 1 |
| Zm00001d032999 | DOF40 | 1 | 0 |
| Zm00001d021208 | EREB115 | 0 | 1 |
| Zm00001d039260 | G2 | 0 | 1 |
| Zm00001d044785 | GLK1 | 0 | 1 |
| Zm00001d007962 | GLK27 | 1 | 0 |
| Zm00001d013202 | GLK8 | 1 | 0 |
| Zm00001d027852 | HB102 | 1 | 0 |
| Zm00001d046223 | HB21 | 0 | 1 |
| Zm00001d035907 | HB38 | 1 | 0 |
| Zm00001d005951 | HB54 | 1 | 0 |
| Zm00001d053151 | HB6 | 0 | 1 |
| Zm00001d034515 | HB65 | 1 | 0 |
| Zm00001d011139 | HDT102 | 1 | 0 |
| Zm00001d012092 | HDT104 | 1 | 0 |
| Zm00001d033859 | KN1 | 0 | 1 |
| Zm00001d035605 | MYB21 | 0 | 1 |
| Zm00001d012337 | MYB79 | 1 | 0 |
| Zm00001d039893 | MYB81 | 0 | 1 |
| Zm00001d024547 | MYBR48 | 1 | 0 |
| Zm00001d005208 | NAC5 | 0 | 1 |
| Zm00001d027530 | NAC53 | 1 | 0 |
| Zm00001d024543 | NAC61 | 0 | 1 |
| Zm00001d040318 | NACTF66 | 0 | 1 |
| Zm00001d006835 | NFYA3 | 0 | 1 |
| Zm00001d025323 | ORPHAN170 | 1 | 0 |
| Zm00001d047732 | ORPHAN193 | 1 | 0 |
| Zm00001d053259 | ORPHAN211 | 1 | 0 |
| Zm00001d053566 | ORPHAN350 | 1 | 0 |
| Zm00001d042965 | PCO075454 | 1 | 0 |
| Zm00001d014863 | RVE2 | 0 | 1 |
| Zm00001d036148 | WHIRLY1 | 1 | 0 |
| Zm00001d033965 | WRKY107 | 0 | 1 |
| Zm00001d049173 | WRKY36 | 0 | 1 |
| Zm00001d006001 | WRKY71 | 1 | 0 |
| Zm00001d005323 | ZF-C2H2 | 1 | 0 |

1 = present.
0 = not present.

TABLE 13
TFs-of-importance to NUE with guideRNA sequences to disrupt the 5' UTR
region by CRISPR/cas9.
We list two possible guide sequence options for each TF based on the predicted
ranked efficiency of each guide RNA.

| | | SEQ ID | | Genomic | |
|---|---|---|---|---|---|
| Gene ID | Gene Name | NO | guideRNA sequence options | location | Efficiency |
| Zm00001d038270 | MYBR3 | 3 | AAGCAUUAAAGAAAGCGGCGAGG | 6:152871918 | 70.39 |
| | | 4 | GAGGAGGUAUUUAUGCAGCGGGG | 6:152872018 | 69 |
| Zm00001d042830 | MYB34 | 5 | GAGAAGAAUCAAAUUCCCGGCGG | 3:181378053 | 76.23 |
| | | 6 | AAGGUAGCGUGAGUAUUUAGGGG | 3:181378075 | 60.72 |
| Zm00001d002234 | HB75 | 7 | UCCAUGUAGAAUUAAUGACGAGG | 2:8465847 | 69.53 |
| | | 8 | CUGCUGGGUAACACUCUCGCCGG | 2:8469584 | 68.85 |
| Zm00001d006293 | NLP17 | 9 | AUCUGCAGUGCUCUACAGGUAGG | 2:203860250 | 66.99 |
| | | 10 | ACCAGUUCCCAGCCGACGUUAGG | 2:203859990 | 50.77 |
| Zm00001d005029 | GRAS37 | 11 | GAUUUCAGCUCUCAACGGCACGG | 2:154743351 | 67.2 |
| | | 12 | GUUGUUGGUUACCCAGCCAGUGG | 2:154743328 | 66.12 |
| Zm00001d042609 | NAC109 | 13 | GAGCGCUUGUUCUUACCCUGAGG | 3:173459356 | 69.07 |
| | | 14 | GAGUAAGUACACUCGAAGCUUGG | 3:173460684 | 60.62 |
| Zm00001d024160 | BZIP107 | 15 | UCGUAACGAGACACCCGCGACGG | 10:51315867 | 73.58 |
| | | 16 | AGGUUCCGUAUGUACGCCUGGGG | 10:51316899 | 69.05 |
| Zm00001d028999 | NAC44 | 17 | AAAUAGGCAACGGACUGCAGGGG | 1:54263649 | 67.15 |
| | | 18 | AGUUUAGCCAGGAGAAACGGAGG | 1:54265646 | 64.01 |
| Zm00001d037607 | WRKY125 | 19 | ACCUCUUGUGACGACUGACGAGG | 6:131562522 | 67.33 |
| | | 20 | GCGAUGAGUAAUCCAAGUGGUGG | 6:131561648 | 67.11 |
| Zm00001d030678 | MYB56 | 21 | UUUUCAUCACAGGAUCGAUAUGG | 1:152669101 | 56.8 |
| | | 22 | AAUCCAGUCUGUUUGGGAGGAGG | 1:152669230 | 53.35 |
| Zm00001d003176 | NLP3 | 23 | ACCUGGAGGUAGUUUAAUCGUGG | 2:34606547 | 67.02 |
| | | 24 | UACCCUUGUAGGUAUACUGGAGG | 2:34607942 | 66.55 |
| Zm00001d028752 | ORPHAN42 | 25 | CCUCGCGAUGUCUUGCACGGUGG | 1:45537970 | 66.97 |
| | | 26 | GGUUGCUUUGGAGUGGUCUGCGG | 1:45537863 | 57.25 |
| Zm00001d034160 | GLK44 | 27 | UGAGGGGAUCUGGAUUCCGGAGG | 1:285335166 | 68.1 |
| | | 28 | CGAGGCGUGGAGCAAGGUACGGG | 1:285335301 | 56 |
| Zm00001d039267 | ARF7 | 29 | UUAUGCAUACCAGUGCUGGGAGG | 3:841346 | 74.65 |
| | | 30 | GACCCAAGAGCUCAUCACAAAGG | 3:841217 | 70.07 |
| Zm00001d034298 | BHLH47 | 31 | CUUCCGUAGAUAGAAUACGGCGG | 1:288722657 | 83.2 |
| | | 32 | UUUCACCGGAACAUGUAGCGUGG | 1:288721377 | 71.04 |
| Zm00001d011187 | BHLH92 | 33 | GAACUACGGUCUAACGACUGCGG | 8:142339532 | 75.56 |
| | | 34 | UCGUUAGACCGUAGUUCCGGCGG | 8:142339538 | 73.16 |
| Zm00001d002143 | BZIP27 | 35 | ACCGUACGUACCCUCUCUGGCGG | 2:6640410 | 74.11 |
| | | 36 | UCCCAAGCUGUAAAGUCCAGUGG | 2:6642199 | 70.96 |
| Zm00001d018178 | BZIP4 | 37 | GUGGACAGGUGUCACCCACGAGG | 5:215871633 | 73.74 |
| | | 38 | UCGAAGCGUGUGAAUCCGGUGGG | 5:215871409 | 60.64 |
| Zm00001d034447 | BZIP41 | 39 | CGUUAAUCAGAAGUUCAAUGAGG | 1:293290788 | 76.86 |
| | | 40 | AGCUAGUGGUUUGCUUAACAGGG | 1:293292741 | 49.67 |
| Zm00001d020025 | BZIP9 | 41 | CGACCAUAAAUCUGGAAAGGAGG | 7:86720489 | 62.1 |
| | | 42 | CUCCUUUCCAGAUUUAUGGUCGG | 7:86720490 | 58.13 |
| Zm00001d021927 | BZR10 | 43 | UCAGACCAUGACGUCCGGGGCGG | 7:165998155 | 70.03 |
| | | 44 | GCAUCAGACCAUGACGUCCGGGG | 7:165998158 | 66.66 |
| Zm00001d006677 | BZR7 | 45 | GAGCAUCAGAGCAUGACGUCGGG | 2:214088286 | 55.28 |
| | | 46 | CGCCCUUUUCUUUGAUGGAGGGG | 2:214088334 | 44.34 |
| Zm00001d036796 | C3H26 | 47 | AAGGCCUAUAUGGUAGCUGAAGG | 6:101179329 | 63.76 |
| | | 48 | GUUCCCCGAGCAAAAUCAUGUGG | 6:101179373 | 63.33 |
| Zm00001d039014 | C3H53 | 49 | GGAAAACAGUGAGCUCUCCGUGG | 6:168714439 | 68.22 |
| | | 50 | GGCUCCUUGCACGCCUUGAGGGG | 6:168714354 | 53.46 |
| Zm00001d007436 | CA5P8 | 51 | ACAGCGUCCGUCGAACCGGAGGG | 2:231673553 | 68.3 |
| | | 52 | GUUUCCAUUUGUAUGCAGCGAGG | 2:231675312 | 63.44 |
| Zm00001d032999 | DOF40 | 53 | GAGCUCACUUCACUUGUAGCCGG | 1:246355130 | 60.06 |
| | | 54 | GGAUGAUAUCUGCUCUGGCCUGG | 1:246355060 | 50.92 |
| Zm00001d021208 | EREB115 | 55 | GACGGCAACACCUCUGAUGGCGG | 7:146038793 | 61.6 |
| | | 56 | GGAGGACUCCCCGCCAUCAGAGG | 7:146038783 | 60.3 |
| Zm00001d039260 | G2 | 57 | CGCUCUCUCGAGACGACAGGAGG | 3:750415 | 68.23 |
| | | 58 | GAUGAUGCUCGAUCUCUGGCCGG | 3:750657 | 62.95 |
| Zm00001d044785 | GLK1 | 59 | UAUAGACACUAGGCUUCGUGUGG | 9:2410267 | 64.94 |
| | | 60 | CAGGAUCGAAUGAAGUGCAGUGG | 9:2410336 | 61.66 |
| Zm00001d007962 | GLK27 | 61 | AGCUCCAUUUAUAGCAGACGAGG | 2:244256093 | 59.6 |
| | | 62 | CGGCGGAUGGGAACUGCCAGAGG | 2:244256058 | 57.99 |
| Zm00001d013202 | GLK8 | 63 | UAGAAGAAUCGGCGACCGGAAGG | 5:6407092 | 57.58 |
| | | 64 | AAUCUUUCGCGUAGAAGAAUCGG | 5:6407081 | 45.26 |
| Zm00001d046223 | HB21 | 65 | UAUAUGGGUAGAUCGCAGGGUGG | 9:73067612 | 80.52 |
| | | 66 | GCUAGUGAGACUCUUACGGGCGG | 9:73067145 | 72.39 |
| Zm00001d035907 | HB38 | 67 | CGGCCAGUAUAAUUGAUCCGUGG | 6:59432426 | 66.59 |
| | | 68 | AGUAUAAUUGAUCCGUGGUGCGG | 6:59432431 | 66.38 |
| Zm00001d005951 | HB54 | 69 | UCGCUGUAAAGAUAGUCCCGCGG | 2:193728650 | 79.09 |
| | | 70 | GCUGUAUGGAUGGUUAACCAUGG | 2:193728206 | 73.7 |
| Zm00001d053151 | HB6 | 71 | AUGACUGCAAGAUCGUAAGCAGG | 4:216321816 | 62.23 |
| | | 72 | CAUCAUCACGCUAUUAGUUGAGG | 4:216321796 | 57.86 |
| Zm00001d034515 | HB65 | 73 | CGUUCAGAGUCGGACACCUGGGG | 1:295335083 | 70.53 |
| | | 74 | GGGUCGAAUUCACUACCAAGAGG | 1:295334966 | 70.39 |
| Zm00001d011139 | HDT102 | 75 | CGGGUAACGGAAAGACCGGACGG | 8:140120567 | 59.43 |
| | | 76 | GAACUAGGGUUUAAAGCCGCCGG | 8:140120587 | 58.37 |
| Zm00001d012092 | HDT104 | 77 | CCACUCGAGUAGGGUUCUGCCGG | 8:168113730 | 60.52 |
| | | 78 | GAGUGGAAGUUGGAGGAUUGGGG | 8:168113713 | 58.7 |
| Zm00001d033859 | KN1 | 79 | UAGAACAAGGGAACUCCCUGGGG | 1:276079194 | 70.33 |
| | | 80 | GCGGCAGCAUUCCUAAUCAGGGG | 1:276079627 | 66.76 |
| Zm00001d035605 | MYB21 | 81 | UAGCUAGUAUAGCCUGCAGGUGG | 6:36428878 | 63.57 |
| | | 82 | CCUUUAAUUUUUCGGCAGCGCGG | 6:36428740 | 62.33 |
| Zm00001d012337 | MYB79 | 83 | AUUCCCGGCGCAUGCCGAAGUGG | 8:172699746 | 57.19 |
| | | 84 | CGAGGGAGAGUCAGAGUAGUAGG | 8:172699812 | 38.91 |
| Zm00001d039893 | MYB81 | 85 | UGUGAUUCCUUCCUACAGGGGGG | 3:18372312 | 68.76 |
| | | 86 | GAGAUAGCGAUCCAGACCGAGGG | 3:18371587 | 68.02 |
| Zm00001d024547 | MYBR48 | 87 | ACUUUUCAAACCCAUCACUGUGG | 10:77722792 | 74.19 |
| | | 88 | UGUCCACCGCUCCCGCUGCUUGG | 10:77723629 | 41.45 |
| Zm00001d005208 | NAC5 | 89 | GAGUUGUUCUUGGCGAUCGGGGG | 2:163613649 | 62.38 |
| | | 90 | CAUUUUUUUCUCACCUCCCGUGG | 2:163613613 | 59.06 |
| Zm00001d027530 | NAC53 | 91 | CUGGAACACGAUCUGCCUGGUGG | 1:7492272 | 73.04 |
| | | 92 | GCACUGGAACACGAUCUGCCUGG | 1:7492269 | 55.92 |
| Zm00001d024543 | NAC61 | 93 | CUUUAGCAGCGAAGGAAGCGCGG | 10:77529464 | 66.85 |
| | | 94 | CCUGUCGCGUGUGUGCUGAAAGG | 10:77530244 | 55.39 |
| Zm00001d040318 | NACTF66 | 95 | ACACCGGAAUUACAAUACGAAGG | 3:37808884 | 69.54 |
| | | 96 | CAGCAACCAAGGUUAUCUCGAGG | 3:37808628 | 67.33 |
| Zm00001d006835 | NFYA3 | 97 | AGAACUGACCAAUCGAGGAGGGG | 2:217600054 | 70.33 |
| | | 98 | GAAAUACGUCGAAGCAACUGAGG | 2:217600167 | 66.31 |
| Zm00001d025323 | ORPHAN170 | 99 | ACACUAGUUUGUUUACCCCGGGG | 10:114057439 | 70.42 |
| | | 100 | AUGCCACCUCGAUAAUGUCACGG | 10:114057384 | 67.49 |
| Zm00001d047732 | ORPHAN193 | 101 | UCACGUCACGAUGUCUUGCACGG | 9:140183689 | 63.72 |
| | | 102 | ACAUCGUGACGUGAGGCCAAGGG | 9:140183698 | 63.54 |
| Zm00001d053259 | ORPHAN211 | 103 | GUAGUAUCUGAUUCUCCGCGUGG | 4:222872654 | 70.7 |
| | | 104 | UGGCUGCCGCUAAUCAAGCGGGG | 4:222860696 | 69.14 |
| Zm00001d053566 | ORPHAN350 | 105 | ACUGUGAGCCGCACCGCACCCGG | 4:234543596 | 52.86 |
| | | 106 | AGUGGCGGGGCGUCUGCUCUCGG | 4:234543422 | 44.45 |
| Zm00001d042965 | PCO075454 | 107 | GGACGGGAAUUGGACUAAGGGGG | 3:185087564 | 72.36 |
| | | 108 | CGGACCAACCUUCGUGAGGACGG | 3:185087581 | 58.28 |
| Zm00001d014863 | RVE2 | 109 | GCUGAGUUUCGUUCUCUGGCCGG | 5:66027763 | 52.2 |
| | | 110 | AUUCGGUCCCUUCUCCGGCCGGG | 5:66027705 | 51.94 |
| Zm00001d036148 | WHIRLY1 | 111 | UCGUACUCAGACGUGGACUGCGG | 6:74548070 | 71.75 |
| | | 112 | AAUGACGCAGUCACGCAGCAGGG | 6:74547803 | 64.61 |
| Zm00001d033965 | WRKY107 | 113 | AACGUGAAGUGUUUCUCAGGGGG | 1:279765031 | 69.71 |
| | | 114 | CACCACAAAGGGUUACACAAAGG | 1:279765657 | 67.88 |
| Zm00001d049173 | WRKY36 | 115 | AGGUAAUAUUACUCACGCAGUGG | 4:19025396 | 65.86 |
| | | 116 | UUAACCCCUCUAUAUAACCCUGG | 4:19028085 | 61.35 |
| Zm00001d006001 | WRKY71 | 117 | CUACCAAAGGCUACACACGAGGG | 2:195050923 | 65.89 |
| | | 118 | CCUACCAAAGGCUACACACGAGG | 2:195050922 | 65.71 |
| Zm00001d005323 | Unannotated: | 119 | AAACCUCCCAAGACACACGAAGG | 2:169614544 | 72.35 |
| | ZF-C2H2 | 120 | AUUCGCUCGUCCGAUCAGGAAGG | 2:169616843 | 66.61 |

TABLE 14
TFs-of-importance to NUE with two guideRNA sequences targeting the first
and last exon regions for disruption by CRISPR/cas9.
We list two guide sequences to be used in combination for each TF based on their
predicted ranked efficiency.

| | Gene | | SEQ | | Genomic | |
|---|---|---|---|---|---|---|
| Gene ID | Name | Exon | ID NO | guideRNA sequence | Location | Efficiency |
| Zm00001d038270 | MYBR3 | 1 | 121 | AGGCCACAAGCUCCAC | 6:152871864 | 72.84 |
| | | | | CACGGGG | | |
| | | 2 | 122 | CAAACAACCAGUCAUC | 6:152873162 | 76.79 |
| | | | | CGAGAGG | | |
| Zm00001d042830 | MYB34 | 1 | 123 | CACGACCUGGAGAACG | 3:181378405 | 77.52 |
| | | | | ACGUCGG | | |
| | | 3 | 124 | ACAGAGUCCAACAACU | 3:181379432 | 68.51 |
| | | | | UCCACGG | | |
| Zm00001d002234 | HB75 | 2 | 125 | UCGCCGCUGGACAUCG | 2:8467383 | 74.48 |
| | | | | ACAGCGG | | |
| | | 11 | 126 | AUGAACGUGGUCCUCA | 2:8469322 | 73.63 |
| | | | | ACGGCGG | | |
| Zm00001d006293 | NLP17 | 1 | 127 | UGACUCCCCUGACCUA | 2:203860002 | 79 |
| | | | | ACGUCGG | | |
| | | 4 | 128 | UGACUCCCCUGACCUA | 2:203860002 | 79 |
| | | | | ACGUCGG | | |
| Zm00001d005029 | GRAS37 | 1 | 129 | GUUGAACACCUGGAAC | 2:154745318 | 72.47 |
| | | | | GCGGCGG | | |
| | | 2 | 130 | UUCAGCUCUCAACGGC | 2:154743354 | 73.6 |
| | | | | ACGGCGG | | |
| Zm00001d042609 | NAC109 | 1 | 131 | GCUAAUGGUAGGAAGA | 3:173458728 | 70.68 |
| | | | | GGGGAGG | | |
| | | 3 | 132 | UCCUUGCAAGUCAACA | 3:173460364 | 73.04 |
| | | | | AUGGCGG | | |
| Zm00001d024160 | BZIP107 | 2 | 133 | CGGGACCCGAUGUCAC | 10:51317579 | 68.74 |
| | | | | GUAGCGG | | |
| | | 8 | 134 | CGAGAGCGAACAGAAC | 10:51315676 | 75.67 |
| | | | | ACGCCGG | | |
| Zm00001d028999 | NAC44 | 1 | 135 | GAACUUGUAGAUGUCG | 1:54265292 | 76.39 |
| | | | | ACGUCGG | | |
| | | 3 | 136 | CACGACCAGGACGAGC | 1:54264133 | 73.84 |
| | | | | ACUGCGG | | |
| Zm00001d037607 | WRKY125 | 1 | 137 | ACGUAGUCCGACAUGA | 6:131561556 | 77.06 |
| | | | | ACGGCGG | | |
| | | 3 | 138 | UACGUCAUCACCACCU | 6:131562224 | 71.13 |
| | | | | ACGACGG | | |
| Zm00001d030678 | MYB56 | 1 | 139 | UCAUUUAUAGGUAAGA | 1:152669338 | 70.97 |
| | | | | GAGGGGG | | |
| | | 3 | 140 | GAGGGACAGGAGCUUC | 1:152668433 | 73.69 |
| | | | | AUGGUGG | | |
| Zm00001d003176 | NLP3 | 1 | 141 | AAUCCAACUGAAGUGG | 2:34610753 | 72.15 |
| | | | | ACAGAGG | | |
| | | 5 | 142 | AAUCGUGGUAGAGAAC | 2:34606532 | 78.08 |
| | | | | UCGACGG | | |
| Zm00001d028752 | ORPHAN42 | 1 | 143 | CAAUGGAAAUUACCAC | 1:45538447 | 73.37 |
| | | | | UUGGGGG | | |
| | | 3 | 144 | GGAUUUGUGGUGAAUU | 1:45539071 | 69.57 |
| | | | | ACCGUGG | | |
| Zm00001d034160 | GLK44 | 1 | 145 | GUCCUGCAUCAGGAGC | 1:285335460 | 75.48 |
| | | | | CUCGAGG | | |
| | | 5 | 146 | AGAGUGUAGAGAUAGC | 1:285336898 | 81.31 |
| | | | | GUCGCGG | | |
| Zm00001d039267 | ARF7 | 4 | 147 | AGGGGCGGUUCAGGUA | 3:841477 | 64.09 |
| | | | | AUUGGGG | | |
| | | 16 | 148 | UGGGUGCUGCUGAAAC | 3:838737 | 66.14 |
| | | | | AUGAUGG | | |
| Zm00001d034298 | BHLH47 | 1 | 149 | GAGGGUGUCCGGGAAC | 1:288724428 | 73.16 |
| | | | | CACGCGG | | |
| | | 7 | 150 | CUUCCGUAGAUAGAAU | 1:288722657 | 83.2 |
| | | | | ACGGCGG | | |
| Zm00001d011187 | BHLH92 | 1 | 151 | GAAGGAGAUGAUCUAC | 8:142339814 | 76.67 |
| | | | | CGCGCGG | | |
| | | 1 | 152 | AGCCUGAAUUGUGAAC | 8:142340149 | 78.74 |
| | | | | AAGGCGG | | |
| Zm00001d002143 | BZIP27 | 1 | 153 | GAGCCGUUCAAGGUCG | 2:6640160 | 73.62 |
| | | | | ACAGCGG | | |
| | | 9 | 154 | AAGCGACCUCACAGUC | 2:6642232 | 73.13 |
| | | | | ACUGGGG | | |
| Zm00001d018178 | BZIP4 | 1 | 155 | GGAGCCAACACAUUGC | 5:215870810 | 76.29 |
| | | | | CAUGGGG | | |
| | | 4 | 156 | CUCUACAAGUUUGGGC | 5:215861767 | 72.27 |
| | | | | ACAGCGG | | |
| Zm00001d034447 | BZIP41 | 1 | 157 | UCAGACGAUGAUGACA | 1:293292701 | 63.62 |
| | | | | UGGAAGG | | |
| | | 5 | 158 | GGACGACGAUUUCGUC | 1:293290159 | 78.21 |
| | | | | AACGGGG | | |
| Zm00001d020025 | BZIP9 | 1 | 159 | CAGAGAAGGACUGGAC | 7:86720525 | 67.66 |
| | | | | AAGGCGG | | |
| | | 2 | 160 | ACACAUCAACAGUGAC | 7:86721912 | 72.06 |
| | | | | ACAAAGG | | |
| Zm00001d021927 | BZR10 | 1 | 161 | CAAGCACUGCGACAAC | 7:165997999 | 73.06 |
| | | | | AACGAGG | | |
| | | 2 | 162 | AUAAUGCAAUGCAAUA | 7:165996767 | 75.38 |
| | | | | CAGGCGG | | |
| Zm00001d006677 | BZR7 | 1 | 163 | CAAGCACUGCGACAAC | 2:214088125 | 73.06 |
| | | | | AACGAGG | | |
| | | 2 | 164 | GAUCAGCUUCCAGAUC | 2:214087344 | 70.71 |
| | | | | GGGGCGG | | |
| Zm00001d036796 | C3H26 | 2 | 165 | CUGCCGAAGGAUGUAA | 6:101180808 | 69.98 |
| | | | | AUGGGGG | | |
| | | 3 | 166 | CUGCCGAAGGAUGUAA | 6:101180808 | 69.98 |
| | | | | AUGGGGG | | |
| Zm00001d039014 | C3H53 | 1 | 167 | CGUGACCGUGAUGACU | 6:168714178 | 76.25 |
| | | | | ACCGUGG | | |
| | | 1 | 168 | GAAGGGCCAAGAUUGA | 6:168714401 | 73.28 |
| | | | | GCAGUGG | | |
| Zm00001d007436 | CA5P8 | 1 | 169 | GCUCGGCACCCGGUUC | 2:231673605 | 70.55 |
| | | | | CCCGCGG | | |
| | | 7 | 170 | ACAGUUCCAGAGAUCA | 2:231676817 | 78.49 |
| | | | | ACAGCGG | | |
| Zm00001d032999 | DOF40 | 1 | 171 | UCCGAGUGAGAAGAAG | 1:246355253 | 69.63 |
| | | | | AUGGUGG | | |
| | | 2 | 172 | GCCCCCGAAGCUGAGC | 1:246356040 | 73.94 |
| | | | | ACCGUGG | | |
| Zm00001d021208 | EREB115 | 1 | 173 | AGUCUCUAGUAGCUCC | 7:146038604 | 73.08 |
| | | | | AGAGCGG | | |
| | | 1 | 174 | CUUACCGGAGCACCAC | 7:146039214 | 69.37 |
| | | | | CAGACGG | | |
| Zm00001d039260 | G2 | 2 | 175 | CGACUCCCAUGCUGCC | 3:753818 | 61.19 |
| | | | | CGUGUGG | | |
| | | 5 | 176 | CGUACCAAAAAUCCCA | 3:754540 | 73.32 |
| | | | | CCGGCGG | | |
| Zm00001d044785 | GLK1 | 1 | 177 | UCGCCAAGGACACCAC | 9:2409896 | 70.71 |
| | | | | AACACGG | | |
| | | 6 | 178 | AAGCUUGUGGAUGAUC | 9:2408261 | 71.59 |
| | | | | CCCGGGG | | |
| Zm00001d007962 | GLK27 | 1 | 179 | GCUCCAUUUAUAGCAG | 2:244256094 | 71.87 |
| | | | | ACGAGGG | | |
| | | 5 | 180 | AGGAACGGGAAUCAUC | 2:244254344 | 70.06 |
| | | | | AGGAGGG | | |
| Zm00001d013202 | GLK8 | 1 | 181 | GUCCUGCAUCAGGAGC | 5:6406903 | 72.84 |
| | | | | CUCGAGG | | |
| | | 5 | 182 | GCUGCUCAGGAGCAAA | 5:6405536 | 78.24 |
| | | | | GCAGCGG | | |
| Zm00001d027852 | HB102 | 1 | 183 | UUGGCCUCAUCAAACG | 1:15196061 | 66.38 |
| | | | | AGGAGGG | | |
| | | 3 | 184 | GAUGCAGCCAGAUGGA | 1:15193692 | 76.49 |
| | | | | ACAGCGG | | |
| Zm00001d046223 | HB21 | 1 | 185 | AGUCGUUCUUGAGCUC | 9:73066644 | 70.39 |
| | | | | AUUGUGG | | |
| | | 6 | 186 | UAUAUGGGUAGAUCGC | 9:73067612 | 80.52 |
| | | | | AGGGUGG | | |
| Zm00001d005951 | HB54 | 1 | 187 | GAGGAGAUGAUGGCGU | 2:193728952 | 67.12 |
| | | | | UCGGCGG | | |
| | | 4 | 188 | GGAACACUGGAACUGA | 2:193729786 | 79.13 |
| | | | | ACCGAGG | | |
| Zm00001d053151 | HB6 | 1 | 189 | AGAUCGUCCUAGCUAG | 4:216322498 | 66.72 |
| | | | | AGAGAGG | | |
| | | 5 | 190 | GGUGGAGGAGAUGUAC | 4:216318680 | 76.01 |
| | | | | CUGGAGG | | |
| Zm00001d034515 | HB65 | 1 | 191 | CAUAACGUGAAAUGUC | 1:295333271 | 77.64 |
| | | | | AAAGCGG | | |
| | | 19 | 192 | ACAGCACCACCUGCAA | 1:295313993 | 74.88 |
| | | | | ACAGCGG | | |
| Zm00001d011139 | HDT102 | 1 | 193 | UCACCCCAGAACUCCA | 8:140120638 | 68.11 |
| | | | | UCGACGG | | |
| | | 9 | 194 | UCCAUGAAGGAGAAGC | 8:140122815 | 75.1 |
| | | | | AUGGUGG | | |
| Zm00001d012092 | HDT104 | 1 | 195 | GUCGUCGCAAUGGAGU | 8:168113763 | 68.31 |
| | | | | UCUGGGG | | |
| | | 8 | 196 | UGUCUAGUUUGGGACA | 8:168116812 | 75.52 |
| | | | | UACGCGG | | |
| Zm00001d033859 | KN1 | 1 | 197 | GAGCCUUCAAGCAUAA | 1:276074920 | 66.85 |
| | | | | GACAUGG | | |
| | | 6 | 198 | ACAACUGGUUCAUCAA | 1:276073924 | 77.72 |
| | | | | CCAGCGG | | |
| Zm00001d035605 | MYB21 | 1 | 199 | AUGAGCAAGAAGCUGC | 6:36428637 | 78.15 |
| | | | | AGGGCGG | | |
| | | 2 | 200 | CUUCUGGAACGACAUC | 6:36427707 | 75.92 |
| | | | | GCGGCGG | | |
| Zm00001d012337 | MYB79 | 1 | 201 | CUUGGUAGCGAAGAAG | 8:172699829 | 72.56 |
| | | | | ACGAGGG | | |
| | | 2 | 202 | CGAGAACAAGACGCCA | 8:172700767 | 68.91 |
| | | | | UGGGCGG | | |
| Zm00001d039893 | MYB81 | 1 | 203 | GAGAGGUUGAGCAAGA | 3:18371707 | 72.33 |
| | | | | ACCGAGG | | |
| | | 3 | 204 | CAGCAUCCACGACAUC | 3:18373321 | 76.22 |
| | | | | ACCACGG | | |
| Zm00001d024547 | MYBR48 | 1 | 205 | GGAGGUGAAUUCCUCU | 10:77724211 | 64 |
| | | | | GGUGAGG | | |
| | | 3 | 206 | ACUUUUCAAACCCAUC | 10:77722792 | 74.19 |
| | | | | ACUGUGG | | |
| Zm00001d005208 | NAC5 | 1 | 207 | GGGAGGUGAGAAAAAA | 2:163613617 | 73.88 |
| | | | | AUGGGGG | | |
| | | 2 | 208 | UCCAUCUCAAGGCUCC | 2:163612236 | 76.43 |
| | | | | ACAGUGG | | |
| Zm00001d027530 | NAC53 | 1 | 209 | CUUCCGGUUCCAUCCC | 1:7492116 | 73.94 |
| | | | | ACCGAGG | | |
| | | 3 | 210 | UCACAUCUAAGCUAGC | 1:7490446 | 76.95 |
| | | | | AUCGCGG | | |
| Zm00001d024543 | NAC61 | 1 | 211 | GCGUCCACGGUGGAAG | 10:77530778 | 71.31 |
| | | | | ACAAGGG | | |
| | | 7 | 212 | GUAUGAUAUGUAUCCU | 10:77533134 | 71.06 |
| | | | | GGUGCGG | | |
| Zm00001d040318 | NACTF66 | 1 | 213 | GAGGGCGGUGUUCGG | 3:37817199 | 73.19 |
| | | | | CCCAGCGG | | |
| | | 3 | 214 | AGGGUACUUCCUGUCA | 3:37815397 | 68.49 |
| | | | | CGAGAGG | | |
| Zm00001d006835 | NFYA3 | 1 | 215 | UGAAGCCAGUAUUGUC | 2:217598189 | 62.04 |
| | | | | CUUGGGG | | |
| | | 7 | 216 | GUUGCUGUACCUGAGA | 2:217596173 | 70.5 |
| | | | | ACUGUGG | | |
| Zm00001d025323 | ORPHAN170 | 1 | 217 | UAGGGCGUUUCAAGAA | 10:114056919 | 74.45 |
| | | | | AGGGCGG | | |
| | | 6 | 218 | CAAUGAAUCCAUGAGC | 10:114052881 | 77.39 |
| | | | | AGAGCGG | | |
| Zm00001d047732 | ORPHAN193 | 1 | 219 | GGAGGCAGAUCCGACG | 9:140183341 | 68.36 |
| | | | | AACGAGG | | |
| | | 3 | 220 | AAUGCCCCUAGACACU | 9:140182509 | 69.13 |
| | | | | GCCAGGG | | |
| Zm00001d053259 | ORPHAN211 | 1 | 221 | GACCUCUCUAACCUAC | 4:222869788 | 74.63 |
| | | | | GUAGGGG | | |
| | | 5 | 222 | GGAGCCAAGAUAUAGC | 4:222874371 | 80.89 |
| | | | | ACGUCGG | | |
| Zm00001d053566 | ORPHAN350 | 1 | 223 | GAUGGCGGCGAAUCGC | 4:234543558 | 78.13 |
| | | | | ACAGGGG | | |
| | | 5 | 224 | CAUCAGUAGAGCAAGA | 4:234541169 | 70.81 |
| | | | | GCGACGG | | |
| Zm00001d042965 | PCO075454 | 1 | 225 | GAGAAGCGAAGGAAGC | 3:185087330 | 73.57 |
| | | | | AACGGGG | | |
| | | 7 | 226 | CUUUUGCAAACAAAGA | 3:185081991 | 70.47 |
| | | | | ACCGUGG | | |
| Zm00001d014863 | RVE2 | 1 | 227 | UGAUGCAACGCAACCC | 5:66027023 | 61.47 |
| | | | | AAGGAGG | | |
| | | 5 | 228 | AGCAUUGUUGUGAAUG | 5:66025609 | 61.99 |
| | | | | GUAGGGG | | |
| Zm00001d036148 | WHIRLY1 | 1 | 229 | CCUAGGAUCGAAUGAC | 6:74550201 | 75.43 |
| | | | | AGCGCGG | | |
| | | 6 | 230 | AGGGCGGACAUUCAUC | 6:74547631 | 75.88 |
| | | | | AAGGGGG | | |
| Zm00001d033965 | WRKY107 | 1 | 231 | AGCAAUCUUGGUGAUC | 1:279764318 | 72.61 |
| | | | | AGGGUGG | | |
| | | 4 | 232 | CUGUAAAAGUGGCUAC | 1:279766499 | 72.23 |
| | | | | ACCUAGG | | |
| Zm00001d049173 | WRKY36 | 1 | 233 | CAGGUCGACAUCCUCG | 4:19027570 | 74.25 |
| | | | | ACGACGG | | |
| | | 2 | 234 | GUGGUGGUGACCACAU | 4:19025688 | 74.66 |
| | | | | ACGAGGG | | |
| Zm00001d006001 | WRKY71 | 1 | 235 | UUUAGAGAGAGAGAGA | 2:195051129 | 70.94 |
| | | | | GACGGGG | | |
| | | 8 | 236 | CUUUGAAGCAAAGAAU | 2:195047291 | 73.19 |
| | | | | GCUGAGG | | |
| Zm00001d005323 | Unannotated: | 1 | 237 | AGAUCAGAUGCAAUUA | 2:169617075 | 75.6 |
| | | | | ACCGCGG | | |
| | ZF-C2H2 | 11 | 238 | UGGAUGUCAAGUUUGU | 2:169611935 | 70.43 |
| | | | | GUUGCGG | | |

TABLE 15
TFs-of-importance to NUE with two guideRNA sequences to create a
frameshift disruption by CRISPR/cas9.
We list the two guide sequences to be used in combination for each TF based on their
predicted ranked efficiency. To ensure a frameshift mutation for each TF, the
nucleotides remaining between the two guideRNA sequences will not be divisible
by three.

| | Gene | SEQ ID | | Genomic | |
|---|---|---|---|---|---|
| Gene ID | Name | NO | guideRNA sequence | Location | Efficiency |
| Zm00001d038270 | MYBR3 | 239 | AGGCCACAAGCUCCACCACGGGG | 6:152871864 | 72.84 |
| | | 240 | GGAGGCCACAAGCUCCACCACGG | 6:152871862 | 69.94 |
| Zm00001d042830 | MYB34 | 241 | CACGACCUGGAGAACGACGUCGG | 3:181378405 | 77.52 |
| | | 242 | GAGAAGAAUCAAAUUCCCGGCGG | 3:181378053 | 76.23 |
| Zm00001d002234 | HB75 | 243 | UCGCCGCUGGACAUCGACAGCGG | 2:8467383 | 74.48 |
| | | 244 | GGCAUGGGUCAUGAUGACGACGG | 2:8467717 | 74.33 |
| Zm00001d006293 | NLP17 | 245 | UGACUCCCCUGACCUAACGUCGG | 2:203860002 | 79 |
| | | 246 | GGCCACCACAGUGACGACGACGG | 2:203859718 | 70.23 |
| Zm00001d005029 | GRAS37 | 247 | GUUGAACACCUGGAACGCGGCGG | 2:154745318 | 72.47 |
| | | 248 | GCACGUCCACCUCCGCCGCGUGG | 2:154746073 | 68.75 |
| Zm00001d042609 | NAC109 | 249 | GCUAAUGGUAGGAAGAGGGGAGG | 3:173458728 | 70.68 |
| | | 250 | GGGUGGAACCGGAACCCCGGUGG | 3:173458834 | 66.79 |
| Zm00001d024160 | BZIP107 | 251 | CGGGACCCGAUGUCACGUAGCGG | 10:51317579 | 68.74 |
| | | 252 | GGGCCAUGGGGAUCUACGAGCGG | 10:51317553 | 64.99 |
| Zm00001d028999 | NAC44 | 253 | GAACUUGUAGAUGUCGACGUCGG | 1:54265292 | 76.39 |
| | | 254 | GUUUAGCCAGGAGAAACGGAGGG | 1:54265647 | 69.6 |
| Zm00001d037607 | WRKY125 | 255 | ACGUAGUCCGACAUGAACGGCGG | 6:131561556 | 77.06 |
| | | 256 | GUUCAUGUCGGACUACGUGGCGG | 6:131561561 | 72.09 |
| Zm00001d030678 | MYB56 | 257 | UCAUUUAUAGGUAAGAGAGGGGG | 1:152669338 | 70.97 |
| | | 258 | AGUAGGCAACUAAGACAAAGUGG | 1:152669295 | 67.99 |
| Zm00001d003176 | NLP3 | 259 | AAUCCAACUGAAGUGGACAGAGG | 2:34610753 | 72.15 |
| | | 260 | AGACGAGGGAUCAUUCAGCAGGG | 2:34610923 | 70.98 |
| Zm00001d028752 | ORPHAN42 | 261 | CAAUGGAAAUUACCACUUGGGGG | 1:45538447 | 73.37 |
| | | 262 | GCGGUGGCUUUGGUACCGAGCGG | 1:45538202 | 68.08 |
| Zm00001d034160 | GLK44 | 263 | GUCCUGCAUCAGGAGCCUCGAGG | 1:285335460 | 75.48 |
| | | 264 | ACAAACGAAAGAGAGAGAGGCGG | 1:285335221 | 67.67 |
| Zm00001d039267 | ARF7 | 265 | AGGGGCGGUUCAGGUAAUUGGGG | 3:841477 | 64.09 |
| | | 266 | GCAGGAGACGUCAAGGCCCGUGG | 3:841454 | 55.99 |
| Zm00001d034298 | BHLH47 | 267 | GAGGGUGUCCGGGAACCACGCGG | 1:288724428 | 73.16 |
| | | 268 | UGUGCCACAGCAGCUCCACGAGG | 1:288724550 | 73.03 |
| Zm00001d011187 | BHLH92 | 269 | AGCCUGAAUUGUGAACAAGGCGG | 8:142340149 | 78.74 |
| | | 270 | GAAGGAGAUGAUCUACCGCGCGG | 8:142339814 | 76.67 |
| Zm00001d002143 | BZIP27 | 271 | GAGCCGUUCAAGGUCGACAGCGG | 2:6640160 | 73.62 |
| | | 272 | CACCAAGCUCGAGACAAGCAGGG | 2:6640225 | 69.07 |
| Zm00001d018178 | BZIP4 | 273 | GGAGCCAACACAUUGCCAUGGGG | 5:215870810 | 76.29 |
| | | 274 | GUGGACAGGUGUCACCCACGAGG | 5:215871633 | 73.74 |
| Zm00001d034447 | BZIP41 | 275 | UCAGACGAUGAUGACAUGGAAGG | 1:293292701 | 63.62 |
| | | 276 | AUCGUCUGACUGCUCACGUGAGG | 1:293292715 | 61.79 |
| Zm00001d020025 | BZIP9 | 277 | CAGAGAAGGACUGGACAAGGCGG | 7:86720525 | 67.66 |
| | | 278 | AGAGAAGGACUGGACAAGGCGGG | 7:86720524 | 65.53 |
| Zm00001d021927 | BZR10 | 279 | CAAGCACUGCGACAACAACGAGG | 7:165997999 | 73.06 |
| | | 280 | GAGGCCCGUGAAGAUCUUGGCGG | 7:165998048 | 70.44 |
| Zm00001d006677 | BZR7 | 281 | CAAGCACUGCGACAACAACGAGG | 2:214088125 | 73.06 |
| | | 282 | GAGGCCCGUGAAGAUCUUGGCGG | 2:214088174 | 70.44 |
| Zm00001d036796 | C3H26 | 283 | CUGCCGAAGGAUGUAAAUGGGGG | 6:101180808 | 69.98 |
| | | 284 | AGAAUGCCUAUGGGACCUGGUGG | 6:101180729 | 69.85 |
| Zm00001d039014 | C3H53 | 285 | CGUGACCGUGAUGACUACCGUGG | 6:168714178 | 76.25 |
| | | 286 | AUGGGCGUUCUGCUUCCAGGAGG | 6:168714080 | 73.78 |
| Zm00001d007436 | CA5P8 | 287 | GCUCGGCACCCGGUUCCCCGCGG | 2:231673605 | 70.55 |
| | | 288 | ACAGCGUCCGUCGAACCGGAGGG | 2:231673553 | 68.3 |
| Zm00001d032999 | DOF40 | 289 | UCCGAGUGAGAAGAAGAUGGUGG | 1:246355253 | 69.63 |
| | | 290 | AGCCGGAACCGGAAGAGACGAGG | 1:246355113 | 65.47 |
| Zm00001d021208 | EREB115 | 291 | AGUCUCUAGUAGCUCCAGAGCGG | 7:146038604 | 73.08 |
| | | 292 | UGUGUUCAAUGACAUGAGCUGGG | 7:146038716 | 69.95 |
| Zm00001d039260 | G2 | 293 | CGACUCCCAUGCUGCCCGUGUGG | 3:753818 | 61.19 |
| | | 294 | GCCAGGUGACGCGGCCACACGGG | 3:753832 | 53.31 |
| Zm00001d044785 | GLK1 | 295 | UCGCCAAGGACACCACAACACGG | 9:2409896 | 70.71 |
| | | 296 | UGGCGGCGUCACGGACCAGGAGG | 9:2409969 | 67.95 |
| Zm00001d007962 | GLK27 | 297 | GCUCCAUUUAUAGCAGACGAGGG | 2:244256094 | 71.87 |
| | | 298 | GCAAGAUCGAGAUGUUCAAGCGG | 2:244255820 | 70.11 |
| Zm00001d013202 | GLK8 | 299 | GUCCUGCAUCAGGAGCCUCGAGG | 5:6406903 | 72.84 |
| | | 300 | UGGGUGACUGAGAGCCAGAGGGG | 5:6407186 | 66.2 |
| Zm00001d027852 | HB102 | 301 | UUGGCCUCAUCAAACGAGGAGGG | 1:15196061 | 66.38 |
| | | 302 | GCCAUUGGCCUCAUCAAACGAGG | 1:15196057 | 66.07 |
| Zm00001d046223 | HB21 | 303 | AGUCGUUCUUGAGCUCAUUGUGG | 9:73066644 | 70.39 |
| | | 304 | UUGGAGCAAUAACUUGACGAUGG | 9:73066570 | 69.84 |
| Zm00001d035907 | HB38 | 305 | GCGGCGGUUCUGGAACCAGACGG | 2:193729118 | 67.12 |
| | | 306 | GGAACACUGGAACUGAACCGAGG | 2:193729786 | 79.13 |
| Zm00001d005951 | HB54 | 307 | GAGGAGAUGAUGGCGUUCGGCGG | 2:193728952 | 67.12 |
| | | 308 | GGACGUGGACGAGGAGAUGAUGG | 2:193728942 | 58.09 |
| Zm00001d053151 | HB6 | 309 | AGAUCGUCCUAGCUAGAGAGAGG | 4:216322498 | 66.72 |
| | | 310 | CCUAGCUAGAGAGAGGCAGGAGG | 4:216322505 | 63.93 |
| Zm00001d034515 | HB65 | 311 | CAUAACGUGAAAUGUCAAAGCGG | 1:295333271 | 77.64 |
| | | 312 | GCAAAGGUCUAAUGACAGUGUGG | 1:295333309 | 71.89 |
| Zm00001d011139 | HDT102 | 313 | UCACCCCAGAACUCCAUCGACGG | 8:140120638 | 68.11 |
| | | 314 | CCGGUCUUUCCGUUACCCGGCGG | 8:140120571 | 67.09 |
| Zm00001d012092 | HDT104 | 315 | GUCGUCGCAAUGGAGUUCUGGGG | 8:168113763 | 68.31 |
| | | 316 | AGAACUCCAUUGCGACGACGAGG | 8:168113759 | 66.03 |
| Zm00001d033859 | KN1 | 317 | GAGCCUUCAAGCAUAAGACAUGG | 1:276074920 | 66.85 |
| | | 318 | GAAAGGGAAGCUCCCCAAGGAGG | 1:276074747 | 65.56 |
| Zm00001d035605 | MYB21 | 319 | AUGAGCAAGAAGCUGCAGGGCGG | 6:36428637 | 78.15 |
| | | 320 | CCUUUGCUAAGAAAUCCCACCGG | 6:36428470 | 73.35 |
| Zm00001d012337 | MYB79 | 321 | CUUGGUAGCGAAGAAGACGAGGG | 8:172699829 | 72.56 |
| | | 322 | CACGCCCUCGAGAAGGACGUGGG | 8:172700080 | 71.69 |
| Zm00001d039893 | MYB81 | 323 | GAGAGGUUGAGCAAGAACCGAGG | 3:18371707 | 72.33 |
| | | 324 | GCCGAGAGAAGAAAGCAGAGAGG | 3:18371649 | 71.41 |
| Zm00001d024547 | MYBR48 | 325 | GGAGGUGAAUUCCUCUGGUGAGG | 10:77724211 | 64 |
| | | 326 | UAUCACCGUUUCCUCACCAGAGG | 10:77724200 | 61.67 |
| Zm00001d005208 | NAC5 | 327 | GGGAGGUGAGAAAAAAAUGGGGG | 2:163613617 | 73.88 |
| | | 328 | GAAGCGGUAGAGGUCGACCUCGG | 2:163613360 | 69.8 |
| Zm00001d027530 | NAC53 | 329 | GCGGGUUCUGGAAGGCCACGGGG | 1:7491781 | 71.39 |
| | | 330 | GAUGGCGAAGAUUGGCGAGAGGG | 1:7491873 | 65.11 |
| Zm00001d024543 | NAC61 | 331 | GCGUCCACGGUGGAAGACAAGGG | 10:77530778 | 71.31 |
| | | 332 | AAGACCCUUGUCUUCCACCGUGG | 10:77530774 | 69.71 |
| Zm00001d040318 | NACTF66 | 333 | GAGGGCGGUGUUCGGCCCAGCGG | 3:37817199 | 73.19 |
| | | 334 | GGCUCCUCUUCCCCGCACGGCGG | 3:37817152 | 69.03 |
| Zm00001d006835 | NFYA3 | 335 | UGAAGCCAGUAUUGUCCUUGGGG | 2:217598189 | 62.04 |
| | | 336 | AAUGAAGCCAGUAUUGUCCUUGG | 2:217598191 | 57.69 |
| Zm00001d025323 | ORPHAN170 | 337 | UAGGGCGUUUCAAGAAAGGGCGG | 10:114056919 | 74.45 |
| | | 338 | AUGGAGGGCUGCUCAAAUCGCGG | 10:114053764 | 69.01 |
| Zm00001d047732 | ORPHAN193 | 339 | GGAGGCAGAUCCGACGAACGAGG | 9:140183341 | 68.36 |
| | | 340 | GUGCAAGACAUCGUGACGUGAGG | 9:140183691 | 66.75 |
| Zm00001d053259 | ORPHAN211 | 341 | GCAGAAUGGAGAUUAUCCAGAGG | 4:222862445 | 74.18 |
| | | 342 | GACCUCUCUAACCUACGUAGGGG | 4:222869788 | 74.63 |
| Zm00001d053566 | ORPHAN350 | 343 | GAUGGCGGCGAAUCGCACAGGGG | 4:234543558 | 78.13 |
| | | 344 | GGGAGAACAGAGCUAAGCAACGG | 4:234543497 | 66.44 |
| Zm00001d042965 | PCO075454 | 345 | GAGAAGCGAAGGAAGCAACGGGG | 3:185087330 | 73.57 |
| | | 346 | GGGUGAGCAGAAGAAGAAGGAGG | 3:185087250 | 71.32 |
| Zm00001d014863 | RVE2 | 347 | GATTGACAAAAGGCTGCGGTCGG | 5:66027071 | 63.05 |
| | | 348 | CAATAAATGGGTACCTTGACAGG | 5:66026986 | 39.69 |
| Zm00001d036148 | WHIRLY1 | 349 | CCUAGGAUCGAAUGACAGCGCGG | 6:74550201 | 75.43 |
| | | 350 | AACGGGCUGAGAGAACGAGGAGG | 6:74550520 | 69.49 |
| Zm00001d033965 | WRKY107 | 351 | AGCAAUCUUGGUGAUCAGGGUGG | 1:279764318 | 72.61 |
| | | 352 | CCUCUUCCUCGAGAGCUCUGCGG | 1:279763942 | 70.17 |
| Zm00001d049173 | WRKY36 | 353 | CAGGUCGACAUCCUCGACGACGG | 4:19027570 | 74.25 |
| | | 354 | CGCGCAACUAGCCAGCAGAGCGG | 4:19027998 | 68.85 |
| Zm00001d006001 | WRKY71 | 355 | UUUAGAGAGAGAGAGAGACGGGG | 2:195051129 | 70.94 |
| | | 356 | CAAGAACACUAAGAGAACAAAGG | 2:195051008 | 64.4 |
| Zm00001d005323 | ZF-C2H2 | 357 | UCAUCUCCGACUGCACACAGGGG | 2:169616501 | 73.72 |
| | | 358 | AGAUCAGAUGCAAUUAACCGCGG | 2:169617075 | 75.6 |

TABLE 16
TFs-of-importance to NUE with available BonnMu maize mutant lines.

| | | Genetic | | |
|---|---|---|---|---|
| Gene ID | Gene Name | Background | Feature | Insertion Identifier |
| Zm00001d038270 | MYBR3 | B73 | exon | BonnMu0175403; |
| | | | | BonnMu0467257 |
| Zm00001d042830 | MYB34 | B73 | exon | BonnMu0088588; |
| | | | | BonnMu0088589 |
| Zm00001d002234 | HB75 | B73 | exon | BonnMu0044799; |
| | | | | BonnMu0044802 |
| Zm00001d006293 | NLP17 | B73 | exon | BonnMu0063459; |
| | | | | BonnMu0063461; |
| | | | | BonnMu0063462; |
| | | | | BonnMu0063464 |
| Zm00001d005029 | GRAS37 | B73 | exon | BonnMu0056915; |
| | | | | BonnMu0056922; |
| | | | | BonnMu0056920; |
| | | | | BonnMu0056926; |
| | | | | BonnMu0056928; |
| | | | | BonnMu0343235 |
| Zm00001d042609 | NAC109 | N/A | N/A | N/A |
| Zm00001d024160 | BZIP107 | B73 | exon | BonnMu0253222; |
| | | | | BonnMu0313225 |
| Zm00001d028999 | NAC44 | N/A | N/A | N/A |
| Zm00001d037607 | WRKY125 | B73 | exon | BonnMu0171468 |
| Zm00001d030678 | MYB56 | N/A | N/A | N/A |
| Zm00001d003176 | NLP3 | N/A | N/A | N/A |
| Zm00001d028752 | ORPHAN42 | B73 | exon | BonnMu0009723; |
| | | | | BonnMu0009727; |
| | | | | BonnMu0009726; |
| | | | | BonnMu0009728; |
| | | | | BonnMu0009731 |
| Zm00001d034160 | GLK44 | B73 | exon | BonnMu0036040 |
| Zm00001d039267 | ARF7 | B73; Co125 | exon | BonnMu0072312; |
| | | | | BonnMu0072314; |
| | | | | BonnMu0072315; |
| | | | | BonnMu0072319; |
| | | | | BonnMu0072318; |
| | | | | BonnMu0359744 |
| Zm00001d034298 | BHLH47 | B73; Co125 | exon | BonnMu0036770; |
| | | | | BonnMu0036773; |
| | | | | BonnMu0036774; |
| | | | | BonnMu0036776; |
| | | | | BonnMu0036777; |
| | | | | BonnMu0036783; |
| | | | | BonnMu0036782; |
| | | | | BonnMu0304092; |
| | | | | BonnMu0304094 |
| Zm00001d011187 | BHLH92 | N/A | N/A | N/A |
| Zm00001d002143 | BZIP27 | N/A | N/A | N/A |
| Zm00001d018178 | BZIP4 | B73 | exon | BonnMu0156420; |
| | | | | BonnMu0156421 |
| Zm00001d034447 | BZIP41 | B73 | exon | BonnMu0037546; |
| | | | | BonnMu0037547 |
| Zm00001d020025 | BZIP9 | N/A | N/A | N/A |
| Zm00001d021927 | BZR10 | B73 | exon | BonnMu0198311; |
| | | | | BonnMu0198313; |
| | | | | BonnMu0491175 |
| Zm00001d006677 | BZR7 | B73 | exon | BonnMu0065921; |
| | | | | BonnMu0065924; |
| | | | | BonnMu0065925; |
| | | | | BonnMu0065927 |
| Zm00001d036796 | C3H26 | N/A | N/A | N/A |
| Zm00001d039014 | C3H53 | B73 | exon | BonnMu0180752; |
| | | | | BonnMu0180751 |
| Zm00001d007436 | CA5P8 | N/A | N/A | N/A |
| Zm00001d032999 | DOF40 | Co125 | exon | BonnMu0028789 |
| Zm00001d021208 | EREB115 | N/A | N/A | N/A |
| Zm00001d039260 | G2 | N/A | N/A | N/A |
| Zm00001d044785 | GLK1 | N/A | N/A | N/A |
| Zm00001d007962 | GLK27 | B73 | exon | BonnMu0071693; |
| | | | | BonnMu0359184 |
| Zm00001d013202 | GLK8 | N/A | N/A | N/A |
| Zm00001d027852 | HB102 | N/A | N/A | N/A |
| Zm00001d046223 | HB21 | B73 | exon | BonnMu0235262; |
| | | | | BonnMu0235261; |
| | | | | BonnMu0235267; |
| | | | | BonnMu0235270; |
| | | | | BonnMu0530066 |
| Zm00001d035907 | HB38 | N/A | N/A | N/A |
| Zm00001d005951 | HB54 | B73 | exon | BonnMu0061120 |
| Zm00001d053151 | HB6 | N/A | N/A | N/A |
| Zm00001d034515 | HB65 | N/A | N/A | N/A |
| Zm00001d011139 | HDT102 | N/A | N/A | N/A |
| Zm00001d012092 | HDT104 | N/A | N/A | N/A |
| Zm00001d033859 | KN1 | N/A | N/A | N/A |
| Zm00001d035605 | MYB21 | N/A | N/A | N/A |
| Zm00001d012337 | MYB79 | B73 | exon | BonnMu0225534; |
| | | | | BonnMu0225535 |
| Zm00001d039893 | MYB81 | B73 | exon | BonnMu0075950 |
| Zm00001d024547 | MYBR48 | N/A | N/A | N/A |
| Zm00001d005208 | NAC5 | B73; Co125 | exon | BonnMu0057716; |
| | | | | BonnMu0057718; |
| | | | | BonnMu0057720; |
| | | | | BonnMu0057722; |
| | | | | BonnMu0057721; |
| | | | | BonnMu0344160 |
| Zm00001d027530 | NAC53 | B73 | exon | BonnMu0002368; |
| | | | | BonnMu0268591 |
| Zm00001d024543 | NAC61 | B73 | exon | BonnMu0314713 |
| Zm00001d040318 | NACTF66 | N/A | N/A | N/A |
| Zm00001d006835 | NFYA3 | N/A | N/A | N/A |
| Zm00001d025323 | ORPHAN170 | N/A | N/A | N/A |
| Zm00001d047732 | ORPHAN193 | B73 | exon | BonnMu0242509; |
| | | | | BonnMu0242511; |
| | | | | BonnMu0242513; |
| | | | | BonnMu0242517 |
| Zm00001d053259 | ORPHAN211 | N/A | | N/A |
| Zm00001d053566 | ORPHAN350 | B73 | exon | BonnMu0121701 |
| Zm00001d042965 | PCO075454 | N/A | | N/A |
| Zm00001d014863 | RVE2 | N/A | | N/A |
| Zm00001d036148 | WHIRLY1 | B73 | exon | BonnMu0164498; |
| | | | | BonnMu0164502 |
| Zm00001d033965 | WRKY107 | B73 | exon | BonnMu0034807; |
| | | | | BonnMu0034817 |
| Zm00001d049173 | WRKY36 | N/A | | N/A |
| Zm00001d006001 | WRKY71 | B73 | exon | BonnMu0061422 |
| Zm00001d005323 | Unannotated: | N/A | N/A | N/A |
| | ZF-C2H2 | | | |

TABLE 17
TFs-of-importance to NUE with available UniformMu maize mutant lines

| Gene ID | Gene Name | Feature | Insertion Identifier |
|---|---|---|---|
| Zm00001d038270 | MYBR3 | N/A | N/A |
| Zm00001d042830 | MYB34 | N/A | N/A |
| Zm00001d002234 | HB75 | N/A | N/A |
| Zm00001d006293 | NLP17 | exon | mu1007743; mu1043614; mu1030337 |
| Zm00001d005029 | GRAS37 | exon | mu1013874; mu1019839 |
| Zm00001d042609 | NAC109 | N/A | N/A |
| Zm00001d024160 | BZIP107 | exon | mu1102129; mu1047032 |
| Zm00001d028999 | NAC44 | N/A | N/A |
| Zm00001d037607 | WRKY125 | N/A | N/A |
| Zm00001d030678 | MYB56 | N/A | N/A |
| Zm00001d003176 | NLP3 | N/A | N/A |
| Zm00001d028752 | ORPHAN42 | exon | mu1043913 |
| Zm00001d034160 | GLK44 | N/A | N/A |
| Zm00001d039267 | ARF7 | exon | mu1094628; mu1090519; mu1017909; |
| | | | mu1076913; mu1086101 |
| Zm00001d034298 | BHLH47 | exon | mu1089017 |
| Zm00001d011187 | BHLH92 | exon | mu1078575 |
| Zm00001d002143 | BZIP27 | exon | mu1008454; mu1018254 |
| Zm00001d018178 | BZIP4 | exon | mu1041361 |
| Zm00001d034447 | BZIP41 | N/A | N/A |
| Zm00001d020025 | BZIP9 | N/A | N/A |
| Zm00001d021927 | BZR10 | exon | mu1092833 |
| Zm00001d006677 | BZR7 | exon | mu1059190 |
| Zm00001d036796 | C3H26 | N/A | N/A |
| Zm00001d039014 | C3H53 | N/A | N/A |
| Zm00001d007436 | CA5P8 | N/A | N/A |
| Zm00001d032999 | DOF40 | N/A | N/A |
| Zm00001d021208 | EREB115 | N/A | N/A |
| Zm00001d039260 | G2 | exon | mu1010107 |
| Zm00001d044785 | GLK1 | N/A | N/A |
| Zm00001d007962 | GLK27 | N/A | N/A |
| Zm00001d013202 | GLK8 | N/A | N/A |
| Zm00001d027852 | HB102 | N/A | N/A |
| Zm00001d046223 | HB21 | exon | mu1030960 |
| Zm00001d035907 | HB38 | N/A | N/A |
| Zm00001d005951 | HB54 | N/A | N/A |
| Zm00001d053151 | HB6 | exon | mu1081217; mu1104053 |
| Zm00001d034515 | HB65 | N/A | N/A |
| Zm00001d011139 | HDT102 | N/A | N/A |
| Zm00001d012092 | HDT104 | N/A | N/A |
| Zm00001d033859 | KN1 | N/A | N/A |
| Zm00001d035605 | MYB21 | N/A | N/A |
| Zm00001d012337 | MYB79 | exon | mu1072393 |
| Zm00001d039893 | MYB81 | N/A | N/A |
| Zm00001d024547 | MYBR48 | N/A | N/A |
| Zm00001d005208 | NAC5 | exon | mu1085854 |
| Zm00001d027530 | NAC53 | N/A | N/A |
| Zm00001d024543 | NAC61 | N/A | N/A |
| Zm00001d040318 | NACTF66 | N/A | N/A |
| Zm00001d006835 | NFYA3 | N/A | N/A |
| Zm00001d025323 | ORPHAN170 | exon | mu1094335 |
| Zm00001d047732 | ORPHAN193 | exon | mu1016599 |
| Zm00001d053259 | ORPHAN211 | N/A | N/A |
| Zm00001d053566 | ORPHAN350 | N/A | N/A |
| Zm00001d042965 | PCO075454 | N/A | N/A |
| Zm00001d014863 | RVE2 | N/A | N/A |
| Zm00001d036148 | WHIRLY1 | N/A | N/A |
| Zm00001d033965 | WRKY107 | exon | mu1085538; mu1055196 |
| Zm00001d049173 | WRKY36 | N/A | N/A |
| Zm00001d006001 | WRKY71 | N/A | N/A |
| Zm00001d005323 | Unannotated: | N/A | N/A |
| | ZF-C2H2 | | |

TABLE 18
TFs-of-importance to NUE with available IlluminaMu maize mutant lines

| Gene ID | Gene Name | Feature | Insertion Identifier |
|---|---|---|---|
| Zm00001d038270 | MYBR3 | exon | mu-illumina_127248.7; mu-illumina_140040.7; |
| | | | mu-illumina_140663.7; mu-illumina_145430.7; |
| | | | mu-illumina_158597.7 |
| Zm00001d042830 | MYB34 | N/A | N/A |
| Zm00001d002234 | HB75 | N/A | N/A |
| Zm00001d006293 | NLP17 | exon | mu-illumina_18624.7; mu-illumina_20761.7 |
| Zm00001d005029 | GRAS37 | exon | mu-illumina_132298.7 |
| Zm00001d042609 | NAC109 | N/A | N/A |
| Zm00001d024160 | BZIP107 | N/A | N/A |
| Zm00001d028999 | NAC44 | N/A | N/A |
| Zm00001d037607 | WRKY125 | N/A | N/A |
| Zm00001d030678 | MYB56 | N/A | N/A |
| Zm00001d003176 | NLP3 | N/A | N/A |
| Zm00001d028752 | ORPHAN42 | N/A | N/A |
| Zm00001d034160 | GLK44 | exon | mu-illumina_36045.7; mu-illumina_46126.7 |
| Zm00001d039267 | ARF7 | exon | mu-illumina_16576.7; mu-illumina_18803.7; |
| | | | ;mu-illumina_21215.7 |
| Zm00001d034298 | BHLH47 | N/A | N/A |
| Zm00001d011187 | BHLH92 | N/A | N/A |
| Zm00001d002143 | BZIP27 | N/A | N/A |
| Zm00001d018178 | BZIP4 | N/A | N/A |
| Zm00001d034447 | BZIP41 | N/A | N/A |
| Zm00001d020025 | BZIP9 | N/A | N/A |
| Zm00001d021927 | BZR10 | N/A | N/A |
| Zm00001d006677 | BZR7 | exon | mu-illumina_15164.7; mu-illumina_119374.7 |
| Zm00001d036796 | C3H26 | N/A | N/A |
| Zm00001d039014 | C3H53 | N/A | N/A |
| Zm00001d007436 | CA5P8 | N/A | N/A |
| Zm00001d032999 | DOF40 | N/A | N/A |
| Zm00001d021208 | EREB115 | N/A | N/A |
| Zm00001d039260 | G2 | N/A | N/A |
| Zm00001d044785 | GLK1 | N/A | N/A |
| Zm00001d007962 | GLK27 | N/A | N/A |
| Zm00001d013202 | GLK8 | N/A | N/A |
| Zm00001d027852 | HB102 | N/A | N/A |
| Zm00001d046223 | HB21 | exon | mu-illumina_112156.7 |
| Zm00001d035907 | HB38 | N/A | N/A |
| Zm00001d005951 | HB54 | exon | mu-illumina_27889.7 |
| Zm00001d053151 | HB6 | N/A | N/A |
| Zm00001d034515 | HB65 | N/A | N/A |
| Zm00001d011139 | HDT102 | N/A | N/A |
| Zm00001d012092 | HDT104 | N/A | N/A |
| Zm00001d033859 | KN1 | exon | mu-illumina_131339.7 |
| Zm00001d035605 | MYB21 | N/A | N/A |
| Zm00001d012337 | MYB79 | N/A | N/A |
| Zm00001d039893 | MYB81 | exon | mu-illumina_646006.7 |
| Zm00001d024547 | MYBR48 | N/A | N/A |
| Zm00001d005208 | NAC5 | N/A | N/A |
| Zm00001d027530 | NAC53 | N/A | N/A |
| Zm00001d024543 | NAC61 | N/A | N/A |
| Zm00001d040318 | NACTF66 | exon | mu-illumina_131998.7 |
| Zm00001d006835 | NFYA3 | N/A | V/A |
| Zm00001d025323 | ORPHAN170 | exon | mu-illumina_111562.7 |
| Zm00001d047732 | ORPHAN193 | exon | mu-illumina_126915.7 |
| Zm00001d053259 | ORPHAN211 | N/A | N/A |
| Zm00001d053566 | ORPHAN350 | N/A | N/A |
| Zm00001d042965 | PCO075454 | N/A | N/A |
| Zm00001d014863 | RVE2 | N/A | N/A |
| Zm00001d036148 | WHIRLY1 | N/A | N/A |
| Zm00001d033965 | WRKY107 | exon | mu-illumina_103988.7 |
| Zm00001d049173 | WRKY36 | N/A | N/A |
| Zm00001d006001 | WRKY71 | exon | mu-illumina_157442.7 |
| Zm00001d005323 | Unannotated: | N/A | N/A |
| | ZF-C2H2 | | |

TABLE 19
TFs-of-importance to NUE: CRISPR/cas13 guide RNAs that target the TF
mRNA for degradation.

| | Gene | SEQ | | Genomic |
|---|---|---|---|---|
| Gene ID | Name | ID NO | guideRNA sequences | Location |
| Zm00001d038270 | MYBR3 | 359 | UUUAUGCAGCGGGGGGCCGGGAGCAGUG | 6:152872004 |
| | | 360 | UUUUGUUUUUGGUUGGGAGGCCACAAGC | 6:152871847 |
| Zm00001d042830 | MYB34 | 361 | UUUGUUCUCCGCCGCCGUCCACGCGUCU | 3:181378269 |
| | | 362 | UUUGAUUCUUCUCCCCUAAAUACUCACG | 3:181378063 |
| Zm00001d002234 | HB75 | 363 | UUUUCUUGAAAAUUGGGUUCAACGGAUG | 2:8466366 |
| | | 364 | UUUCUUGAAAAUUGGGUUCAACGGAUGG | 2:8466367 |
| Zm00001d006293 | NLP17 | 365 | UUUUGUUGUUAGCGUUACCAAGAUCAUA | 2:203859423 |
| | | 366 | UUUGUUGUUAGCGUUACCAAGAUCAUAG | 2:203859424 |
| Zm00001d005029 | GRAS37 | 367 | UUUACGACGUGACCGGCUCCGACUCCAA | 2:154744929 |
| | | 368 | UUUCCUCGCAAGACCACGUCCUCAUCCA | 2:154746318 |
| Zm00001d042609 | NAC109 | 369 | UUUCAAGCUCUCCUCCCCUCUUCCUACC | 3:173458717 |
| | | 370 | UUUUGAUUAUUCUGCUGGUCUCGUCUGA | 3:173460792 |
| Zm00001d024160 | BZIP107 | 371 | UUUUGAGUGGUGUAUCUGCCCCUGGCCU | 10:51327055 |
| | | 372 | UUUGAGUGGUGUAUCUGCCCCUGGCCUU | 10:51327054 |
| Zm00001d028999 | NAC44 | 373 | UUUGGGGACGGGAGGGGAAGGUCUGCUU | 1:54265679 |
| | | 374 | UUUUGGGGACGGGAGGGGAAGGUCUGCU | 1:54265678 |
| Zm00001d037607 | WRKY125 | 375 | UUUGCUGUGCAUAGGUAGUACAUACAUC | 6:131561720 |
| | | 376 | UUUCAUUUCAUCCAUCAGCAGGUGGUGU | 6:131561831 |
| Zm00001d030678 | MYB56 | 377 | UUUUCAUCACAGGAUCGAUAUGGUGACA | 1:152669096 |
| | | 378 | UUUCAUCACAGGAUCGAUAUGGUGACAG | 1:152669095 |
| Zm00001d003176 | NLP3 | 379 | UUUGCCUUGUGGUCAUCUUCGGGGUCAU | 2:34610883 |
| | | 380 | UUUGGAACAAGCCCAUCUGAGUCUUUUA | 2:34610723 |
| Zm00001d028752 | ORPHAN42 | 381 | UUUUUCCGCAGACCACUCCAAAGCAACC | 1:45537858 |
| | | 382 | UUUUCCGCAGACCACUCCAAAGCAACCG | 1:45537859 |
| Zm00001d034160 | GLK44 | 383 | UUUGCGCAGAGAAGAAUCGGCGACCGGA | 1:285335255 |
| | | 384 | UUUUGCGCAGAGAAGAAUCGGCGACCGG | 1:285335256 |
| Zm00001d039267 | ARF7 | 385 | UUUGCGGUUCUUGGCUUGGCCUCCUGGU | 3:842479 |
| | | 386 | UUUCUUUAAUGGAGCGAGGGUGAGCCUA | 3:842587 |
| Zm00001d034298 | BHLH47 | 387 | UUUUGCAGGAGAGAAGACGUUGUUCUCC | 1:288724768 |
| | | 388 | UUUGCAGGAGAGAAGACGUUGUUCUCCG | 1:288724767 |
| Zm00001d011187 | BHLH92 | 389 | UUUCUGCCUAGGUCUGAUUCUUCCAAGA | 8:142340699 |
| | | 390 | UUUCGCCAGCUGCUGCCGGGAACGUGAA | 8:142339418 |
| Zm00001d002143 | BZIP27 | 391 | UUUGCCCCGUGGCCAGCUCCGCCUCGCC | 2:6640029 |
| | | 392 | UUUCCUGAUCCGUACUCCUCUCUGGCUC | 2:6640374 |
| Zm00001d018178 | BZIP4 | 393 | UUUCGAAGCGUGUGAAUCCGGUGGGCGA | 5:215871406 |
| | | 394 | UUUACCUGUUUCCUUUGGCGCGACCUCG | 5:215870516 |
| Zm00001d034447 | BZIP41 | 395 | UUUGUUCUUAAGGCCGGCAAUAAUUUUA | 1:293291124 |
| | | 396 | UUUUUUUAAAUGCAAAUAGGUGAGGCUA | 1:293291678 |
| Zm00001d020025 | BZIP9 | 397 | UUUUAUGUCUUCUCAGUAAGCCGCCAGU | 7:86720565 |
| | | 398 | UUUAUGUCUUCUCAGUAAGCCGCCAGUA | 7:86720566 |
| Zm00001d021927 | BZR10 | 399 | UUUGGCAUCUACUCACUGUUUGUUCUGU | 7:165996689 |
| | | 400 | UUUCGAUCGGAGGCACAUUCGGUGUAGG | 7:165996863 |
| Zm00001d006677 | BZR7 | 401 | UUUCGGUAGGUGGUGCCGUCGUCCUCGA | 2:214088061 |
| | | 402 | UUUCGGUGUAGGUGAUGAUUCUGUUGUU | 2:214086912 |
| Zm00001d036796 | C3H26 | 403 | UUUACUUCAGCUUUUAUUCUUUGCAUAU | 6:101180423 |
| | | 404 | UUUUAUUCUUUGCAUAUAUAUUUUUAGC | 6:101180434 |
| Zm00001d039014 | C3H53 | 405 | UUUUCCCUCCCGUUCACGGUUCCACUGC | 6:168714417 |
| | | 406 | UUUCCCUCCCGUUCACGGUUCCACUGCU | 6:168714416 |
| Zm00001d007436 | CA5P8 | 407 | UUUCUGCUUUAUUUUUUUGGUGACGACG | 2:231673502 |
| | | 408 | UUUUCUGCUUUAUUUUUUUGGUGACGAC | 2:231673503 |
| Zm00001d032999 | DOF40 | 409 | UUUCUUCUCUUCGGCAAGCUGCUGCAAG | 1:246355193 |
| | | 410 | UUUCUGUUUGUUUGGAUCCGAGUGAGAA | 1:246355237 |
| Zm00001d021208 | EREB115 | 411 | UUUUCCGUUUCUUUGCUUGGCCUGUGGC | 7:146038572 |
| | | 412 | UUUCCGUUUCUUUGCUUGGCCUGUGGCG | 7:146038571 |
| Zm00001d039260 | G2 | 413 | UUUGGUACGCCUUGCUUGUGCAGCUCCG | 3:754521 |
| | | 414 | UUUUGGUACGCCUUGCUUGUGCAGCUCC | 3:754522 |
| Zm00001d044785 | GLK1 | 415 | UUUCCCUUCUUCCACGUCUGCGCACGCU | 9:2409856 |
| | | 416 | UUUUCGCCGGCAAAGGCUCCUCCACAGC | 9:2409761 |
| Zm00001d007962 | GLK27 | 417 | UUUAUAGCAGACGAGGGAAAAAACUGGA | 2:244256100 |
| | | 418 | UUUUUUCCCUCGUCUGCUAUAAAUGGAG | 2:244256095 |
| Zm00001d013202 | GLK8 | 419 | UUUCUCAGCUUCUCUCCGGUUCCUUCCU | 5:6407112 |
| | | 420 | UUUCGCGUAGAAGAAUCGGCGACCGGAA | 5:6407085 |
| Zm00001d027852 | HB102 | 421 | UUUGAUGAGGCCAAUGGCCAGCAAUGGC | 1:15196047 |
| | | 422 | UUUGGAGGGAAGAAGGGCGAGGGUGCGG | 1:15196012 |
| Zm00001d046223 | HB21 | 423 | UUUCUGGAAGUCGUUCUUGAGCUCAUUG | 9:73066647 |
| | | 424 | UUUGCGCAGCGCGCGGUGUCAGCGACGC | 9:73066873 |
| Zm00001d035907 | HB38 | 425 | UUUGGCCACCAUGAGGCAUCAGAGGCCC | 6:59432711 |
| | | 426 | UUUGCACUACUACCUAACCACCACCACC | 6:59432771 |
| Zm00001d005951 | HB54 | 427 | UUUAUCGCUCCGAAUUCCUCUAUUCGGU | 2:193729866 |
| | | 428 | UUUGCAGAAAAGCCCCUUCUUUAUCGCU | 2:193729885 |
| Zm00001d053151 | HB6 | 429 | UUUCCCUUGAACAAAAGAAAAUUAGCUG | 4:216322396 |
| | | 430 | UUUUCUUUUGUUCAAGGGAAAGGACAGC | 4:216322389 |
| Zm00001d034515 | HB65 | 431 | UUUUGGAGCAAAAAGGGAUAAUGCAGGG | 1:295335464 |
| | | 432 | UUUGGAGCAAAAAGGGAUAAUGCAGGGG | 1:295335463 |
| Zm00001d011139 | HDT102 | 433 | UUUAAAGCCGCCGGGUAACGGAAAGACC | 8:140120573 |
| | | 434 | UUUCCGUUACCCGGCGGCUUUAAACCCU | 8:140120577 |
| Zm00001d012092 | HDT104 | 435 | UUUAGCGGCAGCUUCGAACUGGGGGGUG | 8:168113664 |
| | | 436 | UUUAGGGUUUAGCGGCAGCUUCGAACUG | 8:168113671 |
| Zm00001d033859 | KN1 | 437 | UUUUUUUUUUGAAAUCUUAAGCCCUGUG | 1:276075658 |
| | | 438 | UUUUUUUUUGAAAUCUUAAGCCCUGUGU | 1:276075657 |
| Zm00001d035605 | MYB21 | 439 | UUUGGUGAACGCCGGUGGGAUUUCUUAG | 6:36428476 |
| | | 440 | UUUCGUUGCAUCUCUUUGUAUAAUACUG | 6:36427391 |
| Zm00001d012337 | MYB79 | 441 | UUUUAGGCCAGAAUAAACUCCAGCCACU | 8:172699723 |
| | | 442 | UUUAGGCCAGAAUAAACUCCAGCCACUU | 8:172699724 |
| Zm00001d039893 | MYB81 | 443 | UUUCACCUGAACUGAACAGCGCCUCCUC | 3:18371793 |
| | | 444 | UUUUGCCUUGUUCUGGGUGGGGUCUCUC | 3:18371821 |
| Zm00001d024547 | MYBR48 | 445 | UUUUUCUACUUACAUAUGUGUGCCACAG | 10:77722809 |
| | | 446 | UUUUCUACUUACAUAUGUGUGCCACAGU | 10:77722808 |
| Zm00001d005208 | NAC5 | 447 | UUUCGUCGACAUCAGCAUCCACCAUAGC | 2:163613831 |
| | | 448 | UUUGACCCCACCCGCUCCCGAGAAGCUU | 2:163613695 |
| Zm00001d027530 | NAC53 | 449 | UUUCGCGGGCUCGCUCGCUUAUAUAGAC | 1:7492297 |
| | | 450 | UUUAUUUCUGCGUGAUGUUGGUACGGGG | 1:7490348 |
| Zm00001d024543 | NAC61 | 451 | UUUCCGUCGCCACCUACCACGUGAACCU | 10:77530215 |
| | | 452 | UUUCAGCACACACGCGACAGGAAGGUUC | 10:77530237 |
| Zm00001d040318 | NACTF66 | 453 | UUUGUGUUUUGUGGUUAAUGCAGACAUU | 3:37816006 |
| | | 454 | UUUACAAUGUCUGCAUUAACCACAAAAC | 3:37816001 |
| Zm00001d006835 | NFYA3 | 455 | UUUUCACUUGAAGUUUGACGAUGCACAA | 2:217597701 |
| | | 456 | UUUGUGCAAAUUAUAGAAAAGAAGAGCA | 2:217597644 |
| Zm00001d025323 | ORPHAN170 | 457 | UUUUGAGUGAGUCGGCGAUGAAUCUCAC | 10:11405844 |
| | | 458 | UUUGAGUGAGUCGGCGAUGAAUCUCACG | 10:114058440 |
| Zm00001d047732 | ORPHAN193 | 459 | UUUUCAAAUGGCCAUUUUCCGCAAUCAG | 9:140183780 |
| | | 460 | UUUCAAAUGGCCAUUUUCCGCAAUCAGA | 9:140183779 |
| Zm00001d053259 | ORPHAN211 | 461 | UUUGACUAGUGAAAGUUGCUUUAGUCAU | 4:222870604 |
| | | 462 | UUUAGUCAUUUUACUAAGUAAGAACGAC | 4:222870623 |
| Zm00001d053566 | ORPHAN350 | 463 | UUUGGUGGGGGCACUGUGAGCCGCACCG | 4:234543584 |
| | | 464 | UUUCUCUGCUGUGGUCUUUCUUCCUCGC | 4:234542695 |
| Zm00001d042965 | | 465 | UUUGGACGUUCUUGCUCUUAUUCUUGUU | 3:185087448 |
| | PCO075454 | 466 | UUUUGGACGUUCUUGCUCUUAUUCUUGU | 3:185087447 |
| Zm00001d014863 | RVE2 | 467 | UUUCUGGGGGCGAAGGGAGGGGAGGGGA | 5:66027938 |
| | | 468 | UUUCCAUUUCCUUGCCCUUGCCAGCCAG | 5:66027875 |
| Zm00001d036148 | WHIRLY1 | 469 | UUUCUGGAGGAGGCAACCGGAGGGACUA | 6:74550388 |
| | | 470 | UUUCAAGGGGCGGAGGUUGGUAUCAUUU | 6:74549547 |
| Zm00001d033965 | WRKY107 | 471 | UUUGCUUCGUUGCAGGCAGGCCCGUCCA | 1:279763673 |
| | | 472 | UUUGAGGGCUCGUCGUCUAAGGAUCCUG | 1:279764045 |
| Zm00001d049173 | WRKY36 | 473 | UUUCAGCCUCCCCUCCCUCACAAGCUCU | 4:19028039 |
| | | 474 | UUUCCUAGCCCAAUAUAAUGAGGGGUGG | 4:19025232 |
| Zm00001d006001 | WRKY71 | 475 | UUUGUGUUCCAGAGAUCUUCGUCGUUCU | 2:195050951 |
| | | 476 | UUUGGUAGGUUGGUGGAAGGGCGAGGCA | 2:195050903 |
| Zm00001d005323 | Unannotated: | 477 | UUUUUUUUUACCGCACCCCUGUGUGCAG | 2:169616511 |
| | ZF-C2H2 | 478 | UUUUUUUUACCGCACCCCUGUGUGCAGU | 2:169616510 |

TABLE 20
Promoters to guide maize NUE engineering

| Gene ID | | | |
|---|---|---|---|
| associated with | | | |
| Promoter | Gene Name | Purpose | Reference |
| Zm00001d015327 | ZmUbi1 | Constitutive | Cornejo, M. J., et al. Plant |
| | | overexpression | Molecular Biology, 23, 567- |
| | | | 581 (1993) |
| Zm00001d038163 | ZmPPDK | Mesophyll specific | Matsuoka, M. et al. |
| | | overexpression | Proceedings of the National |
| | | | Academy of Sciences of the |
| | | | United States of America, 90, |
| | | | 9586-9590. |
| | | | (1993) |
| Zm00001d023215 | bnlg2057 | Root specific | Moisseyev, G., et al. RGPDB: |
| | | overexpression | database of root-associated |
| | | | genes and promoters in maize, |
| | | | soybean, and |
| | | | sorghum. Database, 2020, |
| | | | baaa038 |

TABLE X
XGBoost Scores for Maize ZmMYB34/R3 TF regulon genes and their Arabidopsis orthologs.

| | Maize | Maize | | | Arabidopsis | | |
|---|---|---|---|---|---|---|---|
| Maize | XGboost | gene | Maize | Arabidopsis | XGboost | Arabidopsis | Arabidopsis |
| geneID | score | Name | geneDescription | geneID | score | geneName | geneDescription |
| Zm00001d038270 | 320.53 | MYBR3 | MYB-related- | AT5G58900 | 167.20 | DIV1 | Homeodomain-like |
| | | | transcription | | | | transcriptional |
| | | | factor 3 | | | | regulator |
| Zm00001d042830 | — | MYB34 | MYB-related- | AT5G58900 | 167.20 | DIV1 | Homeodomain-like |
| | | | transcription | | | | transcriptional |
| | | | factor 3 | | | | regulator |
| Zm00001d034160 | 85.92 | GLK44 | G2-like- | AT1G49560 | 52.76 | HHO6 | Homeodomain-like |
| | | | transcription | | | | superfamily protein |
| | | | factor 44 | | | | |
| Zm00001d007962 | 11.64 | GLK27 | G2-like- | AT1G49560 | 52.76 | HHO6 | Homeodomain-like |
| | | | transcription | | | | superfamily protein |
| | | | factor 27 | | | | |
| Zm00001d032999 | 86.57 | DOF40 | C2C2-Dof- | AT5G62430 | 59.55 | CDF1 | cycling DOF |
| | | | transcription | | | | factor 1 |
| | | | factor 40 | | | | |
| Zm00001d006001 | 28.78 | WRKY71 | WRKY- | AT5G56270 | 211.53 | WRKY2 | WRKY |
| | | | transcription | | | | DNA-binding |
| | | | factor 71 | | | | protein 2 |
| Zm00001d052165 | 9.36 | ZmNiR1.1 | nitrite reductase2 | AT2G15620 | 106.62 | NIR1 | nitrate reductase 1 |
| Zm00001d018161 | 97.80 | ZmNiR1.2 | nitrate reductase1 | AT2G15620 | 106.62 | NIR1 | nitrate reductase 1 |
| Zm00001d043845 | 1.79 | GOGAT2 | Glutamate | AT5G53460 | 73.63 | GLT1 | NADH-dependent |
| | | | synthase 1 | | | | glutamate synthase 1 |
| | | | [NADH] | | | | |
| | | | chloroplastic | | | | |
| Zm00001d018031 | 69.77 | EXPA2 | alpha expansin2 | AT1G69530 | 42.02 | EXPA1 | expansin A1 |
| Zm00001d031332 | 1.30 | PZA03531 | Heat shock | AT5G56030 | 74.34 | HSP81-2 | heat shock |
| | | | protein 90-2 | | | | protein 81-2 |
| Zm00001d031332 | 1.30 | PZA03531 | Heat shock | AT5G56010 | 60.37 | HSP81-3 | heat shock |
| | | | protein 90-2 | | | | protein 81-3 |
| Zm00001d009146 | 5.24 | SAMS2 | S-adenosylmethionine | AT1G02500 | 33.68 | SAM1 | S-adenosylmethionine |
| | | | synthetase 2 | | | | synthetase 1 |
| Zm00001d048157 | 328.35 | NA | 40S ribosomal | AT4G34670 | 86.07 | NA | Ribosomal |
| | | | protein S3a | | | | protein S3Ae |
| Zm00001d028622 | 266.27 | NPI242B | 60S ribosomal | AT1G61580 | 110.18 | RPL3B | R-protein L3 B |
| | | | protein L3-1 | | | | |
| Zm00001d035937 | 5.62 | NA | Chaperonin 60 | AT3G13470 | 121.33 | Cpn60beta2 | TCP-1/cpn60 |
| | | | subunit beta 2 | | | | chaperonin family |
| | | | chloroplastic | | | | protein |
| Zm00001d020852 | 19.03 | NA | Cellulose | AT1G55850 | 73.00 | CSLE1 | cellulose synthase |
| | | | synthase-like | | | | like E1 |
| | | | protein E1 | | | | |
| Zm00001d033983 | 48.10 | MAGI87281 | Aluminium | AT3G22850 | 50.43 | NA | Aluminium |
| | | | induced protein | | | | induced protein |
| | | | with YGL and | | | | with YGL and |
| | | | LRDR motifs | | | | LRDR motifs |
| Zm00001d023379 | 1.53 | PYK2 | pyruvate kinase2 | AT2G36580 | 80.87 | NA | Pyruvate kinase |
| | | | | | | | family protein |
| Zm00001d043444 | 151.60 | UMC2272 | plant-specific domain | AT2G20670 | 67.34 | NA | Protein of unknown |
| | | | TIGR01615 family | | | | function (DUF506) |
| | | | protein expressed | | | | |
| Zm00001d031833 | 7.28 | NA | Mitochondrial | AT2G22500 | 65.18 | UCP5 | uncoupling |
| | | | 2-oxoglutarate/ | | | | protein 5 |
| | | | malate carrier | | | | |
| | | | protein | | | | |
| Zm00001d047340 | 4.19 | NA | Allene oxide | AT3G25760 | 50.36 | AOC1 | allene oxide |
| | | | cyclase 3 | | | | cyclase 1 |
| | | | chloroplastic | | | | |
| Zm00001d042443 | 1.76 | NA | BTB/POZ and TAZ | AT3G48360 | 71.10 | BT2 | BTB and TAZ |
| | | | domain-containing | | | | domain protein 2 |
| | | | protein 2 | | | | |
| Zm00001d052194 | 0.39 | HSP22 | heat shock | AT5G51440 | 44.42 | NA | HSP20-like |
| | | | protein22 | | | | chaperones |
| | | | | | | | superfamily |
| | | | | | | | protein |
| Zm00001d020463 | 3.06 | NA | Sugar transport | AT4G02050 | 32.11 | STP7 | sugar transporter |
| | | | protein 7 | | | | protein 7 |
| Zm00001d001947 | 44.11 | NA | TLD-domain | AT5G39590 | 65.90 | NA | TLD-domain |
| | | | containing | | | | containing |
| | | | nucleolar protein | | | | nucleolar protein |

TABLE Y
70 Novel TFs to the NxTime response.

| Row | Maize v4 ID | Name | TF Type |
|---|---|---|---|
| 1 | Zm00001d002369 | ZmOrphan1 | NA |
| 2 | Zm00001d021927 | ZmBZR10 | BES1 |
| 3 | Zm00001d021573 | ZmSBP29 | SBP |
| 4 | Zm00001d029165 | ZmTUB11 | NA |
| 5 | Zm00001d011187 | ZmbHLH14 | bHLH |
| 6 | Zm00001d011560 | ZmHB113 | HB-other |
| 7 | Zm00001d052397 | ZmMYB97 | MYB |
| 8 | Zm00001d018941 | ZmHSF4 | HSF |
| 9 | Zm00001d017778 | ZmbHLH29 | bHLH |
| 10 | Zm00001d023282 | ZmMYB69 | MYB |
| 11 | Zm00001d046778 | ZmOrphan66 | NA |
| 12 | Zm00001d050365 | ZmZIM35 | NA |
| 13 | Zm00001d033396 | ZmGRF4 | GRF |
| 14 | Zm00001d040611 | ZmHB74 | TALE |
| 15 | Zm00001d008794 | ZmWRKY26 | WRKY |
| 16 | Zm00001d051140 | ZmNAC17 | NAC |
| 17 | Zm00001d014989 | ZmMYB161 | MYB |
| 18 | Zm00001d011578 | ZmTUB1 | NA |
| 19 | Zm00001d029934 | ZmHB78 | HD-ZIP |
| 20 | Zm00001d018320 | ZmMYBR25 | MYB_related |
| 21 | Zm00001d004744 | ZmMYB64 | MYB |
| 22 | Zm00001d044662 | ZmZHD9 | ZF-HD |
| 23 | Zm00001d035439 | ZmHB39 | TALE |
| 24 | Zm00001d049581 | ZmMYBR92 | MYB_related |
| 25 | Zm00001d048527 | ZmHB133 | HD-ZIP |
| 26 | Zm00001d020053 | ZmOrphan168 | NA |
| 27 | Zm00001d033553 | ZmC3H51 | C3H |
| 28 | Zm00001d009364 | ZmOrphan173 | NA |
| 29 | Zm00001d016106 | ZmOrphan174 | NA |
| 30 | Zm00001d048083 | ZmYAB7 | YABBY |
| 31 | Zm00001d029875 | ZmMYBR43 | MYB_related |
| 32 | Zm00001d025727 | ZmbHLH133 | bHLH |
| 33 | Zm00001d043922 | ZmARF12 | ARF |
| 34 | Zm00001d024703 | ZmC3H24 | C3H |
| 35 | Zm00001d036796 | ZmC3H26 | C3H |
| 36 | Zm00001d011187 | ZmbHLH92 | bHLH |
| 37 | Zm00001d036148 | ZmWHIRLY1 | Whirly |
| 38 | Zm00001d034515 | ZmHB65 | HB-other |
| 39 | Zm00001d033859 | ZmKN1 | TALE |
| 40 | Zm00001d048437 | ZmOrphan241 | NA |
| 41 | Zm00001d028372 | ZmbZIP115 | bZIP |
| 42 | Zm00001d027623 | ZmMYB120 | MYB |
| 43 | Zm00001d016000 | ZmMYB157 | MYB |
| 44 | Zm00001d032206 | ZmMYB125 | MYB |
| 45 | Zm00001d016000 | ZmMYBR74 | MYB_related |
| 46 | Zm00001d005726 | ZmZIM15 | NA |
| 47 | Zm00001d027757 | ZmHSF13 | HSF |
| 48 | Zm00001d008793 | ZmWRKY80 | WRKY |
| 49 | Zm00001d018056 | ZmbHLH30 | bHLH |
| 50 | Zm00001d051788 | ZmMYB142 | MYB |
| 51 | Zm00001d005692 | ZmHB34 | HD-ZIP |
| 52 | Zm00001d039006 | ZmARF24 | ARF |
| 53 | Zm00001d039014 | ZmC3H53 | NA |
| 54 | Zm00001d031109 | ZmYAB11 | YABBY |
| 55 | Zm00001d020569 | ZmMYB140 | MYB |
| 56 | Zm00001d036356 | ZmMYBR98 | MYB_related |
| 57 | Zm00001d009364 | ZmOrphan343 | NA |
| 58 | Zm00001d017112 | ZmABI35 | B3 |
| 59 | Zm00001d029270 | ZmHSF19 | HSF |
| 60 | Zm00001d020823 | ZmbZIP74 | bZIP |
| 61 | Zm00001d013625 | ZmLBD26 | LBD |
| 62 | Zm00001d047921 | ZmLBD42 | LBD |
| 63 | Zm00001d012498 | ZmOFP36 | NA |
| 64 | Zm00001d034784 | ZmMTERF6 | NA |
| 65 | Zm00001d035609 | NA | C2H2 |
| 66 | Zm00001d006344 | NA | C2H2 |
| 67 | Zm00001d049625 | NA | LSD |
| 68 | Zm00001d043699 | NA | bHLH |
| 69 | Zm00001d005323 | NA | C2H2 |
| 70 | Zm00001d027752 | NA | STAT |

What is claimed is:

1. A modified plant or plant cell, said modified plant or plant cell comprising:
a) a modification that disrupts or inhibits expression of one or more genes encoding a transcription factor (TF) that negatively regulates Nitrogen Use Efficiency (NUE) described in Table 8; or
b) a modification that overexpresses one or more genes encoding a TF that positively regulates NUE described in Table 7; or
c) a combination of modifications in a) and b).

2. The modified plant or plant cell of claim 1, wherein at least one of the genes is MYB34 or MYBR3.

3. The modified plant or plant cell of claim 2, wherein said plant or plant cell is a maize plant or maize cell.

4. The modified plant or plant cell claim 3, wherein the maize plant or maize plant cell is of an elite plant variety.

5. A modified plant of claim 1, wherein said plant is grown under low nitrogen conditions, and wherein said plant exhibits increased nitrogen use efficiency (NUE) relative to NUE exhibited by a control plant that does not comprise a modification of a gene listed in Table 1 or FIG. 29, and wherein optionally the gene that is not modified in the control plant is MYB34 or MYBR3.

6. The modified plant of claim 5, wherein the plant exhibits at least one of an increase in Nitrogen (N) uptake, increased biomass, an increased harvest index, an increased Total nitrogen utilization (NUtE), or an increased total Grain NUtE.

7. The modified plant of claim 6, wherein the increased biomass comprises increased grain mass.

8. Progeny of a plant of claim 5.

9. Seeds obtained from a plant of claim 5.

10. A method comprising modifying a plant such that expression of at least one gene of Table 1 or FIG. 29 is modified to provide a modified plant, wherein said modified plant is optionally an elite variety of maize, and/or wherein said plant exhibits increased nitrogen use efficiency (NUE) relative to NUE exhibited by a control plant that does not comprise a modification of a gene listed in Table 1 or FIG. 29, and wherein optionally the gene that is not modified in the control plant is MYB34 or MYBR3.

11. A modified maize plant, optionally grown under low nitrogen conditions, that exhibits increased nitrogen use efficiency (NUE), comprising:
(a) maize plant cells, and
(b) means for disrupting or inhibiting expression of a transcription factor (TF) that negatively regulates NUE in the maize plant cells,
wherein, the growth under low nitrogen conditions induces increased NUE in the modified maize plant relative to its unmodified parent maize plant when grown under low nitrogen conditions.

12. The modified maize plant of claim 11 in which the transcription factor TF is any of GLK44; G2; NFYA3; CA5P8; or KN1 or any combination thereof, and wherein the combination is optionally G2 and GLK44; GLK44 and NFYA3; G2 and NFYA3; CA5P8 and GLK44; GLK44 and KN1; GLK44 and WRKY107; GLK44 and GRAS37; EREB115 and GLK44; CA5P8 and G2; or bHLH47 and GLK44.

13. The modified maize plant claim 12, wherein the TF is MYBR3, MYB34 or a combination thereof.

14. The modified maize plant of claim 13, wherein the means for disrupting or inhibiting expression of TF in the modified maize plant is:
a) UniformMu transposon insertion in the 5′ untranslated region of the TF gene;
b) TF gene knockout;
c) TF frameshift mutation;
d) a microRNA that targets TF mRNA for degradation; or
e) a clustered regularly interspaced short palindromic repeats (CRISPR) gene inactivation enzyme and a guide RNA that targets the TF mRNA for degradation.

15. The modified maize plant of claim 14 using a CRISPR Cas3, Cas9 or Cas4 based system.

16. A modified maize plant, optionally grown under low nitrogen conditions, that exhibits increased nitrogen use efficiency (NUE), comprising:
(a) maize plant cells, and
(b) a means for overexpressing a transcription factor (TF) that positively regulates NUE in the maize plant cells,
wherein, the growth under low nitrogen conditions induces increased NUE in the modified maize plant relative to its unmodified parent maize plant when grown under low nitrogen conditions.

17. The modified maize plant of claim 16 in which the transcription factor TF is any TF MYB121; MYBR3; NAC61; HB6; GLK1; MYB56; WRKY36; MYB34; or ARF7; or NAC44; or ARF7; or any combination thereof, and wherein the combination is optionally any of MYB121 and MYBR3; MYB121 and NAC61; HB6 and MYB121; MYBR3 and NAC61; GLK1 and MYB121; HB6 and MYBR3; MYB121 and MYB56; MYB121 andWRKY36; MYB121 and MYB34; or GLK1 and MYBR3.

18. The modified maize plant of claim 17, wherein the means for overexpressing TF in the modified maize plant is a promoter that is a ZmUbi1 promoter, a ZmPPDK promoter, or a bnlg2057 promoter.

19. The modified maize plant of claim 18, in which expression of a gene encoding a TF that negatively regulates NUE is disrupted or inhibited, and a gene encoding a TF that positively regulates NUE is overexpressed, and wherein said gene expressions are optionally as follows: overexpression of MYB121 and disrupting or inhibiting expression of GLK44; or overexpression of MYB121 and disrupting or inhibiting expression of G2; or over expression of MYBR3 and disrupting or inhibiting expression of GLK44; or overexpression of MYBR3 and disrupting or inhibiting expression of G2; or overexpression MYB121 and disrupting or inhibiting expression of NFYA3; or over expression of NAC61 and disrupting or inhibiting expression of GLK44; or overexpression of MYB121 and disrupting or inhibiting expression of CA5P8; or overexpression of MYBR3 and disrupting inhibiting expression of NFYA3; or over expression of MYB121 and disrupting or inhibiting expression of KN1; or overexpression of and MYB121 and disrupting or inhibiting expression of WRKY10.

20. The modified maize plant of claim 19, in which the modified maize plant or plant cells are a variety of maize, or wherein optionally the maize is an elite variety of maize.

21. A method of growing a maize plant under conditions that induce increased NUE, comprising growing the modified maize plant of claim 11 under low nitrogen conditions.
